(19) United States
(12) Patent Application Publication
(10) Pub. No.: US 2014/0316269 A1
(43) Pub. Date: Oct. 23, 2014

(54) TRANSDUCERS, SYSTEMS, AND MANUFACTURING TECHNIQUES FOR FOCUSED ULTRASOUND THERAPIES

(71) Applicant: Kona Medical, Inc., Palo Alto, CA (US)

(72) Inventors: Jimin Zhang, Bellevue, WA (US); Michael Gertner, Menlo Park, CA (US)

(73) Assignee: KONA MEDICAL, INC., Palo Alto, CA (US)

(21) Appl. No.: 14/201,851

(22) Filed: Mar. 8, 2014

Related U.S. Application Data
(60) Provisional application No. 61/775,542, filed on Mar. 9, 2013.
(60) Provisional application No. 61/786,420, filed on Mar. 15, 2013.

(51) Int. Cl. A61N 7/00; A61B 8/00

(52) U.S. Cl. CPC A61N 7/00; A61B 8/4494
USPC 600/439; 602/1

(57) ABSTRACT

A system to apply ultrasound energy to a region surrounding blood flow in a blood vessel from a position outside a patient includes: a therapeutic ultrasound transducer comprising a plurality of transducer elements; and a processor configured to control the plurality of transducer elements; wherein the processor is configured to change phase inputs to the transducer elements to move a focus of the transducer at least 1 cm in a first plane which is substantially along a plane of the transducer elements of the therapeutic ultrasound transducer and at least 1 cm in a second plane orthogonal to the first plane; and wherein the processor is further configured to position the focus of the transducer in sequential positions offset from the blood flow in the blood vessel according to a pattern pre-determined by an operator of the system.

### RELATED APPLICATION DATA

This application claims priority to and the benefit of U.S. Provisional Patent Application Nos. 61/775,542 filed on Mar. 9, 2013, pending, and 61/786,420 filed on Mar. 15, 2013, pending. The entire disclosures of all of the above applications are expressly incorporated by reference herein.

### INCORPORATION BY REFERENCE

The following applications are expressly incorporated by reference in their entireties: 61/250,857, 61/256,983, 61/261,741, 61/291,359, 61/303,307, Ser. Nos. 12/685,655, 13/445,903, 13/545,944, 13/663,447, 13/657,725, 61/696,090, Ser. Nos. 12/725,450, 13/111,837, 14/099,834, 61/347,375, 61/377,908, Ser. Nos. 12/902,133, 12/966,943, 12/966,954, 12/966,962, 13/246,775, 13/417,194, 13/487,121, 13/487,118, 13/487,135, 13/535,070, 13/904,853, 13/960,743, 13/966,212, 13/048,830, 13/048,837, 13/048,842, 13/048,844, 13/091,116, 13/896,252, 13/048,842, 12/902,135, 13/019,273, 13/717,401, 13/751,133, 13/228,366, 13/246,763, 13/523,835, 14/015,331, 61/786,313.

### FIELD

The subject disclosure relates to apparatus and methods for ultrasound treatments.

### BACKGROUND

Therapeutic energy delivery from a distance involves transmission of energy waves to affect a target tissue inside a patient's body. Non-invasive or minimally invasive delivery of focused energy may allow for more efficient delivery of energy to the target tissue, improved cost efficiency of treatment, minimize trauma to the patient's body, and improve recovery time.

Delivering energy over a distance affords great advantages as far as targeting accuracy, technologic flexibility, and importantly, limited invasiveness into the patient. In a simple form, laparoscopic surgery has replaced much of the previous open surgical procedures and lead to creation of new procedures and devices as well as a more efficient procedural flow for disease treatment. Laparoscopic tools deliver the surgeon's energy to the tissues of the patient from a distance through minimally invasive pathways. Laparoscopic tools may also provide imaging of the region being treated as well as the ability for surgeons to visualize the region in real-time.

Focused energy can be used to treat a tissue inside the patient body without the therapeutic energy emitter penetrating the patient's body. However, to improve treatment process and outcome of focused energy therapy, improvements in target tissue tracking, energy transmission, energy emitter coupling, treatment planning, treatment monitoring, treatment automation, and therapeutic system reliability are desirable.

### SUMMARY

Systems and methods for delivery of focused ultrasound in medical treatments are described herein. The focused ultrasound may be high-intensity, moderate-intensity, low-intensity, or a combination thereof, depending on system configurations, clinical indications, and treatment goals. In some variations, the systems are configured to track the position of a specific target region or regions during the delivery of the ultrasound therapy. Ultrasound imaging and/or ultrasound signal beacon may be used to track the location of the target region and maintain the focus of the therapeutic ultrasound field at the desired treatment region during a treatment cycle. For example, a beacon may be positioned adjacent to or in the target region for guiding the system to focus the ultrasound on the target region, and maintain the focus on the target region throughout the treatment process. Alternatively, an imaging ultrasound unit may be implemented to locate the target region and track the position of the target region during therapy.

Variations of methods, devices, and systems for target tissue tracking, energy transmission, energy emitter coupling, treatment planning, treatment monitoring, treatment automation, and system reliability, among others, are disclosed herein.

In some embodiments, an ultrasound delivery system with an ultrasound transducer array wherein the ultrasound transducer array is positioned within the applicator and is capable of moving its orientation independent of the orientation of the applicator and an actuator for controlling a movement of the ultrasound transducer array, the actuator being coupled to the applicator; and

a detector coupled to either the applicator or the ultrasound transducer array, wherein the detector is configured to detect a treatment region in a patient's body and track a position of the treatment region while the treatment region moves within the patient's body.

The system may also contain an ultrasound transceiver module connected to the treatment transducer array and the detector and a computing unit connected to the ultrasound transceiver module and the actuator, the computing unit being configured to determine the position of the treatment region relative to the treatment transducer array and track the position of the treatment region during delivery of ultrasound energy from the ultrasound transducer array to the target region.

In some embodiments, the system detector may also contain an ultrasound imaging transducer array.

In some embodiments, the system detector further contains three or more ultrasound receivers for detecting a beacon positioned in proximity of the treatment region.

In some embodiments, the ultrasound transducer array of the system is configured to deliver focused ultrasound energy to a focal point, which is located off-axis from a perpendicular axis extending from a weighted center of the ultrasound transducer array.

In some embodiments, the ultrasound transducer array contains a number of transducer elements having semi-annular shape.

And In some embodiments, the ultrasound transducer array contains a number of ultrasound transducers in a snap fit or press fit configuration in a base substrate which forms the array and was developed by a three dimensional printing process.

In some embodiments, the system comprises a computing unit connected to the ultrasound transceiver module and the actuator, the computing unit being configured to determine the position of the treatment region relative to the treatment transducer array, and track the position the treatment region during delivery of ultrasound energy from the ultrasound transducer array to the target region.

In some embodiments, the detector comprises an ultrasound imaging transducer array.

In some embodiments, the detector comprises a plurality of three or more ultrasound receivers for detecting a beacon positioned in proximity of the treatment region.

In some embodiments, a therapeutic ultrasound system is described in which an applicator is connected to a first mover, the first mover being configured to provide at least three degrees of freedom of movements to the applicator and the applicator is produced from a housing, an ultrasound transducer array positioned within a chamber in the housing and coupled to the housing through a second mover, the ultrasound transducer array is at least partially immersed in a liquid contained in the housing of the applicator, the second mover being configured to provide at least two degrees of freedom in movements to the ultrasound transducer array; and the applicator further contains a membrane for coupling the applicator to a patient's body, the membrane being attached to the housing.

In some embodiments, the therapeutic ultrasound system contains a first orientation sensor coupled to the ultrasound transducer array.

In some embodiments, the therapeutic ultrasound system further comprises the applicator further comprises a second orientation sensor coupled to the housing of the applicator.

In some embodiments, the therapeutic ultrasound system further comprises: a water conditioner, the water conditioner being configured to circulated a cooled fluid through the chamber in the housing of the applicator.

In some embodiments, the therapeutic ultrasound system further comprises a detector coupled to the housing of the applicator, the detector is configured to detect a treatment region in the patient's body and track a movement of the treatment region during delivery of ultrasound therapy.

In some embodiments, the therapeutic ultrasound system comprises a detector with a plurality of ultrasound receivers configured for detecting a position of a beacon based on acoustic time of flight calculation.

In some embodiments, the detector comprises an ultrasound imaging array.

In some embodiments, the therapeutic ultrasound system further comprises a generator, the generator comprises a computing unit and a ultrasound transceiver configured to energize a plurality of ultrasound transducers in the treatment array with varying phases of energy to focused on a predetermined location in the patient's body.

In some embodiments, a method of delivering focused ultrasound to a plurality of treatment regions within a patient's body is described, comprising: determining a treatment plan having a predefined treatment pattern for delivering ultrasound energy within a treatment area in the patient' body, moving the ultrasound array to target a first treatment region in the treatment area, wherein the ultrasound array is configured to deliver a focused ultrasound energy; delivering the focused ultrasound energy on the first treatment region; moving the ultrasound array to target a second treatment region in the treatment area; and verifying a change in an orientation of the ultrasound array as the focus of the ultrasound array is directed form the first treatment region to the second treatment region.

In some embodiments, the method of treating a patient further comprises verifying that the change in the orientation of the ultrasound array is consistent with a relative position between the first and the second treatment regions.

In some embodiments, the method of treating a patient further comprises: calculating a required movement of the ultrasound array for moving the focus of the ultrasound array from the first treatment region to the second treatment region, and verify that the orientation change of the ultrasound array is consistent with the required movement of the ultrasound array.

In some embodiments, the method of treating a patient further comprises verifying that the changes in the orientation comprises: determining the required angular rotation of the ultrasound array to move the focus from the first treatment region to the second treatment region; and verifying that the orientation changes of the ultrasound array is consistent with the required angular rotation.

In some embodiments, a method of delivery of a focused ultrasound field, comprising generating a phase table for a plurality of transducers in a treatment transducer array, the phase table being configured for calculating the treatment transducer array focusing at a target position in a patient's body, generating a power table for various power requirements for the plurality of individual transducers, the power table being configured for calculating the electrical energy to drive the individual transducers and achieving a desired dosing at the target position, combining the phase table, power table, and time control sequence to generate required power, power distribution, and variation sequences; and delivering a focused ultrasound energy to the target position. In some embodiments, the method further comprises tracking the target position prior to the step of generating the phase table.

In some embodiments, the tracking step further comprises using a plurality of ultrasound receiver configured to detect a beacon, and applying acoustic time of flight calculation.

In some embodiments, the tracking step further comprises using an ultrasound imaging detector to track a movement of the target position.

In some embodiments, a method for delivery of a focused ultrasound to a treatment site in a patient's body follows the following steps: providing an ultrasound transceiver having an output drive circuitry configured to delivery electrical current to a plurality of output channels connected to a plurality of transducer elements in a ultrasound array; driving same voltage across all the output channels connected to the transducer elements in the ultrasound array; pulse-width modulating electrical energy driving the individual transducers in the ultrasound array, so as to achieve consistent power intensity across the ultrasound array; and delivering a focused ultrasound field to the treatment site.

In some embodiments, the plurality of transducer elements have varying surface areas and the transducer elements are arranged in a pattern to facilitate delivery of a focused ultrasound energy.

In some embodiments, the plurality of transducer elements are arranged in a random or non-uniform configuration atop a substrate which has been manufactured using a three dimensional printing process.

In some embodiments, the plurality of transducers are arranged in a concentric pattern.

In some embodiments, an algorithm is invoked within the system to make adjustments to the pulse width modulation of electrical energy to take into account the distance from each transducer element to a focal point of the ultrasound array, such that each transducer element delivers same power density relative to the focal point with the pulse width modulation.

In some embodiments, the system algorithm makes adjustments to the pulse width modulation of electrical energy to take into account absorption and interference within a transmission path from each transducer to a focal point of the ultrasound array, such that each transducer element delivers the same power density relative to the focal point.

In some embodiments, the step of making adjustments to the pulse width modulation further comprises making adjustments to the pulse width modulation of electrical energy to take into account absorption and interference within a transmission path from each transducer to a focal point of the ultrasound array, such that each transducer element delivers the same power density relative to the focal point.

In some embodiments, an ultrasound therapeutic system comprises an ultrasound transceiver with output drive circuitry driving same voltage across a plurality of output channels of the ultrasound transceiver, each of the output channels being connected to one or more individual transducer elements in a treatment transducer array and a computing unit configured to control the ultrasound transceiver to generated pulse-width modulated electrical power to drive the individual transducer elements in the treatment transducer array, to achieve consistent power intensity across the treatment transducer array.

In some embodiments, the therapeutic transducer elements in the treatment array are non-uniform, or randomly distributed, and pulse width modulation is utilized by the processor to drive more electrical power to the larger transducer element, and less electrical power to the smaller transducer element, such that uniform power intensity is achieved across the transducer array.

In some embodiments, the computing unit is configured to provide further adjustment to the pulse width modulation to take into account distances from each transducer element to a focal point of the treatment transducer array, such that each transducer element delivers same power density relative to the focal point.

In some embodiments, the computing unit is configured to provide further adjustments to the pulse width modulation to take into account absorption and interference within a path from each of the transducer elements to a focal point of the treatment transducer array, such that each transducer element delivers same power density relative to the focal point.

In some embodiments, the computing unit is configured to provide further adjustments to the pulse width modulation to take into account absorption and interference within a path from each of the transducer elements to the focal point of the treatment transducer array, such that each transducer element delivers same power density relative to the focal point.

In some embodiments, an ultrasound device comprising a housing, an interface on the housing for coupling the housing to a patient's body through a skin on the patient's body wherein the interface comprises an acoustic coupling medium and a first actuator coupled to a base of the housing, the actuator having at least one degree of freedom in movements, including the ability to advance the housing towards the patient's body to increase the pressure applied by the housing on the patient's body, a treatment transducer array and a second actuator, coupling the treatment transducer array to the housing, the second actuator having at least two degrees of freedom in movements.

In some embodiments, the device further comprises an image transducer array and a third actuator coupling the image transducer array to the housing.

In some embodiments, the surface of the treatment transducer array comprises a curved profile.

In some embodiments, the treatment transducer array comprises a curved base member having a plurality of cavities housing individual transducers.

In another embodiment, a therapeutic ultrasound system, comprises an ultrasound transducer unit, wherein the ultrasound transducer unit comprise a treatment transducer array, and three or more ultrasound receivers for receiving signals for a beacon positioned in a patient's body, an actuator coupled to a base of the ultrasound transducer unit, the actuator being configured to move an orientation of the ultrasound transducer unit with at least two degrees of freedom and an ultrasound transceiver module connected to the ultrasound transducer unit, and a computing unit connected to both the ultrasound transducer unit and the actuator, the computing unit being configured to generate a coordinate system and track the position of the beacon in such coordinate system based on signals emitted by the beacon and received by the ultrasound receivers, and an acoustic time of flight calculation.

In some embodiments, the transducer unit comprises five or more ultrasound receivers, and only a sub-set of said five more ultrasound receivers are activated for tracking the beacon, the computing system is configured to detect whether one or more of the active ultrasound receivers have failed, and activates additional ultrasound receivers if failure of one or more of the activated receivers is detected.

In some embodiments, an ultrasound system comprising a treatment transducer array unit, the treatment transducer array unit comprise a plurality of lobes of treatment transducer arrays, wherein at least one of said lobes of treatment transducer array being configured with individual transducers elements arranged in a diced configuration, while at least one other lobe is configured with partial annular rings of transducer elements.

In some embodiments, the ultrasound system comprises lobes with transducer elements in a diced configuration and the diced configuration is in a pie shaped configuration, wherein within a given column, the transducer closer to a narrow segment of the pie shape has larger surface area, while the transducer further away from the narrow segment of the pie shape has smaller surface area.

In some embodiments, a method for delivering ultrasound therapy comprises tracking a position of a treatment region in the patient's body, determining the position of the treatment region relative to a treatment transducer array, generating a treatment plan base at least on the position of the treatment region relative to a treatment transducer array, calculating an expected electrical current consumption by the treatment transducer array base on the treatment plan; delivering focused ultrasound energy from the treatment transducer array to the treatment region as prescribed by the treatment plan, monitoring an actual electrical current being consumed by the treatment transducer array, and determining whether the actual electrical current being consumed is higher than the expected electrical current consumption.

In some embodiments, an electrical current threshold is utilized, beyond which the expected electrical current consumption results in therapy termination.

In some embodiments, a method for therapeutic ultrasound delivery comprises positioning an applicator on an external surface of a patient's body, wherein the applicator comprises a housing, and a treatment transducer array capable of moving relative to the housing, wherein the movement of the treatment transducer array is controlled by a computing unit, and the treatment transducer array comprises a plurality of transducer elements, locating a treatment region within the patient's body, tracking a position of the treatment region as the treatment region moves in the patient's body, calculating a distance between each of a plurality of transducer elements and the target region, and generating a phase aberration correction factor based on at least the distance between each of a plurality of transducer elements and the target region and then generating a therapy plan and generating a therapy based on the plan.

In some embodiments, a method for delivery of an ultrasound therapy to a target area in a patient's body comprises selecting a dose for the therapy, determining an average depth of the target area relative to the treatment transducer array over a period of time while tracking a position of the target area, and calculating a power for the ultrasound energy for the therapy from the average depth and the selected dose; and, delivering ultrasound energy to the target area based on the calculated power.

In some embodiments, an ultrasound system comprises a housing which contains an enclosed wall, a base coupled to a bottom edge of the wall, and an membrane coupled to the upper edge of the wall, forming a chamber therein.

In some embodiments, an ultrasound transducer array positioned in the chamber, the ultrasound transducer array is coupled to the base of the housing through an actuator.

In some embodiments, an ultrasound transceiver module for energizing the ultrasound transducer array is further provided.

In some embodiments, the system further contains a computing unit connected to both the actuator and the ultrasound transceiver.

In some embodiments, the system contains a water conditioner connected to the housing for circulating a fluid through the chamber of the housing.

In some embodiments, the system further comprises a housing which contains a channel in the wall of the housing wherein the channel has an outer port on the outer wall at a proximal portion of the housing, for receiving fluid from the water conditioner, the channel travels under at least a portion of the upper edge of the wall, and exit into the chamber of the housing through an inner port located on the inner wall at a distal portion of the housing.

In some embodiments, the inner port of the housing is positioned to direct the coupling fluid to flow over an emitting face of the ultrasound transducer array.

In some embodiments, a water conditioner cools the coupling fluid prior to returning the coupling fluid back into the chamber of the housing.

In some embodiments, the wall of the housing is removeably coupled to the base of the housing.

In some embodiments, the wall of the housing comprises a polymeric material that is semi-transparent or transparent.

In some embodiments, an ultrasound device comprises a housing having a chamber and an opening, a membrane covering said opening for coupling the housing to a patient's body, an ultrasound transducer array positioned within said chamber for delivering ultrasound energy through the membrane into the patient's body, a coupling fluid filling said chamber, wherein the ultrasound transducer is at least partially immersed in said fluid and an image detector coupled to the housing for detecting a bubble on the membrane.

In some embodiments, the image detector is attached to a wall of said housing and positioned to capture images of the membrane through said coupling fluid.

In some embodiments, the image detector is configured to detect a spectrum of light that comprises infrared.

In some embodiments, the image detector detects reflections from the interface of the membrane and the skin of the patient to determine the depth to the skin from the therapeutic ultrasound transducer.

In some embodiments, a method for delivering ultrasound into a patient's body involves coupling an ultrasound transducer array to a patient's body through an acoustic coupling interface and detecting bubble located between the acoustic coupling interface and the patient's body with an image detector.

In some embodiments. a method for delivering ultrasound into a patient's body, comprising coupling an ultrasound transducer array to a patient's body with an acoustic coupling interface, moving a focus of an image detector across at least a portion of the acoustic coupling interface and a boundary between the acoustic coupling interface and the patient's body; and, detecting a bubble.

In some embodiments, a method for placing an ultrasound treatment module on a patient's body for delivery of a focused ultrasound energy to a treatment region in the patient's body comprises locating a plurality of ultrasound obstructive tissues in a patient's body, identifying a boundary defined by said plurality of ultrasound obstructive tissues, and determining a treatment window based on the boundary in combination with a profile of a treatment applicator, wherein the treatment applicator houses an ultrasound transducer array for generating the focused ultrasound energy.

In some embodiments, the method for placing the ultrasound treatment module includes narrowing a treatment window by taking into consideration an outer limit of the focused therapeutic ultrasound when focused ultrasound field is emitted out of an outer surface of the applicator.

In some embodiments, the step of determining the treatment window comprises applying a template on a patient's body, and marking the treatment window on the patient's body.

In some embodiments, a method for delivering focused ultrasound therapy, comprises identifying a treatment region in a patient's body to be targeted for delivery of a focused ultrasound energy, tracking a position of the treatment region while the treatment region moves inside the patient's body, moving a treatment transducer array to maintain a focal point of the treatment transducer array on the target region, monitoring a targeting quality based on the tracking; and delivery the focused ultrasound energy from the treatment transducer array to the treatment region.

In some embodiments, the method for delivering ultrasound to the patient further comprises terminating the delivery of focus ultrasound energy if the targeting quality indicates inadequate targeting stability.

In some embodiments, a patient platform is described comprising a radiolucent frame configured for positioning on a catheterization table, wherein the frame includes a torso segment for supporting a torso of a patient, and an extension segment for supporting the legs of the patient, wherein the frame elevates the patient above the catheterization table by no more than 12 inches, and weighs less than 85 pounds and the torso segment includes an aperture for receiving a therapy delivery device, the aperture having a variable opening that is controllable by the operator, the aperture is also being accessible from both sides of the catheterization table, and patient platform further includes an opening on top, such that when the therapy delivery device is placed in the aperture, the therapy delivery device can be coupled to the patient's body and ultrasound delivered therethrough.

A system to apply ultrasound energy to a region surrounding blood flow in a blood vessel from a position outside a patient includes: a therapeutic ultrasound transducer comprising a plurality of transducer elements; and a processor configured to control the plurality of transducer elements; wherein the processor is further configured to receive a first input regarding a first position of the blood vessel; wherein the processor is configured to change phase inputs to the transducer elements to move a focus of the transducer at least 1 cm in a first plane which is substantially along a plane of the transducer elements of the therapeutic ultrasound transducer and at least 1 cm in a second plane orthogonal to the first plane; and wherein the processor is further configured to position the focus of the transducer in sequential positions offset from the blood flow in the blood vessel according to a pattern pre-determined by an operator of the system.

Optionally, the plurality of transducer elements are arranged in a substantially random or non-uniform pattern.

Optionally, the therapeutic ultrasound transducer comprises a substrate with cutouts respectively for the transducer elements; and wherein the transducer elements can be placed into the respective cutouts of the substrate and be coupled to the therapeutic ultrasound transducer via snap or press fit.

Optionally, the transducer elements of the therapeutic ultrasound transducer are arranged on a 3D substrate pointing to a defined point, line, area, or 3D region, in front of the therapeutic ultrasound transducer.

Optionally, at least one of the transducer elements of the therapeutic ultrasound transducer has a circle, square, hexagon, or rectangular shape.

Optionally, the transducer elements of the therapeutic ultrasound transducer are packaged with one size, two sizes, or multiple sizes of single element transducers.

Optionally, the transducer elements of the therapeutic ultrasound transducer are packaged with multiple sizes of single element transducers with multiple operational frequencies.

Optionally, the substrate of the transducer is formed from three-dimensional printing process with linear, planar, ellipsoid, spherical or other 3D geometry.

Optionally, the three dimensional printing process comprises one of: selective laser melting, direct metal laser sintering, selective laser sintering, fused deposition modeling, a polymer curing type process, and a stereolithography process.

Optionally, the processor is configured to receive a second input regarding a second position of the blood vessel within about 1-200 ms of the first position; and wherein the processor is configured to adjust the focus of the therapeutic ultrasound transducer according to the second position.

Optionally, the system further includes an imaging probe to image a region including the blood vessel.

Optionally, the imaging probe comprises attached fiducials or 3D position sensors.

Optionally, the processor is configured to receive input associated with reflections or position signals from the fiducials or the position sensors, interpret the input, and produce an output for indicating imaging probe position and imaging probe orientation in a three-dimensional coordinate space.

Optionally, the processor is configured to determine a three dimensional position of the blood vessel based on the imaging probe position, the imaging probe orientation, and a position of the blood vessel.

Optionally, the processor is configured to receive input associated with reflections or position signals from the fiducials or the position sensors, and produce an output for positioning of the therapeutic ultrasound transducer.

Optionally, the processor is configured to associate a three dimensional position of a target on an image from an imaging probe with a three dimensional position of the therapeutic ultrasound transducer.

Optionally, the system further includes a graphical user interface coupled to the processor, wherein the graphical user interface is configured to display a target, and wherein the target as displayed is moveable by an operator of the system.

Optionally, the therapeutic ultrasound transducer comprises a series of ridges in which the transducer elements are press or snap fit with tolerance greater than 100 microns.

Optionally, the therapeutic ultrasound transducer comprises a series of ridges in which the transducer elements are press fit or snap fit with a tolerance greater than 50 microns.

Optionally, the system is configured to be calibrated automatically with a receiver placed at a distance from the therapeutic ultrasound transducer via a calibration procedure that determines an efficiency of the transducer elements.

Optionally, the system is configured to be calibrated automatically with a transmitter placed at a distance from the therapeutic ultrasound transducer via a calibration procedure that determines an efficiency of the transducer elements.

Optionally, the processor is also configured to track a region of interest in an ultrasound image.

Optionally, the region of interest is linked to a user defined target on the ultrasound image.

Optionally, the processor comprises an algorithm to compare a region of interest in an ultrasound frame to a region of interest in a previous ultrasound frame, and update a position of a target with respect to the therapeutic ultrasound transducer.

Optionally, the algorithm utilizes digitization of speckles for the comparison.

Optionally, the algorithm utilizes digitization of anatomic structures for the comparison.

Optionally, the algorithm utilizes both digitization of speckles and digitization of anatomic structures for the comparison.

Optionally, the processor is configured to utilize modulation of an output pulse to the therapeutic ultrasound transducer to account for a regional difference in power density along the transducer elements of the therapeutic ultrasound transducer.

Optionally, the processor is configured to utilize phase modulation of an output pulse to the with the therapeutic ultrasound transducer to account for phase aberration from inhomogeneous tissue structure.

Optionally, the processor is configured to utilize pulse width modulation of an output pulse to the therapeutic ultrasound transducer to account for performance due to transducer element size or a variation from manufacture processing of the transducer elements of the transducer.

Optionally, the system further includes an electromechanical mover coupled to the processor, wherein the electromechanical mover is configured to mechanically position the therapeutic ultrasound transducer to move the focus in response to control signals from the processor.

Optionally, the therapeutic ultrasound transducer is configured to provide high-intensity energy, moderate-intensity energy, low-intensity energy, or a combination thereof.

Optionally, the processor is configured to track a position of a specific target region or regions during a delivery of the ultrasound energy by the therapeutic ultrasound transducer.

Optionally, the processor is further configured to use ultrasound imaging and/or ultrasound signal beacon to track a location of a target region and to maintain the focus of the transducer at the target region during a treatment cycle.

Optionally, the ultrasound transducer is within an applicator and is moveable independent of an orientation of the applicator; and wherein the system further comprises an actuator for controlling a movement of the ultrasound transducer, and a detector coupled to either the applicator or the ultrasound transducer, wherein the detector is configured to detect a treatment region in the patient and track a position of the treatment region while the treatment region moves within the patient.

Optionally, the detector comprises an ultrasound imaging transducer array.

Optionally, the detector comprises three or more ultrasound receivers for detecting a beacon positioned in proximity of the treatment region.

Optionally, the system further includes an ultrasound transceiver module connected to the transducer; wherein the detector is connected to the ultrasound transceiver module and the actuator; and wherein the processor is configured to determine the position of the treatment region relative to the transducer.

Optionally, at least one of the transducer elements has a semi-annular shape.

Optionally, the system further includes a first mover to which the therapeutic ultrasound transducer is connected, the first mover being configured to provide at least three degrees of freedom of movements for the therapeutic ultrasound transducer.

Optionally, the therapeutic ultrasound transducer is located within a housing of an applicator, and is coupled to a second mover, the ultrasound transducer array being at least partially immersed in a liquid contained in the housing of the applicator, and wherein the second mover is configured to provide at least two degrees of freedom in movements for the ultrasound transducer array.

Optionally, the therapeutic ultrasound transducer is a part of an applicator, and the applicator comprises a membrane for coupling the applicator to a patient's body.

Optionally, the system further includes a first orientation sensor coupled to the therapeutic ultrasound transducer.

Optionally, the therapeutic ultrasound transducer is a part of an applicator, and wherein the system further comprises a second orientation sensor coupled to a housing of the applicator.

Optionally, the system further includes a water conditioner to circulate a cooled fluid through a chamber in the housing of the applicator.

Optionally, the system further includes a detector with a plurality of ultrasound receivers configured for detecting a position of a beacon based on acoustic time of flight calculation, the detector being coupled to the processor.

Optionally, the system further includes a generator and an ultrasound transceiver configured to energize at least some of transducer elements in the transducer with varying phases of energy to focus the energy on a predetermined location in the patient.

Optionally, the processor is configured to access a treatment plan that prescribes delivery of energy to a plurality of treatment regions within a patient's body in accordance with the pattern.

Optionally, the processor is also configured to calculate a required movement of the ultrasound transducer for moving the focus of the ultrasound transducer from a first treatment region to a second treatment region.

Optionally, the processor is further configured to determine a required angular rotation of the ultrasound transducer to move the focus from a first treatment region to a second treatment region.

Optionally, the processor is configured to generate a phase table for at least some of the transducer elements, the phase table having values for operating the ultrasound transducer so that energy provided by the ultrasound transducer focuses at a target position in a patient's body.

Optionally, the processor is further configured to determine a power table for various power requirements for at least some of the transducer elements, the power table having values for calculating electrical energy to drive the at least some of the transducer elements so that a desired dosing at a target position can be achieved.

Optionally, the system further includes circuitry for pulse-width modulating electrical energy to drive at least some of the transducer elements in the transducer, to achieve a consistent power intensity across the transducer.

Optionally, at least two of the transducer elements have different respective surface areas.

Optionally, the transducer elements are arranged in a random configuration atop a substrate, the substrate being manufactured using a three dimensional printing process.

Optionally, the transducer elements are arranged in a concentric pattern.

Optionally, the processor is configured to use an algorithm to make adjustments to a pulse width modulation of electrical energy such that two or more of the transducer elements deliver same power density relative to a focal point of the transducer with the pulse width modulation, wherein the algorithm takes into account a distance from two or more of the transducer elements to the focal point of the transducer.

Optionally, the processor is configured to use an algorithm to make adjustments using pulse width modulation of electrical energy such that two or more of the transducer elements achieve a same power density relative to a focal point of the transducer, wherein the algorithm takes into account absorption and interference within a transmission path from each of the two or more transducers to the focal point of the transducer.

Optionally, at least two of the transducer elements have different respective sizes, the at least two of the transducer elements having a larger transducer element and a smaller transducer element, and wherein the processor is configured to use pulse width modulation to drive more electrical power to the larger transducer element, and less electrical power to the smaller transducer element.

Optionally, the processor is configured to track a position of a beacon in a coordinate system based on signals emitted by the beacon and received by ultrasound receivers coupled to the transducer, and based on an acoustic time of flight calculation.

Optionally, the transducer comprises five or more ultrasound receivers, and a sub-set of the five or more ultrasound receivers are activated for tracking a beacon.

Optionally, the processor is configured to use an algorithm to detect whether one or more activated ultrasound receivers have failed, and activate additional ultrasound receiver(s) if failure of the one or more activated receivers is detected.

Optionally, the transducer comprises lobes with the transducer elements being arranged in a diced configuration, the diced configuration forming a pie shape, wherein one of the transducer elements closer to a narrow segment of the pie shape has a larger surface area, while another one of the transducers further away from the narrow segment of the pie shape has a smaller surface area.

Optionally, the processor is further configured for monitoring an actual electrical current being consumed by the transducer, and determining whether the actual electrical current being consumed is higher than an expected electrical current consumption.

Optionally, the processor is also configured for locating a treatment region within the patient, tracking a position of the treatment region as the treatment region moves in the patient, calculating a distance between at least one of transducer elements and the target region, and generating a phase aberration correction factor based at least on the distance between the at least one of transducer elements and the target region.

Optionally, the transducer further comprising a housing which contains a channel in a wall of the housing to direct coupling fluid to flow over a surface of the transducer.

Optionally, the system further includes an image detector attached to the housing and positioned to capture images of a membrane through the coupling fluid.

Optionally, the image detector is configured to detect a spectrum of light that comprises infrared light.

Optionally, the image detector is configured to detect reflections from an interface between the membrane and a skin of the patient to determine a distance to the skin from the therapeutic ultrasound transducer.

Optionally, the system further includes an image detector for detecting bubbles located between an acoustic coupling interface of the transducer and a body of the patient.

Optionally, the system further includes a radiolucent frame having a torso segment for supporting a torso of the patient, and an extension segment for supporting legs of the patient.

Optionally, the algorithm utilizes digitalization of a flow parameter in the ultrasound image for the comparison.

Optionally, the algorithm uses a combination of digitalization of speckles, a flow parameter, and anatomic structural information for the comparison.

Optionally, the region of interest is a user-defined region of interest.

Optionally, the processor is configured to determine whether the region of interest is adequate for use in tracking based on an indicator signal.

Optionally, the sequential positions offset from the blood flow are within 5 mm of one another.

Optionally, the sequential positions offset from the blood flow are within 1 mm of one another.

Optionally, the sequential positions offset from the blood flow is substantially a same position.

Optionally, the system further includes a table for the patient, wherein the table comprises an opening which can be modified in size by an operator.

A system to apply ultrasound energy to a nerve region surrounding blood flow in a blood vessel from a position outside a patient includes: a therapeutic ultrasound transducer comprising a plurality of transducer elements; an ultrasound imaging transducer with attached fiducials configured to indicate an orientation of the imaging transducer; and a processor configured to control the plurality of transducer elements; wherein the processor is further configured to receive data at a first time point from the fiducials to determine a three-dimensional coordinate of a target in an ultrasound image from the ultrasound imaging transducer.

Optionally, the processor is further configured to position a focus of the transducer elements at sequential positions offset from the blood flow in the blood vessel according to a pattern pre-determined by an operator of the system.

Optionally, the processor is configured to receive additional data from the fiducials at successive time points.

Optionally, the processor is configured to utilize the additional data to determine an updated coordinate of the target.

Optionally, the target comprises a user-defined region of interest.

Optionally, the target comprises speckles, anatomic features, or flow signals.

Other aspects and features will be evident from reading the following detailed description.

### DESCRIPTION OF THE FIGURES

FIG. 1 depicts one variation of a therapeutic ultrasound system. This example is configured to deliver focused ultrasound energy into the torso region of a patient's body.

FIG. 2 depicts another variation of a therapeutic ultrasound system configuration, which includes a fluoroscopy unit.

FIG. 3 depicts a generator.

FIG. 4 depicts a water conditioner.

FIG. 5 depicts a treatment transducer array configured with pitch and roll motion capability, and having an axel movement to displace treatment array towards or away from the patient's body. The treatment array is sown with a coupling interface for engaging the patient's body.

FIG. 6 illustrates another variation where the treatment transducer array is couple to a mover with two separate moveable joints.

FIG. 7 illustrates another variation where treatment transducer array, which produces the focused therapeutic ultrasound, is housed within an applicator of a treatment module.

FIG. 8 depicts a treatment module, which includes an ultrasound treatment transducer array located within the applicator of the treatment module.

FIG. 9 depicts a patient placed in the supine position, with his posterior flank located over the treatment aperture of a patient platform.

FIG. 10 depicts a treatment module positioned such that the applicator of the treatment module engages the skin on the posterior flank of the patient.

FIG. 11 is a graphical representation of one variation of an ultrasound therapeutic system, which is deployed next to a conventional C-Arm catheter bed in a catheterization lab.

FIG. 12 illustrates one variation of controlled movements for a treatment transducer array.

FIG. 13 depicts a high level functional diagram for one configuration of an ultrasound therapeutic system.

FIG. 14 depicts a moveable frame for a generator.

FIG. 15 depicts the lower level components in a generator.

FIG. 16 depicts the upper level components in a generator.

FIG. 17 depicts a functional block diagram of an ultrasound therapeutic system.

FIG. 18 depicts an example of an ATOF amplification chain diagram.

FIG. 19 depicts an example of a catheter extension box block diagram.

FIG. 20 illustrates one example of power transmission of a therapeutic system from the AC input to the acoustic output of the treatment transducer array.

FIG. 21 illustrates one example of a treatment process.

FIG. 22 is a graphical representation illustrating a treatment plan with multiple treatment regions forming a ring pattern. The treatment array is shown focusing on a first treatment region.

FIG. 23 illustrate the treatment array moving to a new position, and directing the focus of the treatment array to a second treatment region relative to the first treatment region shown in FIG. 22.

FIG. 24 depicts an example of a treatment module with treatment transducer array, which is positioned within an applicator of the treatment module. The treatment transducer array is independently moveable relative to the applicator.

FIG. 25 illustrates one example of functional steps for an ultrasound therapeutic system.

FIG. 26 illustrates another example of functional steps for an ultrasound therapeutic system.

FIG. 27 is a graphical representation illustrating changes in target depth as the treatment array is tilted about a pivot point.

FIG. 28 illustrates one variation of an interconnection relationship of phase and power controls of a generator subsystems.

FIG. 29 depicts one variation of the geometric structure of a treatment transducer array. In this variation, the transducer's three panels form a fan shaped treatment array.

FIG. 30 is a graphical representation of the treatment transducer array.

FIG. 31 is a graphical representation of a phased-array treatment transducer array positioned over a treatment area, with the focal zone of the treatment array directed towards the treatment area. Multiple target regions within a treatment area are shown.

FIG. 32 is a flow chart illustrating one example of a process in configuring the system for delivering a focused ultrasound energy field to a treatment region.

FIG. 33 illustrates a proposed sample treatment pattern of focal position distributions.

FIG. 34 depicts a pulse width modulated waveform from a transmitting circuit of an ultrasound transceiver.

FIG. 35 illustrates an example of a harmonics energy distribution of a pulse modulation waveform.

FIG. 36 is a top view of a base frame of the transducer unit for housing the ultrasound transducer array.

FIG. 37 is the bottom view of the base frame of FIG. 36, shown with connectors and electronic components.

FIG. 38 is an exploded view of a transducer unit, showing various components that are connected to the base frame of the transducer unit.

FIG. 39 is a top-down view of the face of a transducer array unit.

FIG. 40 is the expanded view of the narrow tip portion of the transducer array unit of FIG. 39, showing the concentric and annular pattern of the transducer array elements.

FIG. 41 depicts one variation of ATOF receiver sensor positions.

FIG. 42 is a graphical illustration showing activation of eight ATOF receiver sensors.

FIG. 43 illustrates one example of geometrical dimensions and coordinate for a treatment array. Also shown, are definition of the spatial location and extent of the ATOF volume. Dimensions are in millimeters.

FIG. 44 depicts a block diagram illustrating one variation of an ATOF subsystem.

FIGS. 45-50 illustrate various fan shaped configurations of transducer array designs.

FIG. 51 depicts another fan shaped transducer array design, with Zone 1 having a diced transducer array pattern, while Zone 2 and 3 having ring or annular patterns.

FIG. 52 illustrates one configuration of the diced pattern for Zone 1 of the array shown in FIG. 51.

FIG. 53 depicts an elemental design for an individual transducer in a transducer array.

FIG. 54 depicts a fan shaped transducer array design with a concaved surface profile.

FIG. 55 depicts a cross-sectional view of the array shown in FIG. 54.

FIG. 56 is a graphical representation illustrating the delivery of focused ultrasound energy from an ultrasound array located over a treatment window, and propagating the ultrasound energy to a focal point located underneath the ribcage.

FIG. 57 shows a side view of the same arrangement displayed in FIG. 56.

FIG. 58 depicts a fans shaped array design with transducer elements in annular ring configuration.

FIG. 59 depicts a fan shaped array design with transducer elements in diced matrices arrangement.

FIG. 60 depicts a transducer array having a concaved profile integrated in a treatment module. A separate imaging transducer array is incorporated in module to provide target imaging and tracking.

FIG. 61A illustrates a concaved array design consisting of a concaved base support having honeycomb structure or a plurality of cavities on the surface, for housing individual transducer elements.

FIG. 61B illustrates a bowl printed with three dimensional printing technique with cutouts for individual piezoelectric transducers.

FIG. 61C illustrates the positioning of the individual transducers placed in a three dimensional printing based substrate.

FIG. 61D illustrates a detailed sketch of the individual transducer elements which fit inside the printed substrate.

FIG. 61 E-I illustrate simulations of a therapy array manufactured with three dimensional printing technique and randomly distributed ultrasound transducers.

FIG. 61J-K illustrates a flat embodiment of a substrate with a random arrangement of openings for individual ultrasound transducers and predicted focal spot.

FIG. 61L illustrates a configuration of the system where an imaging probe and a therapeutic transducer are linked via fiducials on the imaging probe.

FIG. 61 M-N illustrates a curved three dimensionally printed substrate for therapeutic ultrasound inserts including depiction of potential position of focal points.

FIG. 61 O-P illustrates additional embodiments of a three-dimensional substrate for a therapeutic array including one specific for applying therapy to the renal nerves.

FIG. 61Q illustrates a process flow chart to produce a three dimensional printed therapeutic ultrasound array including calibration.

FIG. 62 illustrates a concentric ring design where the center of the annular array is located at the ‘weighted’ center of the fan or pie-segment shaped aperture.

FIGS. 63-66 depict a concaved array design comprising of six lobes that are configured to deliver ultrasound to a focal point

FIGS. 67-68 illustrate another example of a fan shaped design comprising of six individual concaved plates, each with an array of ultrasound transducers.

FIGS. 69-70 illustrates a transducer array design using a combination of fan shaped profiles and circular profiles.

FIGS. 71-72 depict a transducer array design where a plurality of transducer array surfaces are in a staggered or out-of-plane configuration, that forms a larger coherent focus ultrasound delivery array structure.

FIGS. 73-75 illustrate transducer array designs, where a portion the surface area for each of the therapeutic arrays is allocated for placement of imaging array elements.

FIG. 76 depicts one example of a targeting catheter, shown with the distal portion of the catheter inserted inside a protective sheath.

FIG. 77 illustrates the inner construction of the distal portion of one variation of a targeting catheter.

FIG. 78 show an expanded view around the PZT transducer in the targeting catheter of FIG. 77.

FIG. 79 is a cross-sectional view at position A (the mid-point of the PZT transducer) of the FIG. 77 catheter

FIG. 80 is a cross-sectional view at position B (proximal to the PZT transducer) of the FIG. 77 catheter.

FIG. 81 is a perspective view of the PZT transducer from FIG. 77.

FIG. 82 is a side view of the PZT transducer of FIG. 81.

FIG. 83 is a frontal end view down the lumen of the PZT transducer of FIG. 81.

FIG. 84 is a perspective view of the transducer liner from the catheter of FIG. 77.

FIG. 85 is a top view of the transducer liner of FIG. 84.

FIG. 86 is a side view of the transducer liner of FIG. 84.

FIG. 87 is end view down the lumen of the transducer liner of FIG. 84.

FIG. 88 is an expanded view of the distal portion of the targeting catheter of FIG. 77.

FIG. 89 depicts another example of a targeting catheter.

FIG. 90 depicts the distal portion of the targeting catheter of FIG. 89.

FIG. 91 shows an expanded view of Section A, the distal portion, of the targeting catheter of FIG. 89.

FIG. 92 is the cross-sectional view of Section B, the over the guide-wire side entry point, of the targeting catheter of FIG. 89.

FIG. 93 illustrates the lumens and wire connections of the targeting catheter of FIG. 89.

FIG. 94 depicts a partially constructed distal portion of the targeting catheter of FIG. 4N, where adhesives have been placed proximal and distal to the PZT transducer.

FIG. 95 depicts the partially constructed targeting catheter of FIG. 94 having a shrink-wrap catheter position over the distal portion, covering the PZT transducer and connection points.

FIG. 96 illustrates a targeting catheter being positioned within a left renal artery.

FIG. 97 depicts an example of a targeting catheter that comprises two beacons.

FIG. 98 illustrates one example of a treatment transducer array platform, which comprises three ATOF receivers for detecting the position of a beacon located in a patient's body.

FIG. 99 illustrates another example where the ATOF receivers are positioned on the applicator, which houses a treatment transducer array. The treatment transducer array is capable of movements that are independent of the applicator.

FIG. 100 depicts a primary monitor, positioned on top of the generator, displaying the full position applicator screen.

FIG. 101 depicts a remote targeting monitor, which may be positioned in a patient platform, displaying only those elements of the position applicator screen that pertain to targeting.

FIG. 102 depicts the generator monitor displaying the CALCULATE THERAPY PLAN screen.

FIG. 103 depicts the generator monitor displaying the PERFORMING LESION 1 OF 18 screen.

FIG. 104 illustrates generator monitor indicating the treatment progression. In particular, Performing Lesion 3 of 18 Screen is shown.

FIG. 105 depicts the therapy completed display in the generator monitor after the completion of all eighteen lesions.

FIG. 106 illustrates an example of a system configuration for a therapeutic system utilizing ultrasound imaging to track target tissue.

FIG. 107 is a graphical illustration on the use of a one dimensional ultrasound array structure (i.e., a 2D imaging array) to scan and track the position of a target tissue.

FIG. 108 is a flow chart illustrating one example of a treatment process for tracking and treating a tissue with therapeutic ultrasound.

FIG. 109 illustrates an example of a system configuration, where a single array is utilized for both imaging and treatment purposes.

FIG. 110 illustrates an example of a system configuration, where the imaging transducer is independently positioned (i.e., physically decoupled from the treatment transducer), and sensors are used to register the coordinate systems between the image transducer array and the treatment transducer array.

FIG. 111 illustrates an example of a system configuration, where the imaging array is fixedly coupled to the treatment array.

FIGS. 112-115 illustrate various examples for the placement for the image transducer array (for tracking a target) relative to the treatment transducer array and the applicator, which houses the treatment transducer array.

FIG. 116 illustrates an example of the steps for performing a focus ultrasound therapy. (RDC: Renal Double Curve; RA: Renal Artery; BMW: Balanced Middle Weight; GW: Guide Wire; TC: Targeting Catheter; Tx: Treatment; TM: Treatment Module; PP: Patient Platform.)

FIG. 117 illustrates major software components for one variation of a software configuration. Serial Peripheral Interface (SPI) refers to the hardware interface used to communication to some of the system hardware.

FIG. 118 illustrates an example of a concurrency model.

FIG. 119 illustrates an example of a top level state diagram for a therapeutic system.

FIG. 120 illustrates a patient session state diagram.

FIG. 121 illustrates a flowchart one example of a treatment planning process prior to delivery of the therapeutic ultrasound energy.

FIG. 122 illustrates a flowchart of one example of a tracking loop for tracking of the target tissue during a treatment process.

FIGS. 123A-123B depict two tables that list examples of various conditions, and their corresponding actions and notification text.

FIG. 124 illustrates one example of a software flowchart for conditioning handling.

FIG. 125 illustrates an example of a system context diagram for an error handler.

FIG. 126 illustrates one variation of an error resolution state sequence.

FIG. 127 depicts one example of a treatment module.

FIGS. 128-129 depict (a) a top view, and (b) a side view, of the base of the treatment module of FIG. 127.

FIG. 130 is a perspective view of the treatment module shown in FIG. 127. The slots (for directing suction and airflow) located on the underside of the base of the treatment modules are shown.

FIG. 131 depicts an exploded view of the treatment module of FIG. 127, without the base of FIG. 129.

FIG. 132 shows the ball joint of the treatment module.

FIG. 133 shows the piston inserted within the ball joint of FIG. 132.

FIG. 134 is a cross-sectional view of the ball join of FIG. 132.

FIG. 135 is an exploded view of the lower portion of the applicator, which includes an actuation unit for moving the treatment transducer unit (housed on the upper portion of the applicator). The treatment transducer unit is not shown in this figure.

FIG. 136 illustrates selective components on the upper surface of the applicator base plate, for coupling the treatment transducer unit to the applicator base.

FIG. 137 depicts the top view of the treatment transducer unit.

FIG. 138 depicts the bottom view of the treatment transducer unit of FIG. 137.

FIG. 139 depicts a treatment module with the treatment transducer unit tilted up.

FIG. 140 depicts the treatment module of FIG. 139 with the treatment transducer unit rolled to one side.

FIG. 141 depicts push rods extending through the base plate of the applicator to engage the bottom surface of the treatment transducer unit.

FIG. 142 shows detailed constructions of two stepper motors for driving corresponding push rods for moving/displacing the treatment transducer unit. The treatment transducer unit is not shown in this figure.

FIG. 143 illustrates an alternative variation where the push rods are directly integrated within the driving motors.

FIG. 144 depicts another design variation for coupling the treatment transducer unit to the base of the applicator. In this design, a joy stick type configuration is implemented.

FIG. 145 is a block diagram illustrating one variation of an arrangement for interactivities between the modules of a treatment transducer unit.

FIG. 146 is a block diagram illustrating a treatment array positioner (a.k.a., transducer positioner) subsystem's basic input output relationship for driving the movements of the treatment transducer array (i.e., therapeutic transducer array).

FIG. 147 is a graphical representation modeling an example of a focused ultrasound field being projected from the treatment transducer array on the treatment transducer unit.

FIG. 148 is a graphical representation modeling the treatment transducer unit of FIG. 9S being positioned an in the applicator. The figure demonstrates the ultrasound field coverage, and the displacements of the focal point, as the orientation of the treatment transducer array is rotated within the applicator.

FIG. 149 depicts one variation of a nosecone for an applicator.

FIG. 150 is a cross-sectional view of the nosecone of FIG. 149, showing the fluid input path from an input port that receives fluid from the water conditioner.

FIG. 151 is a perspective view of the nosecone of an applicator with graphical representation illustrating the flow of a cooling fluid.

FIG. 152 is another view of the nosecone shown in FIG. 151, showing the output port, where the cooling fluid is drawn out of the applicator.

FIG. 153 is a cross-sectional view of an applicator having an integrated image detector.

FIG. 154 depicts an image detector positioned within an applicator of a treatment module. As illustrated, air pockets are trapped between the membrane of the applicator and the patient's skin, air bubbles are on the inner surface of the membrane, and air bubbles floating in the coupling fluid.

FIG. 155 is a flow chart illustrating one example of a bubble detection process.

FIGS. 156-161 illustrate various configurations for the placement of image detector(s) in an applicator of a treatment module.

FIG. 162 depicts one variation a bubble removing device.

FIG. 163 show one example of a water conditioner with one of its side panels removed.

FIG. 164 depicts a typical catheterization table.

FIG. 165 depicts one example of a patient platform, which is positioned on the catheterization table of FIG. 164.

FIG. 166 depicts one variation of a patient platform in an exploded view, showing various components that comprise the patient platform.

FIG. 167 shows the patient platform of FIG. 166 fully assembled.

FIG. 168 illustrates one variation of a treatment module positioning template.

FIG. 169 depicts one example, where treatment windows has be marked on both sides of a patient's body.

FIGS. 170-173 illustrate an example of a procedure for using a treatment module placement template. (a) Verify that the bony-structure boundaries have been marked. (b) Position the template so that its interior straight edge is aligned with the lateral edge of the spine, and its interior perimeter at the apex just overlaps the inferior edge of the rib, then trace with red indelible ink around the outer perimeter of the template from its apex to its base on the spine side. (c) Position the template so that its contralateral inside straight edge parallels the inferior edge of the 12^{th} rib, proximal to the spine and the inside perimeter at the apex of the template, just overlaps the edge of the spine, then trace with red indelible ink around the outer perimeter of the template from its apex to its base on the costal side. (d) The red tracing can be used during treatment module placement to indicate the boundary beyond which the treatment module nosecone shall not extend.

FIG. 174 is a flow chart illustrating an example of an operator action sequence for treatment planning and control.

FIGS. 175A-175C depict an example of a planned lesion (or therapy treatment) pattern.

FIG. 176 is an graphical representation of FIG. 175A, illustrating the relative position and dimension of the 18 lesions that comprise the treatment pattern.

FIGS. 177-178 illustrate an alternative treatment pattern.

FIG. 179 depicts an example of an intra-lesion cycle.

FIG. 180 illustrates one example of functional steps for Phase Aberration Correction (PAC).

FIG. 181 is an example of a time diagram for phase aberration correction RF signal acquisition.

FIG. 182 is a software block diagram of an example of a computation process for phase aberration correction.

### DETAILED DESCRIPTION

The following detailed description should be read with reference to the drawings, in which identical reference numbers refer to like elements throughout the different figures. The drawings, which are not necessarily to scale, depict selected embodiments and are not intended to limit the scope of the claimed invention. The detailed description illustrates by way of example, not by way of limitation, the principles of the claimed invention. This description will enable one of ordinary skilled in the art to make and use the claimed invention, and describes several embodiments, adaptations, variations, alternatives and uses of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

It is to be understood that unless otherwise indicated, one or more embodiments described herein need not be limited to applications in humans. Although a human patient is referenced herein, one of ordinary skill in the art would appreciate variations of the embodiments may be applied to other mammals as well. Moreover, it should be understood that embodiments may be applied to deliver focused ultrasound into a patient's body for therapeutic and/or diagnostic purposes. Renal innervation is provided herein as an example of a clinical application. One of ordinary skill in the art having the benefit of this disclosure would appreciate that the variations and embodiments may be applied to different clinical applications and indications, including, but not limited to, denervation, tissue ablation, cancer treatments, tissue agitation, tissue heating, tissue denaturation, and drug activations.

It is also noted that, as used in this specification and the appended claims, the singular forms “a,” “an” and “the” include plural referents unless the context clearly dictates otherwise. Thus, for example, the term “a transducer” is intended to mean a single transducer or a combination of transducers, “a fluid” is intended to mean one or more fluids, or a mixture thereof. Furthermore, the words “proximal” and “distal” refer to directions closer to and away from, respectively, a physician operating the catheter with the tip end (i.e., distal end) placed inside the patient's body. Thus, for example, the catheter end placed in the renal artery of the patient would be the distal end of the catheter, while the catheter end outside the patient's body would be the proximal end of the catheter.

### I. System Layout

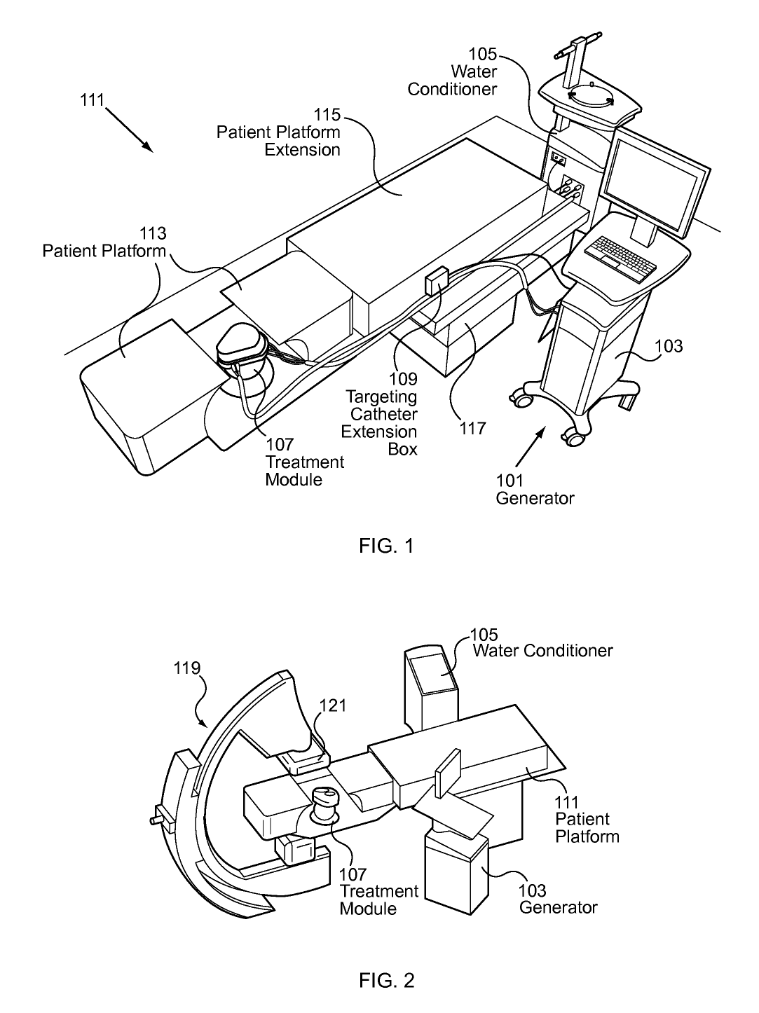

In one example, the therapeutic ultrasound system is configured to deliver focused ultrasound energy to treat tissues within a patient's body. Referring to FIG. 1, one variation of a therapeutic system 101, configured for delivering focused ultrasound energy to the mid and lower torso region of a patient's body, is shown. For example, the system may target the nerve and tissue surrounding renal arteries to denervate a patient's kidney. The system may also be applied to deliver focused ultrasound energy to other tissues or organs in the patient's body. For example, the system may deliver energy to the superior mesenteric blood vessels, the inferior mesenteric blood vessels, the hepatic artery or portal vein, the celiac artery, the splenic vein, the splenic artery, the gastroduodenal artery, the pulmonary artery, the pulmonary vein, the atrium, the ventricle, the vagus nerve, the carotid artery, the jugular vein, any of the ganglia along the spinal column or sympathetic chain, the carotid body, the carotid sinus, and/or any of the peripheral arteries or veins in a body. One of ordinary skill in the art having the benefit of this disclosure will appreciate that variations of the system can be configured to enhance treatment for different tissues or organs in the patient's body, depending on the location and the depth of the tissue within the body.

The therapeutic ultrasound system 101, as shown in FIG. 1, includes a generator 103, a Water Conditioner (WC) 105, and a Treatment Module (TM) 107. The system is configured to connect to a targeting catheter through a targeting catheter extension box 109. The system may be utilized with a Patient Platform (PP) 111 to facilitate the positioning of the treatment module 107 on the patient's body. One variation of a patient platform 111, shown in FIG. 1, includes a torso support module 113 and an extension 115, which are configured to be coupled to a standard patient table 117 in a catherization laboratory of a hospital.

FIG. 2 illustrates one variation of a system configuration, which includes a fluoroscopy unit (also known as a C-Arm) 119 containing an image intensifier 121. The fluoroscopy unit 119 may be utilized to locate and identify the target tissue. It may also be utilized for placement of a targeting catheter or other intervention and/or monitoring tools inside the patient's body during a treatment procedure.

In one variation, the generator includes a power supply, a central processing unit (CPU) an operating system, targeting and dosimetry software, motor controllers, multiple radio-frequency (RF) amplifiers, display drivers, a treatment module nosecone camera display, a system display monitor, a keyboard and a mouse. The generator serves as the overall system controller, energizes the treatment transducer array, powers and processes signals from the targeting subsystem, and drives the applicator positioning mechanisms.

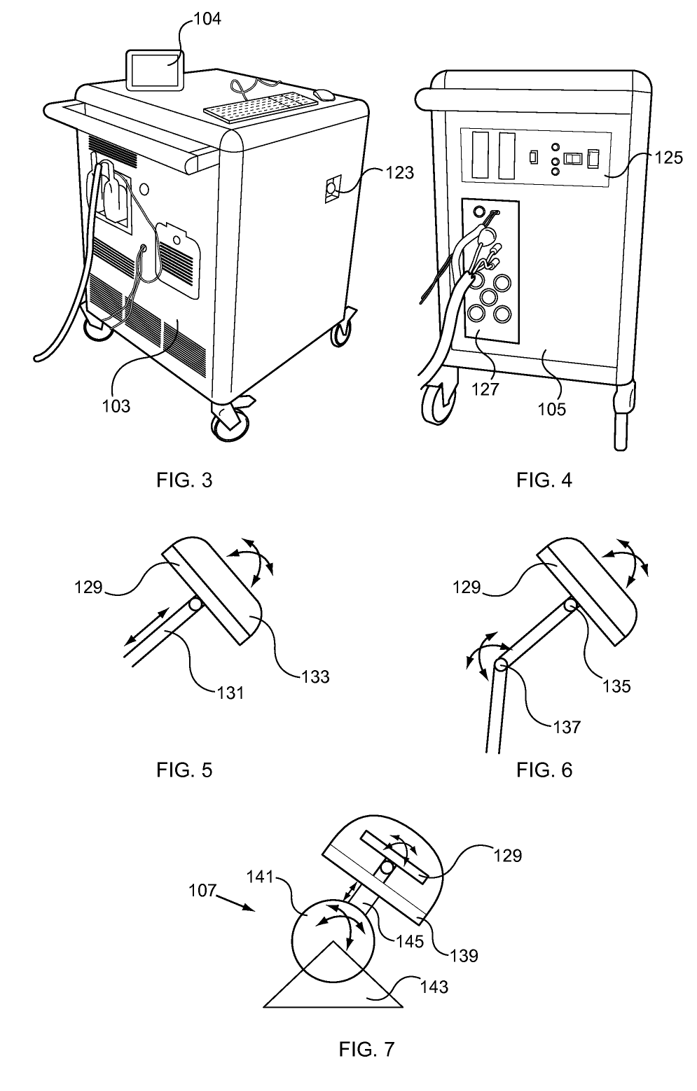

Referring to FIG. 3, one example of a generator 103 is configured with a mobile cart enclosure that houses a computing unit, an ultrasound transceiver, an electronic interface board and a power supply. The computing unit serves as the system controller, and targets and tracks treatments, controls the position and movement of the treatment transducer array, calculates and executes treatment plans, and monitors system performance. The ultrasound transceiver, through the electronic interface board, drives the ultrasound treatment transducer array, monitors the Acoustic Time of Flight (ATOF) receivers, and powers the targeting catheter. The computing unit is connected to a video monitor with Graphical User Interface (GUI), which is the primary medium by which the operator interacts with the system. Keyboard, mouse and/or other input devices may be provided for user input. A video display for monitoring the treatment module nosecone image detector's output images may be provided. The generator may also include a treatment over-ride button 123, located on the side of the generator. The button allows the operator to quickly interrupt and shutdown the system.

In one variation, the overall system is also provided with a water conditioner (also referred to as a water cart) 105, as shown in FIG. 4, which circulates, degasses and actively cools (e.g., to approximately 10 degree Celsius) the coupling fluid (e.g., distilled water) in the treatment module. The conditioned fluid is utilized to cool the ultrasound treatment transducer array and the patient's skin, to minimize of treatment related skin burns. It maintains acoustic coupling of the treatment transducer array, with the patient's anatomy, and thereby, provides an air-free pathway for ultrasound transmission to the target tissue. The water conditioner includes controls and indicators 125, for user control and notifications, and cable and hoses 127, for connecting with the treatment module and the generators.

The treatment transducer array, configured to generate a focused ultrasound for transmission into the patient's body, may be positioned on an actuator that provide movements of multiple degrees of freedom, and allows the generator to actively steer the treatment transducer array in order to adjust the location of the focused ultrasound beam emitted by the treatment transducer array. In one variation, the treatment transducer array comprises a phased-array ultrasound transducer. The generator is configured to power the phased-array, such that the focal depth of the ultrasound field can be adjusted without physical movement of the transducer array.

FIG. 5 illustrates one example where the treatment transducer arrays 129 is able to pitch and roll. In one variation, an actuator is provided to allow the treatment transducer to turn along the axis of the supporting shaft 131. An acoustic coupling interface 133 may be included to provide an ultrasound pathway from treatment transducer arrays to the patient's body. Optionally, the actuator may also be configured to provide advancement/retraction of the treatment transducer array in the Z-direction, along the length of the shaft supporting the treatment transducer array 129 for increasing contact pressure, and/or to mechanically adjust the depth of the ultrasound focused.

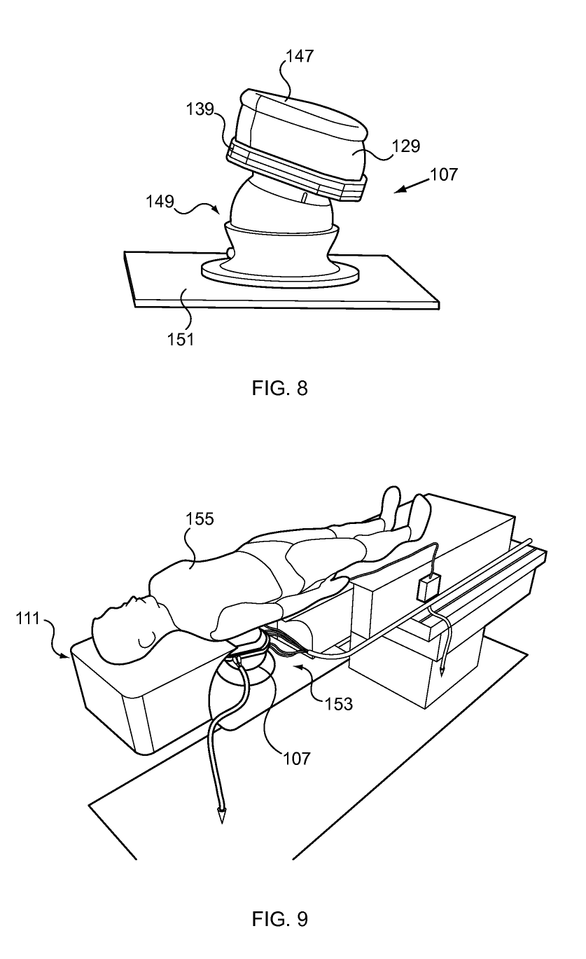

FIG. 6 illustrates another variation where the treatment transducer is coupled to two separate moveable joints 135, 137. FIG. 7 illustrates another variation where treatment transducer array 129, which produces the focused therapeutic ultrasound, is housed within an applicator 139 of a treatment module 141. The treatment transducer array 129 can be moved in the pitch and roll directions, independent of the applicator 139. The applicator 139 is connected to a ball joint 141, which is coupled to a base 143. The ball joint 141 allows the applicator 139 to freely rotate along the ball joint with 3 degrees of freedom. Optionally, the shaft 145 of the adaptor coupling the adaptor 139 to the ball joint 141 may be configured to move in the Z-direction along the length of the shaft 145.

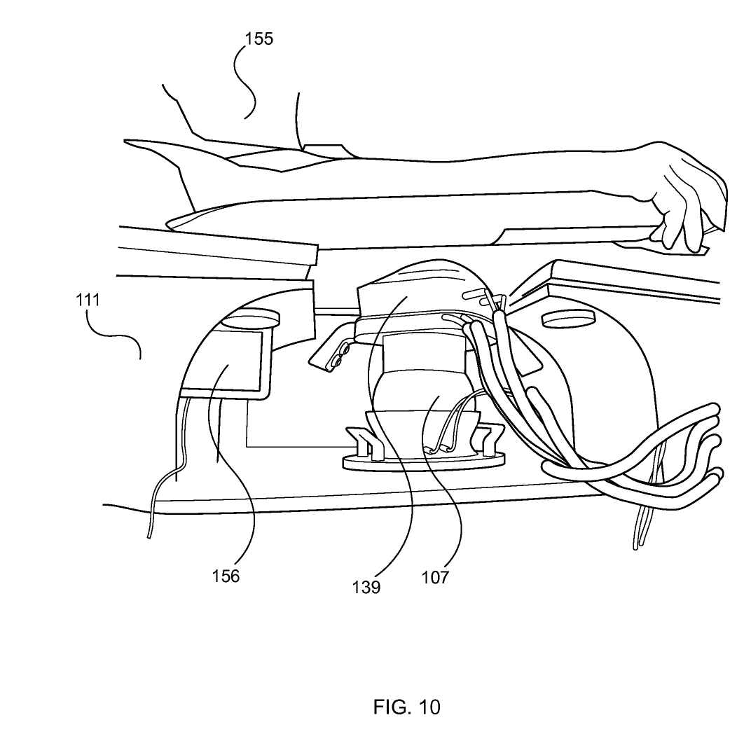

Referring to FIG. 8, another example of a treatment module 107 having a treatment transducer 129 housed within an applicator 139 is shown. The top surface 147 of the applicator includes a membrane for containing coupling fluids and engages the patient's body. An articulating joint 149 is provided to pivot the treatment applicator 139 into position. The bottom surface of the treatment module 107 rests on a patient platform 151, and can glide along the surface of the patient platform 151 for repositioning of the treatment module 107. The treatment module can be physically connected to the generator and the water conditioner. The treatment module also contains a mechanism to maintain positioning of the transducer array on the patient's posterior flank, and through the control from the generator, to automatically track the targeted tissue (e.g., renal artery) during a procedure. In one variation, the tracking function utilizes a series of sensors that communicate with an indwelling beacon (e.g., a beacon in a targeting catheter) to provide real-time spatial information, and allow tracking of a target region in the patient's body. The applicator of the treatment module is configured with a flexible patient interface that facilitates coupling to the patient's anatomy and provides a path for transmission of the focused ultrasound energy. Referring to FIG. 9, a patient platform 111 is a designed with an aperture 153 through which the treatment module 107 can be positioned to couple to the patient's 155 posterior flank and facilitate patient treatments in the supine position. Once the patient 155 lies down on the patient platform 111, the top surface of the treatment module 107 couples to the patient's posterior flank, see FIG. 10. The bottom surface of the treatment module rests on the patient platform. The treatment module includes a ball-joint that can be articulated to pivot the applicator to improve patient contact and adjusting the ultrasound propagation direction of the treatment transducer array, to generally face the direction of the target region of tissue to be treated. Actuators (e.g., electro mechanical movers or motors) within the applicator allow the system to pitch and roll the treatment transducer array, and adjust the position of the focus during treatment in real-time.

The therapeutic systems may be configured for use with or without a tracking beacon to facilitate locating the target tissue to be treated. In one variation, the therapeutic system is configured with acoustic sensors to detect an ultrasound beacon placed in or adjacent to the target tissue. The beacon may be integrated within a catheter and deployed next to the target tissue. In another variation, an imaging system is included as part of the therapeutic system. The imaging system may be utilized to locate the target tissue and track the position and/or movements of the target tissue, such that the treatment transducer array can maintain its focus on the target tissue during the treatment process. Various imaging modalities (e.g., X-ray, MRI, CT, ultrasound, etc.) may be utilized to provide image tracking. In one example, an ultrasound imager is implemented to track the position of the target tissue. In another variation, both ultrasound beacon and image tracking are utilized in combination for tracking of the target tissue.

An example of a treatment process is described below. A patient 155 is placed in the supine position with his posterior flank located over the treatment aperture 153 of the patient platform 111, as illustrated in FIG. 10. For a procedure where a beacon is utilized to track the target tissue to be treated, a catheterization procedure can be followed to place a targeting catheter into the patient's body. In this example, under fluoroscopy guidance, the distal tip of the targeting catheter carrying the ultrasound beacon (e.g., a piezo-electric bead at the tip) is advanced through the vasculature and into the renal artery, for treatment of tissues surrounding the renal artery. The catheter may be deployed using radial or femoral access.

The treatment module 107, which comprises the ultrasound treatment transducer array, is adjusted such that the applicator 139 of the treatment module engages the skin on the posterior flank of the patient 155, as shown in FIG. 10. The treatment module comprises an applicator, which houses the treatment transducer array for generating the ultrasound energy for treatment. The applicator is coupled to a mover assembly. The Applicator is pressed up against the renal region using a dorsal approach. Receivers, which are comprised of an array of ultrasound transducers (in receive mode) positioned in or around the applicator are configured to detect the signal from the ultrasound beacon on the catheter. For examples, the receivers may be placed in a frame surrounding the treatment transducer arrays.

The system is then adjusted to optimize of the targeting system parameters for tracking of the target tissue. For example, the targeting catheter drive voltage and frequency of the beacon, and receiver parameters for the ultrasound receivers configured for detecting the ultrasound beacon signal, can be adjusted to improve tracking of the beacon.

Based on the Acoustic Time of Flight (ATOF) signal transmission from the ultrasound beacon to the various receivers, the position of the beacon, relative to the receivers, can be calculated based on triangulation, and the spatial relationship between the active tip of the catheter (i.e., the beacon) and the treatment array can be determined. Next, the treatment module is adjusted to ensure that the focus of the ultrasound treatment array is focused around the target tissue (e.g., a renal artery). ATOF signals are monitored to confirm proper targeting of the tissue to be treated.

In variations where an imaging array is used for tracking of a target, the imaging information is used to confirm that the focus of the treatment array is properly position over the treatment region. The system then calculates the treatment parameter, such as dosing of ultrasound energy to be applied to the treatment region. For example the physician may enter the desired dosing level for a particular treatment. The system may also take into account other parameters, such as the distance of the target region from the treatment transducer array, and calculate the appropriate ultrasound energy to apply to achieve the desired dosing at the target region. A particular treatment plan, such as a specific treatment pattern (e.g., energizing multiple spots within a treatment area), and a specific dosing routine (e.g., spreading a dose into multiple quantized delivery over a finite period of time to achieve the desired dose) may be selected. The treatment plan is then implemented by the system, and ultrasound energy is delivered to the treatment region based on the treatment plan. In some embodiments, the treatment plan includes sequential lesions offset from the blood flow of a vessel and within 5 mm of one another. In another embodiment, the treatment plan includes sequential lesions offset from the blood flow and within 1 mm of each other. In another embodiment, the sequential lesions do not have an offset from one another and sequential lesions are applied atop one another in substantially the same position.

### II. System Functionality

In one configuration, the ultrasound therapeutic system features two Graphical User Interfaces (GUIs) that provide guidance and management of the therapeutic system. The primary GUI is displayed on a video monitor located on the generator. A row of menus along the bottom of each screen serves to step through the treatment procedures. The procedures are implemented by sequencing through each menu and inputting the requisite information and/or performing the indicated tasks. Such sequencing initiates appropriate system computations, status changes and actions. The operator interacts with the therapeutic system by keying-in information using a keyboard and selecting checkboxes presented on the display using a mouse.

A secondary GUI is displayed on a remote targeting monitor, mounted in the treatment aperture of the patient platform. The remote targeting monitor GUI provides visual feedback to the operator to assist the operator to position the treatment module within the patient's treatment window, target the desired tissue, such as the renal artery, for treatment, and engage the various mechanisms that maintain the treatment module position.

An image detector, such as a camera, may be positioned within the treatment module applicator for monitoring the contact between the treatment module and the patient's body. Images from the camera located within applicator nosecone may be continuously displayed on a video monitor located on the generator (i.e., a generator treatment module camera monitor 104, FIG. 3) and in the treatment aperture of the patient platform (i.e., a remote treatment module camera monitor 156, FIG. 10). The images provided on these monitors may be identical and can be useful for (1) identifying and removing air pockets between the ultrasound treatment array and the subject's skin, (2) detecting air bubbles on the inner surface of the membrane of the applicator, (3) detecting air bubbles in the fluid in the applicator, (4) positioning the treatment module nosecone in the treatment window on the patient's body, and/or (3) detecting patient movement during administration of the investigation treatment. Air pockets and bubbles are used interchangeably herein to mean a confined, enclosed or trapped air or gas. The camera may also be useful for monitoring hotspots on the skin of the patient with various wavelengths of light such as infrared or near infrared light. Reflective acoustics can also be utilized to identify pathlengths from the transducer face to the skin in which a variable pathlength through water might exist. Visualization also can be used position the transducer underneath a marked region of the patient.

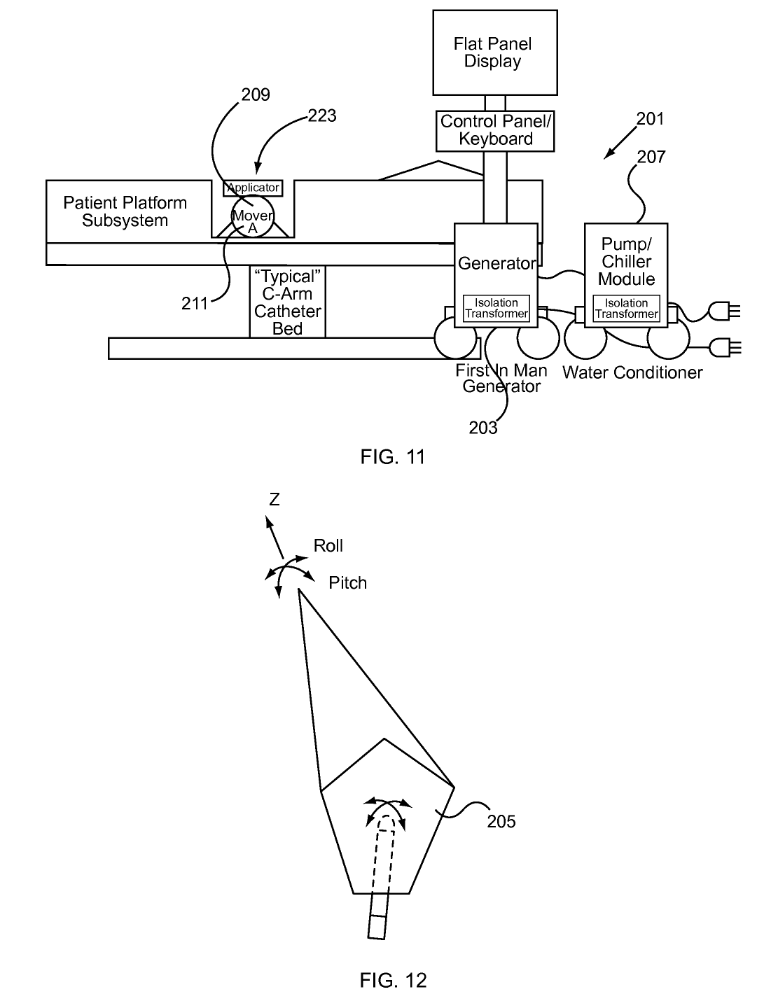

FIG. 11 is a graphical representation of one variation of an ultrasound therapeutic system 201 configuration deployed next to a conventional C-Arm catheter bed in a catheterization lab. A generator 203 provides physical support and mobility for (a) an ultrasound transceiver module (e.g., a source of the electrical power to the treatment transducer array and the targeting catheter, along with ATOF receive signal amplification and digitization), and (b) a computational subsystem, where the ultrasound therapy system's control software operates. The ultrasound transceiver module may be configured to allow the subsystem to energize the ultrasound treatment transducer arrays to emit ultrasound energy, and at the same time having the capability to detect ultrasound energy received by the ultrasound transducers, allowing the transducer array to function as receivers.

The treatment transducer 205 may be configured with two or more degrees of controlled movements. FIG. 12 illustrates one example, where the treatment transducer 205 has two degrees of freedom (i.e., pitch and roll). A system controller (such as a computing unit located within the generator) may be adapted to control the movements of the transducer. For example, the treatment transducer array maybe coupled to a mover, which comprises actuators capable of receiving control signals from the computing unit. Depending on the application, a mover capable of controlling two or more degrees of orientation movements and/or two or more degree of position movements may be utilized.

A water conditioner 207 prepares and manages the water that provides the acoustic inter-path for the ultrasound energy. The water is degassed (reduce the possibility of creating bubbles during treatment) and chilled (provide patient comfort and cooling of the treatment transducer array) prior to the onset of treatment.

A treatment is accomplished by applying therapeutic levels of ultrasound energy, by a topically located applicator 209, to a target tissue, such as the renal arteries and its perivascular tissues. In one variation, this is accomplished over a three-step process, as follows: 1) initial target identification, 2) manual applicator positioning, 3) automated treatment process. The process may be repeated for the other tissue or organ on the contralateral side, if indicated.

In one example, the ultrasound therapeutic system identifies the exact location of the renal artery with the guidance of a strategically placed targeting catheter, which has an ultrasound beacon at its tip. The tip of the catheter is first placed at the renal artery under fluoroscopy guidance. After the catheter is properly situated, the applicator (with Mover A 211) is positioned in the proximity of the catheter. The therapeutic system can then excite the beacon, and listen with a known constellation of matched receivers (e.g., placed on the perimeter of the treatment transducer array), followed by triangulation calculation to establish a spatial relationship between the target tissue and the therapy array.

The therapeutic system 201 will then determine if the applicator 209 is positioned properly, checking for adequate acoustic path, clearance from bony and other obstructions, and also for good acoustic coupling. The operator may be prompted to move the position and/or orientation of the applicator, to improve treatment delivery. With the Applicator in position, the therapeutic system can be activated by the user to begin treatment of the tissue about the identified target area. The treatment may take a few minutes, and during the treatment the therapeutic system can automatically move the therapeutic ultrasound field focus to compensate for respiratory motion or other movement distortions.

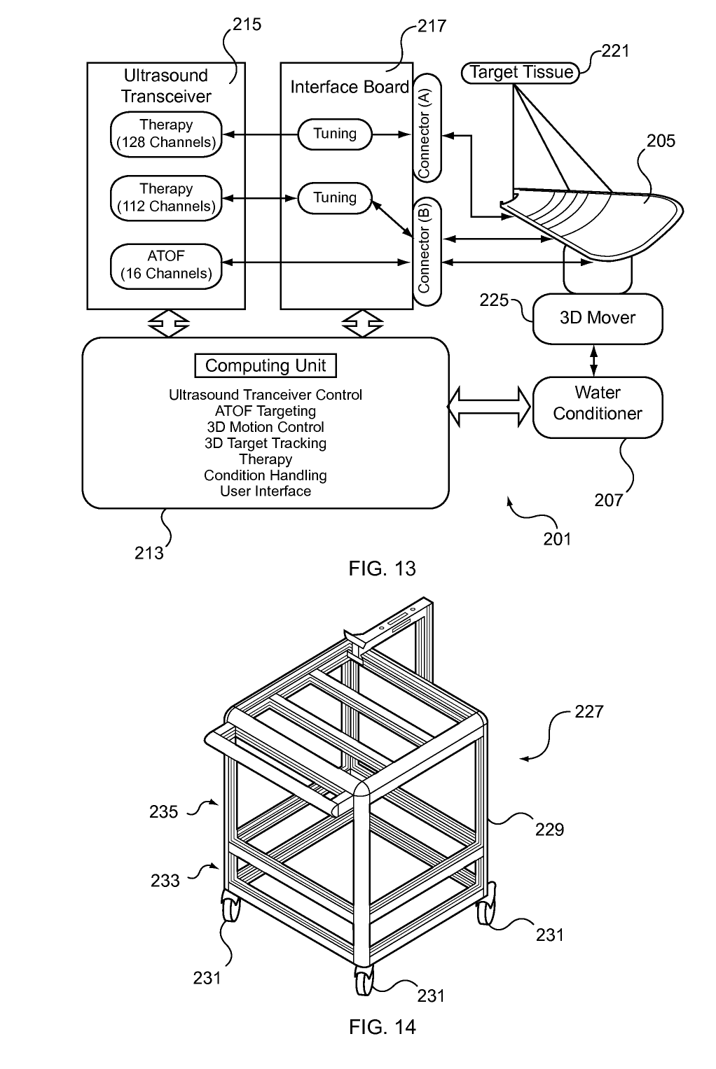

Referring to FIG. 13, one configuration of a therapeutic system 201 includes a generator, which comprises (a) a computing unit 213 having one or more CPU, (b) an ultrasound transceiver module 215, and (c) an electronic interface board 217. The computing unit 213 sends and receives data to and from both the ultrasound transceiver module 215 and the electronic interface board 217. This allows the computer to receive ATOF signals from the ATOF receivers located around a treatment transducer array 205 in the treatment module, and control the delivery of ultrasound energy from the treatment transducer array 205 to the target tissue 221.

The computing unit 213 may also be connected to a water conditioner subsystem 207, allowing it to control the cooling of treatment transducer array 205 and the interface between the treatment transducer array and the patient. In one variation, the treatment transducer array 205 comprises a phased-array of ultrasound transducers. For example, the phased-array may comprise of 230 transducers, which are aligned in a manner capable of generating a focused ultrasound wave. The ultrasound transceiver module 215 delivers multiple channels electrical current 217, through the interface board, to drive each of the plurality of transducers 205 that forms the phased-array.

The treatment module 223, which includes the treatment transducer array 205 and the 3D mover 225, is connected to both the electronic interface board 217 in the generator, and the water conditioner 207. In one configuration, the treatment module 223 comprises a mover coupled to the treatment transducer array. The mover has at least two degree of freedom, which is able to move the focus of the treatment transducer array in at least two directions. For example, as shown in FIG. 12, the mover is configured to move the treatment transducer array 205 with both pitch and roll. The depth of the focus may be adjusted through phase adjustments in energizing the transducers in the phased array of the treatment transducer array. In another variation, the mover comprises a 3D mover capable of provide at least 3 degree of freedom in movements. In another variation, the mover is configured to provide 6 degrees of freedom in actuated and/or controlled movements of the treatment transducer array. In yet another variation, the mover is configured to move the treatment transducer array in a three dimensional space (and therefore, direct the focus of the treatment transducer in a 3D space), and the treatment array may comprise of either a fixed-focused array of transducers or a phased-array of transducers, or a combination thereof.

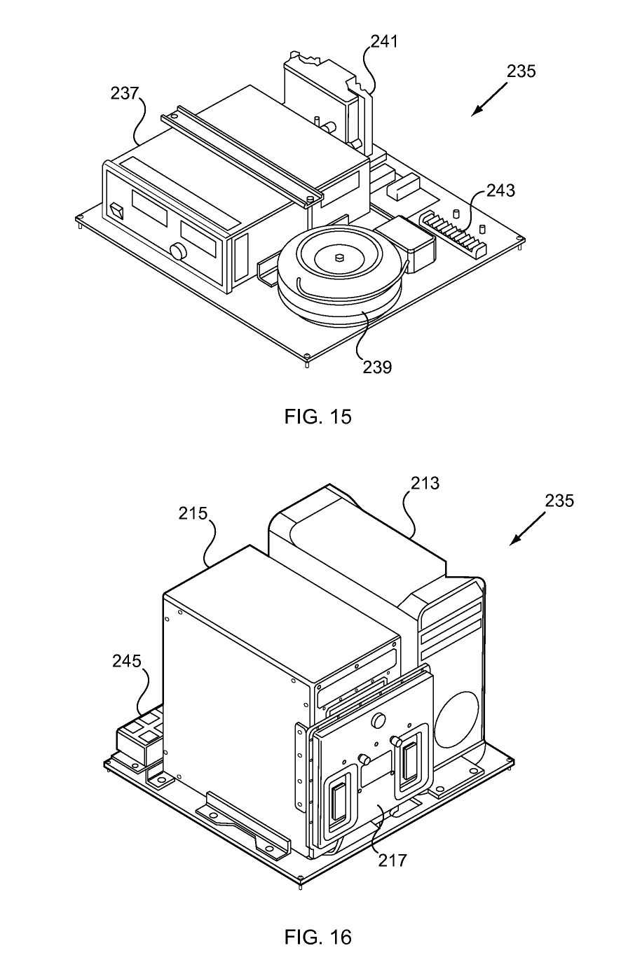

The generator 203, which includes the computing unit 213, the interface board 217, and the transceiver module 215, may be housed within a portable cart 227. In one example, the portable cart 227 comprises a frame 229 with wheels 231, as shown in FIG. 14, as the underlying supporting structure. The components for the generator 203 are housed in two levels 233, 235 in the portable cart. The lower level 233 (a.k.a., the first floor) comprises a power supply 237 (such as a 1200 Watt, Dual DC power supply manufactured by Thurlby Thandar Instruments, Huntingdon, United Kingdom), a medical isolation transformer 239, a battery, a battery board 241, and a terminal block 243 having 12 connections, as shown in FIG. 15. Referring to FIG. 16, the upper level 235 (a.k.a., the second floor) comprises a computing unit 213, an ultrasound transceiver module 215, an electronic interface board 217, and a power strip 245.

In one example, the computing unit is configured to provide (a) ultrasound transceiver control, (b) ATOF targeting, (c) 3D motion control of the treatment module, (e) 3D target tracking of the target region, (f) therapy delivery, (g) conditioning handling (e.g., system operating parameter control, system shut-down control, etc.), and (h) user interface.

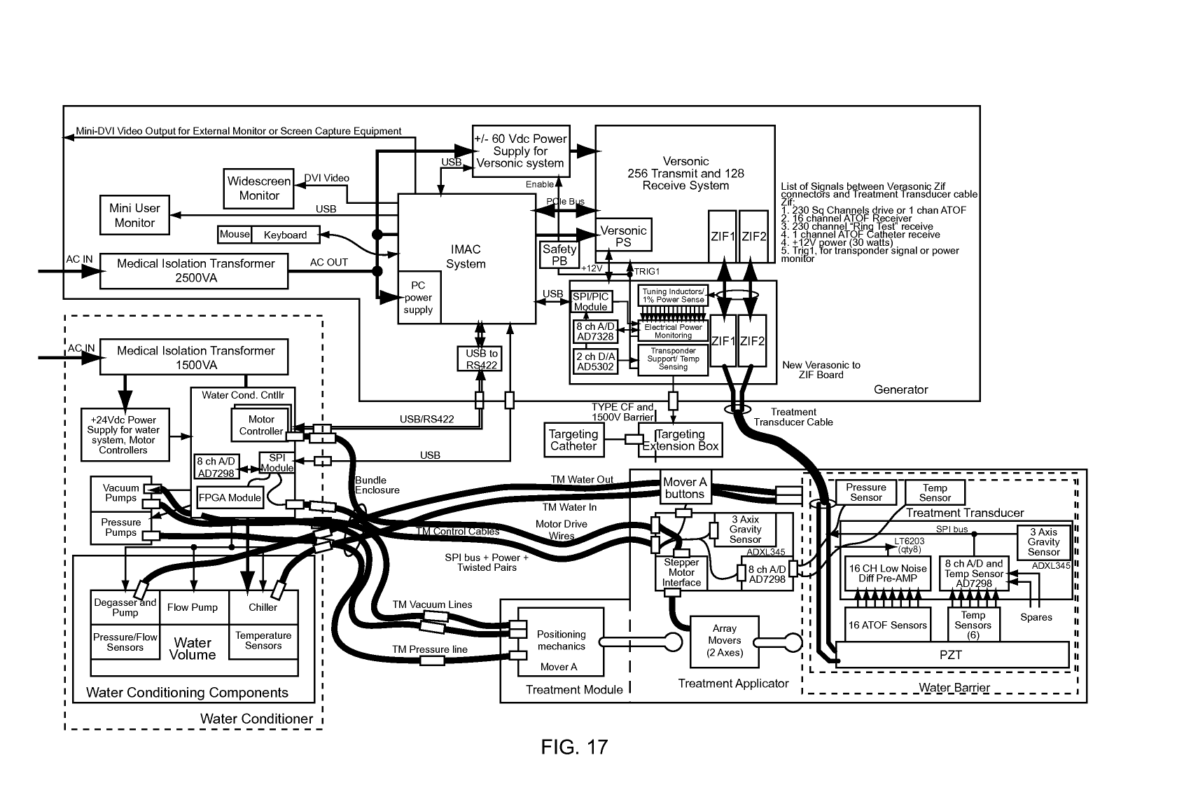

FIG. 17 is a functional block diagram for one variation of a therapeutic system 201. The figure illustrates subsystems, and the electrical and plumbing interconnects. The detailed interconnections between the generator 203, water conditioner 207 and treatment module 223 are also shown. In this variation, the generator 203 comprises a box for holding the following sub-components: (a) An ultrasound transceiver module 215. For example, the module may comprise of a transceiver with up to 256 channel RF drive and 128 channel receive capability, such as an ultrasound transceiver manufactured by Verasonics, Inc. of Redmond, Wash. The ultrasound transceiver may also comprise of a treatment transducer array power supply. For example, the power supply may comprise of a 1200 W power supply unit providing the power for the RF power amplifiers in the transceiver, controlled over USB by the computing unit. (b) A computing unit 213 for controlling the system and handling various computations. The computing unit may comprise of a computer with one or more CPUs and may further comprise one or more GPUs. For example, the computing unit may comprise of an iMac Pro computer (with a PCIe extension card for communicating with the ultrasound transceiver module, and may include other add-on cards for additional USB ports and/or RS-422, display monitor(s), keyboard, touch pad or mouse) manufactured by Apple, Inc. of Cupertino, Calif. (c) An electronic interface board 217. The interface board may provide hardware to impedance match between each treatment array element to the amplifier in the ultrasound transceiver module. The board 217 may also be further configured to provide power monitoring for each treatment array drive channel (allowing the computing unit to monitor the drive channels), support ATOF catheter drive and ATOF sensor receive functions, and other tasks such as temperature monitoring of the treatment transducers (e.g., detect heating of the PZT materials), and movements, such as physical tip and tilt, of the treatment transducer array, etc.

In one variation, the electronic interface board 217 comprises an electrical power monitoring unit. The electronic power monitoring unit may include an electrical power monitoring microprocessor or microcontroller, such as a Peripheral Interface Controller (PIC). As shown in FIG. 17, the electrical power monitoring unit 247 detects the power going to each transducer elements of the treatment array. In one variation, the electrical power monitoring unit is configured to monitor the electrical current going to the transducers. In another variation, the electrical power monitoring unit is configured to monitor the voltage going to the transducers. In yet another variation, both the current and the voltage for the power going to the transducers are being monitored.

In another design variation, a high voltage power supply is provided to drive the treatment transducer array. The high voltage power supply may be located external to the ultrasound transceiver. The output of the high voltage power supply is connected to the electronic interface board, which directs the electrical power from the external power supply into the ultrasound transceiver, and the supplied power is used to drive the transducers on the treatment array. An electrical power monitoring unit on the electrical interface board monitors the power supplied by the high voltage power supply, and monitors the total power being used by the system to drive the treatment transducers. In one variation, the electrical power monitoring unit is configured to monitor the electrical current going from the high voltage power supply through the electronic interface board to the transceiver to drive the treatment transducers. In another variation, the electrical power monitoring unit is configured to monitor the voltage for the electrical power going from the high voltage power supply to the transceiver. In yet another variation, both the current and the voltage for the electrical power going from the high voltage power supply to the transceiver are being monitored.

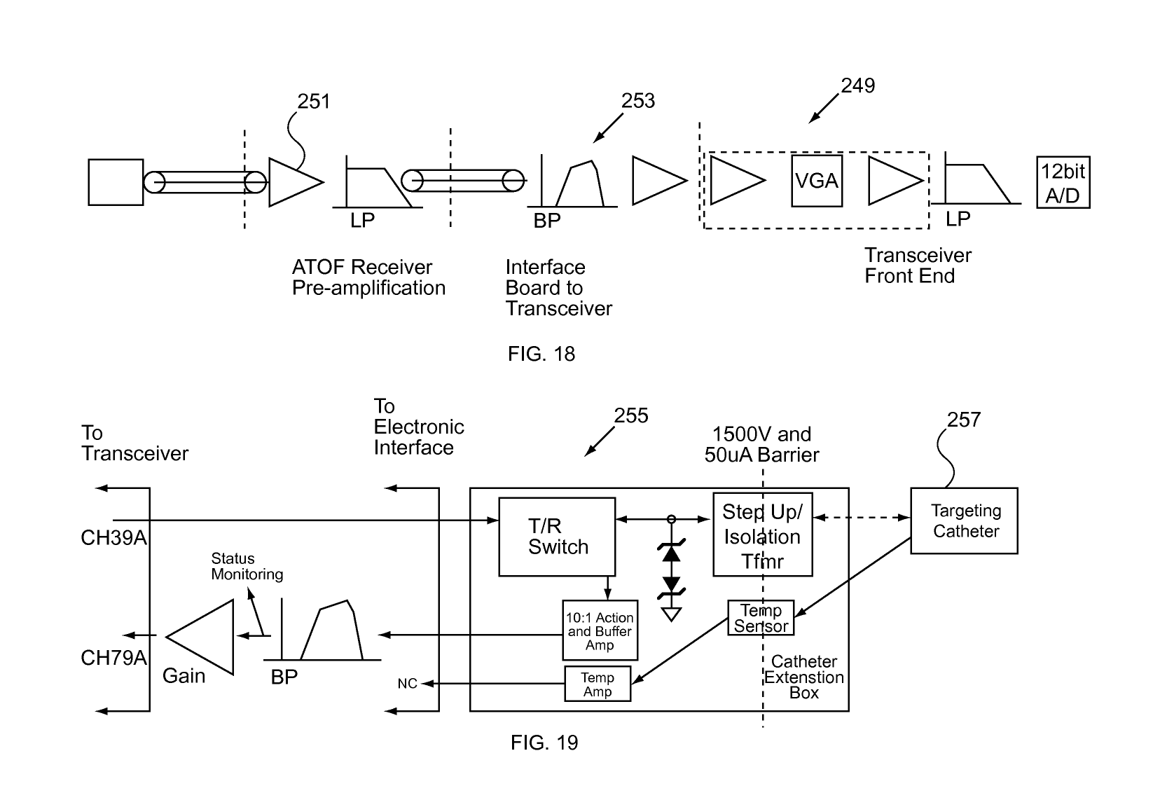

FIG. 18 illustrates one example of an ATOF amplification chain 249, including AC filtering. In this example, in consideration of the minute levels of signal, the detected ATOF catheter signal is immediately amplified 251 at the treatment module, before the signal is transmitted through a coax cable to the electronic interface board in the generator. The signal is then filtered after traveling from the treatment module to the generator by a band-pass filter 253 and amplified. The ultrasonic transceiver module front end may be based on a variable gain amplifier and may have a linear input range limit (for example, of approximately 200 mV Vpeak), at which time there is a soft compression gain roll-off (for example, terminating at a true 250 mV maximum Vpeak limit). This sets the maximum allowed gain for the preamp stages before the ultrasonic transceiver module (for example, using 0.9 mV as the maximum ATOF receiver output). In one variation, the variable gain amplifier has an internal adjustable gain stage of −27 db to +11 db, which can be used to maximize the actual ATOF signal to the 12 bit A/D converter, such that the majority of the digital range may be used. The gain stage on the ultrasound transceiver module to electronic interface board may be set by external resistors and can be changed, such as for testing the operation of the treatment array in a water tank.

Referring to FIG. 19, to provide electronic safely isolation, a catheter extension box 255 may be provided as part of the generator. The catheter extension box 255 comprises drive and receive support circuitry for targeting catheter 257. In one variation, the box includes a signal transformer that ensures the generator will meet type CF (IEC 60601-1 Cardiac Floating level) leakage current protection for the patient. The transformer can be a 1:1 winding, or it can perform a transformer function (i.e. 1:2.4), if higher drive is desired. A pair of Zener diodes may be included in the circuitry to ensure that the overall drive voltage to the targeting catheter is kept at a safe level. The Zener voltage can be dependent upon the transformer turns ratio. A T/R switch allows the return signal from the catheter to be available to the transceiver, at a reduced amplitude. This signal is also used by the system to monitor the status of the ultrasound beacon signal.

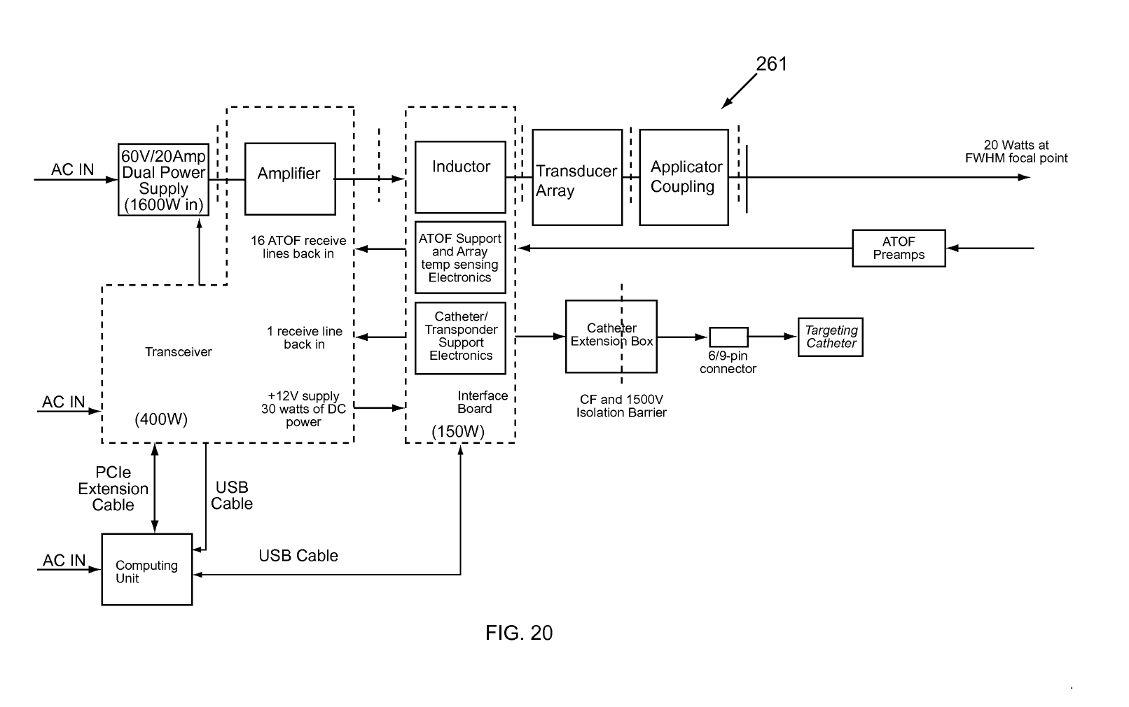

In one variation, power for the generator is provided through a 2500 W medical grade isolation transformer 259, as shown in FIG. 17. FIG. 20 illustrates one example of power transmission 261 from the AC input to the acoustic output of the treatment transducer array. In this example, the power attenuation is managed at each stage such that 20 Watts is achieved at the focal point of the treatment transducer array.

Additional sensors (e.g., temperature sensor, gravity sensor, accelerometer, position sensors, electric current level detectors, etc.) may be included in the system to provide control and safety feedback. In one example, a temperature sensor is positioned to detect the fluid coupling the treatment transducer array to the patient's body. The temperature sensor can be positioned between the membrane containing the coupling water, and the treatment transducer array. The coupling water may be constantly flowing during the treatment procedure to cool the transducer array and the patient's skin. The temperature sensor allows the therapeutic system to monitor the temperature of the coupling fluid and prevent overheating of the patient's skin. In one variation, two sensors are provided, with one placed closer to the membrane and the patient's skin, and the other placed near the treatment transducer array. In another variation, a temperature sensor is position on or within the membrane containing the coupling fluid, such that the temperature sensor is in close proximity to the patient skin to improve detection of the temperature on patient skin and prevent skin burn during the procedure.

A temperature sensor may also be positioned on the treatment transducer array to monitor the temperature of the treatment transducer array. The treatment array may heat up when it is energized. Monitoring of the temperature of the treatment array allows the system to confirm that the treatment array is operating in the desired operation range, and confirm that the coupling fluid is working properly and keeping the temperature of the treatment array low. For example the system may monitor the temperature sensor or sensors, and once one of the sensors detects a temperature exceeding a predefined threshold, a warning is provided to the operator. Once such temperate exceed a second threshold, the system may initiate a shunt down procedure.

The system may also monitor the electric current being drawn by the generator to drive the treatment transducer array. Referring to FIG. 17, an electrical power monitoring component 247 is configured to detect the level of electrical current driving the treatment transducer array 205. The current draw is a parameter that indicates the performance/condition of the transducers in the treatment array. When unusually large amount of current is drawn to drive the treatment transducer array, this may indicate that the treatment transducer array is not operating properly (e.g., overheating, or that one or more of the transducer elements are compromised.). The system may have a predefined current threshold, such that when the current drawn by the transducer array exceeded such threshold, the system will stop the treatment process and alert the operator of such condition.

In another variation, the system also includes one or more position and/or orientation sensors for detection of the position and/or orientation of the treatment array. The position sensor may comprise of a capacitive transducer, a linear variable differential transformer, a piezo-electric transducers, a proximity sensor, a rotary encoder, a linear encoder, or other sensors that are well known to one of ordinary skill in the art for position or displacement detection. The orientation sensor may comprise of a gravity sensor, an accelerometer, an inclinometer, an electoral mechanical orientation sensor, an opto-mechanical orientation sensor, a gyroscope sensor, or other sensors that are well known to one of ordinary skill in the art for orientation detection.

In one variation, a 3D orientation sensor 263, such as a 3-axis gravity sensor, is coupled to the treatment transducer array, as shown in FIG. 17, to determine the orientation of the treatment transducer array. The system may continuously monitor the orientation of the treatment transducer array during the treatment process. The treatment plan may call for focused ultrasound treatment of multiple regions within a treatment area. During the execution of a treatment plan, as one region is treated, and the system moves the focus of the treatment transducer array from a first region to a second region, the system would expect to detect a corresponding change in the orientation of the treatment transducer array based on the signals received from the gravity sensor. If the system gave the instruction to move the focus of the treatment array to a new focus region, but the gravity sensor indicates that the treatment transducer array did not move, or move to an orientation that does not correspond to the new focus region, this may indicate that the treatment module is not operating properly. Depending on a pre-set parameter, the system may alert the operator of such error, shut down the treatment process, or both.

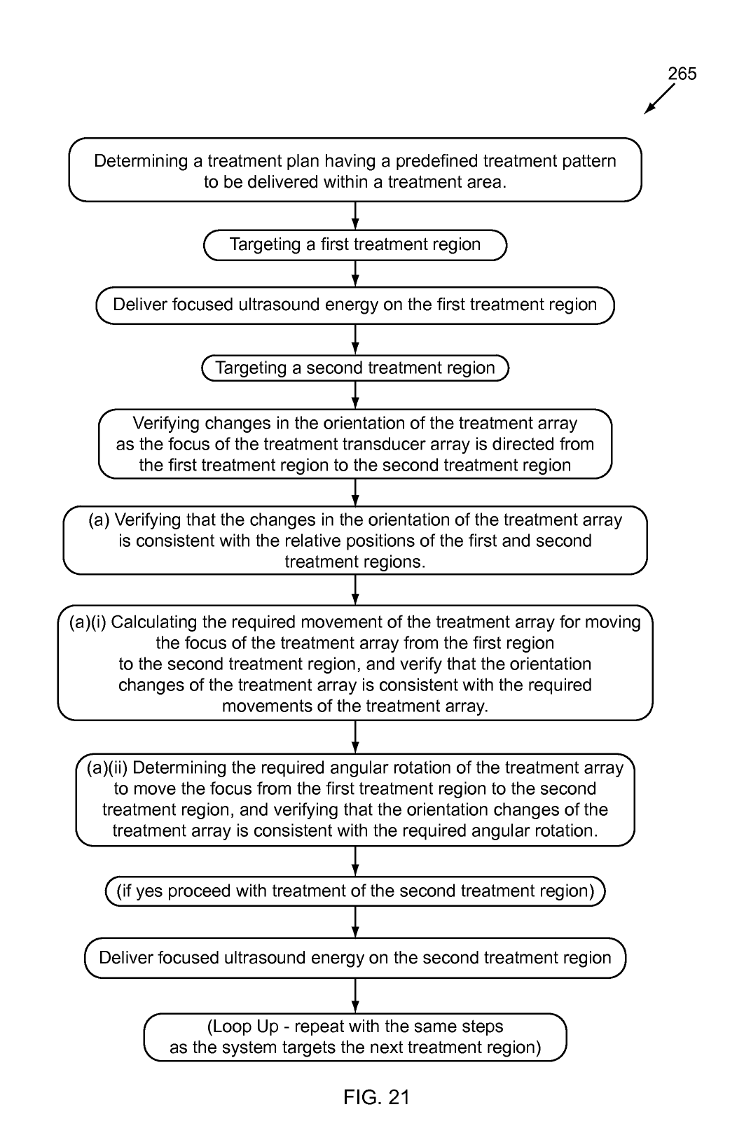

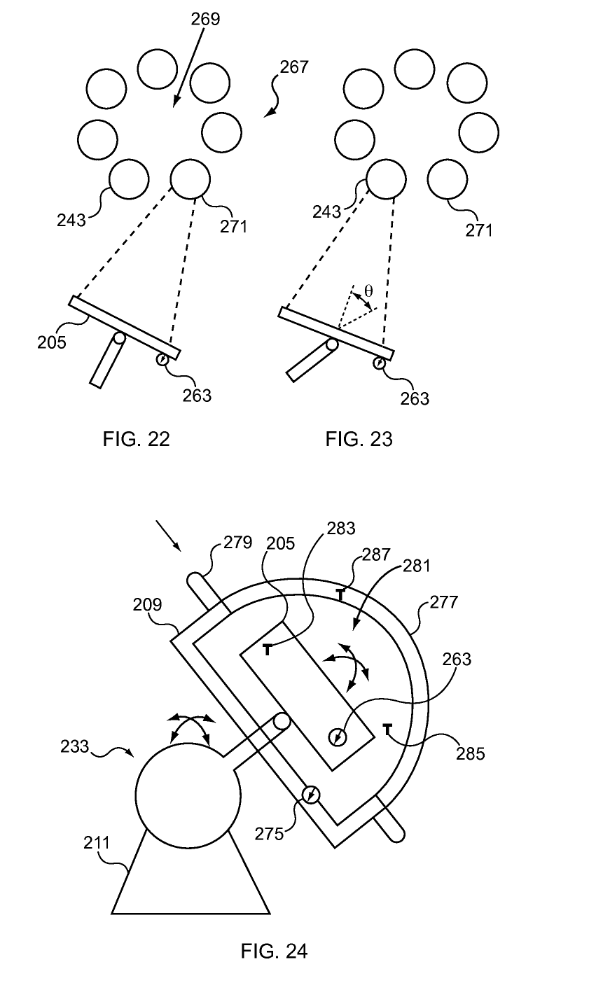

In one example, referring to FIG. 21, the treatment process 265 comprises the following steps. The system first determines a treatment plan having a predefined treatment pattern 267 to be delivered within a treatment area. For example, the treatment area may be a ring pattern surrounding a region 269 determined by the operator, or based on the location of a target beacon, as shown in FIG. 22. The system first targets a first treatment region. Focused ultrasound energy is then delivered to the first treatment region 271. After the first region is treated with the prescribe dosage of ultrasound, the system targets a second treatment region 273. The system then verifies changes in the orientation of the treatment array as the focus of the treatment transducer array 205 is directed form the first treatment region 271 to the second treatment region 273. As the system target the second treatment region 273, it is expected that the treatment array 205 would move accordingly and direct the focus of the treatment array to the second treatment region, as shown in FIG. 23. The verification step may comprise of verifying that the changes in the orientation of the treatment array is consistent with the relative positions of the first and second treatment regions. This step may further comprise of calculating the required movement of the treatment array for moving the focus of the treatment array from the first treatment region to the second treatment region, and verify that the orientation changes of the treatment array is consistent with the required movement of the treatment array. Alternatively, the system may determining the required angular rotation of the treatment array to move the focus from the first treatment region to the second treatment region, and verifying that the orientation changes of the treatment array is consistent with the required angular rotation. The orientation changes may be measured by an orientation sensor 263, which may be coupled to the treatment array 205. Once the system has confirm that the treatment array has move to the desired position, the system can then proceed with treatment of the second treatment region, and deliver focused ultrasound energy on the second treatment region. The above steps are then repeated as the system proceeds with the treatment plan to move from one target region to the next.

In another variation, a 3D orientation sensor 275 is coupled to the applicator 209 in the treatment module 223. This orientation sensor 275 allows the system to monitor the orientation of the applicator. FIG. 24 illustrates an example of a treatment module 223, where a treatment transducer array 205 is positioned within an applicator 209 of a treatment module 223. The treatment array 205 is independently moveable relative to the applicator 209. The applicator is connected to a base mover 211, which allows the operator to adjust the position of the applicator 209. In one variation, the base mover is electromechanically actuated, and its movement and position may be controlled by the computing unit. In another variation, the base mover's position and orientation are manually controlled by the operator.

A first gravity sensor 263 is coupled to the treatment array 205 to detect the orientation of the treatment array, and a second gravity sensor 275 is coupled to the applicator 209 to detect the orientation of the applicator. The applicator has a membrane 277 for interfacing with patient's skin. A first port 279 for injecting degassed and cooled fluid in the chamber 281 to keep the treatment transducer array cool, and to keep the membrane 277 interfacing with the patient cool. In this example, three temperature sensors 283, 285, 287 are provided to monitor the temperature within the applicator. The first temperature sensor 283 is coupled to the treatment transducer array, the second temperature sensor 285 is positioned between the treatment transducer and the member to measure the fluid flowing in between, and a third temperature sensor 287 is embedded within or on the membrane 277.

As the base of the mover 2011 is adjusted to align the applicator 209, which houses the treatment transducer array 205, the system expects to detect a change in the orientation of the applicator. Monitoring such orientation sensor in the applicator allows the system to confirm that such movement and change in orientation has been achieved.

During a certain time frame within a treatment protocol, one may expect the orientation of the treatment transducer array to be move, but expect the applicator to stay stationary. If the system detect that the treatment transducer array failed to move, or the applicator has moved unexpectedly, this may indicate an error has occurred, and the system may prompt the operator and/or shut down the treatment module, as prescribed by the treatment protocol.

In one variation, the treatment transducer array is moveably mounted within an applicator and powered by a multi-channel RF power subsystem (such as an ultrasound transceiver module) located within the generator. The integration procedures of therapeutic array and system, can generate a set of control parameters for the system to produce clinically desired acoustic power with one or more defined power distribution patterns to a targeted area inside a patient's body.

In some variations, the system integration procedures are defined to have a certain flexibility to compensate the undetermined clinical thermal requirements. The control parameters, such as focal positions, power, power distribution and power distribution variation time sequence, may be changed by sets of control parameters, and may be modified during the calibration or optimization process.

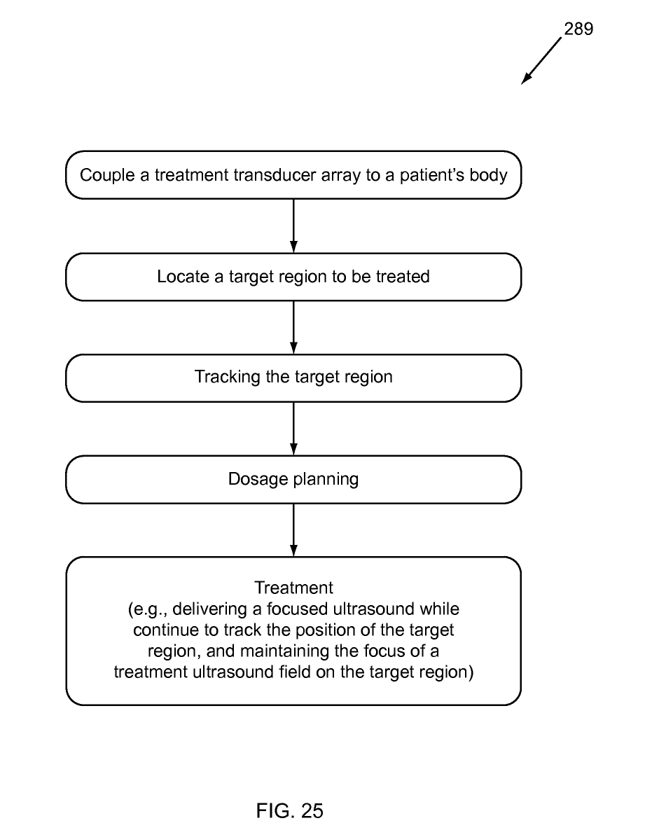

FIG. 25 illustrates one example of functional steps 289 for an ultrasound therapeutic system. The operator couples the treatment transducer array to the patient's body. The system then locates a target region to be treated. In a system where a tracking beacon is used, this may be achieved by the system locating the tracking beacon, which is positioned in or adjacent to the target region. ATOF receivers may be used to locate the position of the beacon. In another variation, the step may be achieved with the use of an image transducer array that allows the user to identify the target tissue and mark the target tissue for tracking. The system starts tracking the target issue and maintaining the focus of the treatment transducer array in the target region. Base on the location and distance of the target region from the treatment transducer array, the system then calculates dosage of focus ultrasound for the treatment. Dosage planning may include determining the number of dosage, the intensity of focus ultrasound for each dose, and the timing for delivery of each does. The dosage planning may also include calculating the phase control parameter and the power output control parameter for driving the treatment transducer array in order to achieve the desired power at the focal point of the treatment transducer array. The system then initiates the treatment and delivers the focused ultrasound to the treatment region based on the planned dosage.

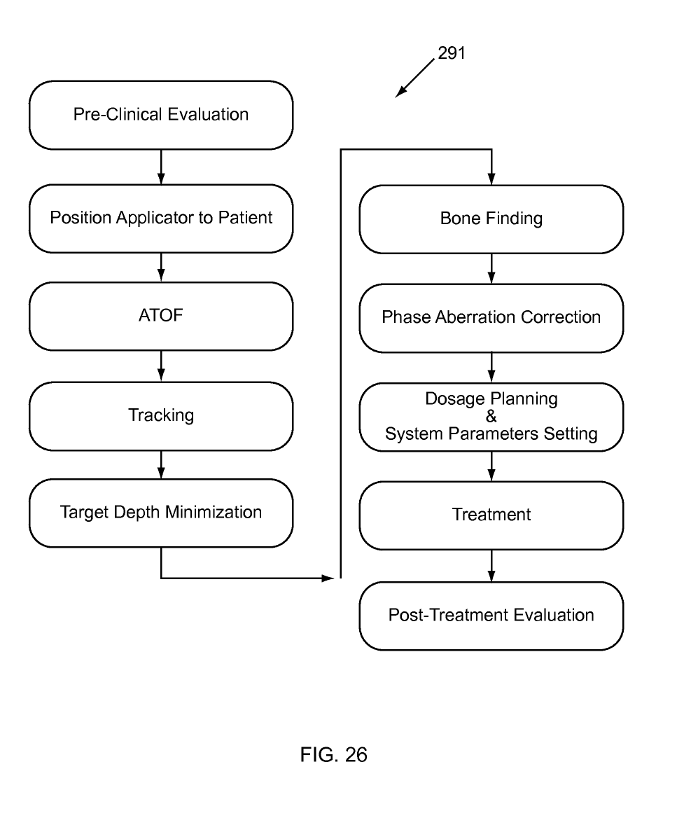

FIG. 26 illustrates another example of functional steps 291 for an ultrasound therapeutic system. A pre-clinical evaluation is conducted to determine the desired location for the placement of the treatment transducer array. The pre-clinical evaluation may also include determining the location for placement of the acoustic beacon. The applicator on the treatment module is positioned to engage the patient's body. ATOF receivers detect the acoustic beacon's signal, and then the system calculates the location of the beacon relative to the treatment transducer array. Based on the ATOF signal, the system adjusts the position of the treatment transducer in order for tracking of the beacon, and therefore tracking the desired target tissue surrounding or adjacent to the beacon. The system may be configured to determine the power and phase delivery of the focused ultrasound. Optionally, the position of the applicator may be adjusted to minimize the target depth (i.e., target depth minimization, minimizing the distance from the treatment transducer array surface to the target).

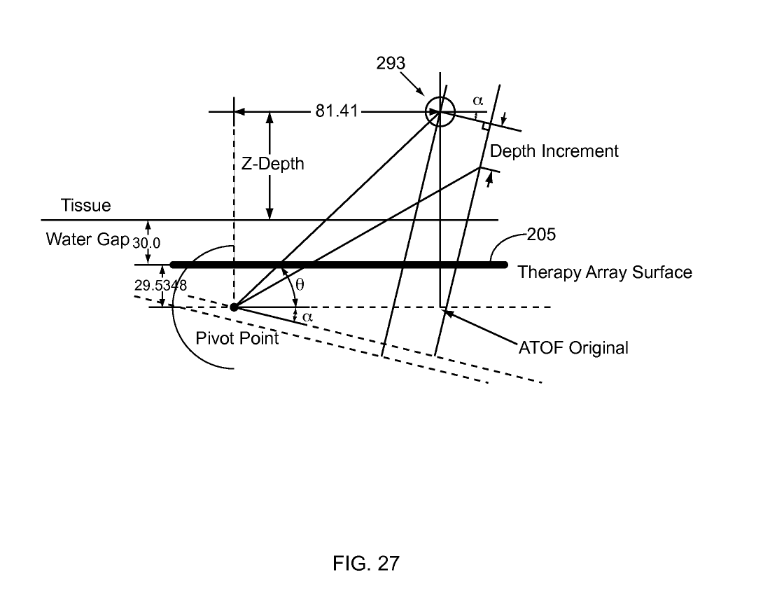

In one variation, during the initial ATOF calculation, the origin of the coordinate is placed at the tip of the array. When the array 205 is tilted, the ATOF depth will be deeper (see FIG. 27). In one approach, the step of “target depth minimization” comprises of adjusting the treatment applicator to make the array surface is as parallel to the skin as possible to minimize the gap between the skin and the transducer surface.

In another variation, target depth minimization comprises moving the applicator around, and the monitor the distance of the treatment transducer 205 from the target region 293 or the target area to locate a position for the applicator on the patient's body, where the distance between the treatment transducer array and the target is determined to be as small as possible (therefore, minimizing energy lost during treatment), while staying within the treatment window and avoid bone structures (ribs and spine) within the path of the therapeutic ultrasound field. In other words, the target depth minimization would minimize the therapeutic ultrasound focal depth under the conditions of having a clear acoustic access window. The target region or target area may be identified using a beacon signal or using an imaging device (e.g., CT, imaging ultrasound, etc.). The monitoring of the distance between the target and the treatment transducer array during the target depth minimization process, may be achieve through manual observation by an operator, or be conducted through an application running on the computing unit. The adjustment of the applicator to identify the position for target depth minimization may be done by manual adjustment of the applicator position, through feedback control by the computing unit if the base mover holding the applicator is configured with electromechanical actuator and/or robotic controls.

Optionally, the steps may also provide for bone finding, which allows the system to detect the interference of bone within the ultrasound path from the transducer array to the target region. Bone finding may be done through the use of an ultrasound imaging probe to locate bone boundaries and marking the window on the patient's body manually, or through the use of either the ATOF receivers and/or the therapeutic transducers receiving signals from the ultrasound beacon, and determine if particular pathway, between a transducer and the beacon, has been obstructed by bone.

As a further variation, the system may include a step of phase aberration correction, which allows the system to adjust the ultrasound phase and power emitted from each of the transducer within the treatment array to compensate for variations in tissue and material characteristics along the transmission path from the transducer to the target tissue. Based on the location and distance of the target region from the treatment transducer array, the system then calculates dosage of focus ultrasound for the treatment.

Dosage planning may include determining the number of dosage, the intensity of focus ultrasound for each dose, and the timing for delivery of each does. The dosage planning may also include calculating the phase control parameter and the power output control parameter for driving the treatment transducer array in order to achieve the desired power at the focal point of the treatment transducer array. The system initiates the treatment and delivers the focused ultrasound to the treatment region based on the planned dosage.

The operator may conduct post treatment evaluation to determine whether the treatment has been successful. Post treatment evaluation may include evaluation or measurement of one or more physiological parameter to determine if the treatment has been successful. For example, in renal denervation procedure, post procedure specific indicator of hormonal secretion of the kidney may be monitored. The patient's blood pressure may also be monitored over time to confirm reduction in blood pressure. In addition, intravascular (e.g. intravascular ultrasound imaging catheter) or non-invasive imaging (e.g., MRI, CAT Scan) may be applied to evaluate the condition of the treated renal artery.

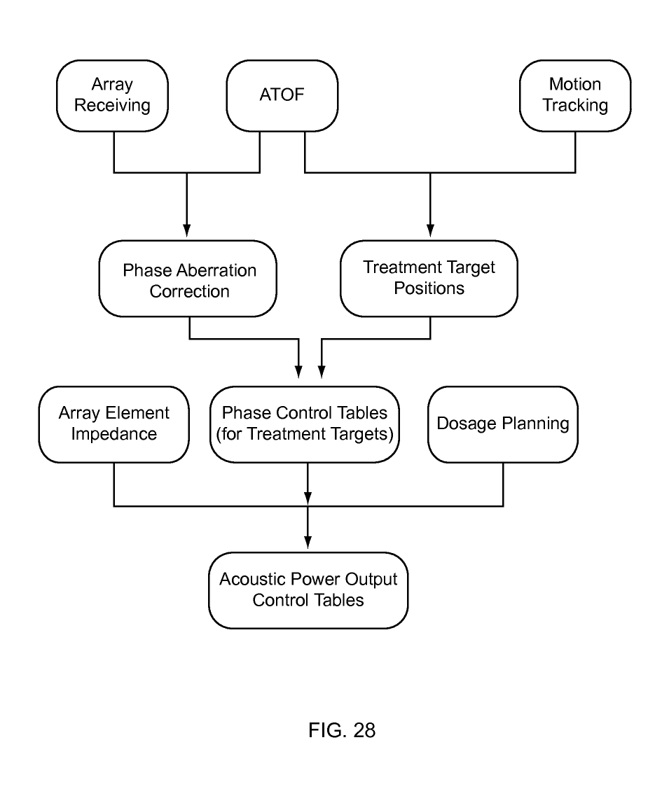

One example of the interconnection relationship 295 of the generator phase control and output power control is illustrated in FIG. 28. The output from the phase aberration correction and treatment target positions subsystems can be used as the input for the generator phase control subsystem. The outputs of the phase control, array impedance measurements, and dosage planning subsystems can be used as the input for the acoustic power control subsystem.

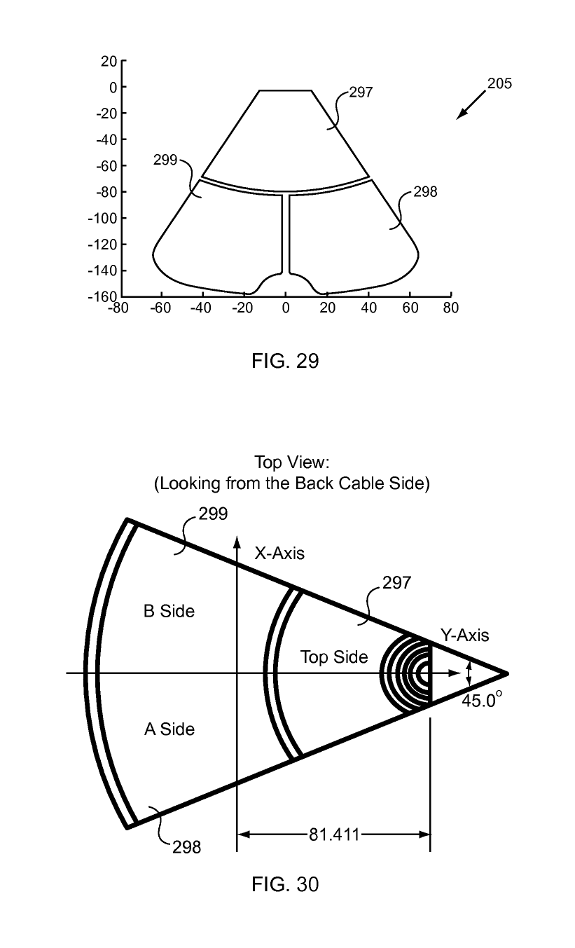

One variation of a geometric structure of the treatment transducer array 205 is shown in FIG. 29. The array is divided into 3 sections 297, 298, 299 by metal frames and electrode isolation cut. Referring to FIG. 30, in this example, there are 76 elements on the Top-Side 297, and 77 elements in both A-Side 298 and B-Side 299 sections. Each array transducer element is connected to a system channel which provides independent phase and power control of the element.

The characteristics of the acoustic field, such as focal position, power and power distribution can be modified by changing the control parameters (phase, power and time sequence) of each element. In one variation, A-Side and B-Side sections are connected together for integration of the system controls. In another variation, A-Side array elements and B-side array elements are driven by separate channels.

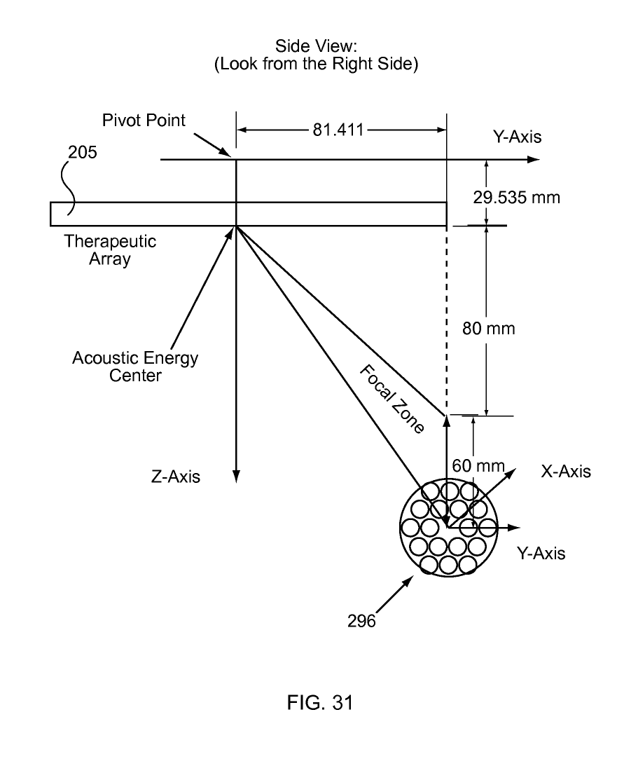

FIG. 31 is a graphical representation of a phased-array treatment transducer array 205 positioned over a treatment area, with the focal zone of the treatment array directed towards the treatment area. Multiple target regions within the treatment area 296 are shown. The geometry of the array 205, including the position of the pivot point (for moving the array to position the focal zone) and the acoustic energy center of the array are also shown.

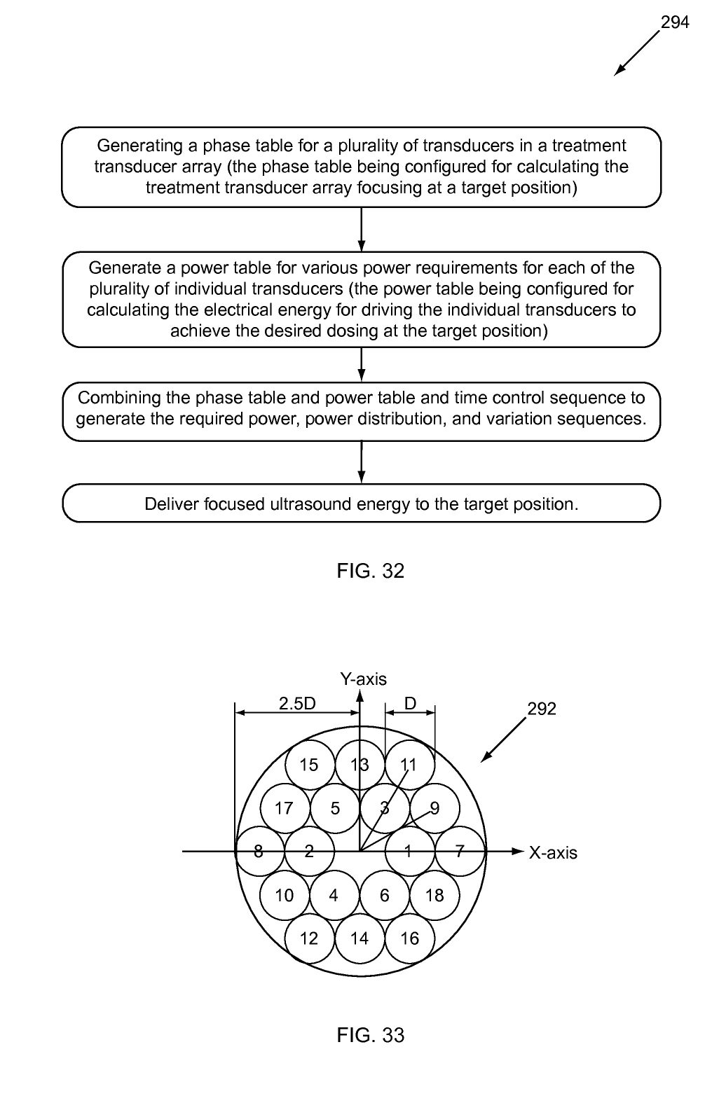

In one variation, the following steps 294 are followed to configure the system for improving the performance of the treatment transducer array in delivering a focus ultrasound energy field to the target region. First, generate a phase table for the various focal positions along the Z-axis, referring to FIG. 32. In one variation, the phase table generation is based on time delay. In another variation, the phase table generation is based on phase shift. Second, generate a power table for various power requirements for each individual transducer in the treatment array. Third, combine the phase tables and power tables, and time control sequences to generate the required power, power distribution and variation sequences. Evaluate the therapeutic array and system integration.

To generate the phase table, first determine the element geometric center of each of the transducers in the array. A geometrical center exists for each transducer element in each section. These geometrical centers of the elements can be determined when the therapeutic array structure is determined. In one variation, the array surface is assumed to be flat, and the Z₀ for all array elements shall be the same, such as zero in an ideal condition. The Z₀ can be also be used to compensate the variation between the therapeutic focus and the targeting depth measured by an ultrasound pulse/echo or imaging method. The A-Side and B-Side side is symmetric to the Y-axis. Therefore, the ring element position in A-Side side can be produced by multiplying “−1” to the X-axis position in B-Side section.

Next, the beam steering area and focal positions are determined. The beam steering area and focal positions are determined by the clinical thermal requirements and thermal dosage simulation. The focal area and focal positions can be determined by an optimization process. One proposed sample pattern 292 of focal position distributions is shown in FIG. 33. To have a flexible control of focal area and focal positions, 18 positions are selected to produce a pattern or mesh of acoustic power distribution for treatment (i.e., a total of 18 target regions). The circles with a diameter of D are packed in hexes shape. The center of the pattern is empty in this example, to minimize ultrasound energy exposure in the central portion of the targeted tissue, such as a blood vessel, while treating the tissue surrounding the blood vessel. All the focal positions are on XY plane and inside a 5D diameter circle. The depth of the focal position in Z-axis is determined from the ATOF position.

A phase table is generated for use in the calculation to determine the phase of electrical energy driving the individual transducers in the treatment transducer array, such that ultrasound field emitted through the various transducers in the treatment array reach a single focal point. By adjusting the phase of the ultrasound emitted through the individual transducers, the focal point of the treatment array can be adjusted along the Z-axis.

The phase angle of an element used to focus the element energy to a focal position in a 3D space can be calculated based on the example described below.

Given:
Target Z-Depth: Z_{T}
A Focal pattern in 3D space: (xᵢ, yᵢ, dzᵢ) (i=1, 2, 3, . . . , N)

Then,
The Focal Position in 3D space: (xᵢ, yᵢ, zᵢ) (zᵢ=Z_{T}+dzᵢ)

Where, dzᵢ=zᵢ−Z_{T} is the Z-axis offset of the focal position (z) relative to the target position Z_{T}. dzᵢ is used to optimize the focal positions in 3D space and make the focal patterns independent of Z-axis depth. The default value of the dzᵢ is zero, i.e. dzᵢ=0. Thus, the propagation time of ultrasound wave from an element to the target relative to the targeted focal Z-axis depth (Z_{T}),

$${T}_{i} = \frac{\sqrt{{( {x}_{i} - {x}_{0} )}^{2} + {( {y}_{i} - {y}_{0} )}^{2} + {( {Z}_{T} + {dz}_{i} - {z}_{0} )}^{2}} - {Z}_{T}}{V} \\ ( i = 1 , 2 , … N ) \qquad ( 1 )$$

TᵢV(xᵢ−x₀)²+(yᵢ−y₀)²+(Z_{T}+dzᵢ−z₀)²−Z_{T} (i=1, 2, . . . N)
V
   (1)

Where, the V is the velocity of the ultrasound propagating medium, which is assigned during the application. In one example, the following two default values are used,
V=1.4473 mm/us for water at 10°
V=1.540 mm/us for water at 37.5° (i.e., tissue velocity at human body temperature)

In one variation, the system uses the time delay method to control the treatment transducer array focusing at the desired target position. In another variation, the system uses the phase shift method to control the treatment transducer array focusing at the desired target position.

When time delay method to control the focusing of the therapy array is used, the relative time of flight delay between each element in the array with the element with the longest distance (i.e. the element located on ring 153) to the target is set to each system control channel.

When the phase angle is used in focusing control, the phase angle of an element with an operating frequency f can be calculated,

θᵢ=2πfTᵢ (2)

transfer the phase angle θ from radius to degree within [0°, 360°] for the system control,

$${θ}_{i}^{′} = [ \frac{{θ}_{i}}{2 π} - Int ( \frac{{θ}_{i}}{2 π} ) ] \times 360 = [ {fT}_{i} - Int ( {fT}_{i} ) ] \times 360 \qquad ( 3 )$$

all phase angles for the system focusing control can be calculated using the above formulation (1), (2) and (3).

The requirements and procedures to generate the gain setting values or voltage control parameters for each system channel when a total output acoustic power (P_{A}) or the total output electrical power (P_{E}) is required in the system, is discussed below.

In some variation, the electrical impedances and phases of a treatment transducer array on the operating frequency (f₀) is extracted from the test data measured using impedance measurement device. The impedances and phases of each element at the operation frequency are stored inside the system.

The operating frequency (f₀) is selected to be close to the resonant frequency through the system and array performance optimization procedures.

In some variation, an ultrasound transceiver with output drive circuitry driving the same voltage across all the output channels connected to the treatment array may be implemented. In such a system, pulse-width modulation may be used to vary the electrical energy driving the individual transducer in the treatment array, so as to achieve consistent power intensity across the face of the treatment array. For example, when a transceiver is configured to drive same voltage across all the channels, each of which is connected to one of the transducers in a treatment array, and the transducer size in the treatment array are non-uniform, pulse width modulation may be used to drive more electrical power to the larger transducer element, and less electrical power to the smaller transducer element, such that uniform power intensity can be achieved across the surface of the transducer array. In some variations, further adjustment may be made to take into account the distance from each transducer element to the focal point, and the absorption/interference within its path, such that each transducer element delivers the same power density relative to the focal point.

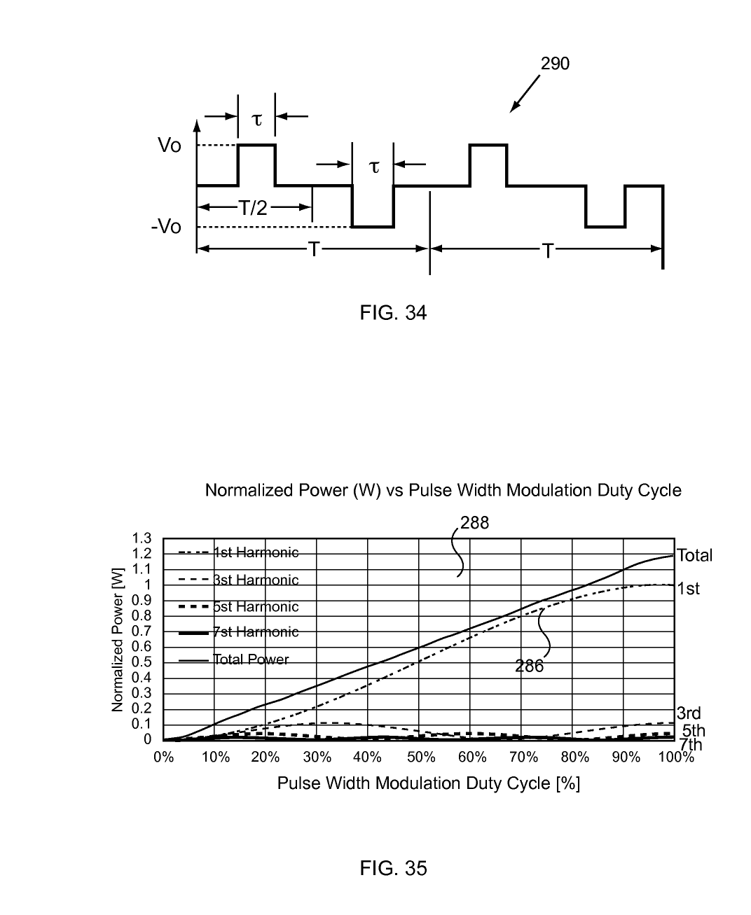

One example of system pulse width modulation, voltage control and ATOF transformer ratio determination are described below. The ultrasound transceiver module transmitting circuit can generate a pulse width modulated waveform 290, as shown in FIG. 34.

The waveform can be described as follows:

$$V ( t ) = ( \begin{matrix}0 & 0 \leq t < \frac{T}{4} - \frac{t}{2} \\ {V}_{0} & \frac{T}{4} - \frac{τ}{2} \leq τ \leq \frac{T}{4} + \frac{τ}{2} \\ 0 & \frac{T}{4} + \frac{τ}{2} < t < \frac{3 T}{4} + \frac{τ}{2} \\ - {V}_{0} & \frac{3 T}{4} - \frac{τ}{2} < t < \frac{3 T}{4} + \frac{τ}{2} \\ 0 & \frac{3 T}{4} + \frac{τ}{2} < t \leq T\end{matrix} ) \qquad ( 4 )$$

where, V is the required output voltage of the system and T is the pulse width of the transmitting waveform.

The V(t) is a period waveform with a period of T. The V(t) can also expressed in the frequency domain as the combination of infinite number of sin and cosine waveforms.

$$V ( t ) = {V}_{0} * \frac{4 τ}{T} {\sum }_{n = 1}^{∞} \frac{sin ( \frac{n π τ}{T} )}{\frac{n π τ}{T}} cos ( \frac{n 2 π t}{T} ) \\ n = 2 k - 1 ; k = 1 , 2 , 3 , … ∞ \qquad ( 5 )$$

Where, the Pulse Width Modulation Duty Cycle (PWMDC) is defined as following.

$$PWMDC = \frac{2 τ}{T} \qquad ( 6 )$$

Therefore,

$$\begin{matrix}V ( t ) = {V}_{0} * 2 * P W M D C * {\sum }_{n = 1}^{∞} \frac{sin ( \frac{n π * P W M D C}{2} )}{\frac{n π * P W M D C}{2}} cos ( \frac{n 2 π t}{T} ) \\ n = 2 k - 1 ; \\ k = 1 , 2 , 3 , … . ∞ \\ Or , & ( 7 ) \\ V ( t ) = {V}_{0} * \frac{4}{π} {\sum }_{n = 1}^{∞} \frac{1}{n} sin ( \frac{n π * P W M D C}{2} ) * cos ( \frac{n 2 π t}{T} ) \\ n = 2 k - 1 ; \\ k = 1 , 2 , 3 , … . ∞ & ( 8 )\end{matrix}$$

In applications where the treatment array is comprised of transducer elements of varying sizes, the element impedance of each of the transducer elements is different. Therefore, pulse with modulation would be adjusted such that same power density is achieved throughout all the transducer elements.

The pulse modulated waveform contains only the odd number of harmonics. The energy distribution among the harmonics 288 is shown in FIG. 35.

The amplitude of the 1^{st} harmonics,

$${V}_{1} = {V}_{0} * \frac{4}{π} * sin ( \frac{π * P W M D C}{2} ) \qquad ( 9 )$$

In some variations of the therapeutic ultrasound applications, only the 1^{st} harmonics energy of the pulse width modulated waveform will resonate with the therapeutic array and generate the desired acoustic energy. Other higher harmonics, such as 3^{rd}, 5^{th} and 7^{th} harmonics will generate heat that dissipates inside the array. In the example show in FIG. 35, the 1^{st} harmonics energy 286 is almost linearly related with the pulse width within the pulse width range from 50% to 85%. The higher harmonics energy is also at minimum in the same range. Therefore, in some variations, the pulse width is limited within the range from 60% to 75% for the therapeutic acoustic energy generation. In some other variations, the pulse width is limited within the range from 50% to 85% for the therapeutic acoustic energy generation. In yet other variations, the pulse width is limited within the range from 45% to 90% for the therapeutic acoustic energy generation.

An example of determining the electrical power distribution of array elements based on element impedance is described below. The electrical power (P) applied on each array element at its 1^{st} harmonics can be calculated from the output voltage (V) of the channel connected to the array element.

The impedance of each element treatment array may be different due to the variation of array element area, and different tuning inductor values. For example, the transducer elements in the array 205 shown in FIG. 29 have different impedance since each array element has different surface area due to the fan shaped structure of the array. In some variations, the transceiver system cannot adjust the voltage of each channel individually. Therefore, pulse-width modulation may be implemented to modify the energy driving each individual channel.

In one variation, once the output voltage is determined, the 1^{st} harmonics energy of the array elements can be adjusted by modulating the pulse width. Assume a total electrical power is going to be applied on the therapeutic array, and each element will be loaded with equal electrical energy density. Then, the electrical power applied on each array element is determined. The electrical voltage of each element can be calculated when its electrical impedance, phase angle, element area, pulse-width and required electrical output power are known.

In one example, it is assumed that the efficiencies of electrical energy to acoustic energy of array elements are about the same, when the array is manufactured. The equal electrical intensity on all array elements is about equivalent to the equal acoustic power intensity on the array surface. To generate equal electrical intensity on each element, a reference element with the average element area and average pulse width of 67.5% (i.e., middle vale between 50% and 85%) is selected. For other elements, the PWMDCᵢ shall be adjusted relative to the reference element.

In one variation,

$$P W M D {C}_{i} = \frac{2}{π} * {sin}^{- 1} [ \sqrt{\frac{{A}_{i} * \vert {Z}_{i} \vert}{cos {ϕ}_{i}} * \frac{cos {ϕ}_{m}}{{A}_{m} * \vert {Z}_{m} \vert}} * sin ( \frac{π * P W M D {C}_{m}}{2} ) ] \qquad ( 9 )$$

If, PWMDCᵢ≦0.5 then, PWMDCᵢ=0.5
If, PWMDCᵢ≧0.85 then, PWMDCᵢ=0.85

PWMDCᵢ is the PWMC of the specific transducer element; Aᵢ is the surface area of the specific transducer element; Zᵢ is the acoustic impedance magnitude of the specific transducer element; φᵢ is the phase angle of the impedance of the specific transducer; PWMDCₘ is the PWMC of the reference transducer element; Aₘ is the surface area of the reference transducer element; Zₘ is the acoustic impedance magnitude of the reference transducer element; φₘ is the phase angle of the impedance of the reference transducer.

Equation (9) demonstrates that the pulse width of the individual transducer element can be determined based on each specific transducer element's (a) surface area, (b) acoustic impedance magnitude, and (c) phase angel of its impedance, relative to the reference transducer element's (a) surface area, (b) acoustic impedance magnitude, and (c) phase angel of its impedance, and (d) the pulse width of the reference transducer element.

One variation for determining the electrical power distribution of treatment array elements based on the element contribution at focus is described below. The contribution of each element at the focus can change due to the element size, distance to the target, inter-path tissue attenuation, and array element to element variation in the manufacture process. One way to compensate for these variations is to use a hydrophone located at the focal position to measure the pressure (or energy) contribution from each element. Then the pulse width of each control channel is adjusted to the proper value. The hydrophone measures pressure from an array element at its 1^{st} harmonics is proportional to the output voltage of the channel connected to the array element. The pressure can be adjusted or compensated to the proper value using the PWMDC.

### III. Treatment Transducer Array

The therapeutic ultrasound system may be configured with various treatment transducer array designs. The array geometry may be shaped to create a specific ultrasound field pattern, depending on the treatment indication and/or clinical application. The system and the transducers may be further configured to deliver specific ultrasound energy to a focal point. In one variation, the therapeutic ultrasound system is configured, such that the ultrasound power intensity at the focus of the treatment transducer array is in the range between 250 W/cm² and 350 W/cm². In another variation, the ultrasound power intensity at the focus of the treatment transducer array is in range between 200 W/cm² and 400 W/cm². In yet another variation, the ultrasound power intensity at the focus of the treatment transducer array is in range between 150 W/cm² and 450 W/cm².

In another design variation, the system is configured, such that selective transducers in the treatment transducer array may be turned off to adjust the ultrasound field pattern. For example, in case of patients with a smaller access window, the system may turn off individual rings of the semi-annular array in the treatment transducer array to accommodate the smaller window.

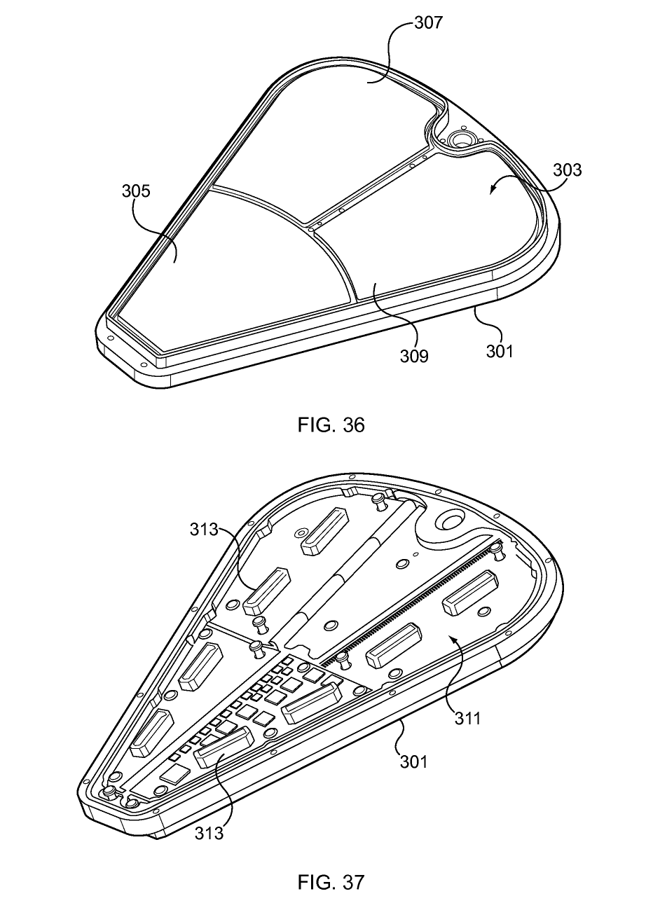

In one example, the transducer unit comprises a base frame 301 for housing the transducer array elements, as shown in FIG. 36. In this example, the front face 303 of the base frame includes three islands 305, 307, 309 for supporting three separate transducer array sections or lobes. The back 311 of the base frame 301 (see FIG. 37) includes a plurality of electrical connectors 313, which are configured for coupling electrical connections to the individual transducer elements to be positioned on the front of the base frame.

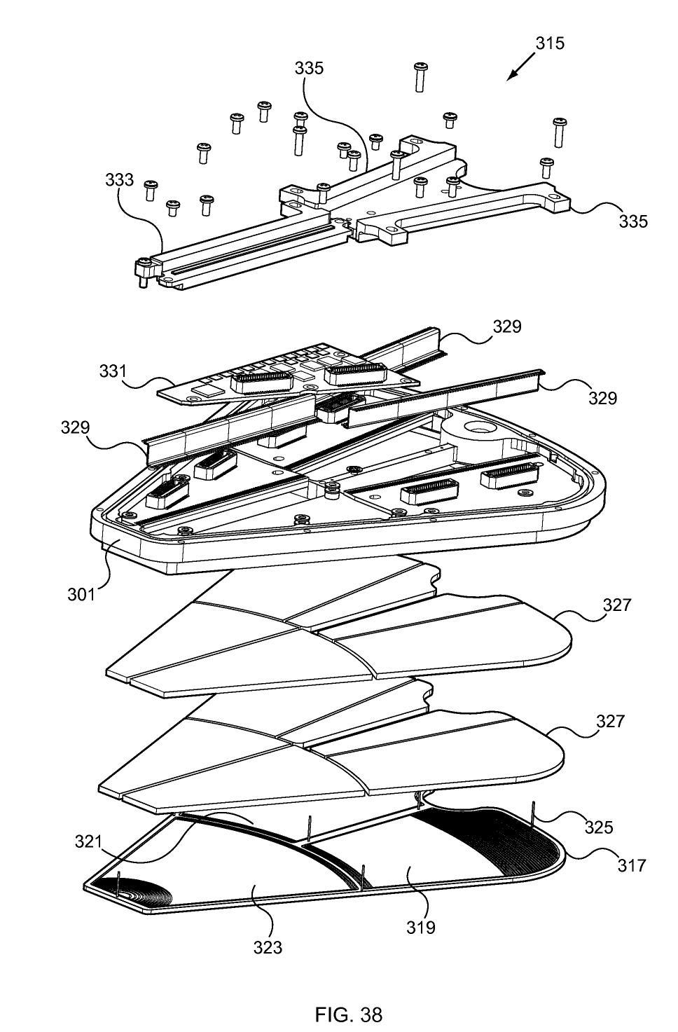

FIG. 38 shows an exploded view of the transducer array unit 315 various components that are connected to the base frame 301. The transducer unit comprises: a transducer frame 317; the left lobe of the PZT treatment transducer array 319; the right lobe of the PZT treatment transducer array 321; the top lobe of the PZT treatment transducer array 323; a plurality of pins 325 for grounding the transducer array; a plurality of thermal pads 327; flexible jumpers 329 for board to board interconnections (for transmitting power and receiving electrical signals to and from the transducer array); printed circuit assembly for interconnection with the transducer array elements; printed circuit assembly for the pre-amplifiers 331 for the ATOF receiver transducer elements; brackets 333, 335 for coupling the transducer unit to a mover or actuator, which can direct movements and position of the transducer unit.

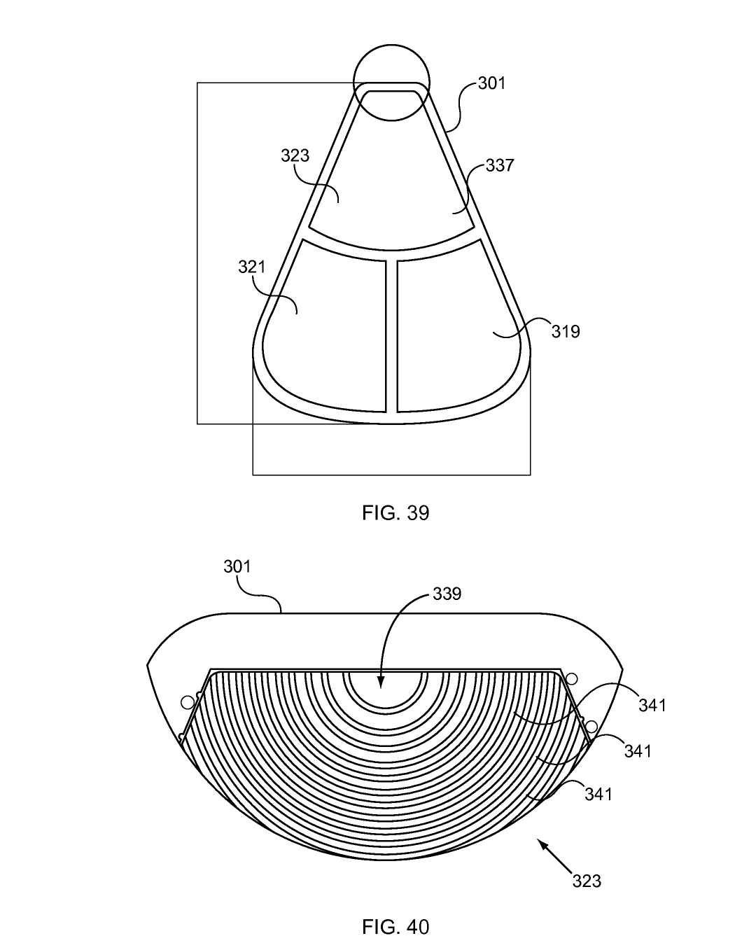

FIG. 39 is a top down view of the face of the transducer array unit 315. In this variation, the treatment transducer array 337 forms a fan shape (e.g., partial circular shape, pie shape, semi-annular, etc.). The fan shape comprises three separate fan shaped array 319, 321, 323 subunits positioned in a staggered formation. Each of the fan shaped subunit comprises a plurality of partial ring shaped ultrasound transducers elements that are arranged in a concentric pattern to form the transducer arrays for the subunit. An expanded view of the tip portion of the top lobe 323 of the array subunit is shown in FIG. 40, which illustrates the concentric pattern 339 of the transducer array elements. As shown, the surface area of the transducer elements 341 increases as distance from the center of the concentric circle increases.

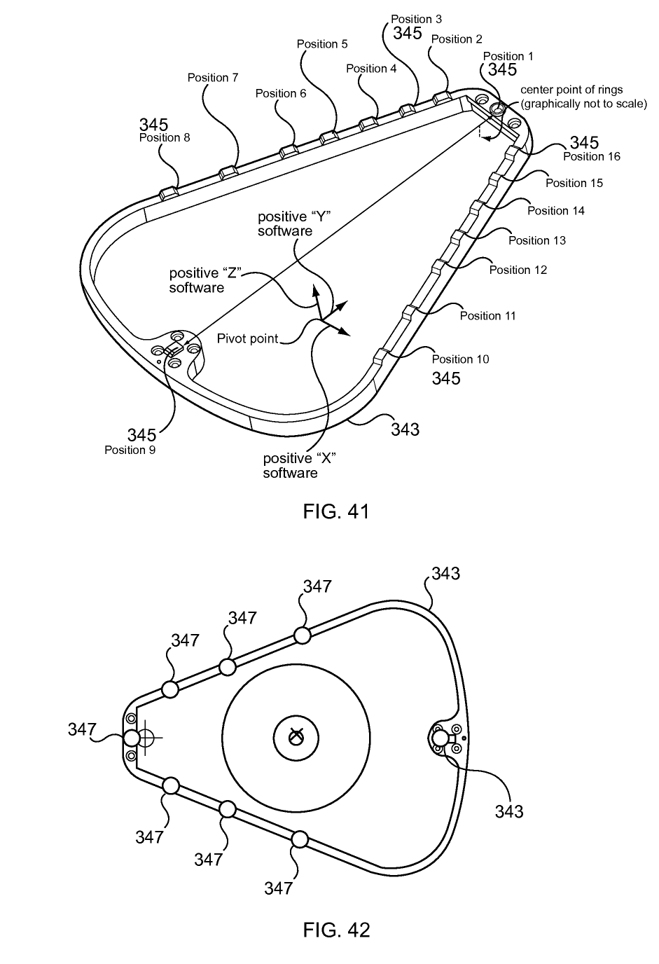

FIG. 41 shows a frame 343 that houses the AFTOF receiver transducer array elements 345. In this example, there are sixteen transducer elements. The system may utilize all sixteen elements or only utilize a fraction thereof, to locate the position of the tracking beacon. FIG. 42 is a top view of the transducer unit, showing one arrangement where eight 347 of the transducers are used for tracking. This arrangement may allow the system to turn on and off selective receivers as needed. For example, if a receiver malfunctions, one or more of the other un-used receivers may be activated.

In this example, the AFTOF receiver transducer array elements provide the ultrasound detections for the Acoustic Time of Flight (ATOF) and tracking functions of the therapeutic ultrasound system. Using ultrasound triangulation, the system can locate the target tissue, and use the target position information relative to the treatment array to position the applicator of the treatment module on the back of the patient, and to track the position of the target during therapy, to keep the focus of the therapeutic ultrasound on the target.

The ATOF subsystem provides one approach for the system to locate the beacon inside the patient's body relative to the applicator and/or the treatment transducer array. For example, the beacon may be placed in a renal artery of a patient, which is targeted for treatment. Since the beacon resides at the renal artery, ATOF can provide position information to assist the operator in aligning the therapy beam from the applicator/treatment array to the renal artery prior to dosing.

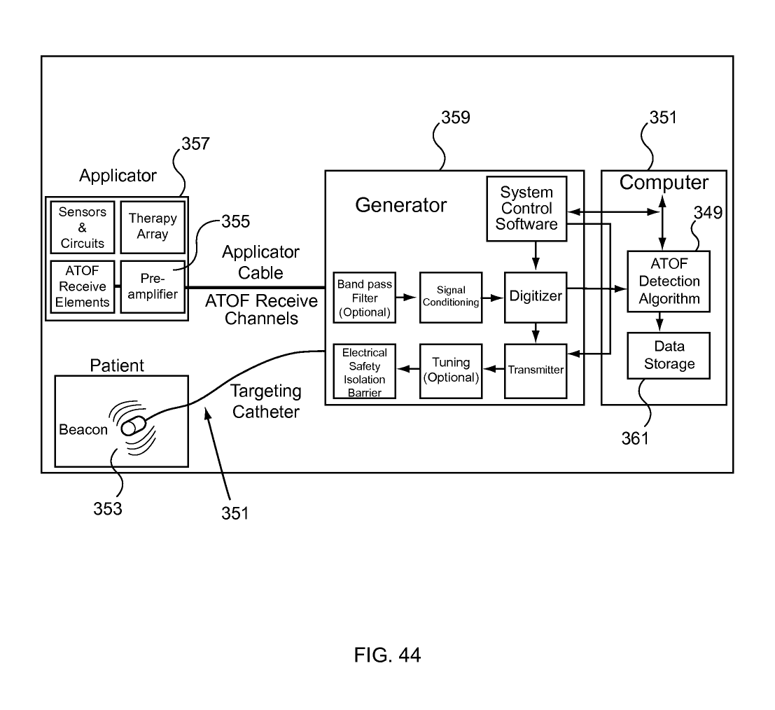

In one example, the ATOF subsystem includes four major function blocks, ATOF electronic system, the targeting catheter, ATOF receive elements, target computation, and data logging. These 4 function blocks are integrated together to locate the beacon in the tissue relative to the position of the applicator, which contains the ATOF receivers and treatment array. FIG. 44 illustrates additional details of the components that are included in an ATOF subsystem. In one variation, the ATOF detection algorithm/process 349 is configured to belong to part of the computational environment subsystem 351.

When ATOF is active, the transmitter sends an electrical pulse down the targeting catheter 351 to the beacon 353. The beacon converts the electrical stimulus to a pressure wave that propagates in the tissue and towards the applicator 355.

ATOF receiver elements may be positioned at known positions on the applicator. An RF electrical signal is generated at the receiver elements as the propagating pressure wave from the beacon hits the receiver surface. The electrical signal from the receiver elements are amplified by pre-amplifiers 355 inside the applicator 357, and then connected to the generator 359 through the applicator cable. In one variation, inductive tuning of the receiver elements is applied before the pre-amplifiers to improve signal-to-noise ratio. The RF signals and the calculated x, y, and z positions may be stored in the generator (data storage 361) or another external computer for further review after the treatment.

A targeting catheter 351, which carries a beacon 353 at the distal end, may be inserted down the sheath into the target tissue, such as the renal artery of the patient. The distal end of the targeting catheter contains an ultrasound transducer, such as a piezoelectric device, that can resonate in various modes.

Ultrasound receiver elements 347, such as PZT transducers, are position on the applicator at known positions relative to the therapy axis. An example is shown in FIG. 42. These elements detect the pressure wave transmitted from the beacon. The total time difference, when the pressure wave was detected, to when the transmit event occurred, is related to the total distance the element is from the beacon. This distance information is combined with distance information from other elements to determine the beacon location relative to the applicator.

An electronic circuit is used to excite the beacon in the targeting catheter. The transmitter pulse duration, pulse frequency, and pulse amplitude may be varied to improve the ATOF performance. Multiple transmissions may be used to improve the signal-to-noise ratio and algorithm/calculation performance.

An electrical isolation transformer may be used as an electrical safety barrier between the ATOF system and the patient. In one variation, the isolation transformer satisfies the IEC electrical safety standard for medical device.

An electronic preamplifier, integrated in the applicator, amplifies the analog signals from the ATOF Receive Elements. The location of the preamplifier may minimize signal-to-noise ratio degradation if the transducer elements were directly coupled into the applicator cable.

An analog-to-digital converter samples the detected RF data, which can be used in the ATOF detection calculation in determining the location of the beacon. The number of bits, dynamic range, and sampling rate may affect the overall ATOF performances.

The ATOF detection algorithm may use the RF data of all the ATOF receivers from digitizer, and detect the time-of-flight information from the beacon to each of the receivers. Then the system then calculates the beacon position (x, y, z) relative to the applicator based on the triangular principle, and the time of flight information of the receivers. A computer with data storage memory may be used to store the detected beacon position (x, y, z), and the raw RF data from each ATOF receivers, for future review and error analysis.

In one variation, the ATOF subsystem for the therapeutic system includes components in the generator, applicator, targeting catheter and data logging.

In one variation, the system is configured with the following parameters and dependencies. Fluid coupling interface, between therapeutic array surface to patient skin, has a thickness that may vary from 10 mm to 25 mm. The energy loss passing the applicator membrane is no greater than 5%. The reflection coefficient is less than 30 dB (two-sides) when coupled to a medium with an acoustic impedance of 1.5 MRayls, such as water. In some variations, the propagation velocity may be in the ranges from 1.48 mm/μsec to 1.54 mm/μsec.

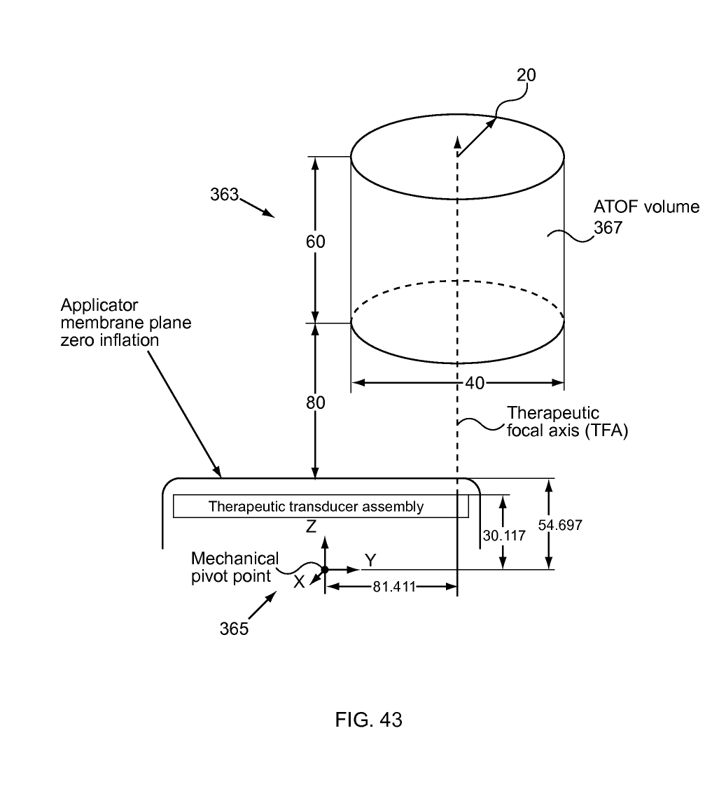

In one variation, the system is configured with at least the following focused ultrasounds delivery parameters. The shallowest target is 80 mm below skin-line. The deepest target is 140 mm below skin-line (see FIG. 43). The average attenuation coefficient in inter-path tissue is no greater than 0.9 dB/(MHz cm).

In one configuration, the ATOF receivers are positioned as shown in FIG. 41, and the ATOF coordinate system is the same as the therapeutic beam coordinate system 363 shown in FIG. 43. The positions of the ATOF transmitter, or the beacon, are defined as the center position of the beacon relative to the center of the ATOF coordinate system (mechanical pivot point). In this configuration, the origin of the ATOF coordinate system is at the mechanical pivot point 365, which is 81.411 mm from the therapeutic axis, and 29.535 mm below the surface of the treatment transducer array. The orientation of the beacon is defined by three rotation angle along the three axes (X-axis, Y-axis and Z-axis).

Sixteen ultrasonic receivers (i.e., location sensors) for use in ATOF targeting and tracking are incorporated on the treatment array base frame of the applicator to facilitate location of the therapeutic focal position. ATOF receiving elements may be manufactured and tested separately and then integrated into the therapeutic transducer assembly (i.e., treatment transducer unit). In one example, the sixteen ATOF receivers 345 are positioned on a frame 343, which is then attached onto the treatment transducer base frame 301. The positions of the sixteen ATOF sensors 345 are shown in FIG. 41. In one variation, the diameter of the ATOF receiver element is approximately 1.2 mm.

In one variation, the ATOF subsystem is configured with the following parameters. The ATOF subsystem calculates the position of the beacon (x, y and z) relative to an ATOF defined coordinate system for a single beacon moving from 0 to 25 mm/sec. ATOF positional information is measured for accuracy and characterized over the “ATOF Volume” 367 (FIG. 43). In one variation, the time required to initiate an ATOF measurement, acquire the signals, digitize, and calculate the position is less than 50 msec. The ATOF position data is recorded by the system. In one configuration, at least 8 ATOF receivers (for example the eight receivers 347 shown in FIG. 42) are mechanically located in the positions as shown in FIG. 41 with, for example, a mechanical accuracy of 0.15 mm in X-axis and Y-axis. An electrical isolation transformer is used as an electrical safety barrier between the ATOF system and the patient. The isolation voltage between the primary side and secondary side of the isolation transformer is at least 1.5 KV. In this example, there are at least 8 preamplifiers, which are used as the first analog amplification stage for the ATOF receiver elements, and these amplifiers reside on the treatment transducer array assembly. The preamplifiers has an amplification gain of at least ×25 with channel to channel variation less than 5% within the frequency range from 0.5 MHz to 4 MHz. The receiving analog circuit is −6 dB bandwidth from 0.5 MHz to 4 MHz. The ATOF receive channels from the preamplifiers is connected to the generator through the applicator coaxial cable(s). The digitizer offers at least 8 bit resolution per sample. The sample rate of the digitizer is at least 20 MHz. The digitizer has a trigger stability of less than 25 nsec.

In one configuration, the ATOF detection algorithm is configured with the following parameters. Up to 8 signals per receiver element are able to be averaged. The beacon position (x, y, z) and the raw RF data can be stored for further review. The data is recorded statically (no transducer movement), dynamically (moving applicator), and/or while tracking.

In one variation, the actuator, coupled to the treatment array assembly of the applicator, comprises two controlled motors that tip and tilt the treatment array assembly around a mechanical pivot point, so as to facilitate tracking a moving volume of tissue during a therapy exposure. A control system reads ATOF positional data or position information from the imaging array sub-unit, and directs servo controls to maintain the Therapeutic Focal Axis (TFA) orientation, such that it points at the moving target volume.

In one example, while tracking at target speeds of up to 25 mm/sec, ATOF positional readings show the X and Y distances from the TFA to be zero. Tracking performance of the therapeutic system can be characterized with regard to tracking error, as measured by ATOF over the ATOF volume and target speed range.

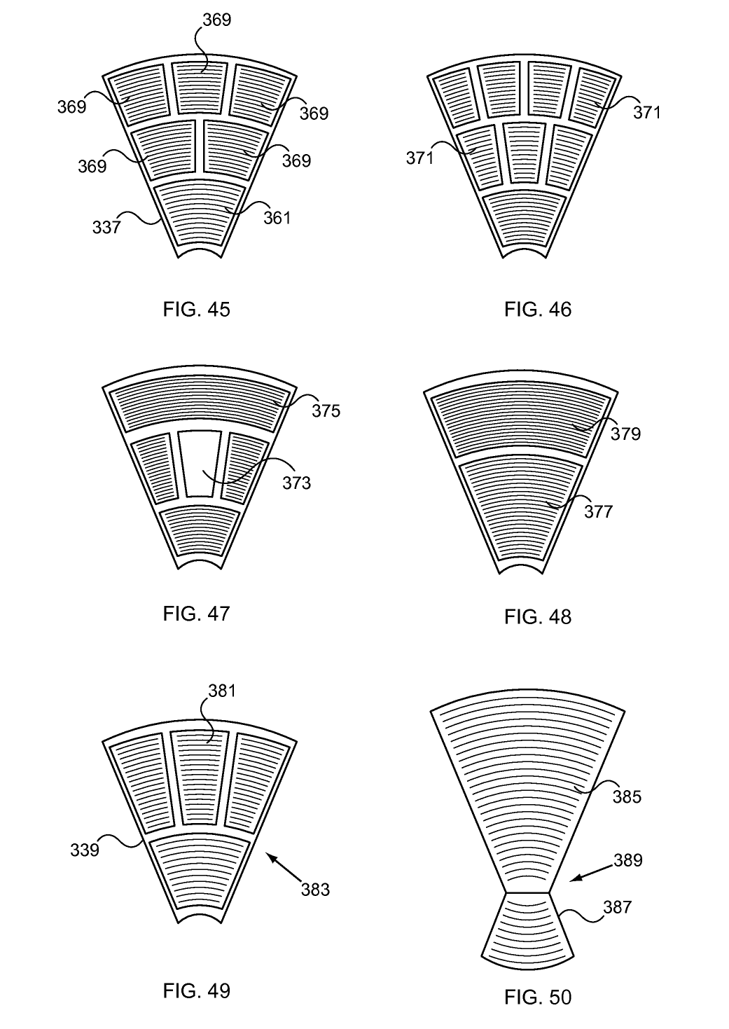

Referring to FIGS. 45-50, various other fan shaped configurations of transducer array designs are shown. These designs may allow transmission of a focused ultrasound wave towards a position off the central axis. Central axis is defined as the perpendicular axis that extends from the weighted center of the surface area on the face of the transducer array. This may allow the focus axis to be shifted towards the tip (or narrow portion) of the fan shaped array. FIG. 45 depicts an array with six sub lobs 369 configured in a staged formation of 1-2-3 configuration. Each of the lobs 369 comprises concentric rings (i.e., partial rings) of transducers. In one variation, all the transducers on the various lobes 371 share the same center for their concentric ring patterns. FIG. 46 shows an staggered 1-3-4 configuration. FIG. 47 shows a staggered configuration where the central lob 373 is concaved. In one variation, the concaved profile is arranged such that the transducer arrays on the surface of the concave surface faces a common focal point. In one variation, the central lobe 373 is used for imaging. In another variation, the top lobe 375 is used for imaging. FIG. 48 depicts an array arrangement where the first (lower) lobe 377 is formed of concentric partial rings, where the second (upper) lobe 379 is in a matrices arrangement (or diced arrangement), such as column and row format. The upper lob 379 may be configured to serve both as therapy ultrasound delivery transducer array, and as an imaging detection array for locating and tracking the position of the target tissue. FIG. 49 depicts another staggered fan shaped configuration with four lobes, with the center upper lobe 381 transducer array 337 forming a matrices arrangement, while the arrays on the rest of the lobes comprises concentric ring patterns, with the center of the concentric ring located at the narrow end 383 of the fan shaped profile. In one variation, the lobe with the matrices arrangement is configured for imaging application, while the rest of the lobes are configured for therapy application. In yet another variation, the lobe with the matrices arrangement is configured for both imaging detection and therapy delivery. FIG. 50 depicts a dual fan profile, where the focal axis of the concentric rings from the two fans 385, 387 meet at on an axis extending from or around the interface 389 of the two fan shaped transducer arrays. In another variation, one of the fan shaped array is configured with a tight focus and used for therapy, while the other one is configured with a broader focal area, and used for imaging around the focal point of the first array.

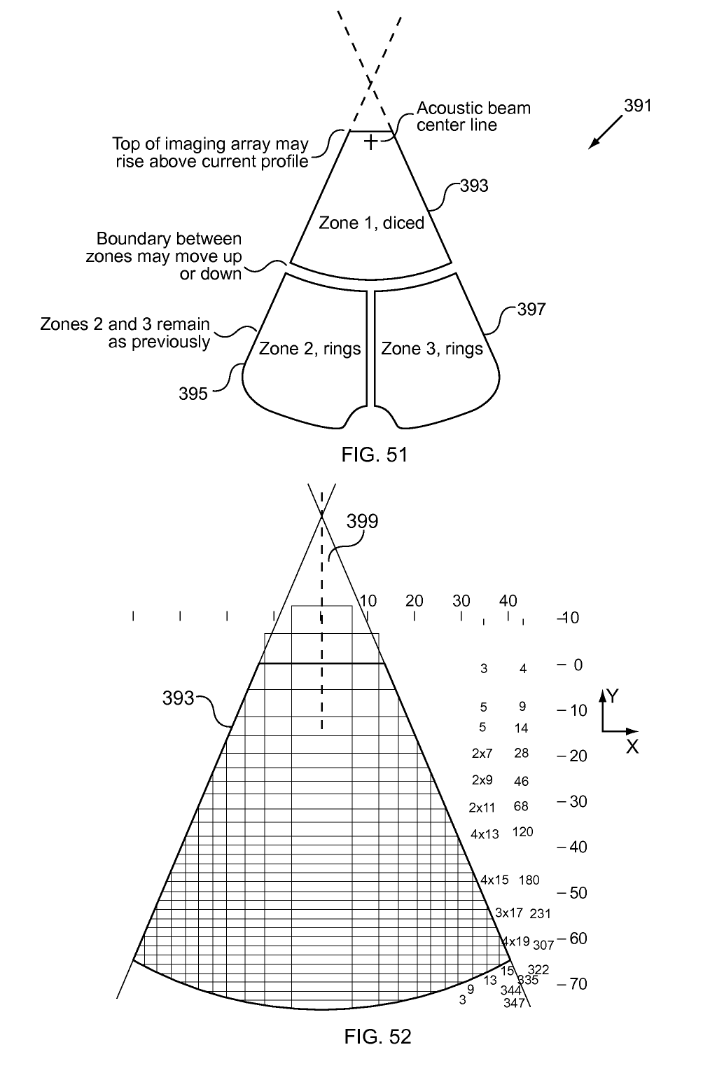

Referring to FIG. 51, another example of a fan shaped treatment array 391 is shown. In this configuration, the treatment array 391 comprises three lobes. Zone 1 393 comprises transducers in the diced configuration. The aperture configuration for Zone 1 393 is shown in detail in FIG. 52. The diced array of transducers may be evenly spaced, on unevenly spaced. In the variation shown in FIG. 52 the array elements are evenly paced on the X direction, but unevenly spaced in the Y direction, with the transducer elements closer to the mid-line having larger surface areas, and the array elements further away from the midline 399 having smaller surface areas. Zone 2 395 and Zone 3 397 comprises ring shaped (i.e., partial rings or annular shapes) transducer arrays. In some variations, Zone 1 is diced to 1 mm increments. In some other variations, the ground plane for Zone 1 is different from that of Zones 2 and 3. Zone 1 may be configured with lower bandwidth than Zone 2 and 3, when Zone 1 is configured primarily for axial imaging. In other variations, Zone 1 is used for both imaging and therapy delivery, while Zone 2 and 3 are used for therapy delivery only. In yet another variation, all three zones are configured with the capability for both imaging detection and therapy delivery. In some variations, the imaging frequency and the therapy frequency are configured to be the same, for example 0.95 MHz. In some other variations, the imaging frequency and the therapy frequency are distinct from each other.

To drive the transducer array for both imaging and therapy applications, one or more multi-channel ultrasound transceiver may be used. In one example two transceivers, one with 154 channels for therapy only, and the other with 347 channels for imaging and therapy, are used. In one variation, the imaging array is configured for at least a 2 cm×2 cm field of view. In one variation, the image array is configured with 6 db on edges of the array. Transceivers with more channels and/or additional transceivers may be implemented to support larger field of view.

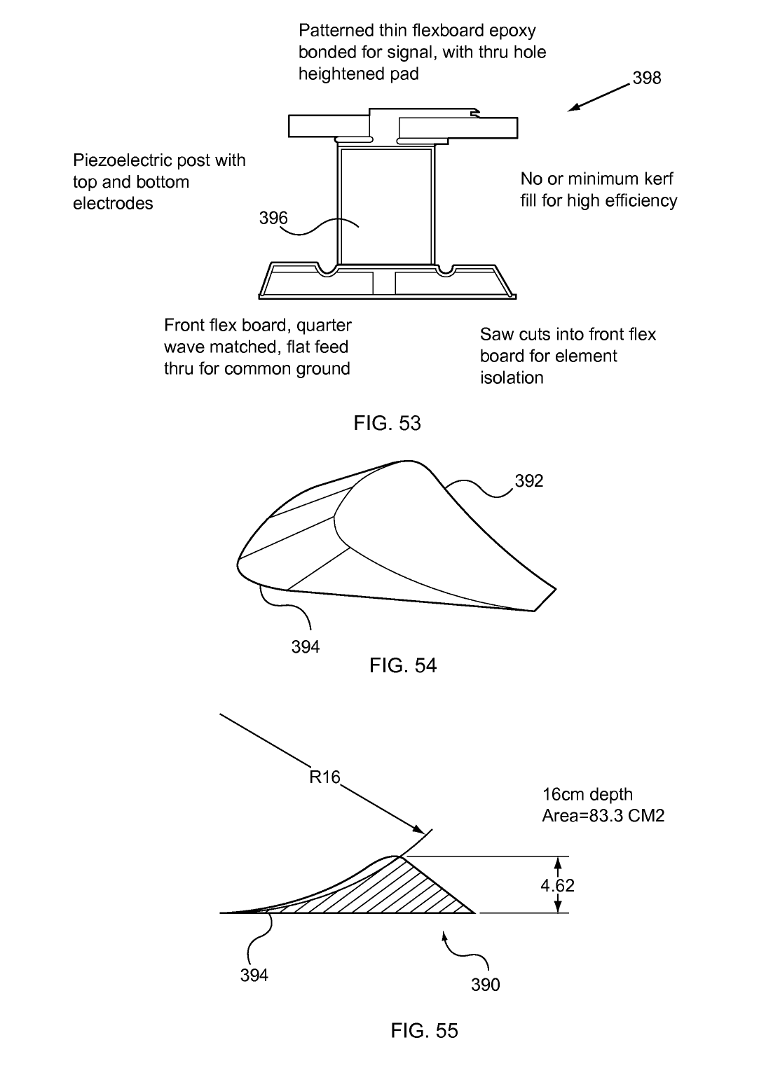

Referring to FIG. 53, one example of an elemental design 398 for an individual transducer in a therapeutic array is shown. An approach to manufacturing the therapeutic array comprises utilizing these individual elements 398 and placing each in a substrate connected together but partially or completely independently controllable from one another. The following is an embodiment of a manufacturing method to produce this single element of a larger therapeutic transducer: on a half wavelength thick ceramic plate, an electrode is plated on both sides; a second electrode is placed on a rigid board, a quarter wavelength thick; a blind feed is drilled through and optionally pre-diced/etched for a pattern or multitude of individual transducers. The plate and the board are then laminated together. The diced array and the flex board are then laminated with low viscosity epoxy. In a preferred embodiment, there is no kerf fill, or there is minimal kerf fill, so that there is isolation between the transducer elements 396.

In another design variation, the transducer array 394 is configured with a concaved surface profile 392, such as the one shown in FIG. 54. This particular design variation combined a fan shaped profile with a concave surface. FIG. 55 depicts a cross-sectional view 390 of the array 394 shown in FIG. 54.

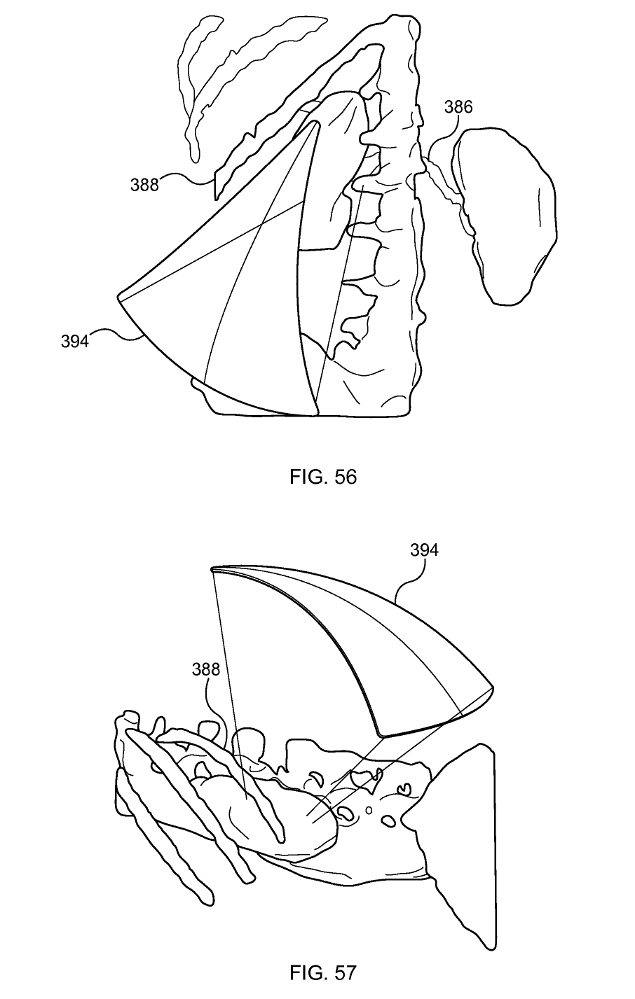

Various array designs disclosed herein, such as fan shaped profile with concentric ring elements, and the fan shaped or pie shaped design with concaved surface, can facilitate delivery of ultrasound from a position on the surface of the patient's body where there is no obstruction (within an ultrasound window) into a target located beneath an obstruction, such as the ribcage. FIG. 56 is a graphical representation, illustrating the delivery of focused ultrasound energy from an ultrasound array 394 located on a treatment window and propagating the ultrasound energy to a focal point located underneath the ribcage 388. In this example, the target is located around the renal artery 386. FIG. 57 shows a side view of the same arrangement illustrated in FIG. 56.

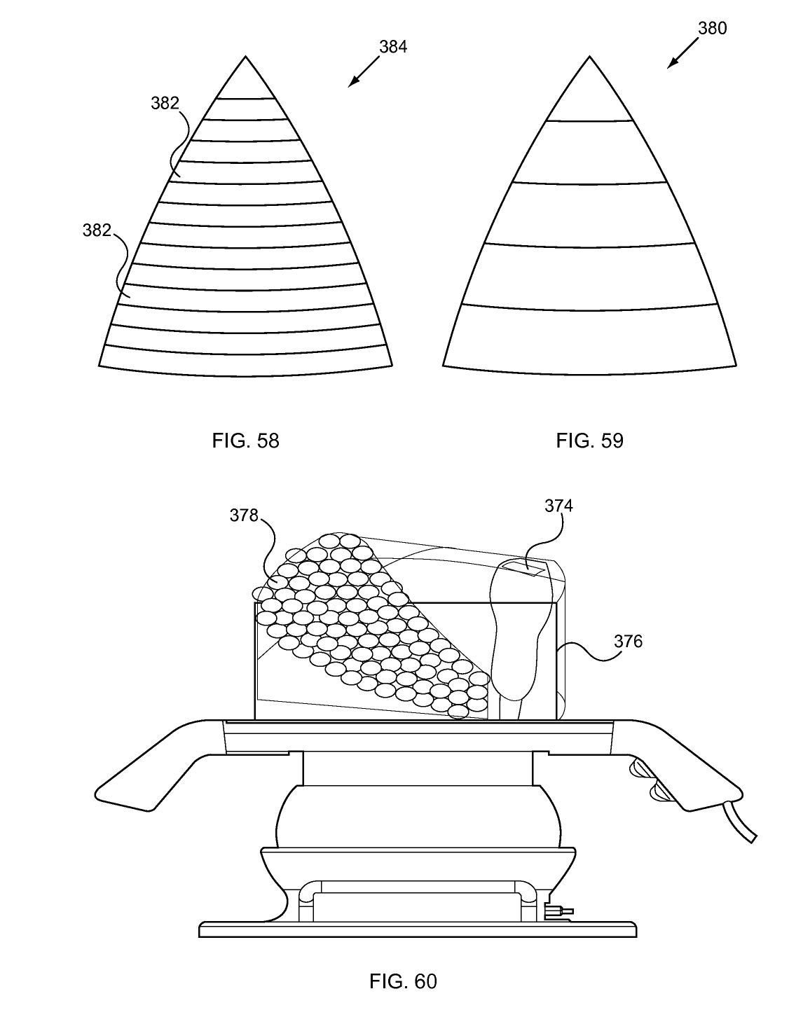

Referring to FIG. 58, another variation of a fan shaped transducer array design 384 is shown. In this variation, the array elements 382 are partially curved in a concentric arrangement along the length of the array, from the narrow portion of the fan shape to the broader portion of the fan shaped profile. FIG. 59 shown another arrangement with diced or matrices arrangement. In the particular design shown, the top portion 380 is arranged in a staggered arrangement. The design shown in FIGS. 58 and 59 may be implemented as a flat surface or a concaved surface array profile.

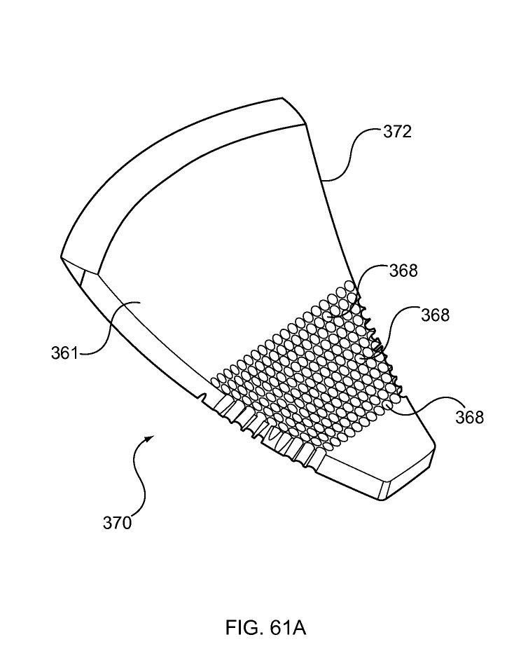

FIG. 60 shows a concaved profile transducer array 378 integrated in a treatment module 376. In this design, a separate imaging transducer array 374 is incorporated in the module 376 to provide target imaging and tracking. In one variation, the imaging transducer is configured to provide a 3-D image of the target region, for example a 2D transducer array may scan a volume to produce a 3D image. In another variation, a 2D ultrasound transducer is used, and the transducer head is configured such that it can be rotated and/or displaced with an actuator or manually coupled to the base of the transducer, such that a 1D transducer array structure would scan and detect a 2D image. One of ordinary skill in the art having the benefit of this disclosure would appreciate that the 2D or 3D ultrasound imaging detectors may be applied with the combinations described herein.

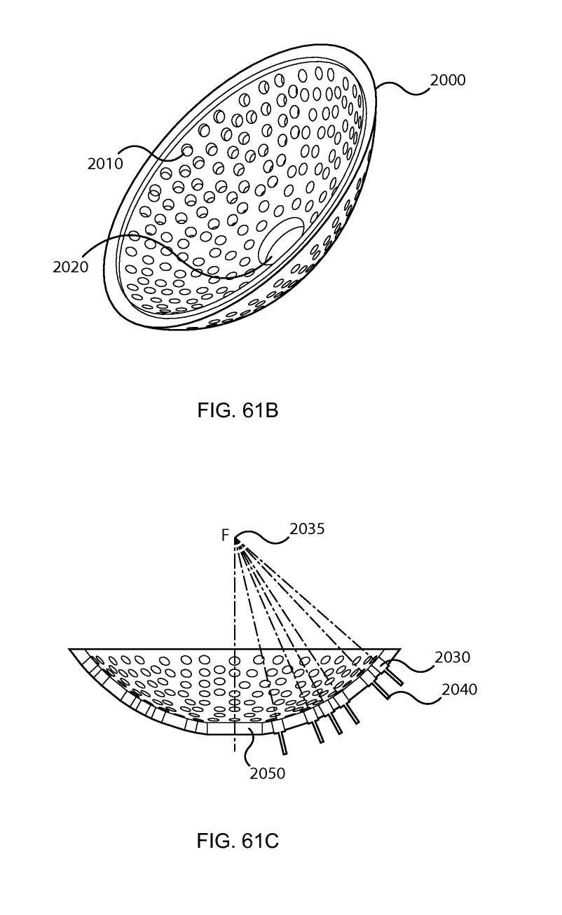

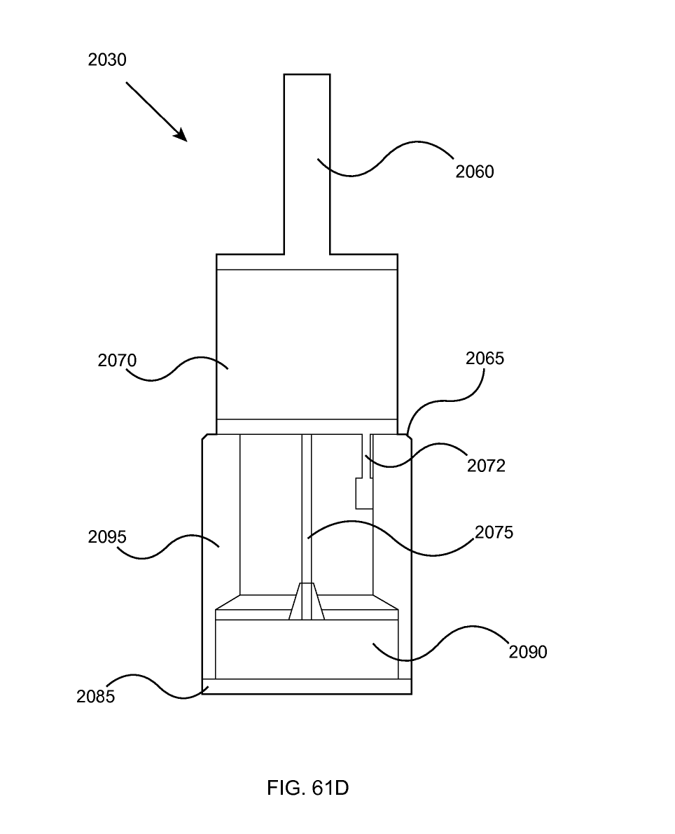

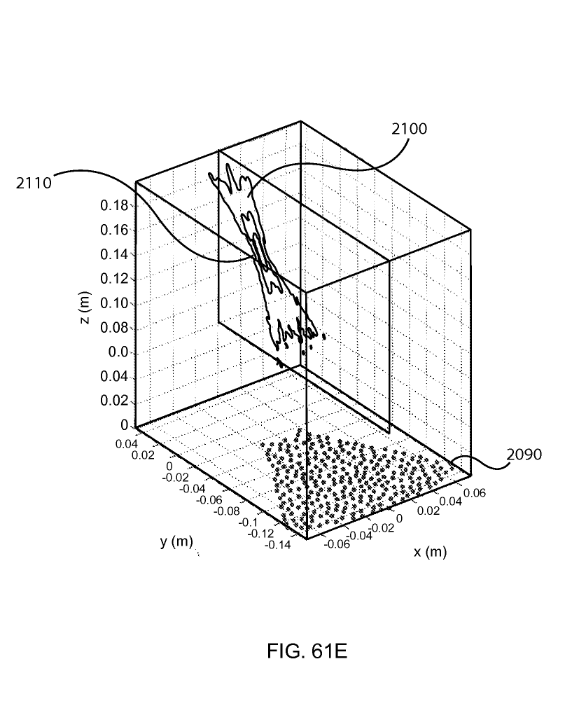

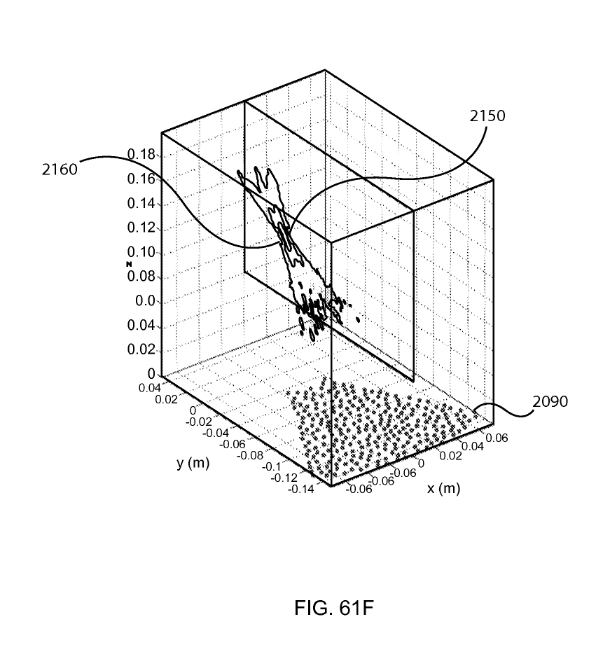

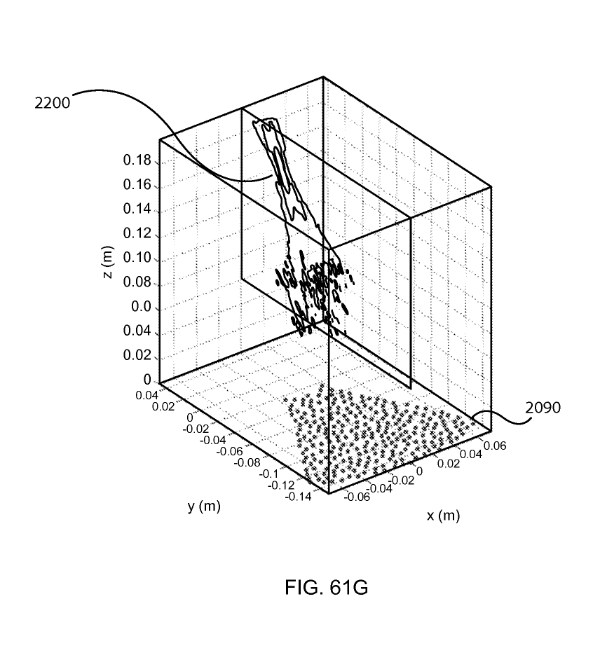

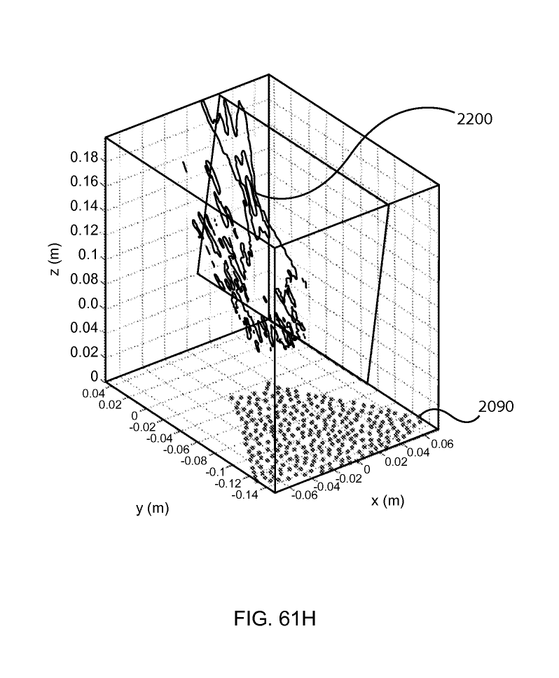

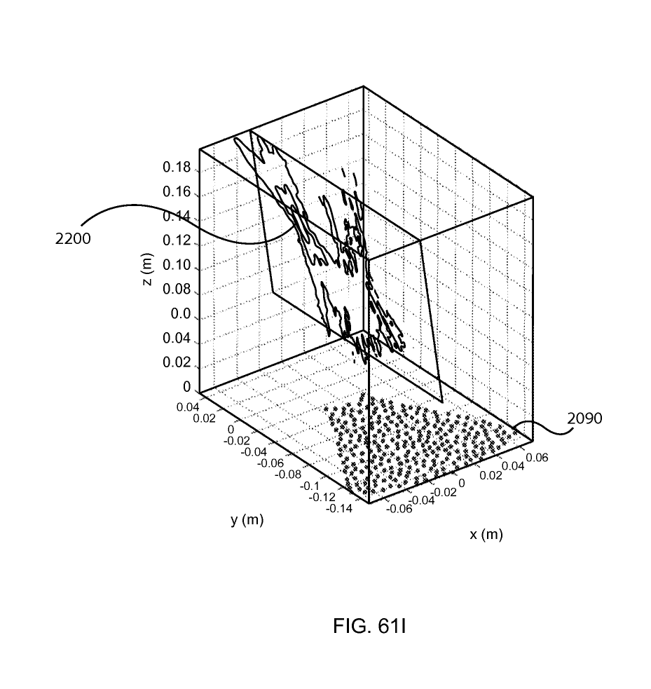

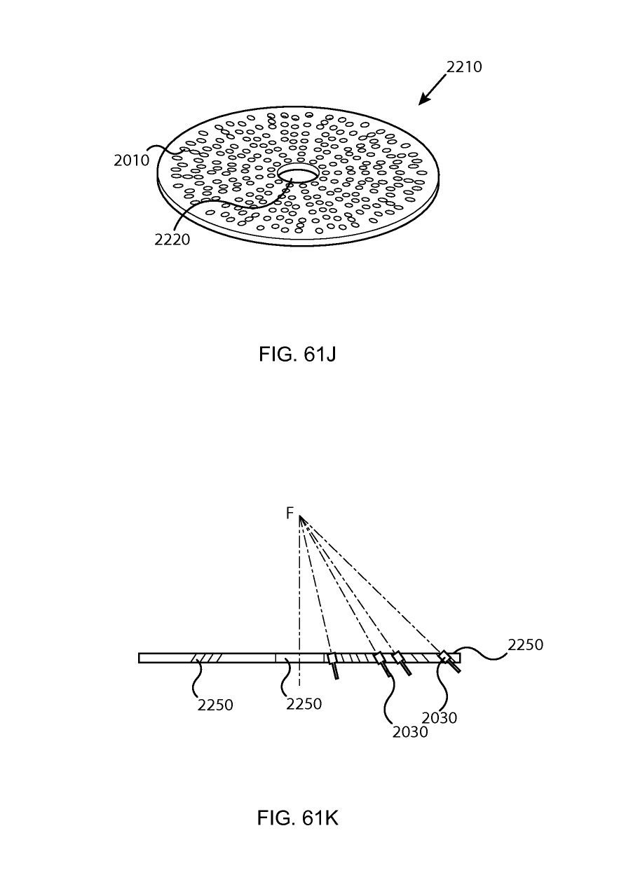

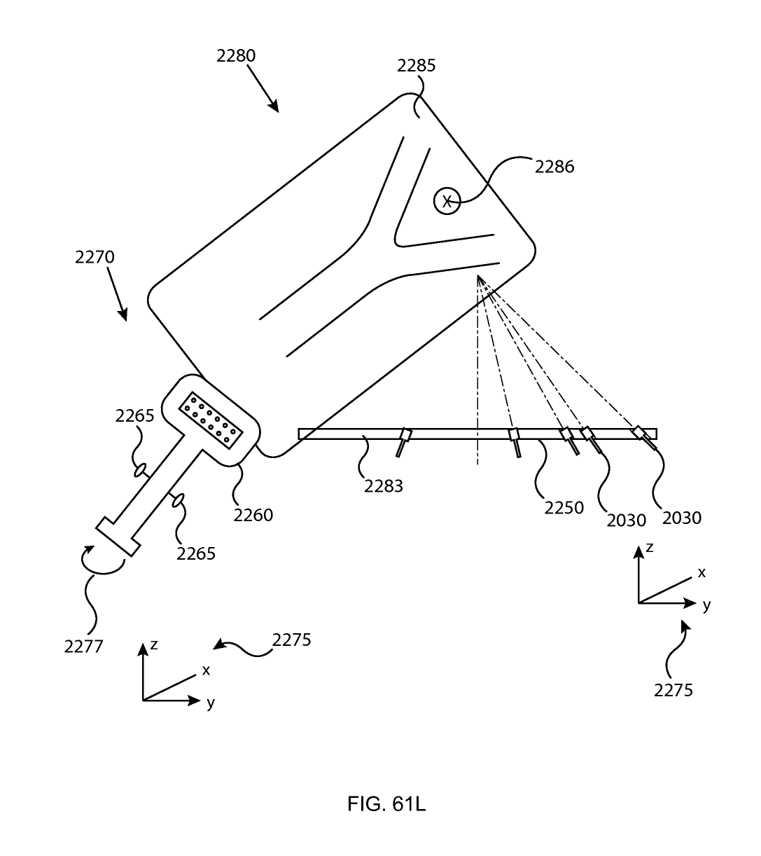

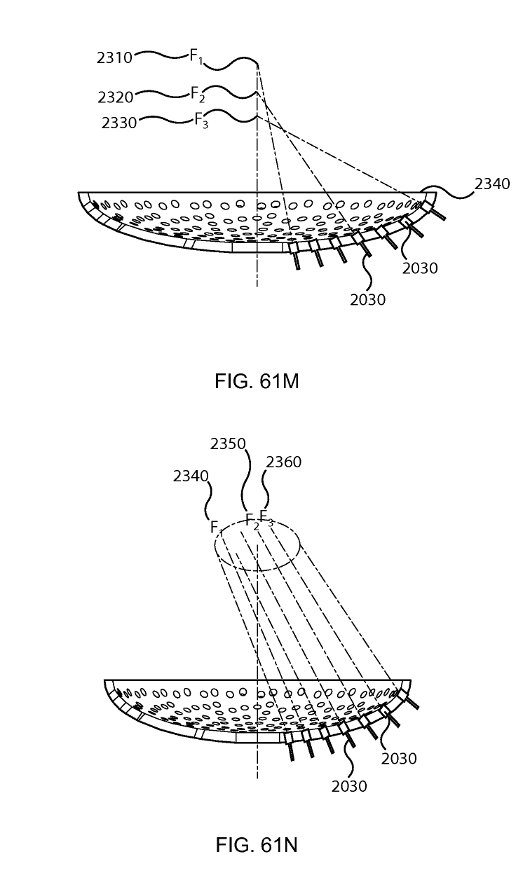

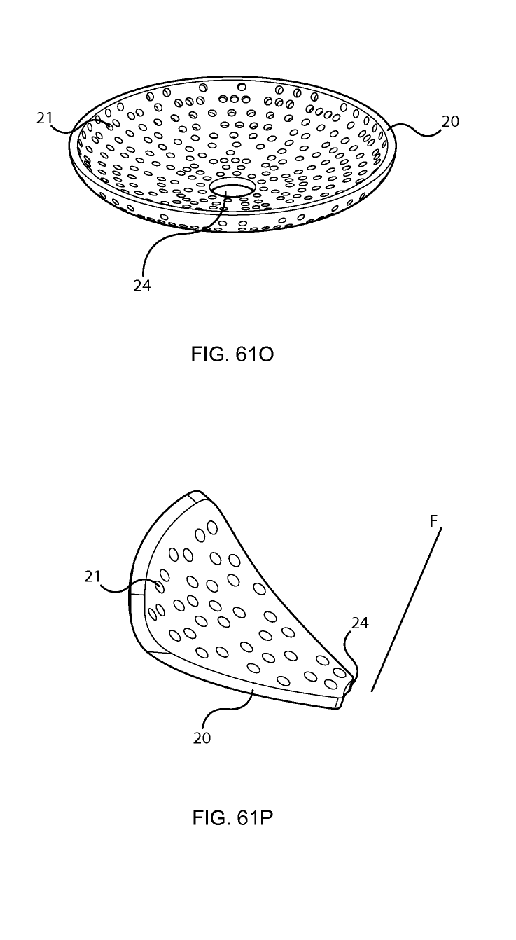

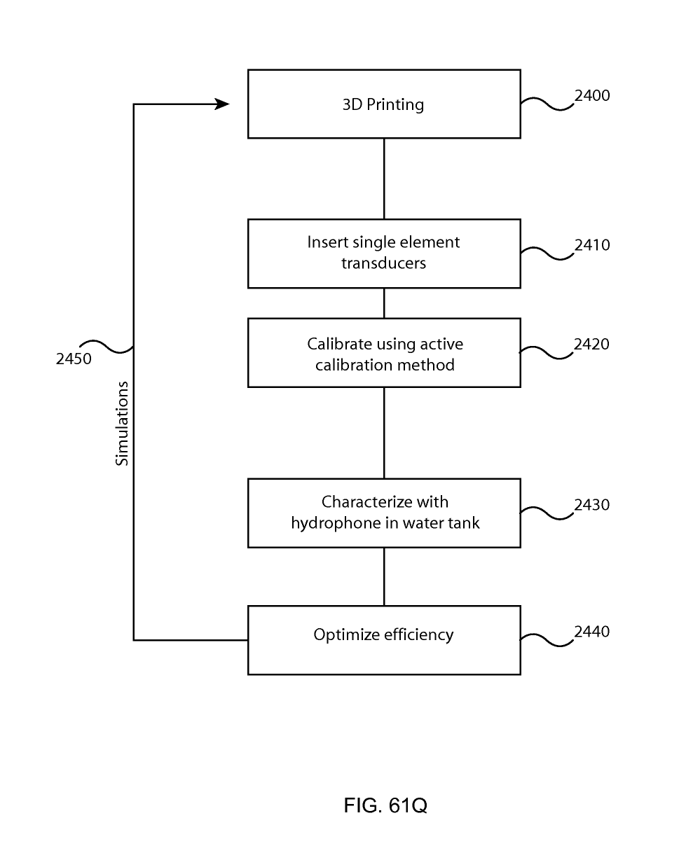

In one variation, the concaved treatment array includes a concaved base support, or substrate, 372 having a honeycomb structure 370 or a plurality of cavities 368 on the surface of the base support 372, for housing individual transducer elements, as shown in FIG. 61A. The base support can also be referred to as a substrate and this substrate can be created from a three dimensional fabrication process as described below. The plane which is substantially planar to the ultrasound elements is the direction along the elements . . . e.g. 2340, 2350, 2360 in FIG. 61N. The axis orthogonal to the planar plane is the depth, or Z, direction represented in FIG. 61M by 2320, 2330, and 2310.

In another embodiment, FIG. 61B-C, a random set of piezoelectric elements 2030 is disposed on a spherical bowl 2050 creating a three dimensional array, which can either used for imaging, therapy, or a combination of both. In its easiest implementation, each of the elements 2030 is identical to one another and each is a single element transducer. Importantly, each of the piezoelectric elements is a single element transducer with an orientation in the spherical substrate 2000 defined by the mechanical design of the substrate 2000. In another embodiment, each of the individual transducers is a different one or there are several groups of individual transducer with more complicated ultrasound generators; for example, each individual transducer might itself have phased array set of elements. The design and manufacture of the substrate is accomplished by a variety of methods including three-dimensional printing techniques. In these techniques, an additive process is used so that almost any type of shape can be created; for example, curved shapes which might otherwise be difficult for a machining process to achieve. Common additive processes in three dimensional printing include (e.g. selective laser melting (SLM), direct metal laser sintering (DMLS), selective laser sintering (SLS), fused deposition modeling (FDM), and polymer curing type processes such as stereolithography (SLA). Each of the single element transducers (FIG. 61D) can fit into a space 2010 in the base substrate 2000. A central connection 2020 can also be created to attach the substrate with piezoelectric elements to any of the other components of the overall system.

FIG. 61C depicts an example of a therapeutic transducer 2050 created via three dimensional printing technique with individual single element transducers 2030 directed into an orientation mechanically by the base structure 2050. For example, three dimensional printing affords the ability to easily create ridges and brackets (2065, FIG. 61D) such that the individual ultrasound transducers 2030 can essentially snap into place. The orientation and position of the individual elements 2030 can facilitate focusing 2035 naturally because of their position and project to the therapeutic axis 2035 and allow for a more efficient array design. Electronic connectors 2040 allow communication with the remainder of the system and are also created in a uniform manner with the rest of the single element transducer. Similarly, the connectors can be snap fit into standard ultrasound cabling.

Portions of the array or all of the array can used to alter the focal spot. For example, some of the elements may be turned on or off depending if there bone or air in the path. To determine if bone might be in the path, a test signal can be sent out from the array. Receivers on the array might then be utilized to determine the type of signal which returns. If there is a high degree of backscatter, it may indicate that an air laden structure or other large impedance mismatch structure (e.g. bone) may exist in the path. In addition, various phasing patterns can be used to accommodate differences in acoustic velocities in tissue due to heterogeneities. These heterogeneities would otherwise cause defocusing of the focal spot and results in lower intensity at the focus than would be present otherwise. Having ability to control phasing spatially to account for different tissues and sound velocities would allow for higher degree of focus and more efficient wall power to focal spot power ratio.

FIG. 61D depicts a close up of the single (or multiple) element transducer 2030. This unit 2030 is the building block of the therapeutic array. 2060 is a coaxial cable which connects the transducer to the rest of the system and power electronics. 2070 is the impedance matching network. A ridge 2065 may be in the form of a bracket, screw thread, etc and is key element in the creation of the therapeutic array as it enables simple and quick manufacture of a therapeutic array. Its pitch and ability to lock the individual transducer 2030 into the substrate simplifies the manufacturing process for therapeutic ultrasound arrays. The ground connection 2072 and the electrical connection from power source to the PZT 2075 is shown. 2090 depicts the PZT which is the material which generates the ultrasound. 2095 is the transducer housing and 2085 is the acoustic matching layer. The PZT may be any shape or size depending on its function such as therapy, imaging, environmental sensing, or all of these. In the current embodiment, the PZT is a flat disc. In another preferred embodiment, the individual PZT element is in the shape of a bowl. In some embodiments the individual transducers are multi-element transducer which also have phasing control.

Further with respect to the novelty of this three-dimensional array is the ability to almost entirely use phase control to move the focus as opposed to mechanical movement as discussed elsewhere in this application. FIGS. 61 E-I depict simulations of the focal spot of the array with evidence for its ability to be controlled with phasing alone. FIG. 61E depicts the array 2090 with randomly distributed elements focused at approximately 12 cm 2110. This simulation represents the base case with treatment along the treatment axis of the array with a tight focus 2110, insignificant side lobes, and a balanced guassian type 2100 intensity distribution close to the focus 2110. FIG. 61F depicts a second simulation using the same array 2090 but with the focus in closer to the array 2090 at approximately 8 cm from the array; the direction of focus control in this case is orthogonal to the first case. As described below, a discovery during these simulations is that by providing a random distribution of single element transducers on the surface of the array, the potential for side lobe generation is extremely small as shown in the simulation. In essence, any organized side lobe focusing cancels out and what is left is a more pure focus. The clinically relevant distance to ablate renal nerves is approximate 8-15 cm. FIG. 61G simulates a focus distal to 12 cm from the array 2090, now focusing at 14 cm, again with a tight focus and without meaningful side lobes. FIG. 61H-I depict the same array 2090 with phasing simulations from side to side in the field. Even at the extreme positions 1-4 cm each direction from the central axis, there is minimal scatter from the focus 2200. Therefore, in this embodiment, adjusting the phase of the transducer elements allows for movement in a plane parallel to the transducer elements, the movement being at least 1 cm and even up to 4 cm in either direction. Based on prior work, this degree of movement is sufficient to track and apply therapy to blood vessels such as the blood vessels leading to the kidney.

A discovery and novel feature of this array is its ability to generate an ultrasound focus without side lobes of extraneous ultrasound or alternative foci (i.e. sidelobes) within the ultrasound field as shown in the simulations (e.g. 6 E-F).

FIG. 61J-K depicts a substantially flat, or two dimensional array. The individual elements are arranged randomly once again but the array is now flat. In this embodiment, the plane of the elements is easy to recognize as it's the flat plane containing the elements. The individual PZT elements 2030 are placed in slots but the slots now have more curvature or angling 2030 than the more substantial curved three dimensional substrates (e.g. FIG. 61B). The flatter design 2250 allows for a more compact design of the substrate 2250 with respect to the patient table discussed earlier . . . and is therefore a preferred embodiment. Although the substrate 2250 is flat, as can be seen in FIG. 61K, the individual transducer elements 2030 are in fact angled. In this angled condition, there is an optimal focus for the transducer elements. By setting this optimal focus at a clinically relevant depth (e.g. the deepest of the depths expected clinically), treatment efficiency is optimized. That is, the most efficient focal efficiency occurs at about the deepest clinical depth so as to minimize the power requirement at the deepest treatment levels. This optimal focus is referred to as the “optimized focal depth.”

FIG. 61L depicts a multi-element therapeutic ultrasound transducer with individual elements 2030 press fit into a substrate 2250, which was created by a three-dimensional printing process. An associated ultrasound imaging probe 2270 is also depicted. The imaging probe 2270 in this embodiment provides a two dimensional image 2280 of a blood vessel 2283 and its branch 2285. The head 2260 of the imaging probe contains an array of piezoelectric transducers which focus imaging energy at a region between 9 and 15 cm deep in tissue. A target 2286 is shown in the ultrasound image and this target would be shown on the screen which the operator sees. The blood vessel typically moves with respiration or discomfort of the patient or pulsatile blood flow. Therapeutic ultrasound elements 2030 are individually controlled by a processor in a system such as shown in FIG. 1 and the focus of the therapy can be controlled to within 1-4 cm on either side of the transducer and from 8-15 cm as shown and described below. Fiducials 2265 on the imaging transducer 2270 allow for detection of its position in absolute coordinate reference frame 2275. The fiducials may be active or passive. They may be optical, inertial, or electromagnetic in nature. This coordinate frame 2275 can be linked to the therapeutic ultrasound transducer 2250 and its coordinate reference frame 2255 which has similar fiducials for determining physical position in a coordinate reference frame 2255.

Beyond the design features above, FIG. 61M depicts some of the functionality of the arrays. Multiple focal points 2320, 2310, 2330 are possible along the axis of the therapeutic transducer by changing the phase of the elements 2030. Similarly in FIG. 61N, multiple foci 2340, 2350, 2360 are possible through different phasing patterns. Patterns can be created and electronically controlled and tracking can be performed using various phasing differences programs.

FIGS. 61 O-P depict two different configurations which might be created through three dimensional processing 2000. In one case, a bowl shape is shown and in the other case, a pizza slice type shape is depicted. In other embodiments, a flat substrate is created. Both shapes can be created using three dimensional printing equipment. Region 2010 is the indicated region where the piezoelectric elements fit into the ultrasound array. Space 2020 is optionally created somewhere on the array to account for the requirement

Therefore, in a process for manufacturing an ultrasound transducer (FIG. 61Q), a three dimensional substrate of any shape and contour is produced by a three dimensional printing process 2400. The substrate includes ridges and is contoured such that singe or multi-element piezoelectric transducers can be press or snap fitted, or glued into place on the substrate 2410. The combination of the three dimensional printing, identical “easy to snap in” transducers, and simple calibration allow for quick and very flexible design changes of the ultrasound transducer to maximize efficiency 2440 for the clinical task at hand. Varying designs for any body region can quickly be simulated with numerical simulation 2450 and then prototypes can be quickly build and tested. Further to the process of production of this unique and novel array, is straightforward calibration 2420. To calibrate the complete therapy array subsequent to placement of the individual transducers in their spot, a piezoelectric sensor can be placed at a focus 2035. Each individual element 2030 can then send a signal which is detected by the sensor at the focus. Based on the acoustic time of flight between the transducer and the sensor. The orientation and efficiency of power delivery can be determined for the individual transducers in the array. This equates to almost self- or auto-calibration of the array which relaxes the requirements and tolerances for placement of the individual transducer elements 2030. That is, tolerance to exact placement and exact machining dimensions is not as much an issue as it would be without the self-calibration technique. For example, in some embodiments, the tolerance for position might be greater than 100 microns or greater than 50 microns which without this novel auto calibration technique would not be acceptable. Each array is calibrated after manufacture and further characterized with a hydrophone in a water tank. The characterization proves that the auto calibration was in fact successful. Therefore, the novel calibration technique of the array allows for a manufacturing process such as three-dimensional printing which might not be as exact as other standard or typically utilized machining processes in use for focused ultrasound.

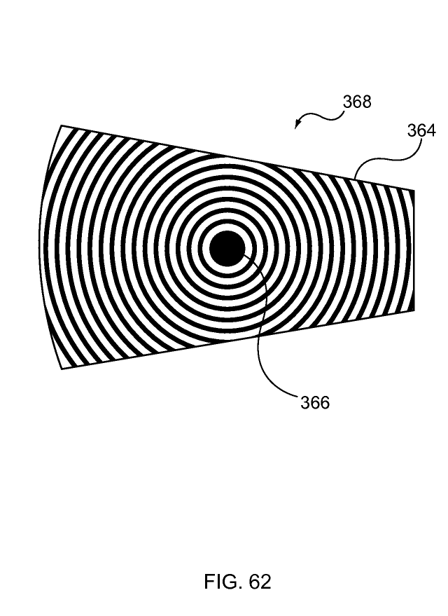

In an alternative design, as shown in FIG. 62, a concentric ring design 368 is implemented where one centers 366 the annular array at the ‘weighted’ center of the fan or pie-segment 364 shaped aperture. This centered annular array within a pie segment shaped aperture may effectively focuses in all directions with symmetric performance. It may be used to track target movement in 3D. Dose delivery may also be more symmetric in 3D. In one variation, the surface of this array is flat. In another variation, the surface of this array is concaved.

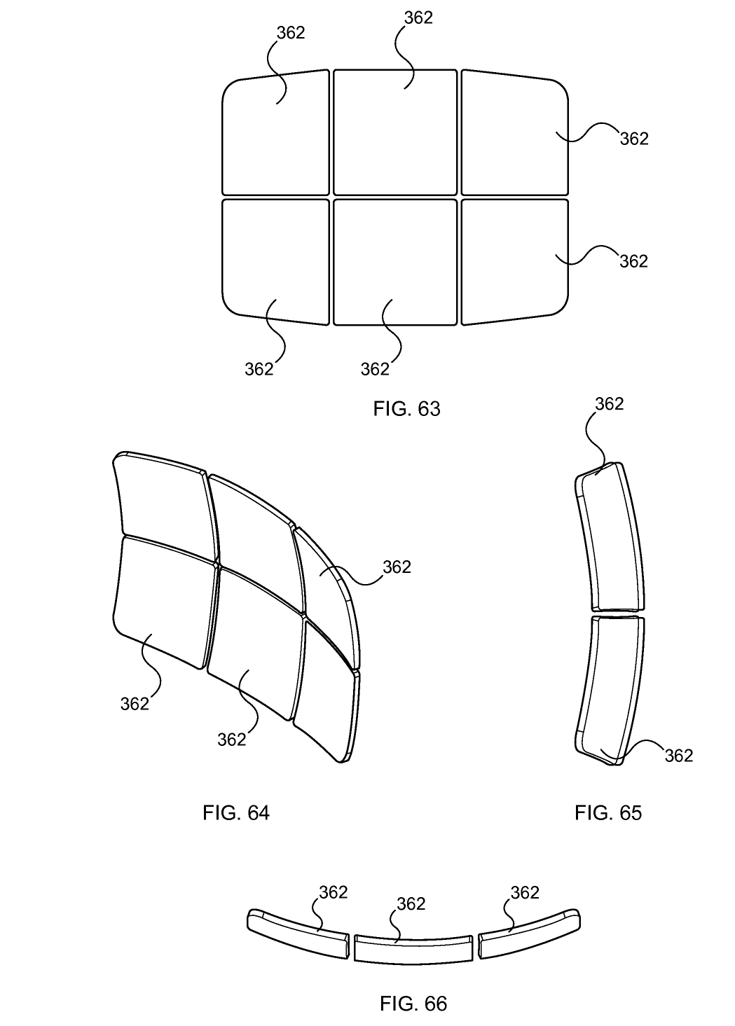

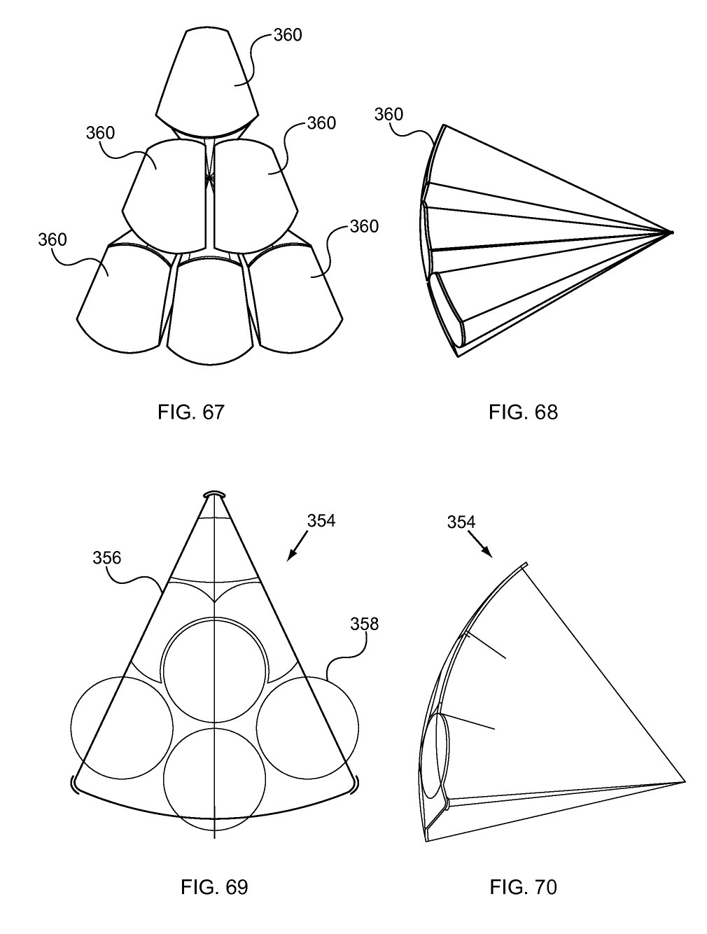

FIGS. 63-66 illustrate another example of concave array designs. In this design, the array is comprised of six lobes 362 that are configured to deliver ultrasound to a single focal point. FIGS. 67-68 illustrate another example of a fan shaped design comprising of six individual concaved plates 360, each with an array of ultrasound transducers. The six individual plates are configured to direct ultrasound to a single focal point. FIGS. 69 and 70 illustrates another example using a combination of fan shaped and circular profile in the array design 354. Each of the circular subunit may be further configured with concentric rings 358 or partial annular rings 356 of array elements. Each of the circular subunit may be further configured with a concaved profile with the center of the concave positioned at the center of the circle. The individual circular subunit may be separately actuated, such that the focal position for each of the circular array can be adjusted such that they are synchronized to deliver ultra sound energy onto a singular target position.

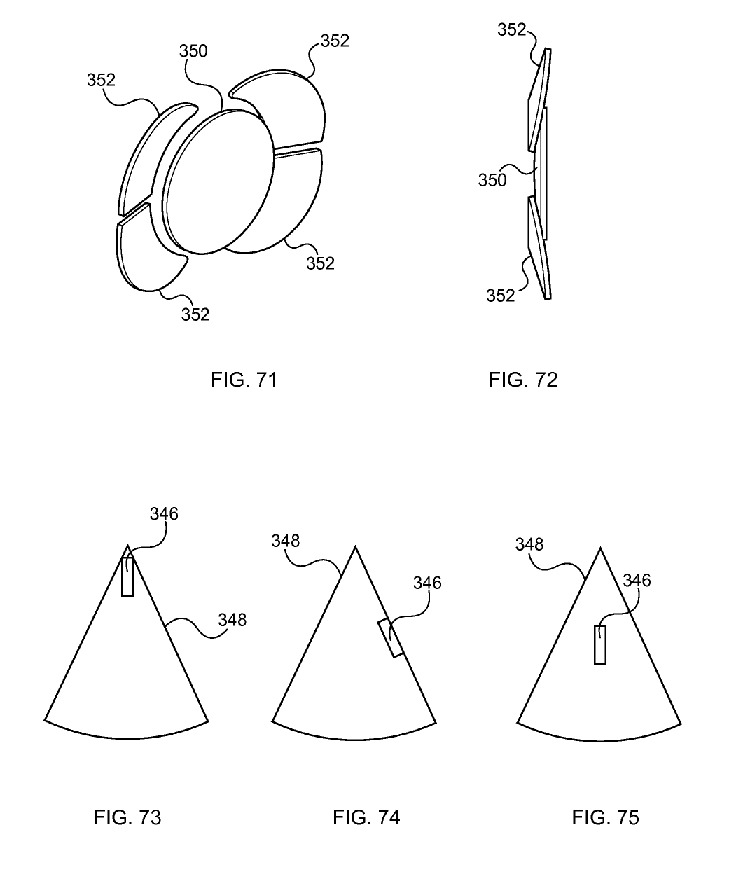

FIG. 71-72 illustrate yet another example, where a plurality of transducer array surfaces are configured in a staggered or out-of-plane configuration, that forms a larger coherent focused ultrasound delivery array structure. In this example crescent shaped arrays 352 are combined with a circular shaped 350 array that is centrally positioned. In one variation, the center circular array 350 is configured for imaging detection and therapeutic delivery, while the four crescent shaped arrays are configured for therapeutic delivery only. In another variation, the center circular array 350 operates at one frequency, while the four outer crescent shape arrays 352 operates at a different frequency.

FIG. 73-75 illustrates another design where a portion of the surface area for each of the therapeutic arrays 348 is allocated for placement of imaging array elements 346. Therefore, the array would have the capacity for of both image detection, and therapy delivery. In these examples, the imaging elements are positioned in the rectangular shaped areas on the fan shaped array profiles. The exact position of the imaging array 346 depends on the particular anatomy at hand. In some embodiments, there are one or more imaging arrays in multiple positions on the therapy array 348. Such an arrangement might be desired when a structure is moving or when a greater amount of three dimensional spatial resolution is desired.

### IV. Tracking Beacon

An ultrasound emitter or emitters may be positioned in or adjacent to a targeted tissue region for ultrasound treatment. The emitter serves as a beacon for the system to locate the treatment region and track the movement of the treatment region during the treatment procedure to ensure that the focus of the ultrasound wave is maintained in the treatment region during therapy. The beacon may also serve as a reference point, such that a specific treatment pattern may be delivered relative to the position of the beacon. This allows the system to deliver multiple doses of ultrasound serially in time to establish a treatment pattern that is predetermined, even if the target tissue is not stationary during the treatment process. The system tracks the position of the beacon and delivers the ultrasound dosage to positions referencing the beacon position.

As described below, the same, or similar type of beacon is used to self- or auto-calibrate the treatment array which may have been put together with relatively low assembly tolerances.

In one example, the tracking beacon is configured as an ultrasound transducer positioned in a catheter (also referred herein as the “targeting catheter”). The targeting catheter can be advanced to the desired target region through vasculature, or other orifice or lumens within the patient's body, in order to position the distal portion of the catheter, which carries the beacon, in the target region.

In one variation, the targeting catheter comprises a sterile, 3 F-6 F (French), catheter that can be placed into the blood vessel and advance to the target organ to facilitate targeting (i.e., position of the focal point of the treatment ultrasound). For example, the catheter can be advanced up the arterial system and into the renal artery, for guiding the delivery of ultrasound therapy around the renal artery. The targeting catheter features a small ultrasound transducer located in its distal tip or distal region that transmits ultrasonic pulses to signal its position relative to a cluster of receivers in the treatment module. This locating and positioning system ensures definitive targeting of the treatment tissue (such as the renal artery) and robust target region tracking during treatment.

The catheter can also be used in an interstitial position, allowing focused ultrasound to be targeted to virtually any tissue such as liver, prostate, thyroid, colon, and rectum; and, it can be positioned in other arteries or veins such as the aorta, the portal vein, the hepatic artery, the carotid artery, the mesenteric artery, and the vena cava.

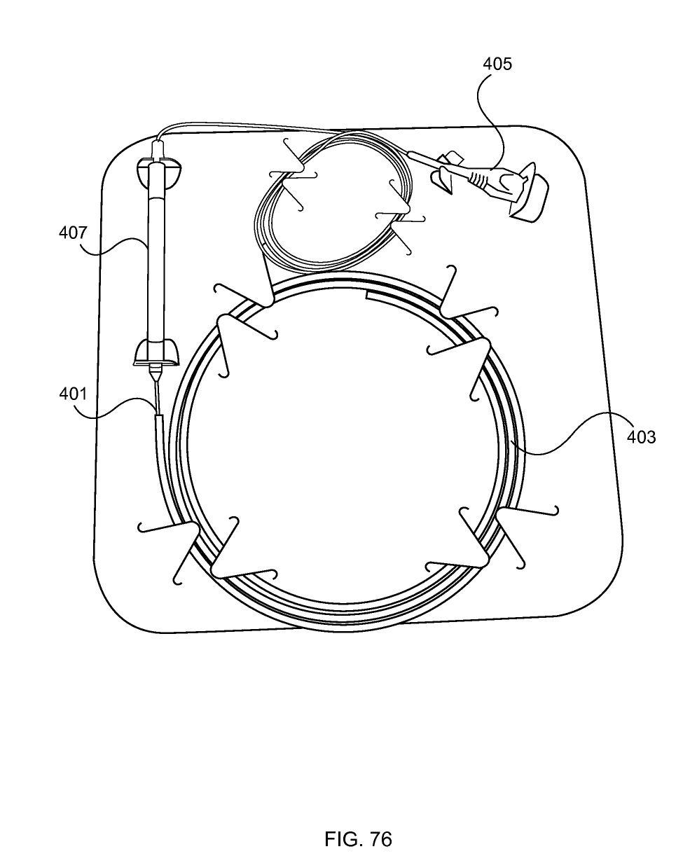

Referring to FIG. 76, an example of a targeting catheter 401 is shown with the distal portion of the catheter inserted inside a protective sheath 403. The proximal end of the catheter includes an electrical connector, such as an RJ45 connector 405. The proximal portion may further comprises an electronic tuning module 407 located along the length of the catheter.

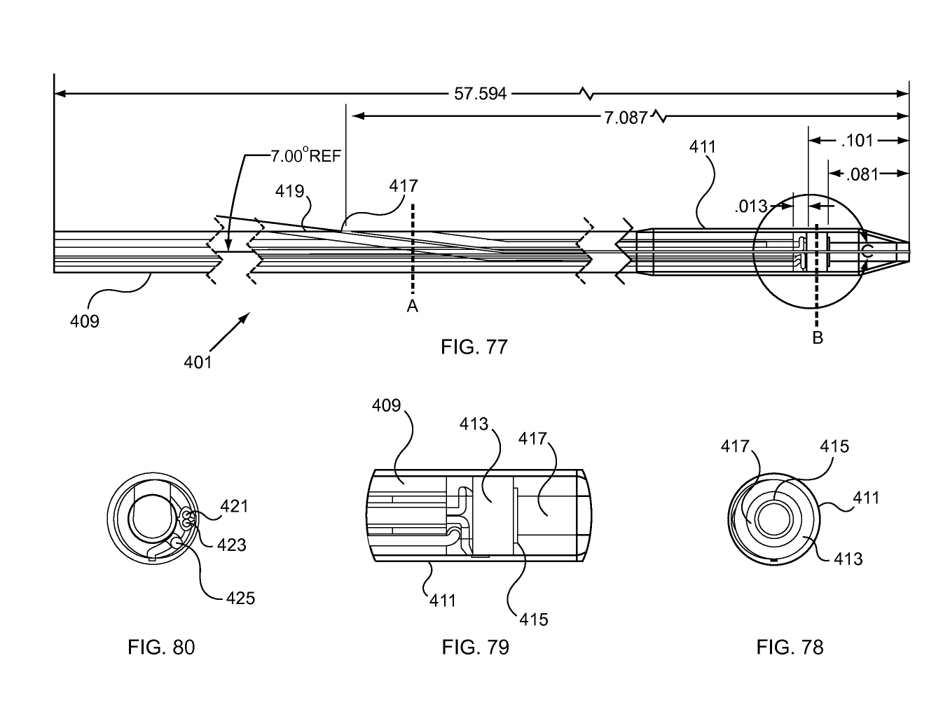

FIG. 77 illustrates the construction of a targeting catheter 401. The catheter comprises an elongated primary sheath 409. An outer sheath 411 connected to the distal end of the primary sheath. The proximal end of the primary sheath is connected to the distal end of the electronic tuning module. The proximal portion of the outer sheath 411 overlaps with the distal portion of the primary sheath 409. An ultrasound emitter 413, comprising a PZT transducer is position in the distal portion of the catheter within the lumen of the outer sheath 411. A PZT transducer is a transducer that comprises active materials, Lead, Zirconate, and Titanate. A transducer liner 415 is inserted through the PZT transducer, and further inserted into the primary sheath 409, such that the distal portion of the transducer liner is position within the lumen of the outer sheath 411, as show in FIG. 78, an expanded view of the distal portion of the targeting catheter. A guide tube 417 extends from the distal tip of the catheter through the distal portion of the primary sheath and exit 419 through the side wall of the primary sheath 409. The guide tube allows the targeting catheter to be deployed in a patient through over the guide-wire insertion technique. The guide-wire can enter the targeting catheter through the mid-shaft where the proximal portion of the guide tube exits, and advanced towards the distal tip and exit the catheter at the distal tip. This configuration allows the targeting catheter to ride along a guide-wire that has been placed within a patient's vascular system.

Two wires 421, 423 extends form the distal end connector through the length of the catheter to the distal end of the catheter, for energizing the PZT transducer 413. One wire is connected to the inner wall of the hollow cylindrical shaped PZT transducer, and the other wire is connected to the outer wall of the PZT transducer.

FIG. 79 depicts the cross-sectional view of the catheter 401 at the mid-point of the PZT transducer 413. The outer wall surrounding the PZT transducer comprises of a material that provides good acoustic transmission at the same time provides good electrical isolation. In one variation, the outer sheet 411 allows efficient transmission of acoustic energy from the PZT transducer through the outer sheet into the patient's body tissue with less than 20 percent loss of acoustic energy. At the same time, the outer sheath electrically isolates the electrical wires energizing the PZT, such that electrical current does not leak into the patient's body.

FIG. 80 is a cross-sectional view of the catheter 401 proximal to the PZT transducer 413. The two electrical wirings 421, 423 for energizing the PZT transducer 413 are position within one of the side lumen of the primary sheath, while an elongated safety line 425 is position in the second side lumen of the primary sheath. The safety line extends from the proximal end of the catheter to the distal portion, where it is connected to the transducer liner.

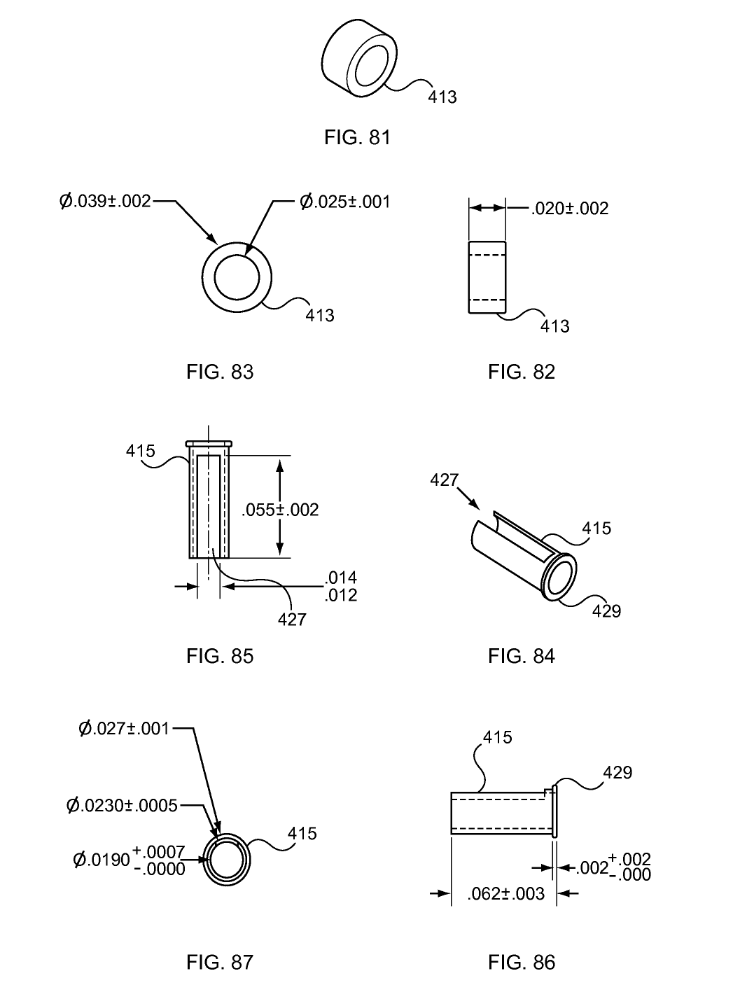

FIG. 81 is a perspective view of the PZT transducer 413. The PZT transducer has a hollow lumen extending from the distal end to the proximal end. The length is 0.02 inches, as shown in FIG. 82. Its inner diameter is 0.025 inches, and the outer diameter is 0.039 inches, as shown in FIG. 83.

Referring to FIG. 84, the transducer liner 415 comprises partial stainless steel tubing (e.g., SS 704), which flares outward at the distal end. A trough 427 runs partially along one side of the tubing wall, as shown in FIG. 85. FIG. 86 is the side view and FIG. 87 is the viewed from the proximal end down the length of the tubing. The flare 429 at the distal end of the transducer liner 415 helps secure the PZT transducer in-place, and prevent the PZT transducer from dislodging from the distal end. The flare further ensures that when the safety line is pulled towards the proximal direction, and as the liners is pulled along towards the proximal direction, the PZT transducer will be captured by the liner and exit the body along with the liner.

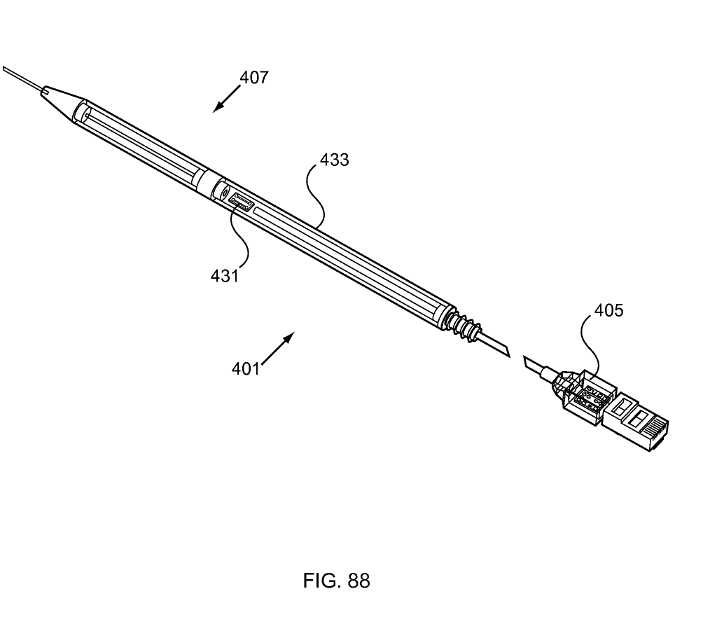

FIG. 88 is an expanded view of the distal portion of the targeting catheter 401, which includes an electronic tuning module 407. The tuning module comprises an electronic board 431 housed within a proximal tubing 433. The proximal tubing 433 may comprise of a stiff material to provide structure support and protection to the electronic board. The proximal tubing may also comprise a transparent or partially transparent material, such that the operator is able to see the electronic board housed within the proximal sheath. The proximal end of the electrical wiring for energizing the PZT transducer is connected to the distal portion of the electronic board. Two additional electrical wiring connects the proximal portion of the electronic board to the connector located at the proximal end of the catheter. The electronic board 431 may include electronics for tuning and/or optimizing the performance of the PZT transducer. In one variation, the board comprises an inductor for capacitance matching. In another variation, the board is configure to provide impedance matching, such that electrical current transmitted from an ultrasound transceiver or transmitter, that is connect to the targeting catheter, can be efficiently transmitted down the electrical wiring driving the PZT transducer located at the distal portion of the targeting catheter.

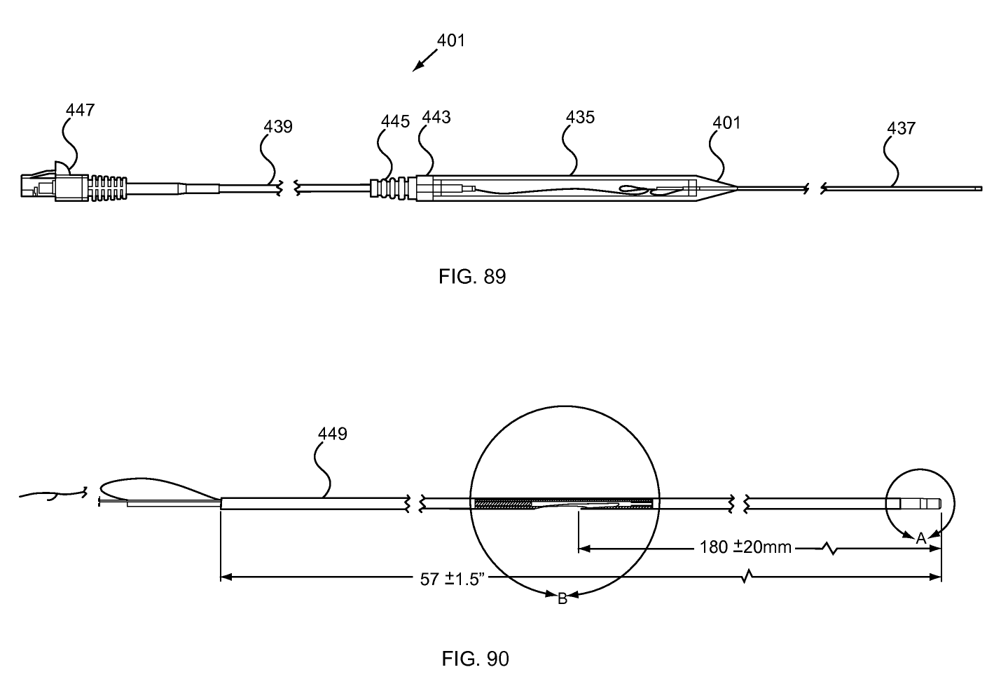

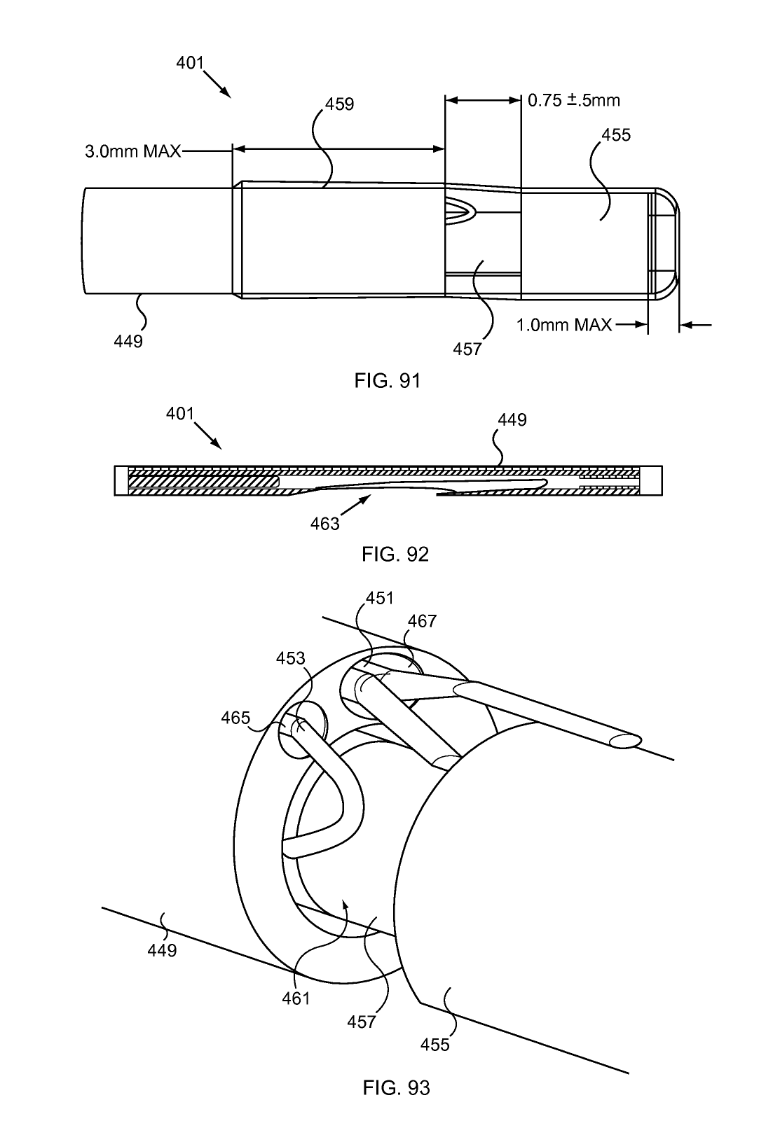

Referring to FIG. 89, another example of a targeting catheter 401 is shown. In this example, the target catheter comprises a handle 435 with a catheter shaft 437 extending distally, and a coax cable 439 extending proximally. The handle includes a handle tip 441, a handle connector 443, and a strain relief 445. The proximal end of the coax 439 is connected to a RJ-45 connector 447. A pair of electrical wiring extends through the handle, and through the catheter shaft to the transducer at the distal end of the targeting catheter. A service loop of the electrical wiring is provided within the handle. The distal portion of the catheter, as shown in FIG. 90, comprises a primary sheath tubing 449, a Nitinol mandrel, a twisted pair of electrical wiring 451, and a safety wire 453. The distal end of the catheter 401, as shown in FIG. 91, comprises a PZT transducer 455, a guide tubing 457, and a heat shrink tube 459. The safety wire 453 extends to the distal end, and forms a “J” hook 461 proximal of the PZT transducer 455. In this example, the PZT transducer comprises a cylindrical body, having an axial length of about 0.049 inches, an outer diameter of about 0.039 inches, and an inner diameter of about 0.024 inches.

The mid-shaft of the catheter has a side opening 463 for allowing guide-wire access to the lumen of the primary sheath tubing 449, also refer to as the outer tubing. The configuration allows for over the guide-wire insertion of the targeting catheter 401. Referring to FIG. 92, the guide tubing 457 (also referred to as the inner tubing) is inserted into the proximal end of the primary sheath tubing 449, and extending in the lumen of the primary sheath tubing to a position proximal of the side port on the primary sheath tubing.

The primary sheath tubing 449 includes three lumens, a primary lumen 465, a circular shaped side-lumen for the safety wire, and an oval shaped side-lumen 467 for the electrical wiring, as shown in FIG. 93. The three lumens are plugged with epoxy (e.g. 353ND-T epoxy). The electrical wiring exits the oval side-lumen at the distal end of the primary sheath tubing. One of the twisted pair wiring is attached to the outer face of the cylindrical transducer, while the other is attached to the inner surface of the cylindrical transducer 455. The safety wire exits the circular side-lumen and forms a J-hook, with the distal tip of the hook inserted into the primary lumen.

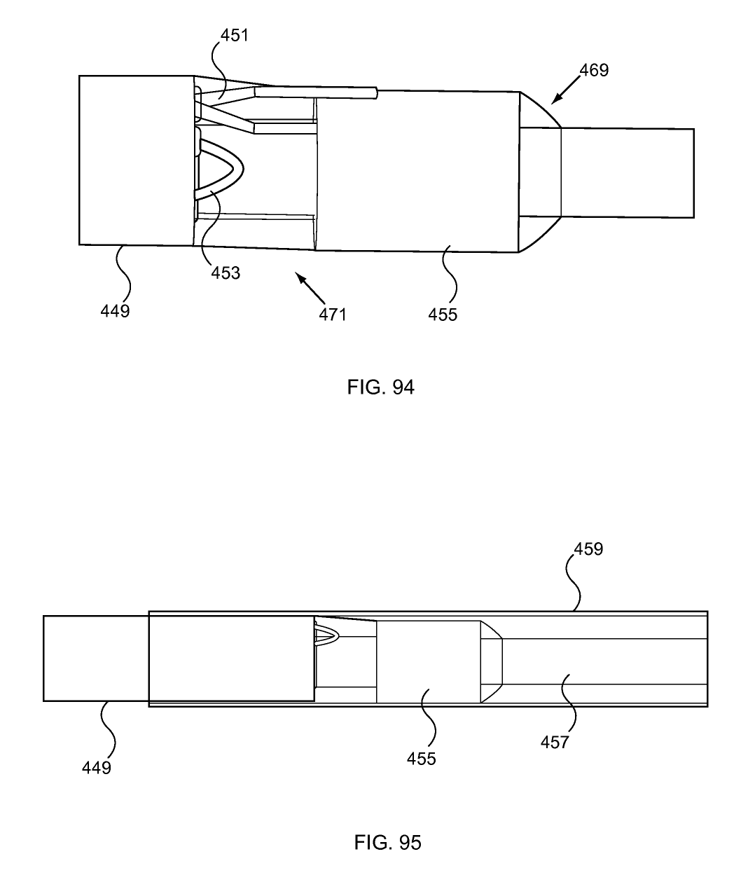

In one variation, epoxy is placed between the PZT transducer and guide tube to secure the transducer to the guide tube. Adhesive (e.g., UV activated adhesive) is placed at the distal edge of the PZT transducer to create a taper 469. Adhesive is also used to fill the gap 471 between the PZT transducer 455 and the primary sheath 449, as shown in FIG. 94. A shrink-wrap tubing 459 is placed over the distal portion of the catheter (see FIG. 95), and shrunk down to cover and secure the components. The excess distal portion is then cut off.

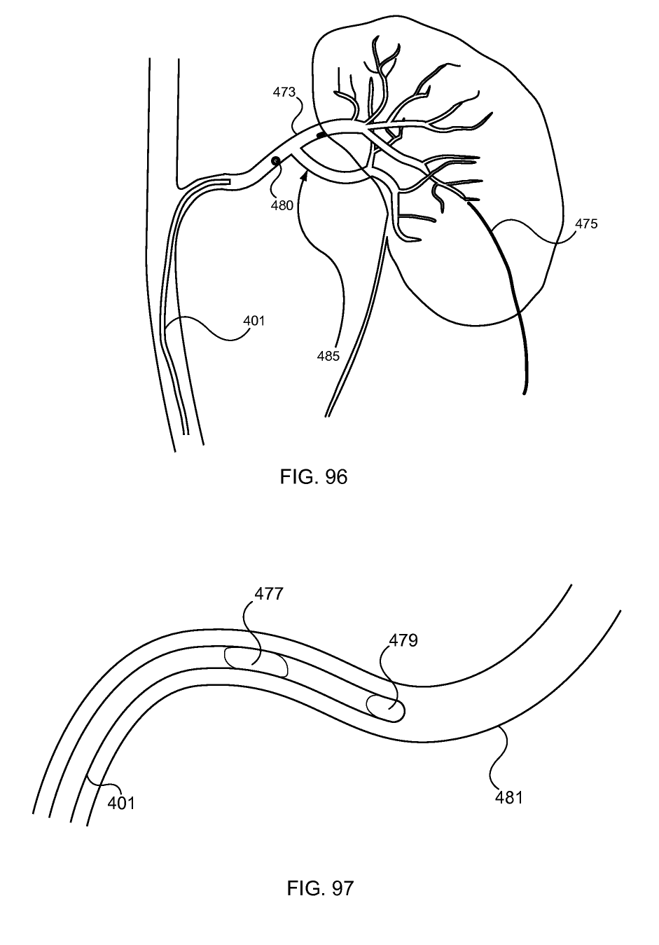

Referring to FIG. 96, an example demonstrating the placement of a targeting catheter 401 in the renal artery is shown. A guide-wire is inserted in the femoral artery in the patient's leg, and advance up the aorta, and then turn into the renal artery 473. Once the distal portion of the guide-wire 475 is positioned within the renal artery, the targeting catheter 401 is place over the guide-wire 475 and advanced into the patient's body until the distal portion of the targeting catheter is positioned within the renal artery. Through the fluoroscope, the physician can adjust the targeting catheter to place the beacon within the catheter at a desire location. For example, in one variation, the physician may choose to retract the targeting catheter until the beacon is positioned immediately proximal to the major branching the renal artery. The beacon is then energized to provide the ultrasound signal for the therapeutic ultrasound system to locate the treatment region surrounding the renal artery.

One of ordinary skill of the art having the benefit of this disclosure would appreciate that the targeting catheter can be built with different sizing, such as 3 F or 4 F (French), and/or different lengths, depending on clinical indications and particular application requirements. In one variation, the Outer Diameter (OD) of the targeting catheter is 4 F. It is able to fit into a 6 F guide catheter or guide sheath.

In addition, the catheter may be configured with two or more beacons. In one example, the targeting catheter 401 is configured with two beacons 477, 479 as shown in FIG. 97. The physician may advance the catheter and position the two beacons across a target region to be treated. For certain applications, the two beacons may provide better indices for marking the treatment regions in a three dimensional space. In one variations, where tissue surrounding a vessel 481 is to be treated, the catheter may be advanced to the desired vessel with the two beacon positioned across a section of the vessel to be treated. The two beacons allow the ultrasound therapeutic system to determine an angular direction of the vessel, as it lies within the treatment space, which may allow the system to calculate location of a treatment plan surrounding the vessel and taking into account the angular orientation of the segment of the vessel located between the two beacons. The two beacon arrangement may also allow the system to treat a region between the two beacons while avoiding directing the focus of the therapeutic ultrasound directly on one or both of the beacons.

In another variation, three or more beacons are position along the length of the distal portion of the targeting catheter. The three or more beacon allow the system to calculate and/or model the path and curvature of the targeting catheter within the patient's body, and model it in a three dimensional reference system for treatment planning.

In another variation, radio opaque markers may be included on a catheter to enhance the visibility of a specific portion of the targeting catheter. For example, markers may be positioned on the distal tip, or adjacent to the location of the beacon on the catheter.

In another variation, one or more temperature sensors, such as a thermal couple or a thermistor, may be placed in the distal portion of the targeting catheter for detection of temperature changes surrounding the treated tissue. The temperature of the tissue in the target region may be monitor to avoid excessive heating. For example, if the targeting catheter is positioned within a blood vessel, and the tissue surrounds the blood vessel is to be treated with therapeutic ultrasound, once the system detects that the temperature in the blood vessel has exceeded a pre-determined threshold, the system may (a) terminate the treatment, (b) modify the treatment plan by extending the overall treatment time and spread out the treatment intervals over time, or (c) decrease the treatment ultrasound intensity, while extending treatment time to compensate for overall dosage, to avoid over-heating the blood vessel and thereby avoiding damaging the blood vessel.

In another variation, the ultrasound transducer is utilized to serve as a temperature detector. A low power electrical excitation may be delivered to the PZT transducer to ping the transducer in order to detect changes to the PZT transducers characteristic as temperature surrounding the PZT transducers changes. With calibration, the user can then determine the temperature changes based on the temperature induced changes on the characteristic of the PZT transducer as it response to the low power excitation. The ultrasound transceiver connected to the PZT transducer may time multiplex the higher power excitation for driving the beacon signal, with the lower power excitation for temperature termination, such that the system continuously send out a series of beacon pulses, while detecting temperature changes in between.

In another example, the tracking beacon is configured as an ultrasound transmitter positioned within a pill or pellet. The pellet is deposited within or adjacent to a target tissue to provide a beacon for the ultrasound treatment system to hone in onto the target. The tracking pellet may be deposited into the target region through a catheter, a trocar, or through needle injection. In one variation, the tracking pellet has no external physical connection once it is deposited at the target site. In another variation, the tracking pellet includes electronic wiring extending from the pellet to provide power supply. In yet another variation, the tracking pellet is configured to be moved within the patient's body to a desired location, without external physical contact. For example, the pellet may be metallic or magnetic such that magnetic field may be used to adjust the position of the tracking pellet. In yet another variation, the tracking pellet may be charged or excited through non-invasive transmission of energy. For example, a capacitive circuit in the pallet is excited or energized by an RF wave, once energized it will emit an ultrasound frequency for a period of time and allow it to serve as an ultrasound beacon signal.

In another variation, the beacon is positioned at the distal tip of a needle or trocar. For example, an ultrasound transducer may be positioned in the distal tip of the needle or trocar, while electrical wirings extends along the length of the needle or trocar through an inner lumen of the shaft towards to proximal end for energizing the transducer. The physician would insert the needle or trocar into the patient's body, and maneuver the distal tip of the needle or trocar to the target region. The distal tip of the needle or trocar can then emit a tracking/honing signal and allow the ultrasound therapy system to locate the treatment region.

In one variation, three or more beacons are used to identify a volume within a patient's body to be treated. The three or more beacons may positioned within the patients through different modality. For example, two beacons are positioned with a catheter, and a third beacon is position through the insertion of a needle with a beacon at the distal tip.

When two or more beacons are utilized, the two or more beacons may all emit the same frequency. By time multiplexing the beacon signal emission, the ultrasound therapeutic system would detect the position of the beacons one at a time. In another variation, each of the beacon utilizes a different signal frequency, such that the beacon signals from the different beacons may be emitted at the same time and detected by the system for position calculation.

In another embodiment, the beacon serves as a sensor for the intensity of the ultrasound. When the beacon senses the focused ultrasound, it can be used to send feedback to the processor and power supply to alter the output, creating a closed loop system to apply power to the region of the blood vessels or tissue. In this embodiment, the focused ultrasound is applied to the beacon region and the actual intensity at the focus measured. With knowledge of the actual intensity and the desired intensity, the output of the system can be adjusted to reach the desired intensity.

An example of a process for placement of a targeting catheter is described in detail below. Establishing a vascular access by placing a 4 F-6 F introducer sheath retrograde in a femoral artery using standard percutaneous techniques. Selecting an appropriate guide catheter (for example a 6 F Renal Double Curve (RDC) guide catheter) capable of accommodating a 3.0 F catheter. Attach a hemostasis valve to the lure port and attach a stopcock to the side-port of the hemostasis valve. Attach a syringe of heparinized saline to the stopcock on the side-port of the hemostasis valve and flush the lumen. Using fluoroscopic guidance, advance the guide catheter into a coaxial orientation in the renal ostium. The operator may obtain and record a Renal Artery (RA) angiogram.

Using sterile technique, remove the Targeting Catheter (TC) from its package. Before removing the targeting catheter from its protective coil, fill the coil with sterile heparinized saline and allow the catheter to hydrate. Prepare a 0.014″ Balance Middle Weight (BMW) universal guide-wire (GW) for insertion into the patient's vasculature. Insert the straight end of the BMW guide-wire into the distal end of the targeting catheter and advance it through the lumen until it emerges from the Rx port (located approximately 18 cm from the distal tip) and its curved, flexible end is fully enclosed within the targeting catheter. Insert the targeting catheter and preloaded BMW guide-wire through the hemostatic valve into the guide catheter and advance the assembly to the distal end of the guide catheter. Using fluoroscopic guidance, advance the BMW guide-wire into the renal anatomy as far distally as safely possible. Using fluoroscopic guidance, advance the targeting catheter over the guide-wire and position the targeting catheter beacon approximately 10 mm proximal to the first renal artery bifurcation (FIG. 90). Rotate the hemostatic valve to secure the targeting catheter to the guiding catheter. Using a sterile towel clamp or other suitable means, secure the targeting catheter hub to the sterile drape to minimize the potential for the targeting catheter beacon to be dislocated from the target site. Then pass the targeting catheter electrical connector out of the sterile field through a small slit cut in the sterile drape in the vicinity of the targeting catheter extension box.

FIG. 96 illustrates the targeting catheter being positioned within a left renal artery 473. In one variation, the targeting catheter 401 beacon 483 is placed approximately 10 mm proximal to the first renal artery branch point 485 to define a treatment area. In the example shown in FIG. 96, the beacon is shown located well distal to the treatment location 487, and the operator would then retract the catheter 401 until the beacon 483 is positioned over the region 10 mm proximal to the first renal artery branch 485.

Using fluoroscopy, the operator may evaluate and record the stability of the targeting catheter beacon in the artery lumen over a period multiple respiratory cycles. The operator would then determine and record if the beacon location remains fixed or is displaced relative to the adjacent artery wall. The operator may also determine and record the location of the targeting catheter beacon within the artery lumen relative to the vessel wall (i.e., superior, medial or inferior).

In another embodiment, the targeting catheter is a shaped device which conforms to the tissue around it or makes the tissue around it conform to its shape. For example, the targeting catheter comprises a fluid fillable balloon which when expanded conforms to the wall of the blood vessel. In another embodiment, the catheter tip contains sensor to detect the ultrasound energy (hydrophone) and delivers a signal outside the patient, the signal being proportional to the intensity of the focused ultrasound. In this way, the targeting might be interrogated and the focused ultrasound transducer position altered with respect to the catheter and the signal. The targeting catheter might also allow for complex patterns to be placed around the tissue or blood vessel. For example, there might exist a spiral ring of sensors on the catheter which enables the external system to deliver energy in a spiral pattern around the blood vessel.

V. Tracking of Target Tissue with a Beacon

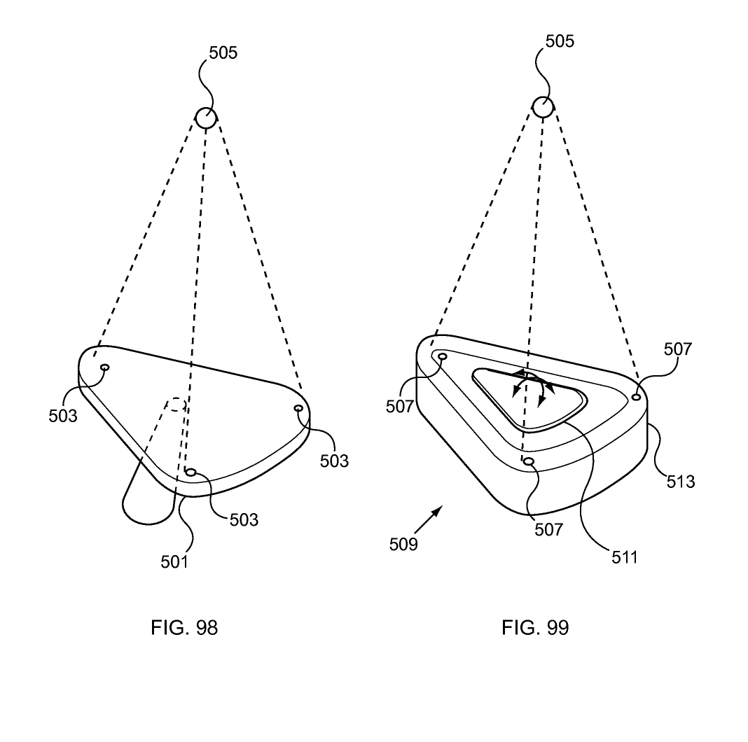

Once the beacon is positioned in-place within the patient's body, the operator may launch a treatment procedure software application, which runs on a computing unit within the generator. In one variation, the system is configured to prompt the user to identify the patient and enter patient information. The treatment transducer array may be coupled to three or more ATOF receivers for detecting ultrasound signals emitted by a beacon positioned in the patient's body. Based on the ATOF signals the system can then calculate the position of the beacon relative to the treatment transducer array, and adjust the position and/or orientation of the treatment transducer array such that the focus of the treatment transducer array tracks the movement of the beacon. In one example, as shown in FIG. 98, a treatment transducer unit 501 (a.k.a., treatment transducer platform), which houses the treatment transducer array, comprises three ATOF receivers 503 for detecting the position of a beacon 505. In another example, as shown in FIG. 99, the ATOF receivers 507 are positioned on the applicator 509, which houses a treatment transducer array 511 that is capable of moving relative to the housing 513 of the applicator 509, such that the face of the treatment transducer array 511 can track the position of the beacon 505 detected by the ATOF receivers 507. The applicator may be positioned on a based mover which would allow the operator or the system to separately adjust the position and/or orientation of the applicator.

After selecting a patient for treatment, the system may ask the operator to select a transducer array for the treatment. Some systems are configured to use one specific treatment transducer array. Other systems may be configured to support two or more treatment transducer arrays. The system may be pre-programed with specification and/or calibration data for the different transducer arrays supported by the system. The operator may select a transducer to be used for the treatment. Once the operator selected the desired transducer, the system would then verify that the appropriate treatment transducers has been installed in the in the treatment module. If the system detects a different transducer array is in the treatment module, it would single the user to replace the transducer array. In one variation, based on the patient information entered into the system, the system would suggest one of the treatment transducer arrays supported by the system that is suitable for the particular patient based on a predefined decision process and/or parameters.

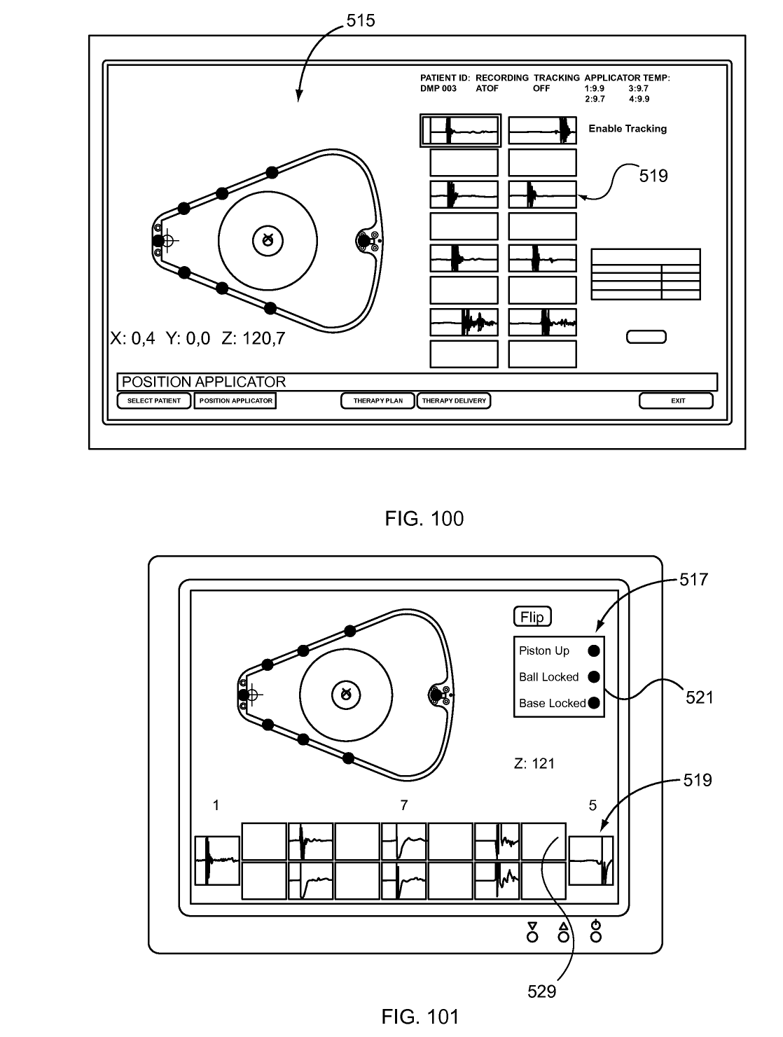

After the treatment transducer array selection has been completed, a position applicator screen 515 is displayed, referring to FIG. 100. This screen allows the operator to adjust the targeting catheter drive voltage and frequency, adjust the targeting catheter signal (ATOF) receiver sensors' parameters, and target the desired tissue, such as the renal artery, for therapy. The targeting-related GUI elements 517 are concurrently displayed on the remote targeting monitor, as shown in FIG. 101, located in the patient platform to provide continuous feedback to the operator while positioning the treatment module for treatment.

In this variation, eight different ATOF receivers are activated on the treatment transducer array housing, for detecting the location of the ultrasound beacon. Eight receivers With the remote targeting monitor activated, ATOF signals would be evident in each of the eight wave display boxes 519, as shown in FIGS. 100 and 101. The status of the treatment applicator “Z Piston” as well as the ball and base locks is indicated on the right side of the remote targeting monitor screen—open circles indicate off and red-filled circles indicate on 521. In one variation, the state of these functions is controlled by momentary rocker switches on the treatment module handgrips as well as by push buttons on the water conditioner. Pressing once, turns the function on, and pressing again, turns the function off.

At this juncture, the “Z” Piston would be ON and the Ball and Base locks OFF. Manipulate the treatment module on the subject's skin so as to target the targeting catheter beacon in the renal artery, and to simultaneously obtain strong ATOF signals from all of the ATOF receivers. This can be accomplished by trying various combinations of base translations and ball angulations to achieve accurate targeting and stable ATOF signals. Targeting accuracy may be gauged by the proximity of the “X” on the screen to the center of the bull's-eye on the POSITION APPLICATOR screen. If the signal from the beacon is not strong, then a few possibilities might exist. For example, there may be a structure in the way of the signal such as bowel or bone. Alternatively, the position of the array may be out of range with respect to the

In one approach, the apex of the treatment module nosecone is positioned proximal to the costovertebral junction and the straight side parallel to the spine, taking care to not extend beyond the red treatment-window-boundary line. Also, angulate the plane of the nosecone to be approximately parallel with the plane of the skin, and seek to maintain strong ATOF signals in all eight wave-display boxes, and endeavor to minimize the Z (depth) distance, which is displayed on both position applicator screens.

Using the remote treatment module camera monitor, inspect the membrane-skin interface for bubbles trapped in the coupling gel. If bubble is present the user can then proceed to eliminate these bubbles.

In one example, with nominal targeting achieved and adequate ATOF signals displayed in each of the eight wave-display boxes, the targeting catheter and ATOF parameters can be optimized. In the event that one or more of the ATOF signals is weak (low amplitude) or unstable (signal leading edge jitter), the operator may tune the targeting catheter beacon frequency (“Pinger Frequency”) to reduce any spurious noise observed on the ATOF signal traces, especially near the leading edge of the main waveform; adjust the targeting catheter transmit voltage (“Pinger Voltage”) to achieve overall higher amplitude ATOF signals; and/or modify the ATOF TGC (Time Gain Compensation) to increase the signal or to reduce noise; in order to establish strong, stable signals.

In one configuration, if a particular ATOF signal stability continue to be elusive, the offending ATOF sensor(s) can be switched off by unchecking the checkbox adjacent to and left of its wave-display box(es). The system may then rely on the remaining ATOF receivers to track the position of the ultrasound beacon.

In one application, the targeting catheter with the ultrasound beacon is positioned within the renal artery for treatment of nerve surrounding the artery. One of ordinary skill of art having the benefit of this disclosure would appreciate that beacon may be placed with various approaches for treatment of different tissues in the patient's body.

Using the steps describe above, manipulate the treatment module on the subject's skin so as to target the targeting catheter beacon in the renal artery. When the targeting crosshair (X) is centered in the targeting bull's eye, lock the treatment module ball and base using the appropriate handgrip switches. Assure that these movements are locked by noting their status light in the status box 521 in FIG. 101.

When targeting has been achieved, click the position applicator button at the bottom of the screen. In one variation, once the position applicator button has been clicked on, the system locks the position of the base mover and the orientation of the applicator, which prevents the operator from accidentally moving the position of the treatment module and the orientation of the applicator.

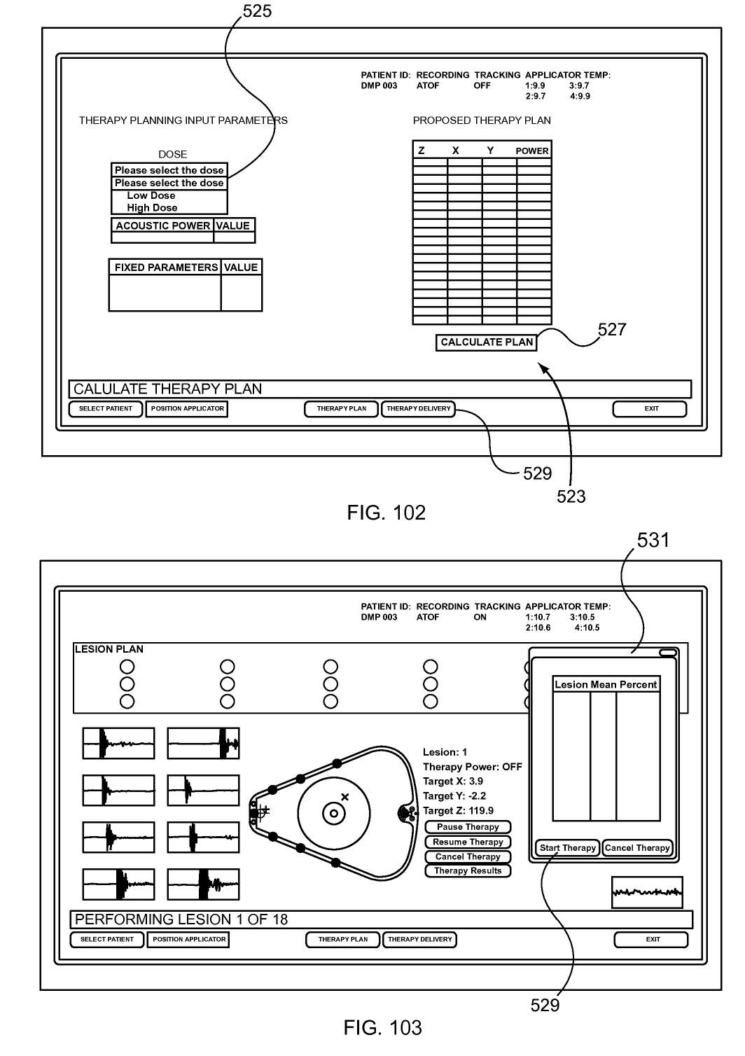

Once the targeting has been completed, the operator may then specify a therapy plan. This can be accomplished using a calculate therapy plan screen 523, FIG. 102. Dose selection can be accomplished be clicking on a drop-down table 525 and selecting the desired dose per protocol. The computing unit may be preprogramed with different dosage protocol. Following the dose selection, click on the “Calculate Plan” button 527 to produce the PROPOSED THERAPY PLAN table. In the event that the acoustic power required to achieve the prescribed dose exceeds a predetermined wattage (e.g., 320 Watts, or the maximum power that the system can produce), a warning is displayed that indicates the power that would be required to achieve the prescribed dose and asks if you would like to continue treatment at a recommended maximum dose (e.g., 320 Watts) can deliver. Depending on the clinical application and the system design different max wattage warning threshold may be provided.

To initiate therapy delivery, click the “THERAPY DELIVERY” button 529 at the bottom of the screen. Prior to the start of focused therapeutic ultrasound delivery, the system steps through each of the calculated treatment positions to assess the targeting quality anticipated to occur at each location during treatment (FIG. 103). If the ATOF Quality Check yields adequate targeting stability, the operator may perform a pre-treatment angiogram to verify that the targeting catheter is in the desired position in the renal artery. Then, using fluoroscopy, perform a renal artery angiogram to determine if the location of the targeting catheter beacon is still adequate. If not, reposition it to the desired location, and return earlier steps to adjust ATOF signal received from the targeting catheter.

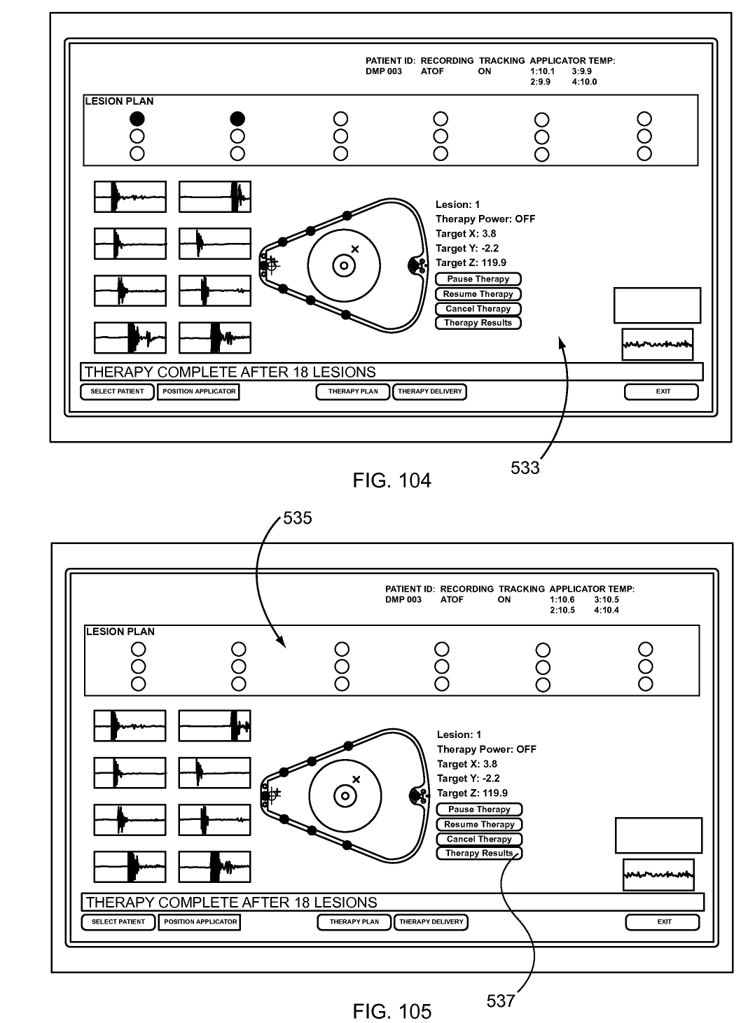

To initiate Administration of the Therapeutic ultrasound treatment, click the “Start Therapy” button 529 in the ATOF Quality Check Dialog 531 to initiate treatment delivery (FIG. 103). Therapy progress is tracked and monitored 533 throughout the treatment session (FIG. 104). At the conclusion of therapy delivery, the quality of each sonication is displayed in the LESION PLAN box 535. Red circles indicate delivery of the specified dose, and gray circles indicate less than the specified dose was delivered (FIG. 105). To end the treatment session, click the “Therapy Results” button 537.

Each treatment session or treatment process may comprise delivery of focused ultrasound energy into a plurality of regions in a given treatment area to form a pattern within the treatment area for therapeutic purposes. The treatment regions may also be refer to as lesions in certain applications, since high energy delivered to the tissue over a period of time can result in lesions in the tissue.

Using fluoroscopy, perform a renal artery angiogram to determine if the location of the targeting catheter beacon is unchanged from that in the pre-treatment angiogram. If not, record the targeting catheter beacon location in the GENERAL THERAPY COMMENTS box on the SESSION COMPLETE screen. Click the “End Session” button in the SESSION COMPLETE screen to shut down the treatment procedure application.

VI. Tracking of Target Tissue with Imaging Transducer Array

In some variations, the ultrasound therapeutic systems disclosed herein are implemented with ultrasound imaging capability for locating and/or tracking the target tissue to be treated. The imaging functionality may be implemented alone or in combination with a tracking beacon, to assist with the placement of the treatment transducer array and for tracking the position of the target tissue during therapy delivery.

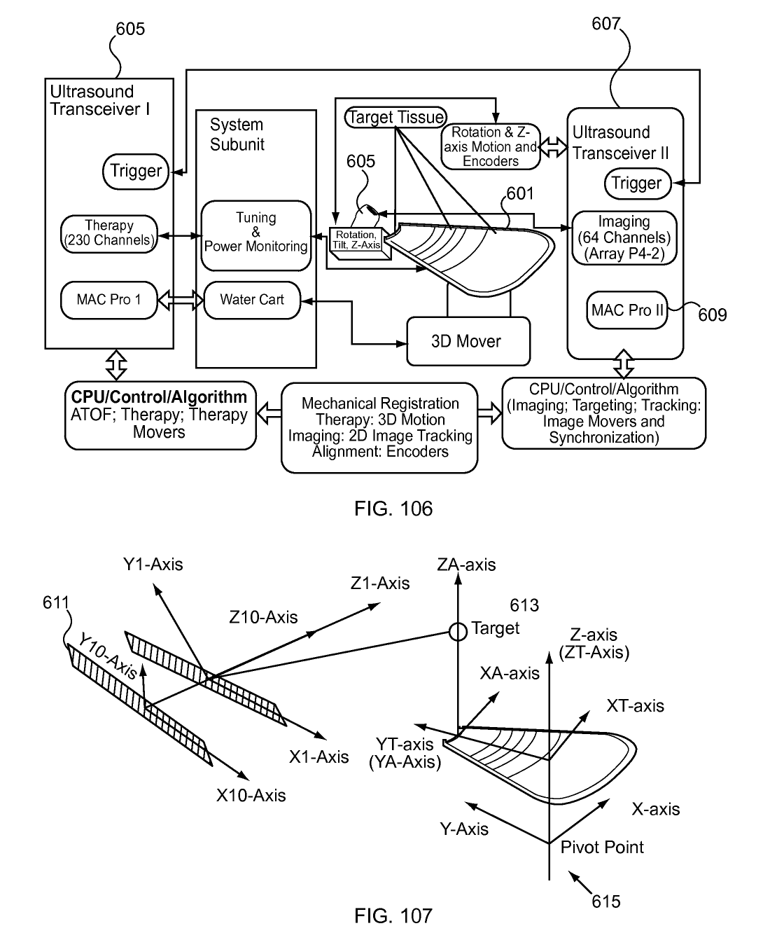

In one preferred embodiment, as shown in FIG. 106, the system comprises a treatment array 601 and a separate imaging array 603. The imaging array may be a three dimensional (3D) ultrasound imaging array, or a two-dimensional (2D) imaging array configured with actuation to scan a volume of tissue. For example, the imagining array may be attached to an actuator controlled by the system's control unit and capable being moved (e.g., rotation and/or tilt) independent of the treatment array. In this example, a first ultrasound transceiver 605 is configured to drive the treatment array 601, and a second ultrasound transceiver 607 is configured to drive the imaging array 603. A computation unit 609 is optionally configured to control the actuator that is coupled to the imaging array to direct rotation and Z-axis motion of the imaging array 603. Encoders are provided on the actuator for feedback control. In one variation, the base of the imaging array is mechanically coupled to the treatment array, such that the system is able to determine the relative position and orientation of the two ultrasound array.

In another embodiment, the imaging array is two-dimensional and the movement in and out of plane is minimized by a combination of anesthesia and understanding the location for probe placement . . . that is, which orientation of the probe approximates a plane wherein the only movement occurs (i.e. the principal plane) and no out of plane movement exists. In this embodiment, a relatively simple 2D probe and image can be utilized to track and correlate motion of the target with the motion seen on the ultrasound image. In another embodiment, an interface is provided for the user in which directionality for the principal plane guides the user to position the ultrasound imaging array into the correct orientation to maximize usability and efficiency.

FIG. 107 is a graphical illustration for using a one or two dimensional ultrasound array structure 611 to scan and track the position of a target tissue 613. The 2D image tracking information of the target is registered to the 3D spatial coordinate 615 of the therapy system, allowing the system to position the focus of the treatment array on the target, and continue to adjust the position of the focus to track the movement of the target.

Imaging probe 605 can be independently moved in 1, 2, 3, 4, 5, or 6 degrees of freedom. Its orientation relative to the therapy array 601 is determined using an electromagnetic or optical sensor. During therapy, a target on the imaging screen is located by a user and the moving target is tracked by the system through treatment. The position of the target on the screen of the imaging probe is related to the therapy position of the therapeutic array 601 by the electromagnetic or optical sensor. The slice of the imaging probe projected through the tissue can be related to the three dimensional coordinate of the therapeutic array by the same optical sensor (e.g. see FIG. 61L). Fiducials on the imaging probe and fiducials on the therapeutic array allow the two to be easily related to one another in space. The optical or electromagnetic tracking of position of the target is updated at least every 200 ms (milliseconds) and preferably under 100 ms. Most favorably, the target position is updated in under 20 ms (e.g. 1-5 ms). The faster the update rate of position, the better tracking will be with respect to movement of the patient and the target area for treatment. Moreover, in some embodiments, therapy of the target cannot take place while imaging and tracking are occurring because the therapeutic ultrasound interferes with the imaging ultrasound. Therefore, the shorter the target acquisition time, the more time there is for therapy. This type of sequence is called multiplexing.

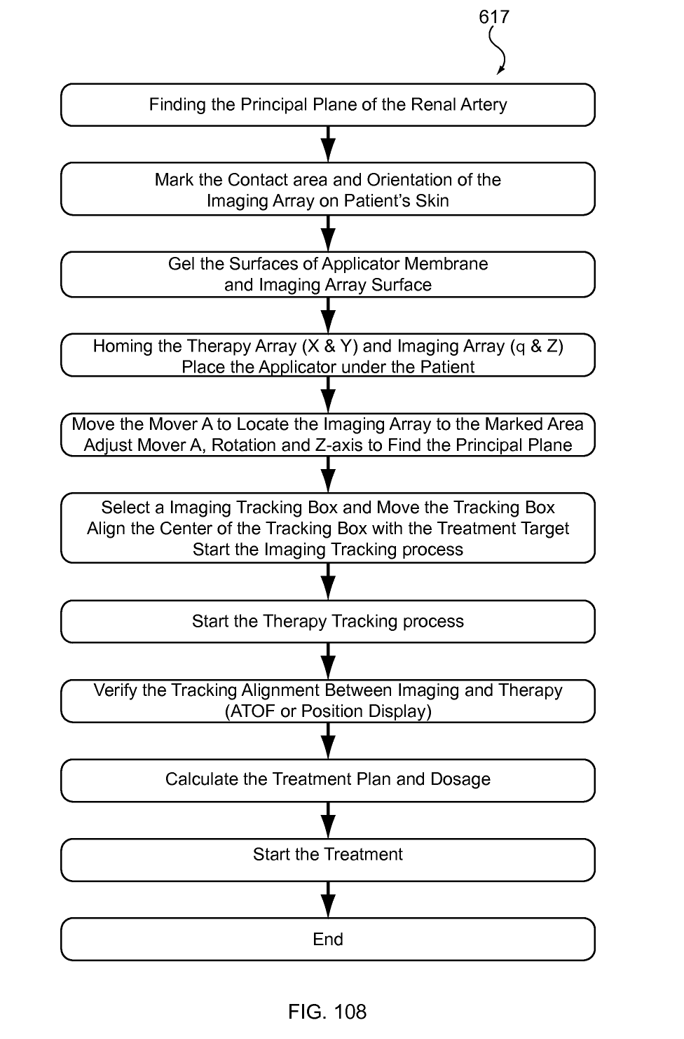

In one variation, where the movement of the target stays within or close to a principle plane or a primary plane, the therapeutic system may identify such principle plane first, and align the imaging array to track movements within such a principle plan. Once the system is able to track the target tissue in the principle plane, the system can then proceed with delivery of therapy, and keeping the focus of the treatment array on the target throughout the treatment process. One specific example of treatment steps 617 that utilizes the principle plane approach is illustrated in FIG. 108. In this example, the user first finds the renal artery and associated movement of the renal artery The principal plane might be exact or approximate but nonetheless, movement of the renal artery can be followed visually on the user screen and this movement is the major movement of the treatment region with little movement occurring in the other planes. In the next step, the imaging array is locked into position on the patient's skin. The lock is typically a mechanical lock, in the form of an arm attached to the patient bed. A spring lock may also be a part of the mechanism to lock the imaging transducer to the patient. With the imaging array locked into place, a virtual treatment region can be projected from the imaging probe and the coordinates of this virtual treatment region can be translated to the real coordinate space and related to the therapy transducer. These steps are accomplished via an algorithm in the software of the system. The next step is placement of the therapy transducer so that it is within the virtual treatment region of the imaging array. Software inside the system can also assist with this step. The next step is to start tracking. At this point, a tracker box with a region of interest is placed in a region with quality tracking. The tracker box and region of interest can be selected by the operator of the system or it can be chosen automatically by the system. Quality tracking refers to how well the speckle pattern or anatomic pattern of a current imaging frame resembles a previous frame or frame set. Finding the same region of interest in the current imaging frame or frame set in the current image and correlating it with a previous image or image set equates to good tracking. With good tracking, the updated position of the target can be determined. In some embodiments, multiple region of interest are marked. In some embodiments one region of interest will track speckles and another region of interest will track anatomic features. Anatomic features can also include color flow or Doppler flow components of the anatomic image. As the tracking box and regions of interest moves, so does the target. The consistency of the target movement relative to the tracking of the region of interest is assessed by the processor and the system. If this consistency over time remains relatively constant, then the tracking algorithm is deemed to be working correctly (adequate for tracking) and treatment may commence after the treatment plan and dosage are determined.

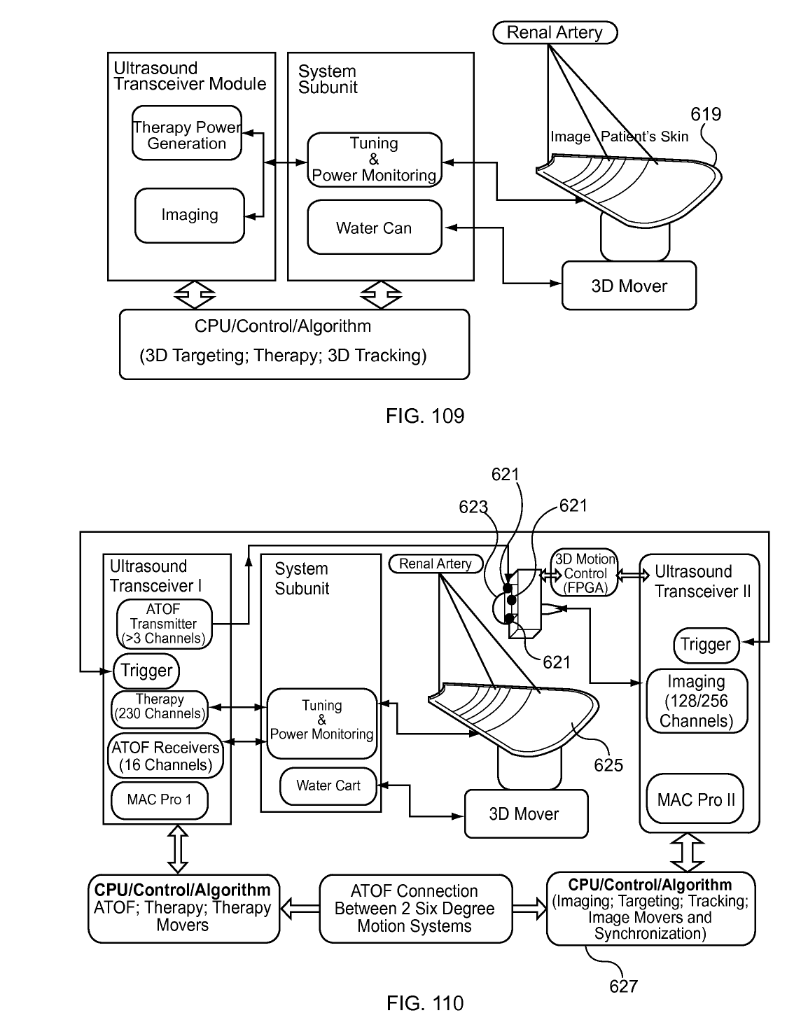

FIG. 109 illustrates another example where a single array 619 is utilized for both imaging and treatment purposes. Imaging and treatment functions may be time multiplexed to allow for continuous tracking of the target during the treatment cycle. In one variation, the imaging and treatment utilizes the same, or approximately the same, ultrasound frequency. When the frequencies are close, multiplexing is necessary because the therapy and imaging will interfere with one another. Multiplexing involves switching therapy off during imaging and vice-versa. In another variation, the transducer array is driven at one frequency for imaging and another distinct frequency for treatment so that imaging, treatment, and tracking continue without interference because the frequencies are distinct.

In another example, shown in FIG. 110, three or more ATOF beacons 621 are positioned on the imaging array 623, allowing the ATOF receivers that are located on the treatment array 625 to determine the position and orientation of the imaging array. That is, the beacons on the imaging array fire an ultrasound signal and the receivers on the therapy array detect the signal and can then determine the orientation and register the imaging probe relative to the treatment array. The computing unit 627 can then register the movement of the imaging array to the system coordinate used by the treatment array 625. One of ordinary skill in the art having the benefit of this disclosure would appreciate that other sensors, such as magnetic sensors, RF position sensors, or imaging registration of known fiducial marks, may be used to determine the position and orientation information of the imaging array, in order to register the position and movements of the imaging array and the treatment array onto a single coordinate system.

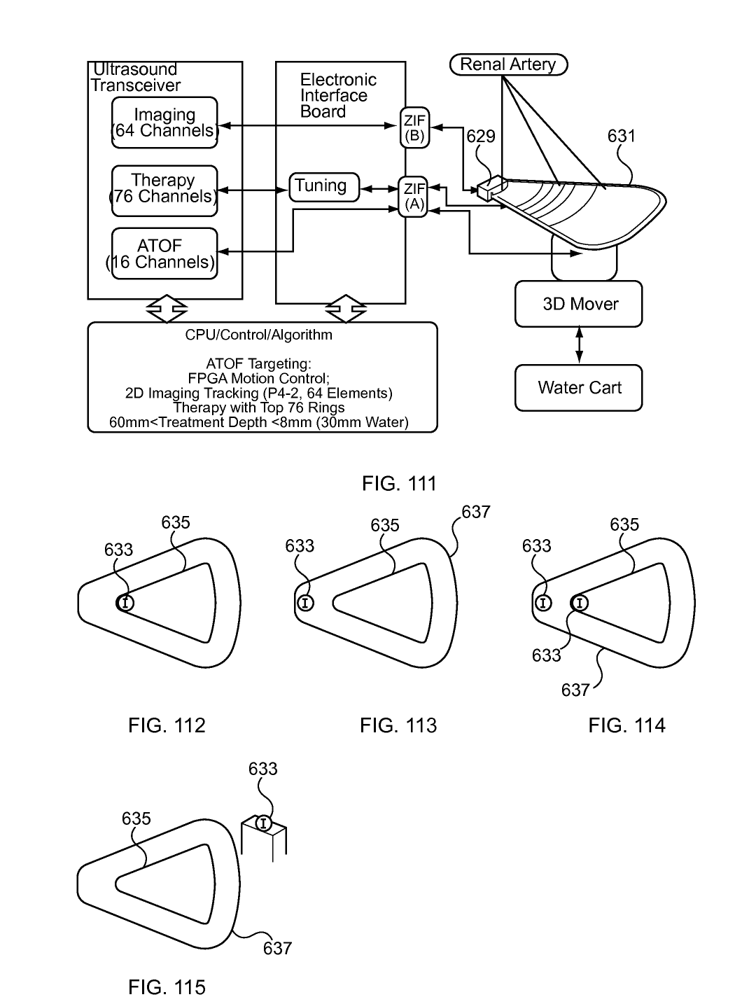

FIG. 111 illustrates another example where the imaging array 629 is fixedly coupled to the treatment array 631. In this variation, since the two arrays 629, 631 are physically coupled together, their movements are in lock step and can be track within the same coordinate system without the need for further registration.

The therapeutic system may utilize one or more imaging arrays for the tracking of the target tissue, and the imaging array may be coupled to the treatment array, or separately positioned to detect the treatment region. For example, the imaging array 633 may be coupled to the treatment array 635 (see FIG. 112), or coupled to the applicator housing 637 (see FIG. 113). Alternatively, two imaging arrays 633 can be used. For example, one coupled to the treatment array 635, and one coupled to the applicator housing 637, as shown in FIG. 114. In yet another example, the imaging array 633 is de-coupled from the treatment array 635 and the applicator 637, as shown in FIG. 115.

One of ordinary skill in the art having the benefit of this disclosure would appreciate that various image processing approaches may be implemented to facilitate the ultrasound image construction and deconstruction. For example, pixel oriented processing and/or voxel oriented processing disclosed in U.S. patent publication US 2009/0112095A1 (application Ser. No. 11/911,633) entitled “Ultrasound Imaging System with Pixel Oriented Processing,” which is incorporate herein by reference for all purposes, may utilized with the system and processes disclosed herein.

### VII. Control

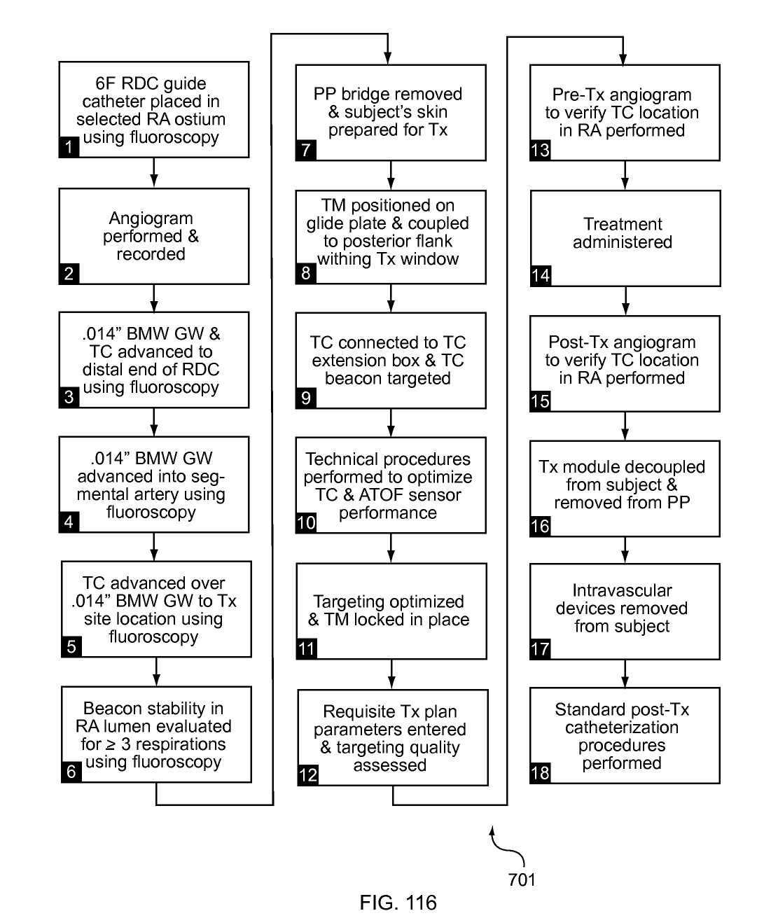

Referring to FIG. 116, an example of steps 701 to perform a focus ultrasound therapy using a targeting catheter to guide the positioning of the treatment transducer focus is illustrated. As disclosed herein, many of these steps may be controlled, monitored, and/or automated through computer control. For variations relying on imaging for targeting, the steps for introducing the targeting catheter may be removed, and additional steps for identifying the target region using imaging and tracking of the target with imaging may be introduced.

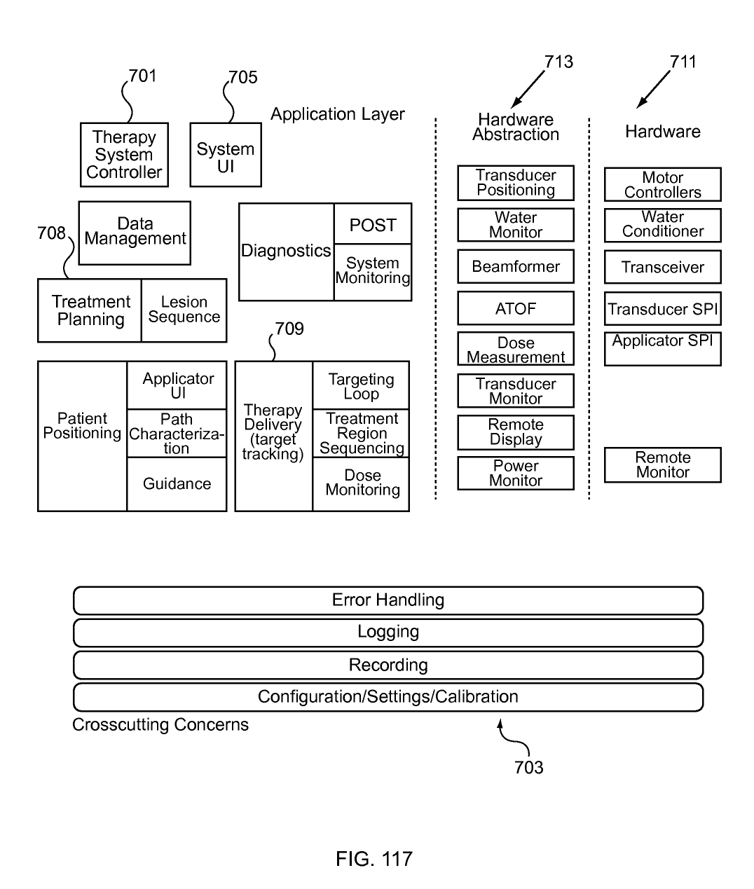

A variation of a software configuration 703 for a therapeutic system is illustrated in FIG. 117. The major components of this software system include Treatment Planning and Delivery, and GUI. The major sub-systems are the UI 705, Therapy System Controller 707, and Treatment Planning 708 and Therapy Delivery 709. The hardware layer 711 wraps the detailed interfaces of the multiple hardware sub-systems. The Hardware Abstraction layer 713 combines one or more hardware sub-systems into a system level abstraction. For example the Motor Controller handles the details of communicating with the motors, and the Transducer Positioning uses the Motor Controller to provide motion. The application layer uses one or more of the hardware abstractions to perform a specific function. For example, the Targeting Loop uses the ATOF, Transducer Positioning and Beamformer abstractions to provide tracking of the therapy target during therapy delivery. The cross-cutting layer provides services that are used by the other layers. Logging for example provides the facility for any other function to log internal operating messages for use in development, debugging and servicing.

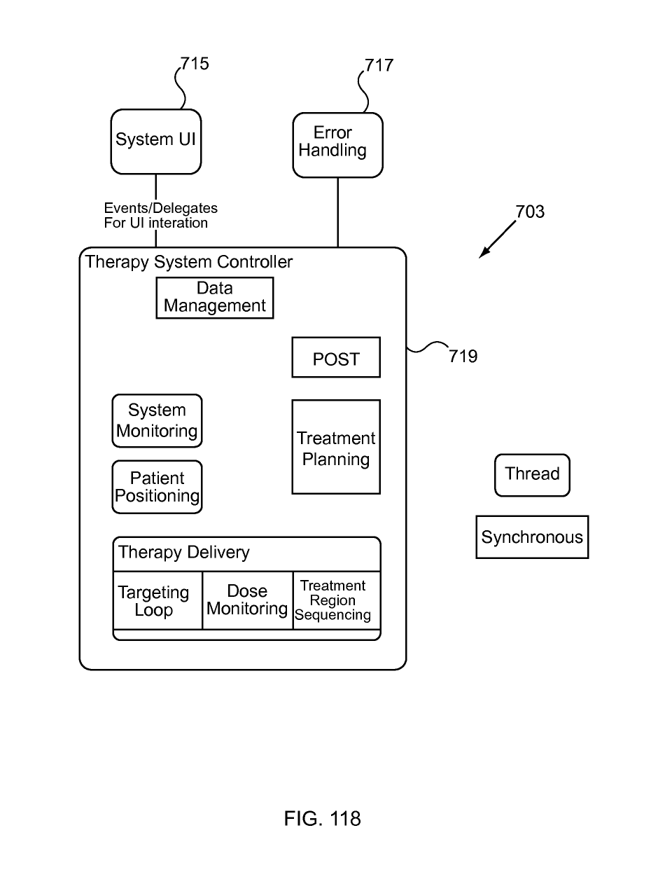

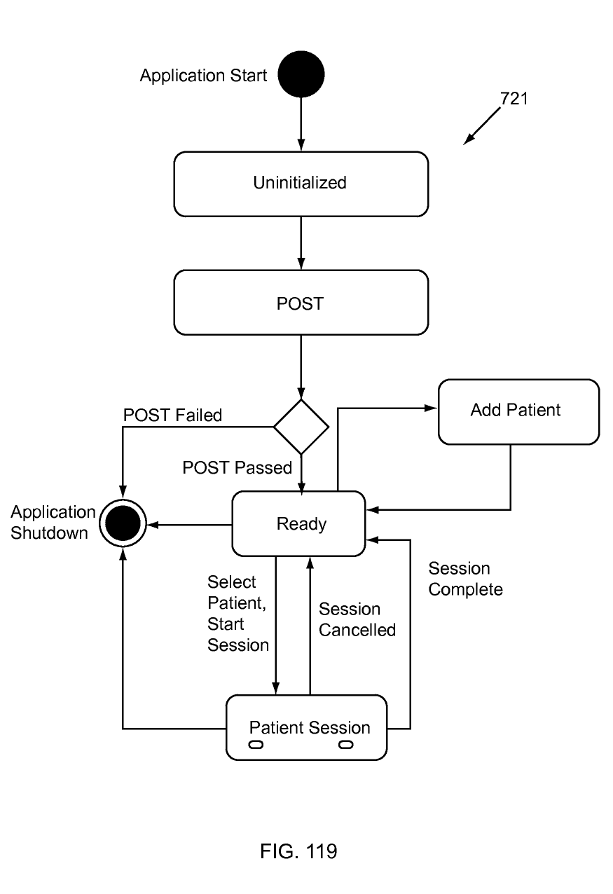

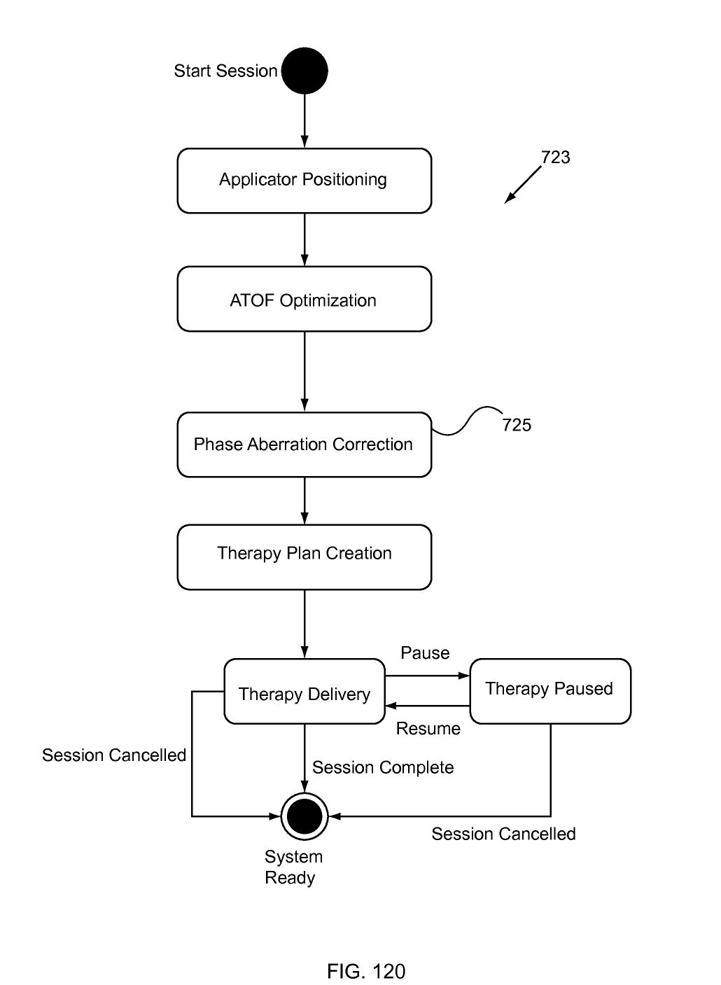

FIG. 118 shows a dynamic view of one variation of a software system 703. The UI 715, Error Handling 717, and Therapy System Controller 719 are independently executing threads and communicate via method and delegate calls. Within the Therapy System Controller 719 several functions are implemented as threads due to their long running nature. In variations where the Therapy loop is a separate thread, this soft real-time loop is given the priority it needs. FIG. 119 shows the top level states 721 of one variation of the therapeutic system. One implementation of patient session states 723 is illustrated in the state diagram in FIG. 120. In another implementation, the steps shown in FIG. 120 are executed without the “Phase Aberration Correction” step 725.

Condition handling is the processing of conditions outside of the normal workflow that require attention. In one variation, a central condition handler is implemented to allow a condition that may occur at any point, to be sent to the condition handler where a comprehensive response can be executed. In one example, the stages in condition handling are detection, user notification, and recovery, based on the user's response or in some cases, a system required response.

In one variation, upon system power-up, the system performs at least the following tests (i.e., the Power On Self-Test (POST)): (a) generator self-test, (b) motor controllers self-test, (c) communications with the system's monitoring functions. The system software is configured to support operation in Startup mode. During startup, the system readiness is checked via a POST. If the POST does not pass, the system will be required to be re-started.

In one variation, the system software is configured to support patient setup. Patient setup allows the selection and verification of the patient to be treated. Patient setup also allows some input of treatment parameters that are expected to vary from treatment to treatment, for the same patient. Patient setup guides the clinicians in positioning the applicator against the patient prior to therapy. The system software plans treatment using the patient specific data, and any system data acquired during field test and/or applicator positioning.

The system software supports therapy delivery. The delivery of therapy applies the treatment as described by the treatment plan. The system software delivers therapy as described by the treatment plan. During application of therapy, the current treatment location on a target tissue or target region can be tracked to insure the therapy is delivered to where it is intended.

As shown in FIG. 117, in this example, there are multiple hardware sub-systems, each with its own unique interface. These include: (a) The system software supports for the motor controllers that drive the motors in response to serial commands from the system software, which may be sent via USB to RS-422 converters. (b) The system software supports for the water conditioner's interface, such as through a USB link. (c) The system software supports for the power and transducer monitors, such as through a USB link. (d) The system software supports for the High Voltage power supply, such as through a USB link. (e) The system software supports for the remote displays. (f) The system supports for the ultrasound transceiver, such as via a PCIe interface.

A therapeutic system may be configured for continuous acquisition and checking of system operational parameters. In one variation, the following operation parameters are monitored: (a) treatment transducer temperature (for example one, two, three, four, or more temperature sensors may be position on or around the treatment transducer array); (b) transducer forward and reverse electrical power (for example one or more of the channels driving the transducers may be monitor; in some variations only the channels driving the treatment transducers are monitored; in some variations all the channels, including ones driving the treatment transducers, ones driving the ATOF receivers, and the one driving the transducer within the targeting catheter, are monitored); (c) water conditioner water temperature; (d) treatment module water pressure; (d) treatment module switches; (e) treatment module inclinometer (i.e., orientation sensor); and (f) treatment transducer array inclinometer.

In some variations, the system is configured to notify the operator when a condition, if any, monitored value exceeds its pre-determined value. The system may also be configured to execute a predefined process or protocol when certain condition is detected. In some variations, the operator is given the option to proceed or abort an existing operation in response to an out-of-limits notification. In some variations, all the monitored values are logged at a minimum rate of once per second. In some variations, the computing unit comprises 2 or more microprocessors running 2.5 GHz or more, at least 16 GB of RAM, at least 1 TB of disk space.

In some variations, the system is configured with a centralized condition handling facility that can be used to notify the operator of conditions, and allow the operator to attempt recovery. In some variation, the system is configured to pause therapy delivery, until the operator acknowledges the notification. The system shall resume or stop therapy delivery after the user acknowledgement is received. The system shall take further actions in an attempt to recover from the condition. The therapeutic system may continuously publish the status to the “condition handling” (a.k.a., heartbeat to conditioning handling) during a treatment procedure. The system may also continuously monitor the status of the “system controller” and publish the status to the system controller. If the therapy system controller or condition handling subsystem does not detect the other's heartbeat (i.e., status report/indication from other subsystems and functions indicating the subsystems or function is functioning properly), an error shall occur and the other subsystem shall stop therapy if in progress. Therapy System Controller may also monitor the status of the various hardware subsystem and components, and publish a heartbeat to the hardware watchdog (i.e., hardware status monitoring). If hardware heartbeat stops, indicating hardware malfunction or out of bound operation, the system shall shut down the electrical power driving the focused ultrasound therapy. Data collected during a therapeutic procedure may be recorded. The recorded information may be utilized post-session to analyze, debug or service the system. In one variation, the water conditioning unit provides chilling and degassing of the water. It also routes the applicator switches and the applicator inclinometer and transducer pressure sensor signals. The water conditioning status can be made available to the system's computing unit for system monitoring.

In some variations, the system software is configured to assist the operator in positioning the applicator, to obtain a desired view of the treatment area. In examples where a beacon is placed at the treatment site, during applicator positioning, the operator may be presented with visual guidance indicating the ATOF detectors field of view of the target. Before presenting guidance, the system may drive the transducer to a “home” position. Once the operator has positioned the applicator, the system can move the transducer through its operating range of motion to show the ATOF detectors field of view of the target.

In one variation, the therapeutic system is configured such that it is capable of targeting and dosing tissues in humans by placing the focus of the ultrasound field from 80 mm to 160 mm in depth from the surface of the patient's body (measured from the shortest distance from the tissue to the skin surface). In another variation, the therapeutic system is configured such that it is capable of targeting and dosing tissues in humans by placing the focus of the ultrasound field from 60 mm to 200 mm in depth from the surface of the patient's body. In yet another variation, the therapeutic system is configured such that it is capable of targeting and dosing tissues in humans by placing the focus of the ultrasound field from 100 mm to 140 mm in depth from the surface of the patient's body. In some variations, the targeted tissue is located under ultrasound obstructive tissue (e.g., bone), such that the target is not directly under a treatment window (i.e., outside the direct perpendicular view of the window. The treatment array is configured such that when the treatment array is positioned over the treatment window, it is capable of propagating a focus ultrasound at an angle (from the normal to the array surface) to a target under the obstructive tissue. In one variation, the array is configured such that when the applicator is positioned parallel to the patient's skin surface, the array is capable of propagating a focused ultrasound at an angle (from the normal to the array surface) to reach the target under the obstructive tissue. In yet another variation, the array is configured such that when the array is positioned parallel to the patient's skin surface, the array is capable of propagating a focused ultrasound at an angle (from the normal to the array surface) to reach the target under the obstructive tissue.

In some variations, the system is configured such that it is capable of targeting and dosing a non-stationary target tissue with focused ultrasound energy with up to 2 cm of movements (such as respiratory related movement). In other variations, the system is also configured such that it is capable of targeting and dosing a non-stationary target tissue with focused ultrasound energy with up to 3 cm of movements. In yet other variations, the system is also configured such that it is capable of targeting and dosing target tissue with focused ultrasound energy with up to 4 cm of movements. In some variation, the system is configured such that it is capable of automatically determining and delivering the proper intensity, duty cycle, exposure time and distribution of ultrasound to achieve therapeutic effect based on operator and measured system inputs. The system may also be configured to estimate in real-time the total focused ultrasound energy dose deposited at the treatment site in the patient.

In one variation, the system continuously computes an ATOF quality factor during the treatment process. In one example, the ATOF quality factor is deemed unacceptable if the ATOF algorithm is unable to compute a position from its inputs for more than 20 updates of the inputs. In addition, the system may continuously compute a tracking quality factor. In one example, the tracking quality factor is deemed unacceptable if the position computed by the ATOF differs from the position computed by the predictive algorithm by more than 1 mm, for more than 5 updates of the position. In one variation, when either the ATOF or the tracking quality factors are unacceptable the system shall shut off therapy power. When the system resumes, the system may be configured such that it extends the treatment time for the target tissue being treated by the amount of time the factor(s) are unacceptable. In another variation, when either the ATOF or the tracking quality factors are unacceptable, but such unacceptable status is within a defined period of time, the system would extends the treatment time for the target tissue being treated by the amount of time the factor(s) are unacceptable when the system status for both ATOF and tracking quality factures are back to functioning in the acceptable range. When the unacceptable status is extended beyond the defined period of time, the system shall shut down.

The tracking quality factor may also comprise a quantitative factor measuring the effectiveness of the movement of the treatment transducer array, in keeping the focal point of the treatment transducer array on the treatment region as the treatment region is displaced during the treatment process. The location of the treatment region may be determined through a beacon or through imaging, which tracks the position of the treatment region.

In one variation, if the ATOF and/or tracking quality factors become acceptable after the therapy power has been shut off due to them being unacceptable, the system shall re-enable the therapy power. If the amount of time a lesion is extended, exceeds 25% of the original lesion on time, the system shall create a condition and notify the user. If the number of lesions that have been extended is greater than four the system shall create a condition and notify the user. The system may be configured to log all the interruption in the delivery of the treatment.

In some variation, the system is configured to store calibration data for multiple treatment modules. The treatment planning uses the treatment module calibration data for the treatment module selected during patient selection. In one example, during the treatment planning phase, if it is determined that the treatment module calibration file is corrupt when the file is opened for use, a condition shall occur and the operator is notified. The system calculates the distance between transducer and target. The system shall limit the number of treatment regions through the treatment process to a predefined number (for example, 18 treatment regions may be defined as a limit for treatment surrounding a vasculature). The system calculates locations for all the treatment regions. The system calculates power dosage for each treatment region. The system then calculates the total treatment time. The system may be configured to limit the maximum treatment time to a pre-defined period of time (for example, the total treatment time may be limited to 15 minutes).

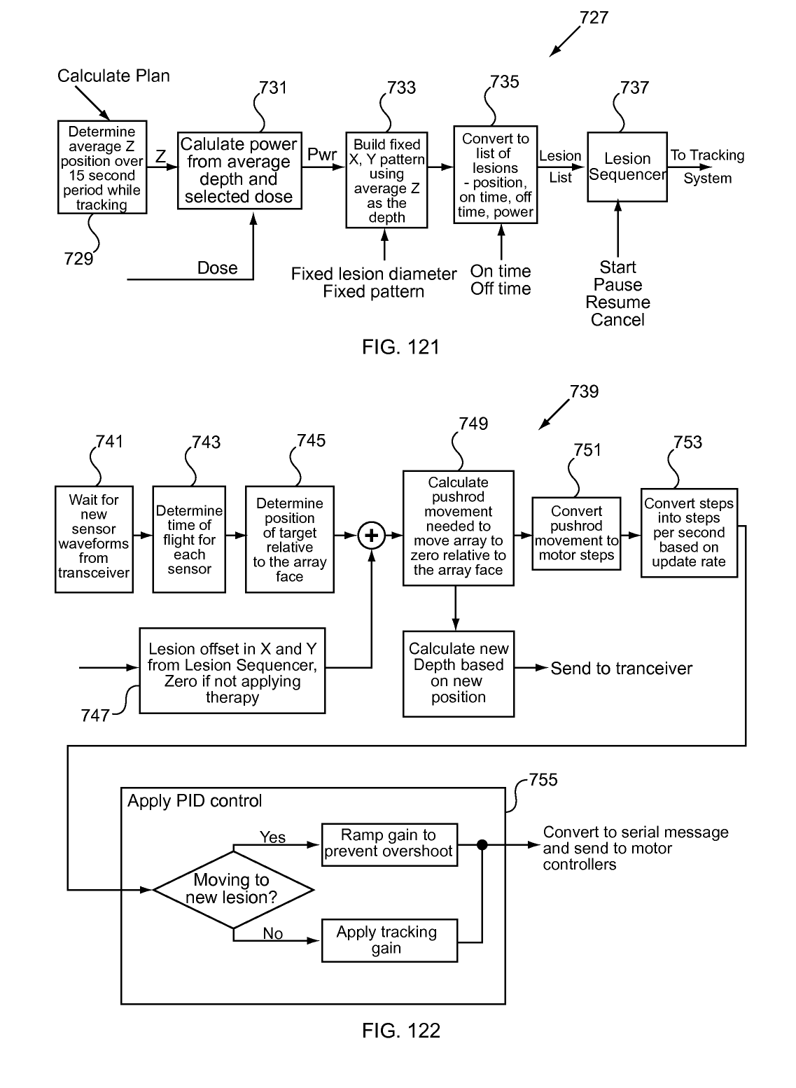

Referring to FIG. 121, an example of a treatment planning process 727 is illustrated in a flow chart. In this example, the system calculates the average Z position (focal depth of the therapeutic for focused treatment ultrasound delivery) over a fifteen second period while tracking the position of the target region 729. The system then calculates power from the average focal depth, and the selected dose information 731. The system builds fixed X, Y pattern using the average Z as the focal depth, using fixed lesion diameter (i.e., diameter for the treatment region), and fixed treatment pattern 733. The information is then converted to generate a list of lesion-positions (i.e., position of the treatment regions), on time, off time, and power 735. The list is then sequenced for execution (i.e., delivery of the therapeutic ultrasound) 737.

In some variations, the system is configured to continuously monitor the three-dimensional location of the treatment region during the entire treatment process or treatment session. The system reports the status of eight ATOF receivers before treatment. The system also calculates the acoustic attenuation factor before treatment. The system measures and records beacon movement when the targeting catheter moves out of treatment zone before treatment. Optionally, the system displays the waveforms from all the ATOF sensors being used for tracking. The system also calculates in real-time the location of catheter beacon. The system records patient respiratory motion during the treatment process. The system also predicts real time treatment zone location according to the beacon's location. The system provides graphical display of the relative position of the real-time target location and treatment transducer array's focal point location. The system continuously relocates the focal point of the transducer during the entire treatment session. In some embodiments, the balloon further contains sensors which can give a sense of the intensity of the focused ultrasound. The sensors may also direct the focused ultrasound therapy in a pre-defined pattern relative to the blood vessel and the target nerves surrounding the vessel. The system controls the movements of the treatment transducer array, and causes the transducer array's focal point to track the movement of the treatment region location in real-time, during the entire treatment session within a defined motion tracking error range.

In some variations, the system is configured to sequentially target the entire treatment area with multiple power shots (in each of defined treatment regions in the treatment area) according to the calculated treatment planning information. The system may be configured to continue a partially-completed treatment plan if therapy power is shut down, and then restarted within a single treatment session. In one variation, the system monitors the power delivered on each of the transducer elements during therapeutic ultrasound energy delivery (i.e., power shots). The system may be configured to shut off therapy power if the accumulated power/energy delivered exceeds the treatment plan limits to prevent over dosing.

The system can also be configured to track in real-time the accuracy performance of a given treatment being delivered. In one example, the system updates the treatment focal location at least every 30 milliseconds, and the duty cycle of therapy power during each update period is no less than 70%.

Referring to FIG. 122, an example of a tracking loop 739 of the tracking system is described in detail. The system waits for the new sensor waveforms from the ultrasound transceiver 741. The system then determines the time of flight for each sensor (i.e., ATOF receiver) 743, followed by determining the position of the target relative to the treatment array face 745. Combing this information with the lesion offset in X and Y from Lesion Sequencer 747, the system calculates push rod movement needed to move the treatment array to zero relative to the array face 749. The system then coverts the push rod movement to motor steps required for the step-motors to displace the push rod in the corresponding amount 751, followed by converting steps into steps per second based on update rate 753. The system also calculates new depth based on new position, and sends such information to the transceiver. The step motor control information is the applied to the PID (Proportional, Integral, Derivative) control 755. The information is processed to electrical signal, and is sent to the motor controllers to move the push rods in the treatment module to move the treatment transducer array to the desired position.

### VIII. System Conditioning and Boundary Conditions

A condition occurs when an unexpected situation arises such that the normal workflow may need to be interrupted and/or the user apprised of the situation. In many cases the user and/or the system will need to take an action to allow the workflow to continue. An example of a condition: if the workflow has progressed to the therapy delivery stage and the treatment array temperature exceeds its upper limit, it may be desirable to pause the treatment process until the array cools enough. An alternative could be that an array over temperature condition is so extreme, that the treatment must be stopped, and the cause of the over temperature corrected.

In one configuration, the conditions are grouped as: (A) Operational Conditions, for example: Power On-Self Test failure; out of limits temperatures or voltages; missing or corrupted information necessary for treatment such as transducer definition files; cable disconnect; loss of power; (B) Performance Condition, for example: target tracking or beacon tracking outside of limits for too long; ATOF unable to detect a usable signal for too long; dosimetry out of limits; (C) Error Condition, for example: interruption in the workflow caused by an unhandled software exception.

Some situations that arise during the workflow execution are not truly conditions. The first type is referred to as workflow errors and is the result of operator input to the graphical user interface. Examples of this would be mistyping a patient ID or trying to move ahead in the workflow, when not all user entered input has been entered. These are expected, and may be dealt with immediately by the operator at that point in the workflow. They do not need system action. A second type is referred to as a normal system correction. These may be handled as part of normal system operation and do not require user notification or action. An example would be the loss of target tracking at any time in the workflow. If the loss is limited to a small amount of time, the system may log the situation, pause therapy if it is on, and then resume therapy and tracking once it can track again. Loss of tracking for an extended period of time on the other hand, would cause a condition as it is assumed that user action may be required.

In one configuration, the following steps are taken to address a condition: (A) Detection—the occurrence of a condition detected by the software or hardware. (B) Workflow interruption—the workflow is paused until the condition is resolved. (C) Operator notification—the operator is notified that a condition has occurred and what action, if any, needs to be taken by them. Any system actions may be described as well, so the operator may anticipate them when they occur. (D) Operator Acknowledgement—the workflow remains interrupted until the user acknowledges they have read the notification, and any recovery instructions it may contain. (E) Resolution—after the operator has acknowledged the condition, the system will either resume the workflow where it was left off, or move to a previous point in the workflow. In some cases, the system may shut down so that it may be re-started. Any other system actions required may be done at this time also.

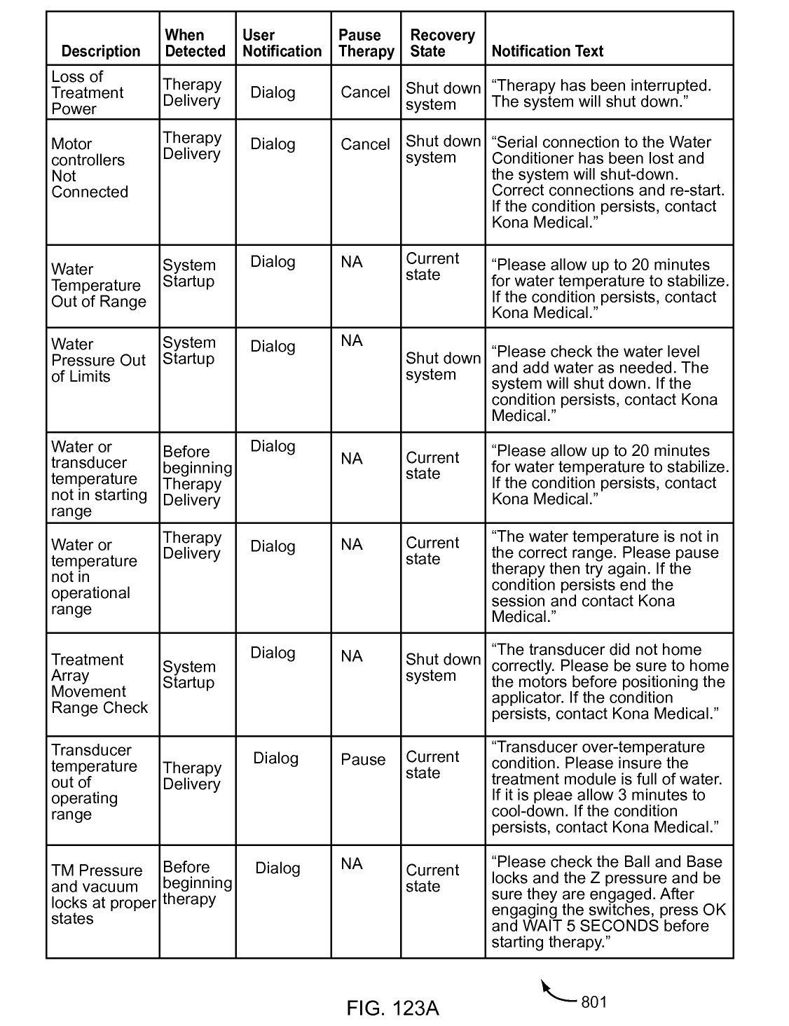

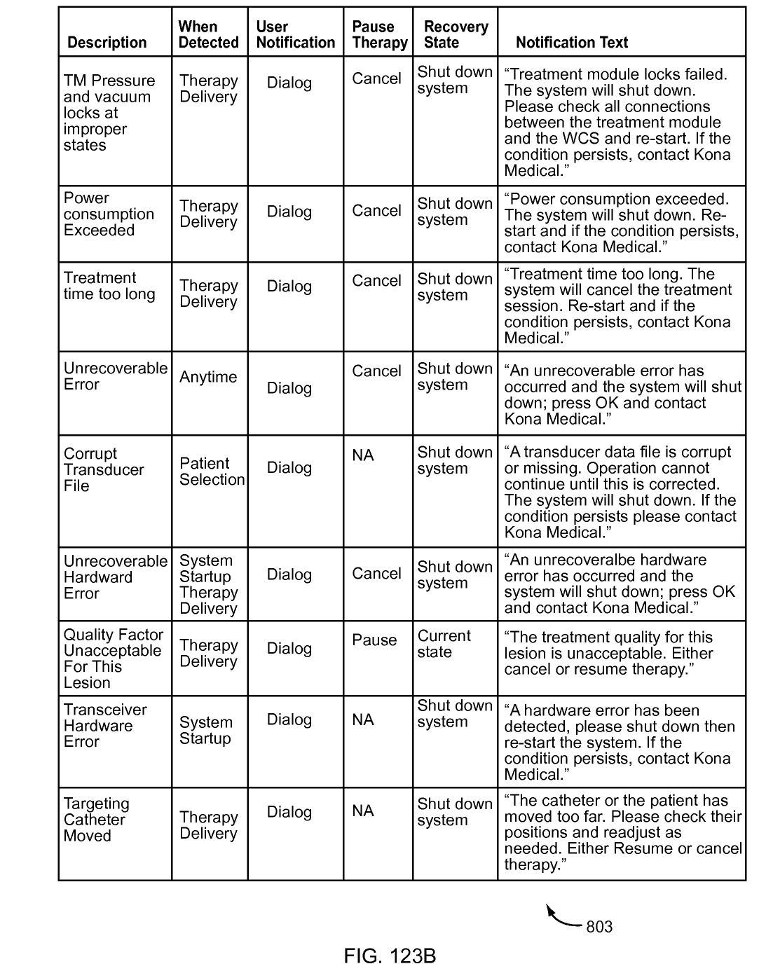

In accordance with the steps needed to handle a condition, a definition of a condition may contain some or all of the following items: (A) Condition number and description—each condition has a summary description and a number to distinguish among the multiple conditions that shall be handled. (B) When detected—some conditions can occur at different times during the workflow. Depending on the workflow step, the condition may or may not need to be handled at that time. (C) Dialog text—a dialog is used to notify the operator of what the condition is and contains any instructions to the user needed to resolve the condition. (D) Audible—some conditions are critical and/or may occur when the operator is not focused on the graphical user interface. In these situations it may be helpful to provide an audible notification in addition to the dialog (i.e., visual indication) that accompanies the conditions. (E) Pause/stop therapy—if therapy delivery is occurring when a condition happens, it may need to be paused. In some cases the condition is non-critical and therapy can continue. (F) Recovery state—after the condition is acknowledged by the operator, the system state may change. (G) Can resume therapy after shutdown—in the case where the system should be shut down, while therapy delivery is occurring, the therapy may or may not be able to be continued after the system is re-started. (H) Other system actions—in some cases requirements defined in the reference documents will require the system to take other actions such as saving data to a file or resetting operational data values to their defaults. Examples of conditions and their corresponding definition and actions are listed in the tables 801, 803 in FIGS. 123A and 123B.

In one configuration, the conditions are prioritized in the following order from highest to lowest: (A) Those that require system shutdown; (B) Those that require therapy to be paused; (C) All others. The recovery states are defined as: (a) Current state; (b) Patient selection; (c) Positioning; (d) Treatment planning; (e) Therapy delivery; (f) Shutdown system. The Detected states are defined as: (a) Anytime; (b) System startup (POST); (c) Patient selection; (d) Positioning; (e) Treatment planning; (f) Therapy delivery. The pause therapy states are defined as: (a) Pause; (b) Cancel.

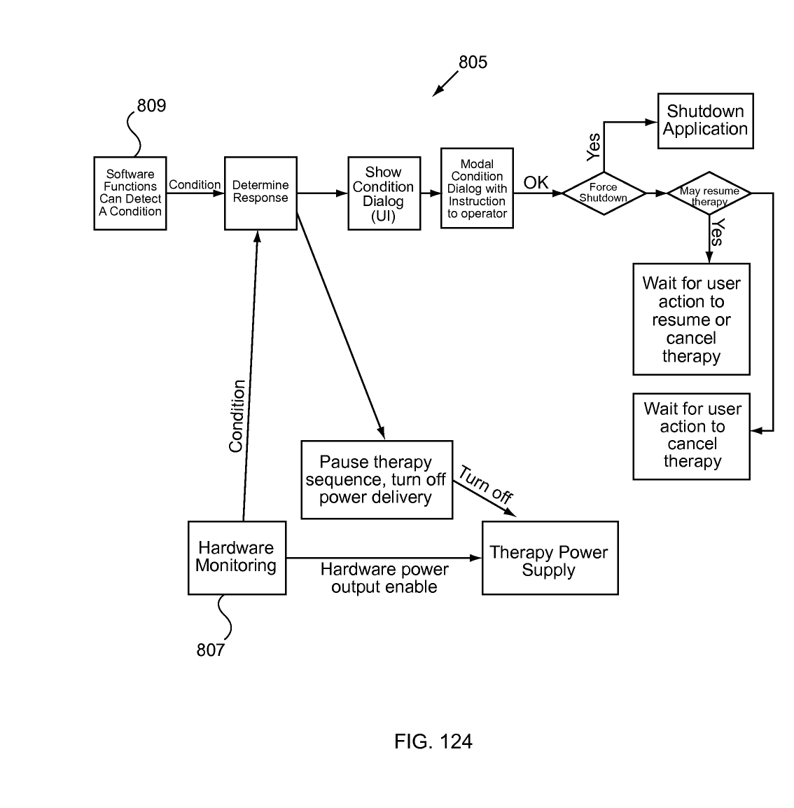

A flowchart illustrating one example of a conditioning handling process 805 is show in FIG. 124. In this example, both the hardware 807 and the software 809 of the system are able to detect conditions, and initiate the conditioning determination and response process. Once the software functions running on the computing unit detect a condition, depending on the condition, the system determines what action is required. At the same time, the hardware operation is also being monitored, such that when hardware monitoring detects a condition, it also prompts the system to determine a proper response. The system prompts the operator with a condition dialog, and pause the therapy, and turn off power delivery to the treatment transducer array. A signal is sent to the therapy power supply to cut-off power supply to the treatment transducer array.

The operator is provided with instruction to address the condition. The system then requests the operator to determine whether a force shutdown of the complete system is necessary. If the operator did not elect a forced shutdown, the system then prompts the user to resume the therapy when he/she is ready.

In some variations, the therapeutic system also comprises an error handler software running on a computing unit. In one configuration, the error handler software is implemented as a central error handling function for the system. In this example, the primary functions provided by the error handler software are: (A) accept error detections from other system functions; (B) pause or stop therapy depending on the error; (C) notify the user and wait for the user response; (D) after the user response, request a system state change which could range from resume therapy to system shut-down; (E) monitor therapy system controller status (e.g., the heartbeat); (F) provide status info (e.g., heartbeat) to hardware to shutdown therapy if the error handler or the therapy system controller ceases functioning.

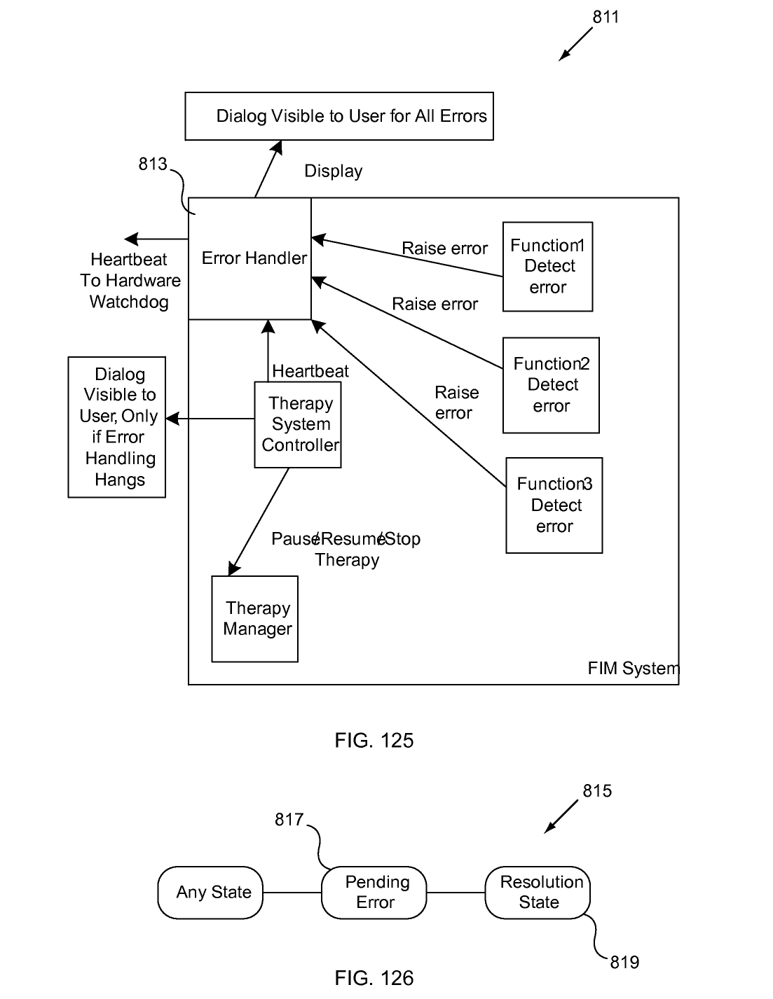

FIG. 125 shows an example of a system context diagram 811 for the error handler. The diagram represents the actors outside the error handler software module 813 that could interact with the module. The error handler interfaces to the rest of the system functions through an error handler interface. In this example, the error handler itself is a singleton to make it accessible to all system functions without the need to pass around a reference and to insure that there is a single point of error handling. In this variation, the therapy system controller serves as the central control, and directs the operation of both hardware functions and other software functions. The error handler and the therapy system controller may continuously exchange system status information during the operation of the system, such that each subsystem can verify that the other is operating property during the treatment process. The status information may comprise a generic signal indicating the subsystem is functioning (e.g., heartbeat) or it may comprise of additional data to provide more details of the subsystem status. The error may also provide a status information, such as an heartbeat, to the hardware monitoring unit (i.e., hardware watch dog), such that when the error handler may notify the hardware monitoring unit to stop therapy deliver if the error detected by the error handler requires the therapy to be terminated or halted. In one variation, the electrical power monitoring unit is configured to serve as the hardware monitoring unit. When the electrical power monitoring unit detects that error handler or the therapy system controller is not functioning properly or has failed to function, the electrical power monitoring unit will terminate the delivery treatment ultrasound energy into the patient's body. This may be achieved by either terminating the delivery of electrical power from the high voltage power supply to the transceiver unit that drives the treatment array, or terminating the electrical power going from the output channels of the transceiver going to the treatment array.

The therapy manager may comprise a lesion or treatment region sequencer. In one example, the lesion sequencer directs the treatment module and the transceiver to deliver a series of focused ultrasound energy, and sequentially treat one treatment region after the other, until a treatment pattern directed by the treatment plan is formed within the treatment area.

In response to an error, the error handler first pause the therapy if it is occurring. Depending on the error, the therapy may or may not be resumed after the user acknowledges the error. The system state may change to some other state than the current state. The error handler will request this state transition via the therapy system controller. Each error has a set of items such as text fields, and system states that describes the system response when the error occurs. In addition, each error is associated with one or more triggers (causes). The definitions of the responses and the triggers are stored in these files on the computing unit.

In variations where the error handler is a singleton, it may be available as soon as any function needs to access it. In one variation, the first access is via the therapy system controller heartbeat. When an error is not present, the system operates per the pre-defined workflow. The therapy system controller heartbeat and the error handler heartbeat interact to provide aliveness checking.

The therapy system controller heartbeat thread, continually updates its aliveness to the error handler. Should the therapy system controller heartbeat cease, the error handler will stop petting the hardware watchdog (i.e., stop sending status single/update indicating that the function is operating properly). The error handler in turn, updates its aliveness to the therapy system controller. Should the error handler hang, the therapy system controller will notify the user via a dialog, before shutting down the application to force a restart.

The therapy system controller's heartbeat is used to insure the therapy loop is operating, to insure the error handler loop is operating, to check the system monitoring function, and to update the therapy system controller aliveness in the error handler.

When a function detects an error, it calls the error handler via a new error protocol through the error handler interface. On receipt of a new error, the error handler will put up a user notification in the form of a dialog. In some variations, access to the user interface during the error handler sequence is prevented. The error handler tells the therapy system controller to go to the pending error state when a new error occurs. The therapy system controller shall not respond to UI action or data requests as long as it is in the pending error state. After the error has been resolved, the therapy system controller will be told to leave the Pending Error state and go to which ever state is required by the error resolution. The therapy system controller will then be able to respond to UI requests.

Referring to FIG. 126, one variation of an error resolution state sequence 815 is illustrated. The diagram shows the states of the therapy system controller during the error handler sequence. The pending error state 817 allows the therapy system controller to ignore UI requests, and to perform which ever action is needed to pause system operation, while the error dialog is being shown. Once the operator acknowledges the dialog, the error handler will tell the therapy system controller to go to the appropriate resolution state 819. The resolution state may be any valid system state including shut-down. The therapeutic system may also be provided with hardware failsafe. For example, the error handler may provide a periodic signal to the hardware watchdog. In the event that the therapeutic controller or the error handler hangs, this signal will cease, and the watchdog will prevent the hardware from delivering therapy.

### IX. Treatment Module

The treatment transducer array may be positioned within a treatment module, which controls the movement and orientation of the treatment transducer array, provide the interface for coupling the treatment transducer array to the patient's body, and cools the transducer array and the contact surface with the patient's skin. The cooling of the treatment transducer array keeps the temperatures of the transducers in the array and within the operating temperature range. The cooling of the coupling interface with the patient can help to prevent skin burns and keeps the patient more comfortable.

In one variation, the treatment module 901 includes a treatment applicator 903 connected to a mover 905 (also referred to as ‘Mover A’ herein, or the ‘base mover’), as shown in FIG. 127. Mover A holds the treatment applicator against the body of the patient during setup and treatment. Mover A will allow the operator to slide the applicator to the appropriate position against the target tissue, lock it in-place, and then provide upwards force so that the treatment applicator can be securely coupled (physically and acoustically) against the patient. Mover A positioning components include a flat base 907 (for effecting an air cushion, enabling easy movement of the treatment module across the patient platform working surface), a round ball joint, a pressure activated piston, and vacuum actuated lock mechanisms.

Mover A 905 supports and positions the applicator 903. In use, it may be secured to a glide plate or other flat surfaces, that rests on the depressed cavity between the two elevated portions of the patient platform. In some variations, the glide plate may be easily removed from the patient platform. In some variations, Mover A is configured to be manually adjusted. It may have six degrees of mechanical freedom to permit the applicator membrane face to be positioned and maintained in appropriate contact with the patient, in both the right and left treatment positions. Its pneumatic interface may be located within the water conditioner. The electrical controls for several pneumatic control elements (e.g., solenoid valves) in the water conditioner are mounted on the applicator. These communicate directly between the water conditioner and other subsystems.

In some variations, the treatment applicator includes a plastic shell, and further comprises of a treatment transducer array (enclosed in a watertight volume), and a stepper motor driven mechanical rocker (the treatment transducer array mover, also referred to as ‘Mover B’ herein, or ‘array mover’). In one variation, the membrane of the applicator comprises a clear polymeric material, such that when the applicator is not engage on the patient's body, the operator can visually inspect the treatment transducer array. In one example, the system may implement a test protocol, which sends control signals to Mover B, and the operator may visually verify that the treatment transducer array is moving through a full range of motion, and there is not obstruction on its movement. In some variations, at least a portion of the side-wall of the applicator is constructed of transparent or translucent material (e.g., clear or partially clear materials), such that when the applicator is applied on the patient, the operator can visually inspect the movements of the treatment transducer array during the treatment process. In some variations, the nose cone is completely or partially made of clear materials, to allow visual inspection of the treatment transducer array and/or the treatment transducer unit.

The array mover is used to maintain registration of the treatment transducer array to the target region to be treated, such as the renal artery target. In one variation, the treatment transducer array is positioned, such that the target exist on a line normal to the surface of the treatment transducer array, intersecting at the geometric center of the arc of the transducer array elements.

As shown in FIG. 127, the treatment module comprises a membrane 909 for coupling to the body of the patient, the membrane stretches over the top opening of a cone shaped border (i.e., the treatment module nosecone 911) that is connected to the base of the of the applicator. A first handle 913 protrudes from the distal (narrow) end of the applicator, and a second handle protrudes from the proximal (wide) end of the applicator. The first handle includes a first switch 919 (i.e., the ball lock/release switch) which controls the lock and release of the ball joint 915 for the treatment module ball joint of Mover A. The ball joint 915 sits in the cup of the treatment module base 907. The treatment module base 907 includes a set of handles 917 for moving and positioning the treatment module. When the ball lock/release switch is on, suction is applied from the treatment module base to lock the ball joint in-place. A second switch 921 (i.e., Z-Motion extend/release switch) is also provided on the first handle, which controls the Z-direction extension and retraction of the applicator, such that the applicator is able to advance away from the ball joint (i.e., when the switch is on the “Extend” position), or be compression towards the ball joint (i.e., when the switch is on the “Release” position). When the switch is on the Extend position, positive air pressure is applied to the chamber in the piston located within the ball joint. The positive pressure pushes the piston outward and therefore advancing the applicator coupled to the piston outward until the applicator engages a surface, such as the patient's body, that provide a counter pressure to prevent further advancement of the applicator. When the Release button is activated, pressure in the piston is released, and pressure applied downward on the applicator would cause the applicator to contract, and retracts back towards the ball joint.

The second handle includes a third switch 923 (i.e., inflate/deflate switch for inflating and deflating the treatment module membrane). When the “inflate” switch is applied, pressure in the chamber of the nosecone housing the coupling fluid is increase. When the deflate switch is applied, pressure in the chamber of the nose cone housing of the coupling fluid is decreased. Pressure sensor may be provided in the treatment module and/or within the fluid path or fluid chamber of the water conditioner to provide feedback control of the fluid pressure in the chamber of the nosecone. A fourth switch 925 (i.e., the base lock/release switch) is also provided on the second handle. When the base lock/release switch is applied, a negative suction is applied to the channels in the bottom face of the treatment module base. This suction secures the treatment module base onto the surface that it is positioned over. When the switch is turn to the release position, a positive pressure is applied, which release the suction and applies a positive pressure into the channels in the bottom face of the treatment module. As the result, a positive pressure lifts treatment module from the surface that it sits on, and allow for easier lateral movement and repositioning of the treatment module.

The treatment module also includes a first tubing 927 supplying the coupling fluid from the water conditioner to the treatment module nosecone, and a second tubing 929 for removing the coupling fluid from the nosecone and back into the water conditioner. A third tubing 931 provides an air channel from the water conditioner to the inner cup of the treatment module, that engages the ball of the ball joint. This channel allows the water conditioner to apply a suction to the ball joint. The water conditioner may also be configured to apply a positive pressure to release the ball joint. A fourth tubing 933 provide another air channel from the water conditioner to the bottom surface of the ball joint. Suction or positive pressure may be applied through this channel to lock or release the base of the treatment module from the surface that it sits on.

Momentary-rocker switches, located on the treatment applicator hand grips, are used to inflate and deflate the patient coupling membrane, lock and unlock both the treatment module base and ball join, and raise and lower the treatment module applicator along with its nosecone to facilitate stable coupling with the patient. In the example shown in FIG. 127, the treatment module allows for movement of the array in the X-Y-Z and 3 angular coordinates prior to locking.

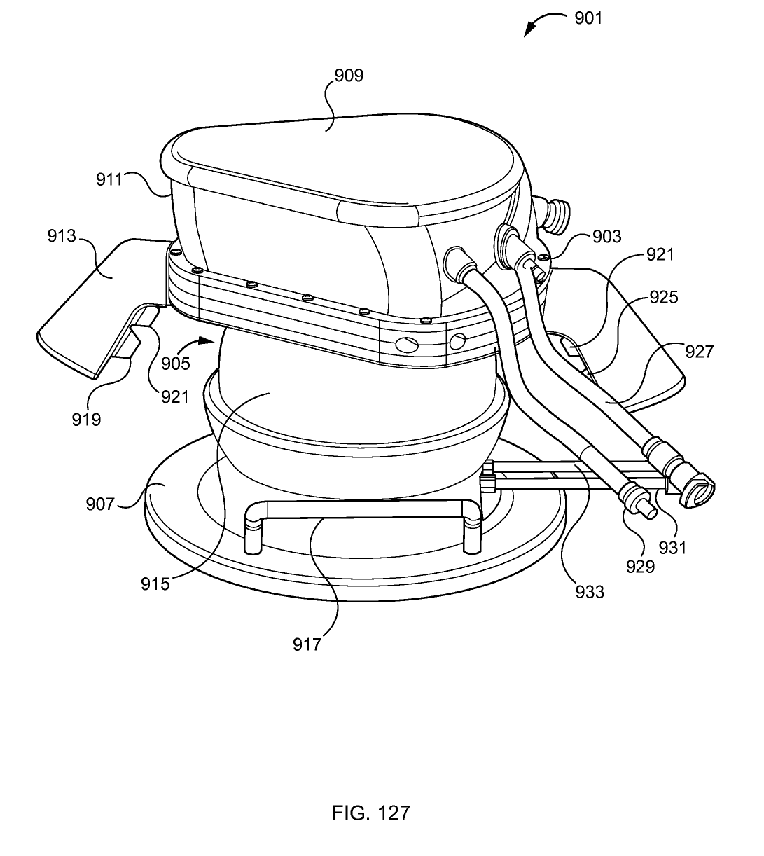

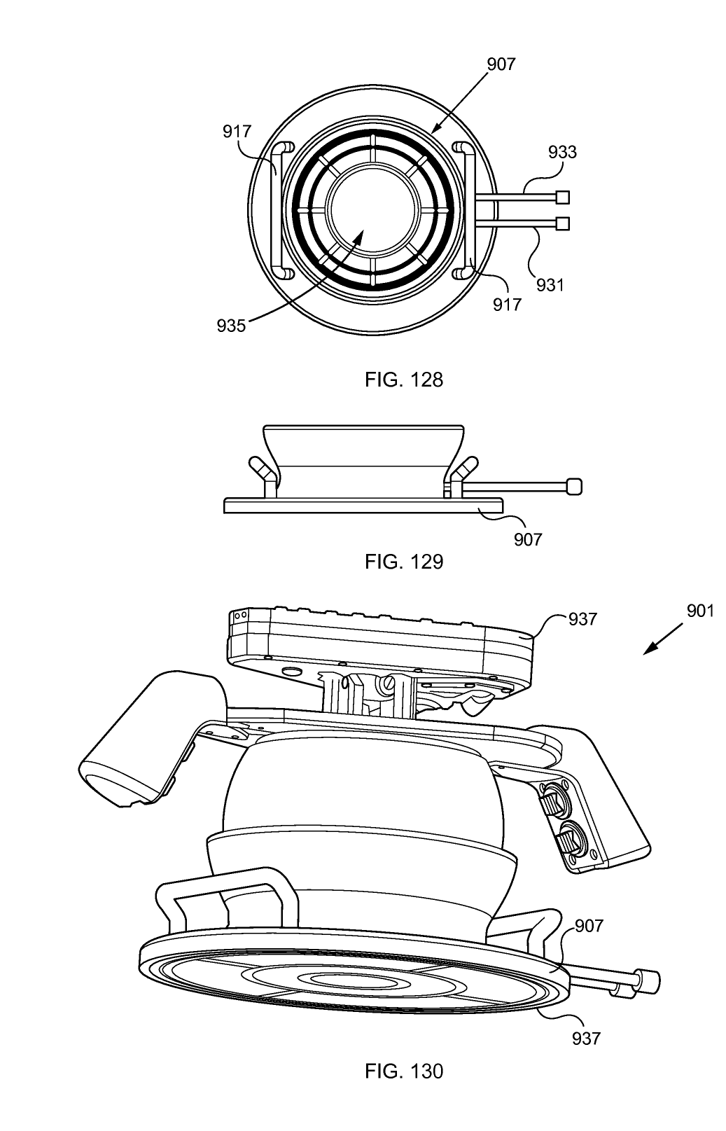

FIG. 128 is a top down view of the base 907 of the treatment module showing the cup or concave recess 935 for receiving he treatment module ball joint. FIG. 129 is the side view of the base 907 of the treatment module. FIG. 130 is a perspective view showing the bottom surface of the treatment module 901. Grooves 937 are embedded within the bottom of the treatment module base 907, to allow positive airflow and suction to be applied evenly across the bottom surface of the base. In some embodiments, the hydraulic piston is replaced with a stepper motor so that its movement can be tightly controlled by the operator.

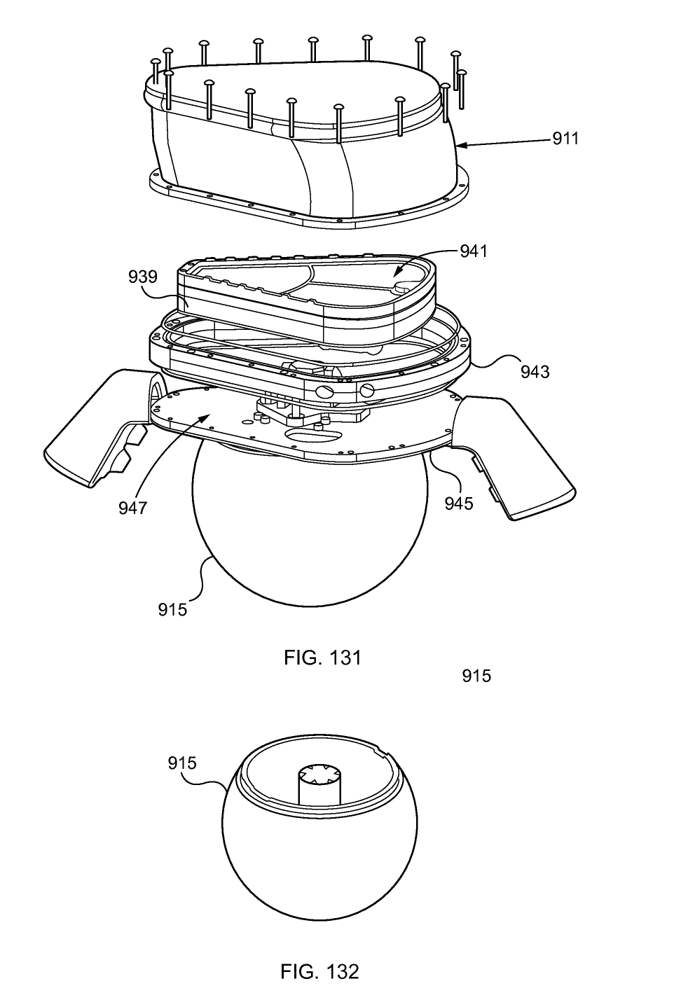

Referring to FIG. 131, an exploded view of the applicator 903 of the treatment module is shown. In this example, the applicator 903 comprises a nosecone 911, a treatment array unit 939 housing the treatment transducer array 941, a frame 943, a base plate 945 forming the base of the applicator 903, and an actuation unit 947 that is positioned below the center of the base plate 945. The actuation unit is coupled to the treatment array unit 939 for controlling the position and orientation of the treatment array unit. Nosecone 911 is disposable in some embodiments. That is, the membrane and housing structure are created out of low cost materials and are supplied to the operator of the system

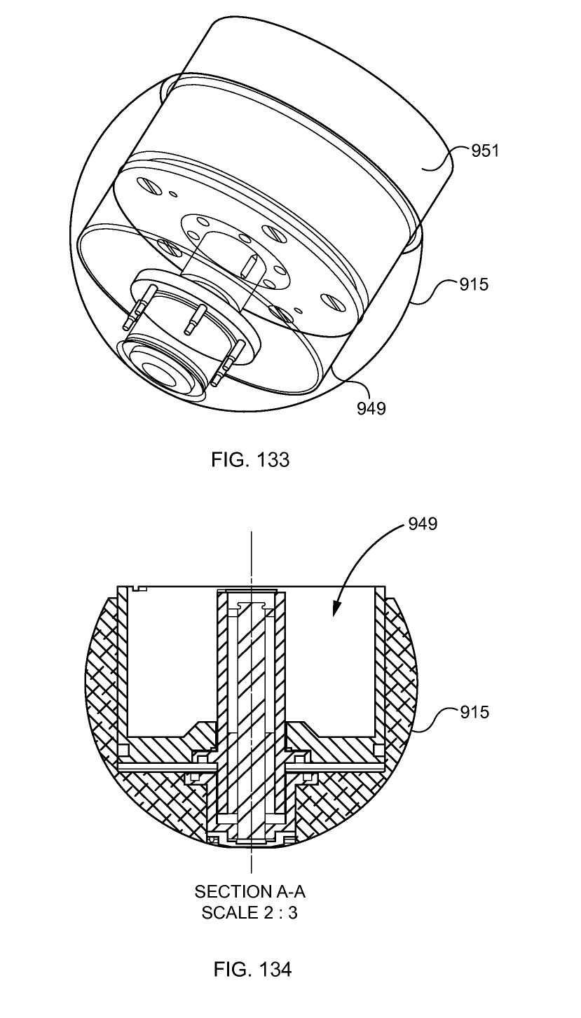

FIG. 132 shows the ball joint 905 of the treatment module. The ball joint has a cavity 949 for receiving a piston 951 insert that is coupled to the base of the applicator, as shown in FIG. 133. The piston allows the system to advance and retract the application along the Z-direction, by varying the presser inside the piston chamber. FIG. 134 is a cross-sectional view of the ball joint 905 shown in FIG. 132. The piston can be replaced by a stepper motor which will incrementally advance the transducer toward the patient.

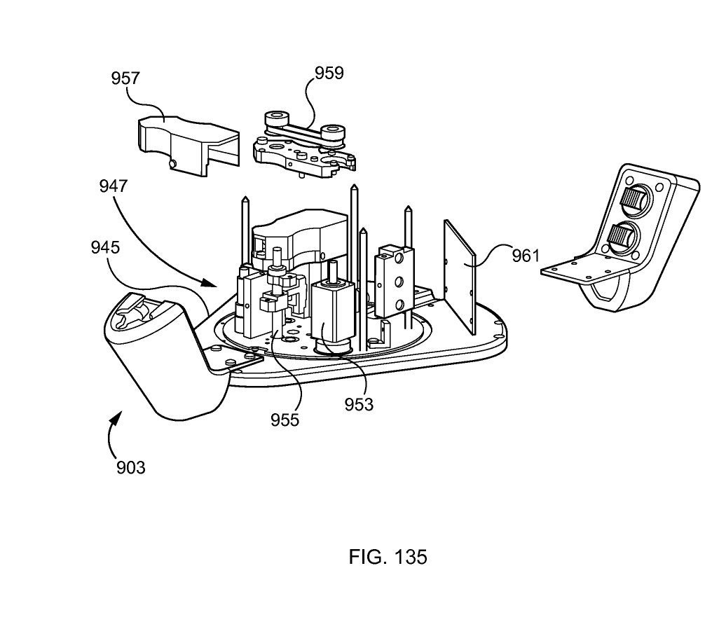

FIG. 135 is an exploded view showing the components that comprises the lower portion of the applicator 903, which includes the actuation unit. In this example, the actuation unit comprises two stepper motors 953, each driving a push rod 955 that is couple to the treatment transducer unit, to control the movement/orientation of the treatment transducer module. In this figure, only one of the stepper motors 953 is shown. The other is cover under a belt cover 957. Two belts 959, one for each stepper motor 953, couple the stepper motors to their corresponding push rods 955. Each of the push rod 955 has embedded thread and a corresponding screw bearing. By rotating the crew bearings the corresponding posh rods are advance or retracted in and out of the base plate 945 of the treatment applicator. A printed circuit assembly 961 is connected to both the computing unit and the two stepper motors, allowing the computing unit to control the stepper motors. Thermistor sensors may be incorporated in, or attached to the stepper motors, to monitor the temperature and operation of the stepper motors.

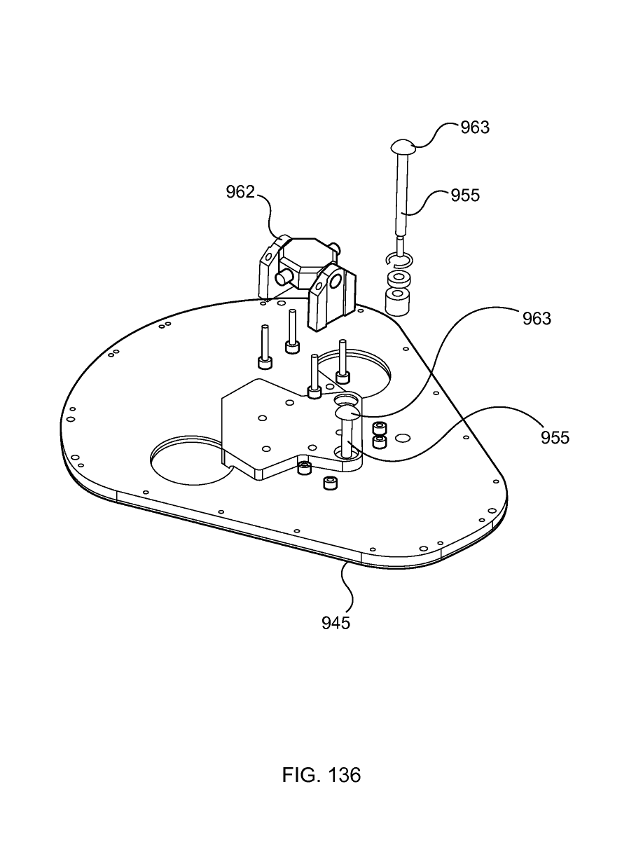

FIG. 136 shows a mechanical joint 962, having two degrees of freedom, attached to the upper surface of the applicator base place 945. The joint couples the treatment transducer unit to the base plate of the applicator, provides two degrees of freedom (i.e., pitch and roll) movement for the treatment transducer unit (which includes the treatment transducer array) relative to the base of the applicator. The push rods 955 extending from the actuators position below the base plate 945, through the holes on the base pale. The distal tips 963 of the push rods 955 are coupled to the treatment transducer unit. In one example the coupling is achieve through magnets 965 placed in the bottom surface 967 of the treatment transducer unit 939. In another example, coils, semi-compliant or elastic materials may be used to couple the distal end of the push rod to the treatment transducer unit. The compliant/elastic materials allows the push rod to move the treatment transducer unit, while allowing for some give or buffer when the movement of the treatment transducer unit experience some unexpected resistant. In yet another example, the push rods are coupled to the treatment transducer unit through a moveable mechanical joint. The system, by controlling the displacement of the push rods, can then control the position/orientation of the treatment transducer unit.

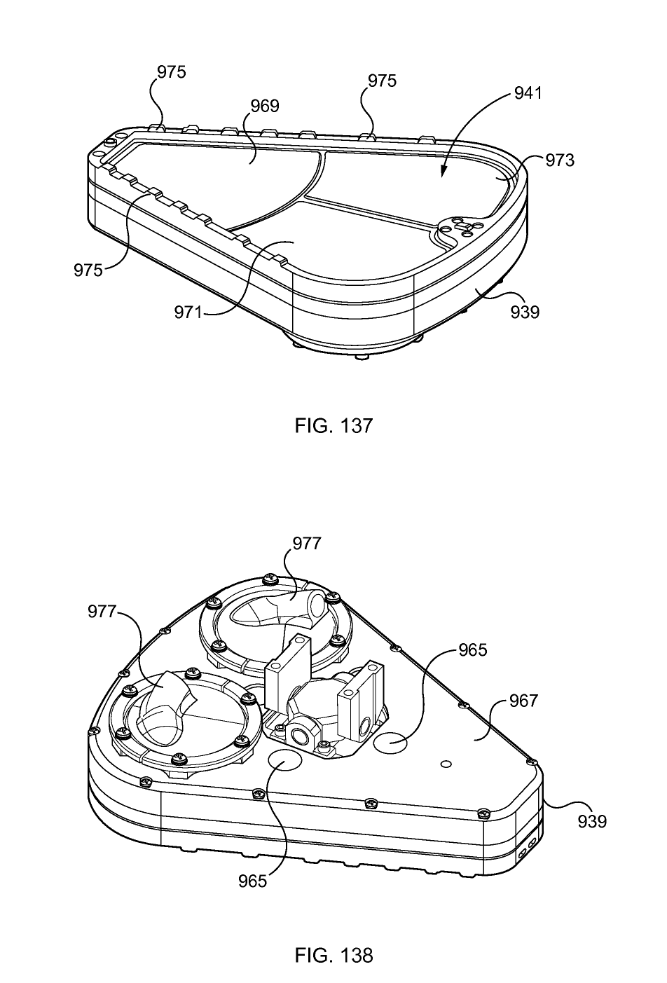

FIG. 137 shows the top view of the treatment transducer unit 939. In this example, the treatment transducer array 941 comprises three lobes 969, 971, 973, forming a fan shaped or pie shaped structure on the top surface of the treatment transducer unit 939. ATOF receivers 975 are position in a frame on the outer ridge of the treatment transducer unit 939. In this example, the treatment transducer unit 939 of the therapeutic system is part of the applicator that generates and delivers the therapeutic ultrasound energy to the targeted region in patient. The treatment transducer unit is moveably mounted within the applicator, and powered by the multi-channel RF power from the transceiver module (also referred to as the RF subsystem) through the interconnects and cabling subsystem.

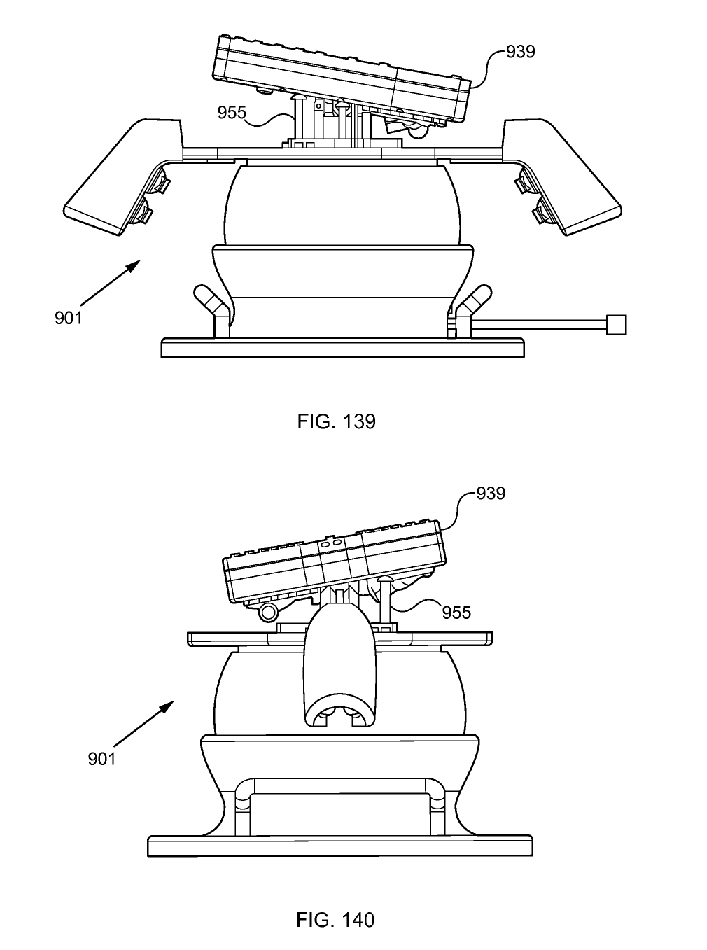

FIG. 138 shows the bottom view of the treatment transducer unit 939. The bottom of the unit includes two connector ports 977, for connecting wiring into the unit to drive the treatment transducer array and the ATOF receivers. A rotatable joint 962 is position at the pivot point of the treatment transducer array. This joint couple the treatment transducer unit, along with the treatment array housed therein, to the applicator base frame. The joint allow the system to tilt and roll the treatment transducers unit, by displacing the push rods that are couple to the treatment transducer unit. FIG. 139 shows the treatment transducer unit 939 tilted up. FIG. 140 shows the treatment transducer unit 939 rolled to one side.

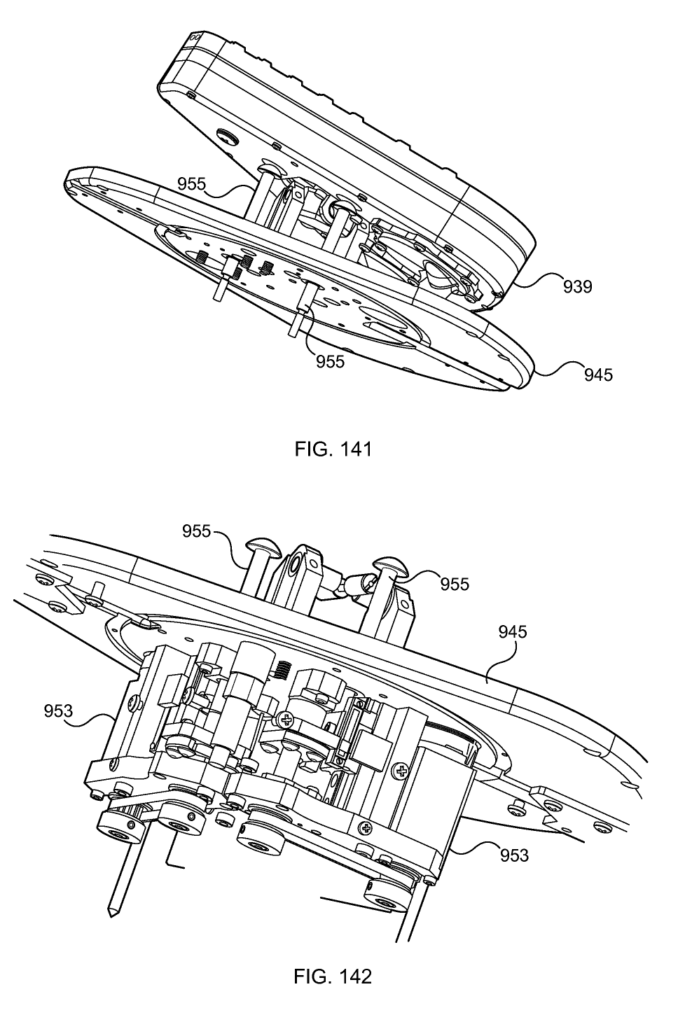

FIG. 141 shows the push rods 955 extending through the base plate 945 of the applicator to engage the bottom surface of the treatment transducer unit 939. In this example, two magnets 965 are embedded in the bottom surface of the treatment transducer unit. The rounded heads 963 of the push rods engages each of its corresponding magnets 965, which couple the treatment transducer unit 939 to the push rods 955. FIG. 142 is another view that shows the details constructions of the two stepper motors 953 driving corresponding pus rods 955.

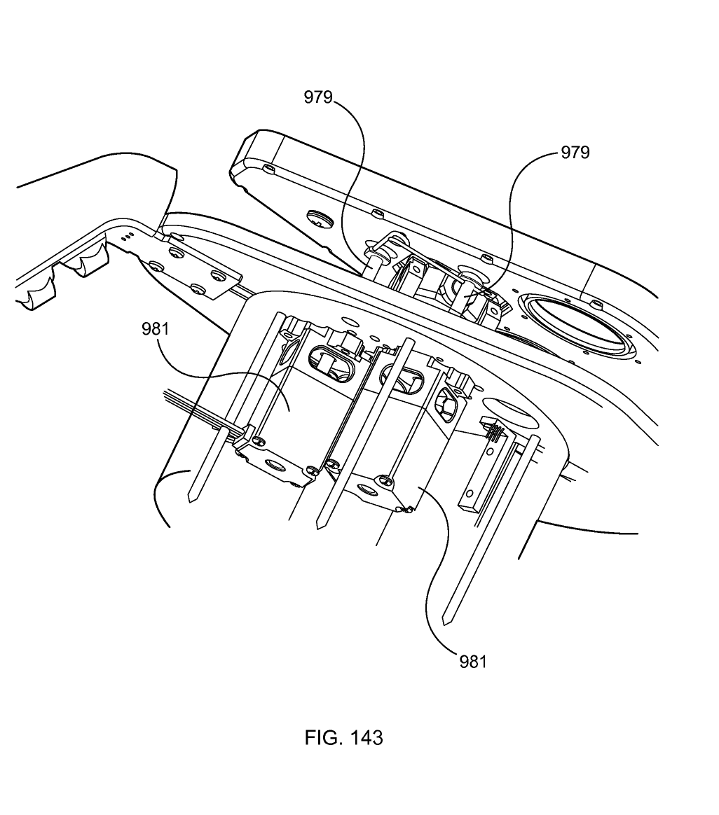

FIG. 143 illustrates another variation, where the push rods 979 are directly integrated within the motors 981, such that the motor can directly drive the displacement of the push rods without additional coupling mechanisms, such as belts.

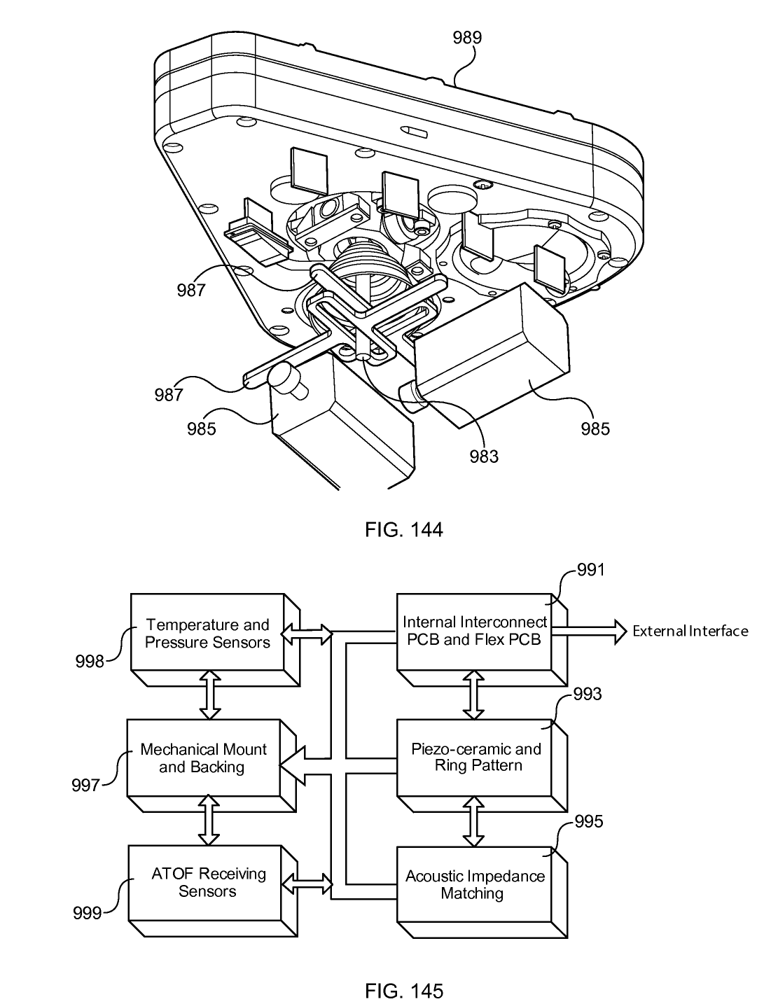

FIG. 144 shows another variation, where a joy stick 983 coupled to the base of the treatment module is manipulated to control the movements/orientations of the treatment transducer unit 939. In this example, two laterally placed motors 985 are coupled to the joy stick through slotted plates 987. Each of the plate allows free movement of the joy stick in one direction, but constrains the movement of the joystick in the other direction, to control the movement of the joystick in the constrained direction. Therefore, one motor controls the X-direction displacement, while the other motor controls the Y-direction displacement of the joystick. A spring 989 is incorporated to provide mechanical bias or buffering.

In one configuration, the treatment transducer unit 939 comprises the following modules: (a) internal interconnect PCB (Printed Circuit Board) and flex PCB, 991 (b) piezo-ceramic and electrode pattern, forming the transducer array, 993 (c) acoustic impedance matching, 995 (d) mechanical mount, backing and tooling, 997 (e) positioning ATOF receiver and pre-amplifiers, 999 (f) temperature sensors and pressure sensors 998. FIG. 145 illustrates one variation of an arrangement for the interactivities between the modules.

The therapeutic system may include a cooling and inflate-deflate subsystem (which may be incorporated within the water conditioner) that controls the inflate-deflate process of the membrane, maintains a constant volume, and provides cooling for the therapy transducer.

A membrane on the nosecone of the applicator provides an acoustical clear barrier between the patient and therapy transducer. It also facilitates the positioning of the applicator on varying patient topography.

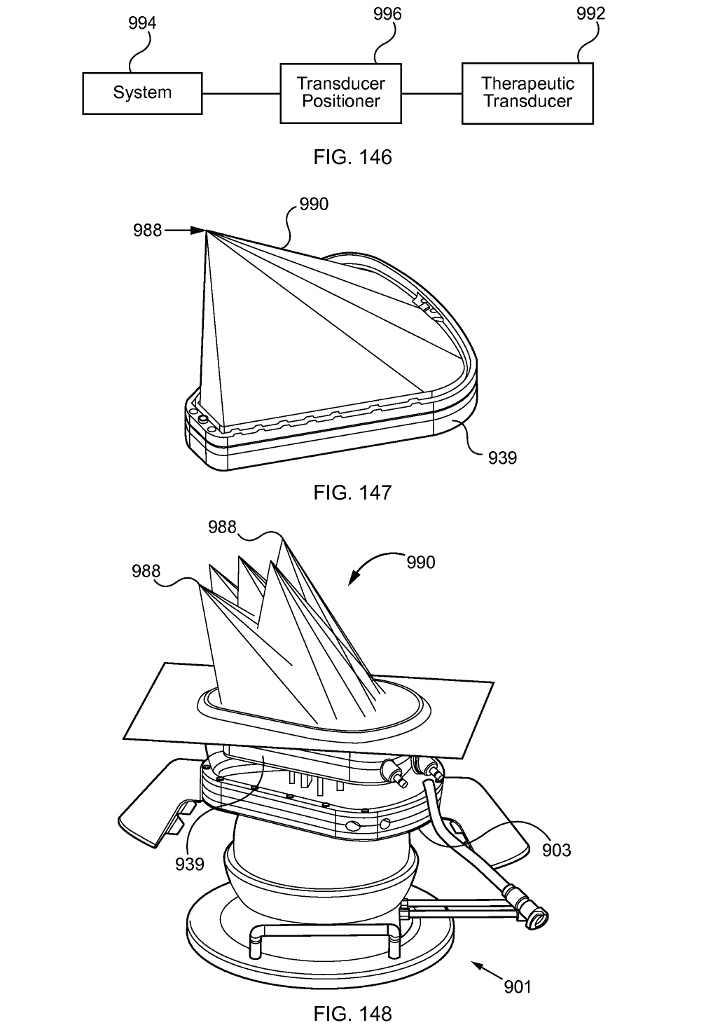

A treatment transducer array (or treatment transducer unit) positioner subsystem articulates the treatment transducer array in order to track and treat the target tissue. In one variation, the treatment array positioner provides rotational motion about the X and Y axis of the treatment transducer array, which is mounted within the applicator. The treatment array positioner 996 receives move commands from the system 994 and translates these commands into rotational movements of the treatment transducer array 992, as illustrated in FIG. 146.

FIG. 147 is a graphically representation modeling an example of a focused ultrasound filed 990 being projected from the treatment transducer array, on the treatment transducer unit 939. In some variation, the system is configured such that it is capable of focusing 988 the ultrasound field at a depth of 16 cm from the face of the treatment transducer array. In some variations, the system is configured such that it is able to focus 988 the ultrasound field at a depth of at least 10 cm from the face of the treatment transducer array. In some variations, the system is configured such that it is able to focus the ultrasound field at a depth of at least 14 cm from the face of the treatment transducer array. In some variations, the system is configured such that it is able to focus the ultrasound field at a depth of at least 16 cm from the face of the treatment transducer array. In some variation, the system is configured, such that the focus of the ultrasound field is off axis from the perpendicular axis (i.e., normal) extending from the weighted center of the treatment transducer array surface.

FIG. 148 is a graphical representation modeling the treatment transducer unit 939 of FIG. 147 being positioned in the applicator 903. As shown, by rotating the treatment transducer unit 939 on a pivot within the applicator 903, and/or changing the focal depth with phase modification during the electrical excitation of transducers in the treatment array (in this example phased array may be implemented), the therapeutic system is able to direct the focus of the ultrasound field 990 emitted by the treatment transducer array to a desired location. In some variation, the system is configured to displace the focal point 988 of the therapeutic ultrasound field, without altering the contact between the treatment applicator and the patient (for example, maintaining the same contact pressure, contact surface area, and/or the orientation of the applicator relative to the patient's body).

In one configuration, a 3D orientation sensor and an A/D converter (mounted to Mover A and electrically connected to a local thermistor and pressure sensor) are configured for sensing of the orientation, and the water temperature and pressure of the treatment applicator. Another 3D orientation sensor and A/D converter is mounted behind the treatment transducer array, connected to local thermistors at the actual PZT material. This allows the therapeutic system's application to monitor the motion of the treatment transducer array and the heat rise of the PZT ceramic during therapy.

An example of a process for positioning the treatment module on the patient platform and engaging the treatment module with the posterior flank of the patient's body is described below. The operator may first mark the patient's posterior flank to identify a treatment window. The patient's is placed on the patient platform such that treatment window is centered in the treatment aperture of the patient platform.

One or more lights may be provided in the aperture in the patient platform. Verify that the remote targeting monitor and the remote treatment module camera monitor are each positioned for viewing, and will not obstruct placement of the treatment module and its associated cables and hoses. An atomizer bottle may be used to spray the subject's posterior flank, which is to be treated with water to moisten the skin. Apply ultrasound gel, spray the gel with water to reduce its viscosity and massage it uniformly onto the skin over the full treatment area.

Verify that no bubbles are evident within the treatment applicator nosecone. Spray the treatment applicator membrane with water to moisten it. Apply ultrasound gel, spray the gel with water to reduce its viscosity and massage it into a uniformly thin layer over the entire surface of the membrane. Take care to avoid entrapment of air bubbles in ultrasound couplant layer. Using the “Inflate” button on the treatment module handgrip, inflate the treatment module membrane (for example, inflate to such extent that the apex of membrane is approximately 1 cm above the surface of the treatment module nosecone (i.e., the plane defined by the rim of the nose cone).

Transfer the pre-gelled treatment module onto the patient platform and slide it under the posterior flank that is to be treated. Arrange the treatment module cables and hoses so that free movement of the treatment module is not constricted. Connect the remote monitor cables, emanating from the treatment module wire loom, into their respective connectors on the remote targeting monitor and the remote treatment module camera monitor.

Confirm that acoustic coupling gel covers the membrane surface uniformly. With the treatment module nosecone oriented vertically, couple the treatment module to the patient's selected posterior flank within the marked treatment window, by pressing the “Z” button on the treatment module handgrip. When the treatment module membrane contacts the patient's skin, a constant pressure will be applied that maintains contact with the skin. Orient the apex of the nosecone to point toward the costovertebral junction.

In one application, a patient platform is utilized with one treatment module. The treatment module may be configured to be manually moved within the aperture of the patient platform for treating both sides of the body. Mechanical actuators (e.g., robotic arms or movers, linear motors, etc.) may be applied to move the treatment module through electromechanically control, such that the treatment module may be moved in the X-Y plan within the aperture of the patient platform, and raised or lowered in the Z direction to engage the treatment applicator, and couple the treatment transducer array to the patient's body. The treatment applicator may be configured with a ball join, such as one described above, and further enhance with actuators to control the movement of the ball join. This configuration may allow the therapeutic system electromechanically pivot the applicator and provide pitch, yaw, and roll motion to control the orientation of the actuator in engaging the patient' body.

In another application, two treatment modules are positioned within the aperture of the patient platform, with one configured to treat the left side of the patient's body, and one configured to treat the right side of the patient's body. Optionally, each of the treatment modules may be electromechanically actuated, such that the therapeutic system can control the positioning of the actuator by moving each treatment modules in the X-Y plan, and moving each in the Z direction to couple the treatment transducer arrays to the patient's body. Each of the applicators in the treatment modules may be further configured with an electromechanically controlled joint for pivoting the applicator to provide two or more degrees of freedom of motion (e.g., pitch, yaw, roll, and Z-advancement), and allows for independent control of the orientations of the applicators.

### X. Interface Cooling and Monitoring

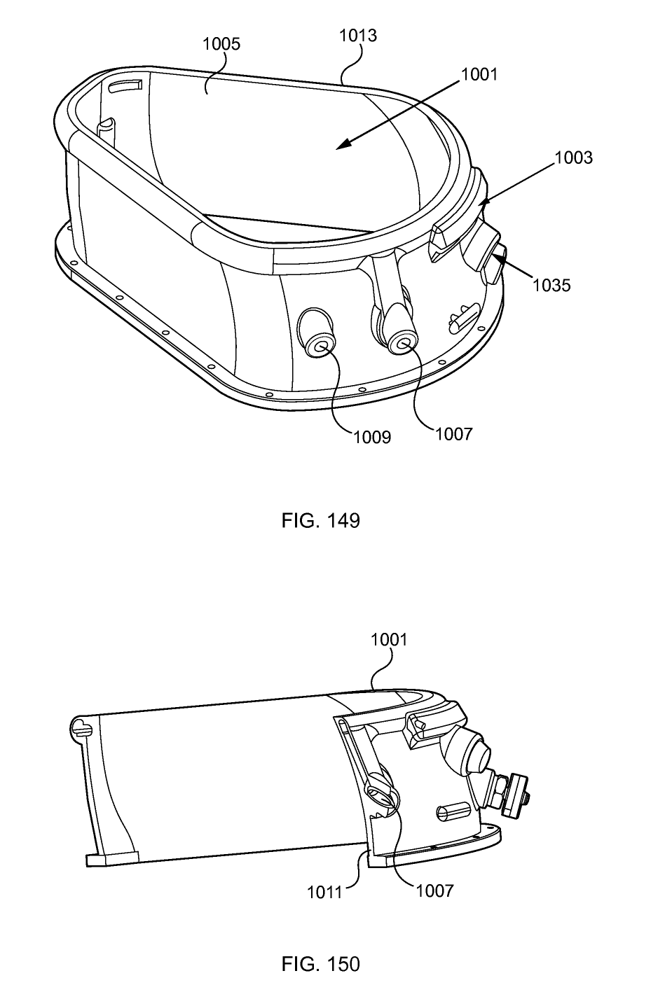

Referring to FIG. 149, one example of a nosecone 1001 for an applicator is shown. Is this drawing, the membrane that is attached to the top rim of the nosecone is not shown. In some variations the nosecone is connected to the base of the applicator and is reusable from patient to patient. In some variations, the nosecone is removably attached to the base of the applicator, such that it may be replaced during or between procedures. The nosecone may be fabricated primarily of plastic or other suitable polymeric materials, such that it would be disposable after the completion of each procedure. In some variations, the nosecone includes an embedded image detector, such as a camera, for monitoring the coupling interface between the applicator and the patient's skin. The nosecone may further include a light 1003, such as an LED (Light emitting Diode) light strip, for illuminating the internal chamber of the nosecone and enhancing visualization within the chamber 1005.

FIG. 150 is a cross-sectional view of the nosecone 1001 shown in FIG. 149. In this example, the nosecone 1005 has a fluid input port 1007 and a fluid output port 1009. Fluid for coupling the ultrasound transmission, and for cooling the transducer array and the patient's skin, is injected into the input port 1007 and drawn out from the output port 1009. A fluid pathway is built into the wall 1011 of the nosecone, which allows the injected fluid to travel upward toward a channel located in or adjacent to the rim of the nosecone. The channel travels through at least part of the circumference of the rim 1013. In one variation, the channel is embedded within at least 50% of the rim. In another variation, the channel is embedded within at least 70% of the rim.

The rim of the nosecone holds the membrane that contains the coupling fluid in the nosecone chamber, and comes into contact with the patient's skin. The rim is in close proximity to the patient skin, and may contact the patient's skin when the applicator is position to deliver the focus ultrasound energy.

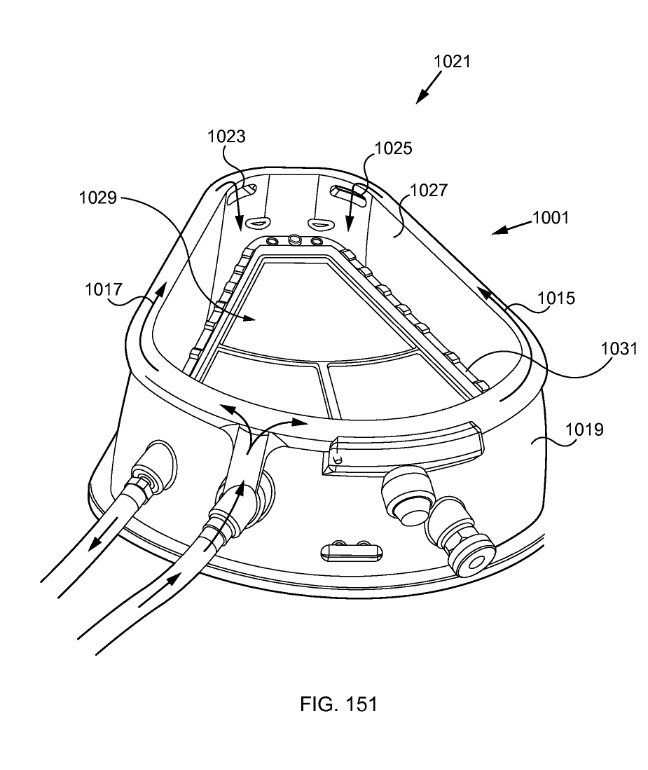

The fluid enters the embedded channel 1015, 1017 in the rim of the nosecone 1001, and travels around the rim from the proximal (wide) side 1019 of the nosecone to the distal (narrow) side 1021, and exit through two inner ports 1023, 1025 that are positioned on the inner wall 1027 of the nosecone, and configured to direct fluids to flow over the surface 1029 of the treatment transducer array 1031, as shown in FIG. 151. Fluid is directed to flow form the distal end of the inner chamber 1005 of the nosecone 1001 towards the proximal end. On the inner wall of the proximal side of the nosecone, an output port 1033 is provided to drawl the fluid out of the chamber 1005, as shown in FIG. 152. The water conditioner may be configured to provide (a) a positive pressure to inject the fluid through the input port into the nosecone chamber of the applicator, or (b) a negative pressure to suction the fluid out of the nosecone chamber of the applicator through the output port. Alternatively, both positive injection and negative suction may be applied to facilitate the flow of the fluid in and out of the applicator. In one variation, one or more temperature sensors may be applied to detect whether the transducer array is overheating, whether the surface of the patient's skin is overheating, and/or whether the coupling fluid in the nosecone chamber is overheating. If overheating is detected, the water conditioner may increase the fluid flow rate over the treatment transducer and/or lower the temperature of the fluid being delivered into the nosecone chamber.

In one variation, the treatment module further includes an image detector 1035 positioned to monitor the interface between the applicator 1037 and the patient. In one configuration, the image detector 1035 is positioned within the nosecone 1011 of the applicator 1037. The image detector may comprise of a wide angle CCD camera, positioned at the wide end of the nosecone, and angled upward, such that it is able to detect air bubbles on the interior of the membrane, and air pockets in the interface between the membrane and the patient's skin. In one variation, the image detector 1035 is capable to detect air bubbles larger than 3 mm diameter on the interior of the membrane 1039, and capable of detecting air pockets larger than 3 mm diameter in the interface between the membrane and the patient's skin. One or more lights, or other illuminating devices, may be provided in the applicator to provide illumination for the image detector. The light may be positioned to illuminate the inner surface of the membrane, and transmit through the member to illuminate the skin. The image detector 1035 can be positioned such that in addition to monitoring air bubble and air pocket at the member, it is able to provide visual feedback on the orientation of the treatment transducer array 1031 (e.g., monitoring the pitch and roll motion of the transducer array during treatment cycle).

In one example, the image detector is capable of viewing at least 50% of the membrane area from within the housing of the nosecone when the membrane is inflated to 1 cm. In another example, the image detector is capable of viewing at least 75% of the membrane area from within the housing of the nosecone when the membrane is inflated to 1 cm.

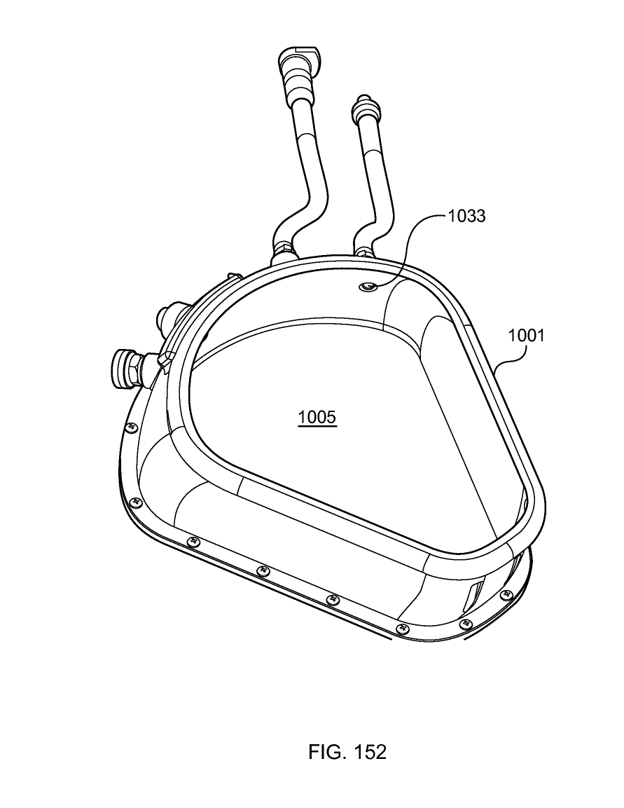

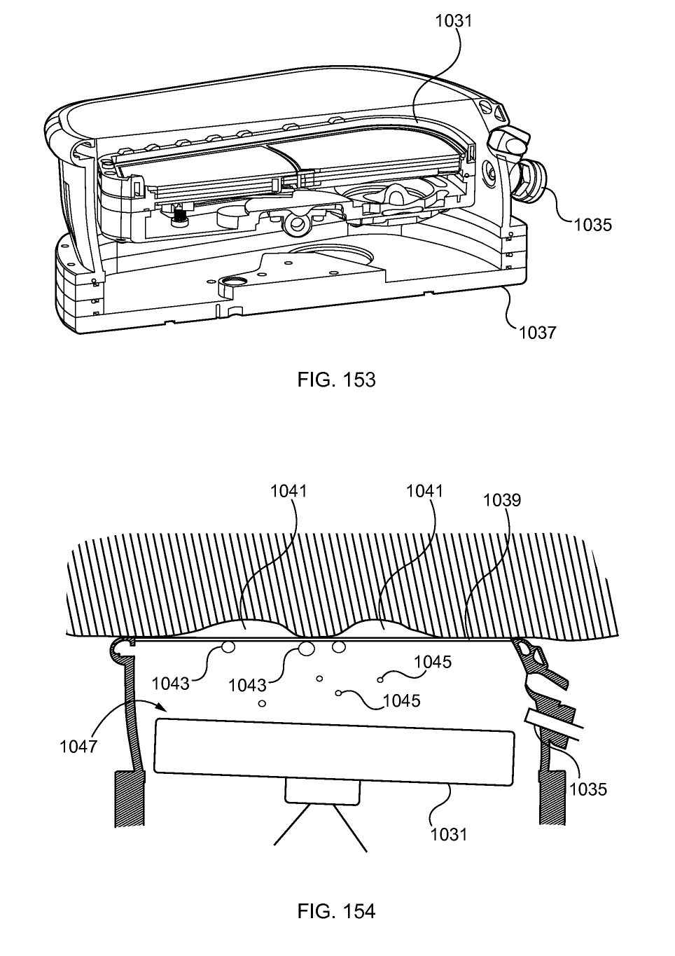

Referring to FIG. 153, an example of an applicator 1037 having an embedded image detector 1035 is shown. The image detector 1035 comprises a wide angle lens, and it is oriented such that it can monitor the underside of the membrane and the transducer simultaneously within its field of view, when the transducer is at a nominally flat position. In some variations, the camera is configured to detect at least 50% of the surface area of the membrane, and at least 50% of the surface area of the face of the treatment transducer array. In some other variations, two image detectors are utilized, with one positioned to monitor the membrane, while the other one is positioned to monitor the face of the array.

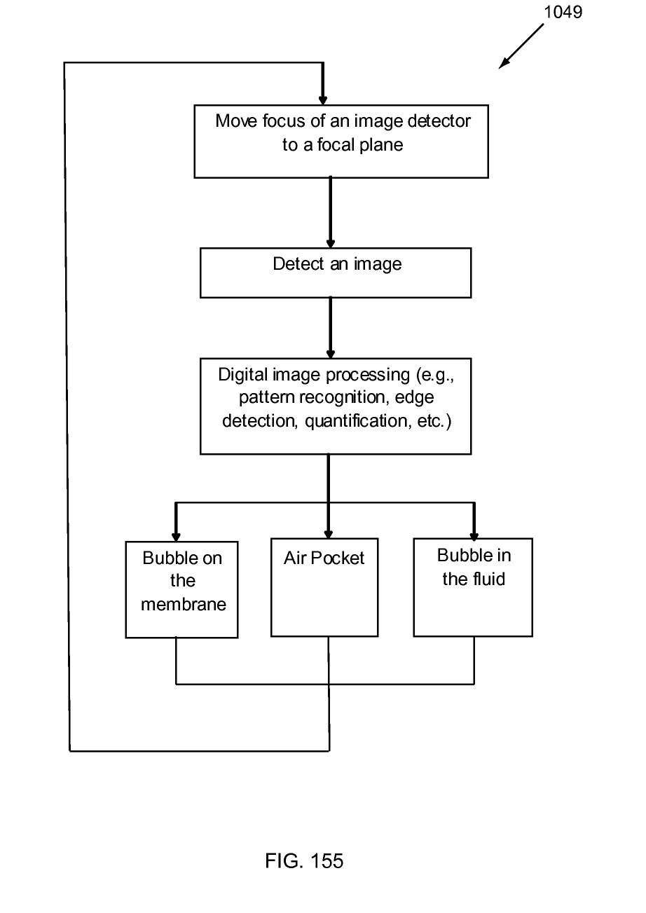

In addition to bubble and air pocket detection on the membrane, the imaging detector can also be applied to facilitate treatment module placement on the patient, bubble detection inside the water bath confined within the nosecone, monitoring patient's skin during treatment, and monitoring of the movements of the treatment transducer array. For example, as shown in FIG. 154, the image detector 1035 having a wide angel lens can be positioned to monitor the air pocket 1041 between the membrane 1039 and the patient's skin, the air bubble 1043 in the inner surface of the membrane 1039, the air bubble 1045 floating in the coupling fluid, and the surface 1047 of the treatment transducer array 1031. In one variation, the image detector is configured with a focusing lens that allow the image detector to change its focus plane, such that the system controller may control the image detector to focus on an area just above the membrane to detect the air packet and any objects (e.g., hair or particles) that may be trapped between the patient and the membrane first, then move the focus right below the membrane to detect any air bubble on the inner surface of the membrane. The focus can then be adjusted to a region between the transducer array and the treatment transducer face, to detect any bubble in the coupling fluid. In one variation, the image detector is configured to scan through this volume of fluid between the membrane and the transducer array to detect any bubble or particles. Pattern recognition process (either hardware and/or software) may be implemented to evaluate the image captured at each focusing plan in order to detect the air packets or air bubbles. This computing process may also further quantify the air pockets and bubbles detected by the system in terms of numbers by individual count and/or total volume. If the system detects an air bubble or an air pocket, it may provide the overall system a condition notice, and trigger the system to alert the operator on the existence and/or location and/or volume the air bubble and/or air pocket. One example of the bubble detection process 1049 is show in the flowchart in FIG. 155. In this variation, based on the location of the focal plan, the pattern, and/or formation of the detected air trapped in the fluid, the system then identifies and notifies the user whether bubbles on membrane, air pocket, or bubble in the coupling fluid has been detected. In one variation, the detection threshold is set at 2 mm, such that any detected bubble larger than 2 mm in diameter triggers an alert to the operator. In another variation, the detector threshold is set at 3 mm, such that any detected bubble larger than 3 mm triggers an alert to the operator.

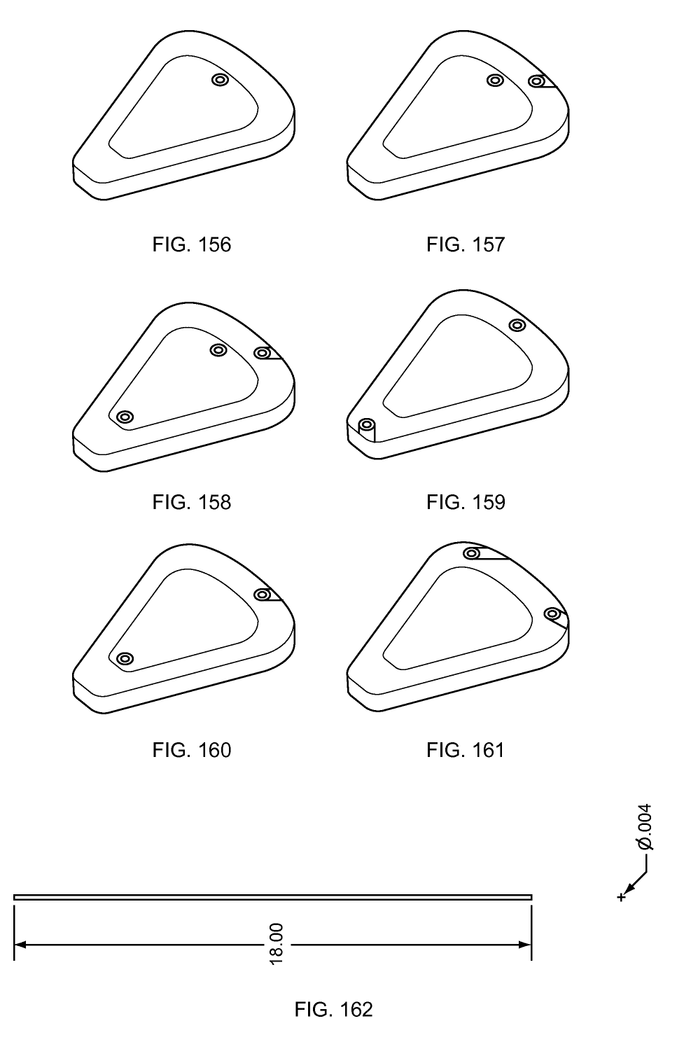

The treatment module may be configured with one or more image detectors, depending on the application and clinical demands. An image detector 1051 may be placed on the treatment transducer array unit 1053 itself, such that the view of the image detector 1051 will move with the treatment array, as shown in FIG. 156. The array mover may also be used to move the image detector to scan the surface of the membrane. This configuration allows the image detector to see the same view as the direction of the treatment array energy delivery. FIG. 157 shows a variation where one image detector 1055 is position on the treatment array unit 1053 and a second image detector 1057 is fixedly positioned on the applicator 1059. FIG. 158 shows a variation where three image detectors 1061, 1063, 1065 are provided. As compare to the design in FIG. 157, an additional image detector 1065 is provided a the tip (narrow) section of the treatment transducer array unit. FIG. 159 shows a variation where two image detectors 1067, 1069 are positioned on the applicator 1059. The image detector may be position within the nosecone structure or extend form a base frame in the applicator 1059. FIG. 160 shows another variation, where one image detector 1071 is located on the tip of the treatment array unit 1053, and the other 1073 is positioned on the applicator 1059. In yet another variation, as shown in FIG. 161, two image detectors 1075, 1077 are position on the two sides of the applicator 1059. The image detector may be directed to view the same surface area of the membrane. In one configuration, one of the image detector comprises an broad spectrum color image detector, while the other one comprises an IR spectrum detector. The IR spectrum detector may be used to monitor heating on the patient's skin and/or to locate hot spots. Image detectors that are positioned to monitor the face of the array may also comprise one that can detect within the IR spectrum, to monitor heating on the face of the treatment array, measuring the heating of the transducers, and/or locating any hotspots on the treatment array.

An image detector may be used for transducer array orientation detection (e.g., verify that the transducer array mounted on a mover mechanism (i.e., Mover A) has changed its orientation during targeting and tracking of the treatment process). Image detector may also be used to detect the inflation level of the membrane. In one example, this is achieved by placing markings on the membrane, and imaging the correlating and expanding distance between the markings to detect and/or measure membrane inflation. In another example, an image detector is position to obtain a side view of the membrane. Edge detection may then be imposed on the detected image to identify the boundary of the membrane, which can then be used to determine the extent of the member inflation.

An image detector may also be applied to detect patient movement relative to the treatment module or its applicator. For example, markings may be applied on the patient's skin within the treatment window, and by evaluating the position/displacement of markings on the patient's skin the system can determine whether patient movement has occurred, and/or calculate the amount of movements. Imaging may be used to visualize marking (e.g., marked treatment window) on the patient's skin through the membrane to facilitate applicator placement. In one example a commercial ultrasound probe is used to locate bony structures, and the skin marked with indelible ink to define a treatment window. These markings could then be visualized with the camera to aid in treatment module placement. In some variations, the markings are used for automated guidance and placement of an electromechanically actuated treatment module.

An image detector may be used in the placement of the treatment module relative to existing anatomical structures. For example, if energy is being delivered through the eye of the patient, the brow and bridge of the nose could be used as fiducials. Imaging may also be used in the placement of the treatment module relative to other fiducial marks (such marks may exist on the patient platform, on the glide plate, etc.).

In some variations, the image detector is monochromatic. In some other variations, the image detector has broad spectrum and may visualize in color—this may permit using varying colored inks to better assist in treatment module placement.

One or more light source may be placed to enhance images captured by the image detector. In one variation, the light source may be a strip light (e.g., a band of LED lights). In some variations, the light sources have differing colors to illuminate only specifically colored markings on the skin (e.g. if red and blue markings are drawn on the skin, using red illumination would only reveal the blue markings—the red marking would be camouflaged). In another variation, UV activated ink are used for applying fiducial marking, and an UV light located in the treatment module is used to activate and visualize the fiducial marks. In yet another variation, the image detector comprises an infrared (IR) camera. Infrared imaging may be more effective in detecting bubbles (since air is a poor conductor of heat). Infrared image may also be used to sense heat distribution on skin. Through image processing pattern recognition, local hot spots can be located and overall heat changes on the detected surface can be calculated. Infrared imaging could be used to detect localized heating on the array (potentially alerting the user to array damage, or impending array damage). Image detector may comprise of a camera capable of imaging in both the infrared spectrum and visible spectrum.

Image detector may be used to image the ultrasound therapy beam. This may be achieved by observing and noting the index of refraction change of the water in the path of the focused ultrasound beam (the pressure change causes a perceptible shift in the index of refraction of the water). This may also be achieved by introducing an additive to the circulating water, that becomes perceptible when absorbing ultrasound energy. Beam imaging may be used to confirm the beam if focusing as expected. Beam imaging may be used for other diagnostic purposes.

Image detector imaging may be used in conjunction with robotic movers to position the treatment module for the delivery of therapy. Image detector may also be used to note existing defects in the patient's skin in the treatment area. Images may be saved as a record for demonstrating the therapy was delivered to an expected location inside the body. For example, this could be achieved by observing the orientation of the transducer (e.g., tip and tilt), and by estimating focal depth of the beam shape emanating from the transducer array.

Images may be processed to signal when a bubble (inside the membrane or at the membrane-skin interface) is sufficiently large to require remediation. For example, <3 mm OK, if >=3 mm then corrective action is required.

IR imaging may be used to detect the temperature of the water, for example, determining if the water is chilled sufficiently at the skin prior to treatment initiation. IR imaging may be used to detect if the temperature of the patient's skin is sufficiently cooled prior to the start of therapy. For example, perhaps the skin contacting the membrane could be <15 degrees C. prior to the start of therapy to diminish the chance of burns. This may protect tissue beneath the skin (if skin is 15 degrees C., then the adjoining tissue is also cooled to certain degree).

In another variation, a grid is drawn on the patient's skin as reference, and camera visualization with motorized motion is used to automatically place the treatment module. Image detector may also be used to evaluate the integrity of the membrane that couples to the patient's body.

When air pockets are detected between the membrane of the treatment module and the patient's skin, a bubble removing device can be used to remove the air pocks, or the bubbles forming the air pockets. In one variation the bubble removing device comprises an elongated flexible member 1079, as shown in FIG. 162. In one example, the bubble removing device is made of a plastic tube having a lumen extending from a distal end to a proximal end. In one variation, the two ends of the elongated body are open. In another variation, the two ends of the elongated member are sealed.

One example of an air pocket removal step comprises: beginning at the apex (i.e., the narrow end) of the applicator of the treatment module, place the elongated bubble removal device transversely between the skin and the membrane, and, using one hand on each side of the treatment module, slowly draw the bubble removal device caudally to sweep the bubbles and air pockets out of the field. The effectiveness of this procedure can be verified by observing the process on the remote treatment module camera monitor, which display images captured by the image detector positioned in the treatment module.

### XI. Water Conditioner

A water conditioner may be provided to supply cooled and degassed water to the treatment applicator. A coupling fluid, such as water, is circulated from a chamber in treatment applicator to the water conditioner, where the returned water is degassed and cooled. In one variation, the water is cooled to approximately ten degree Celsius is circulated by the water conditioner.

In one variation, the water conditioner comprises an enclosure holding the following components: (a) Water conditioning components that chill and degas the water which is used as a coupling means for the transmission of acoustic energy from the treatment applicator to the patient. This may include a number of flow pumps, a high vacuum pump, flow solenoids, a water reservoir, particulate filter, and tubing. (b) Air pressure and vacuum components that interface to the Mover A positioning components of the treatment module. These components include a vacuum pump, a pressure pump, pressure regulators, and vacuum and pressure solenoids and hose. (c) Electrical components, such as a local water conditioner controller electronic circuit board (for autonomous operation of the treatment module positional controls and water temperature control) and support for the Mover A motors inside the treatment applicator (stepper motor drive controller units, including the communication port, stepper motor drive wires and encoder cables).

In one configuration, the water conditioner is configured with its own 1500 W medical grade isolation transformer and AC power cord. AC power is used to develop the secondary +24Vdc. The +24Vdc may be used to drive everything except the water chiller units, which can be powered directly from the secondary windings of the isolation transformer.

An USB communication port may be included in the design of the water conditioner, so as to provide information, such as water temperature and treatment module positional state, to the computing unit in the generator. Additional RS-422 ports may also be provided, for example, one for each motor controller unit.

The water conditioner may be configured to control and modify the pressure of coupling fluid circulated to the applicator. By adjusting the pressure, the system can control the level of inflation (and therefore the depth of the coupling fluid from the face of the treatment array to the membrane) of the applicator membrane, which contacts the patient's body. In one example, pressure sensors are positioned in the water conditioner to minor the fluid pressure. For examples, pressure sensors may be placed in the fluid reservoir, fluid line, and/or pressure pump of the water conditioner. In another variation, a pressure sensor is positioned in the applicator, and electrically connected to the water conditioner to provide the water conditioner sub-system with inflation/pressure information.

The water conditioner may also be configured to control the air pressure and suction going to the base mover of the treatment module. Air flow and/or air pressure can be measure with sensors placed within the water conditioner to ensure that (a) sufficient suction is provided to immobilize the ball joint of base mover when demanded; (b) sufficient suction is provided to secure the base treatment module in-place on the patient platform when required; and (c) sufficient air pressure is provided to float the treatment module on the patient platform when the operator desires to move and reposition the treatment module. For example, pressure sensors may be provided in the airflow channels in the water conditioner to monitor positive air flow to the treatment module and negative suction applied to the treatment module.

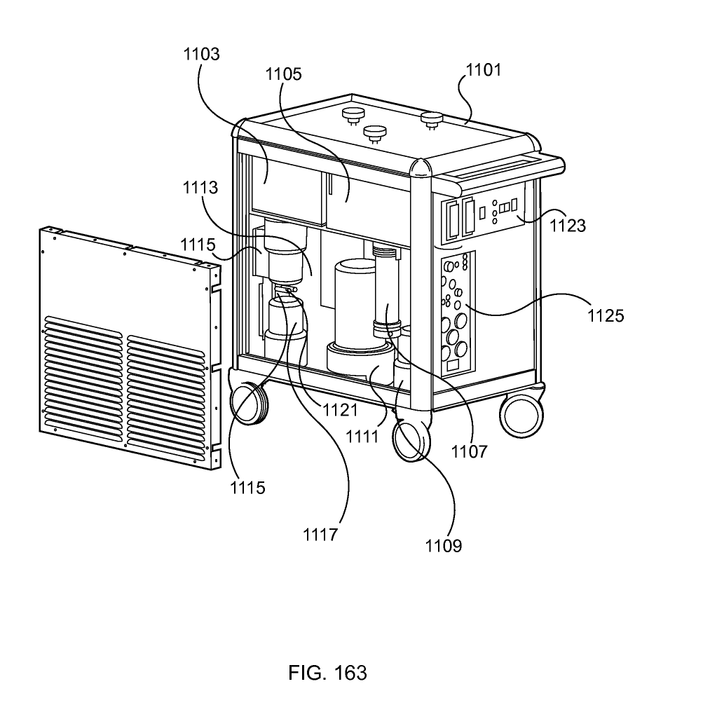

Referring to FIG. 163, an example of a water conditioner sub-system 1101 is shown. In this example the treatment module sub-system comprises: a dry box assembly 1103 which house components for supplying positive airflow and negative suction to and from the base movers of the treatment module; a wet box assembly 1105 which houses components for controlling coupling fluid flow to and from the applicator of the treatment module; membrane contactors 1107 for degassing liquids; vacuum and pressure chamber 1109; water reservoir 1111; water chiller 1113; electromagnetic interference filter 1115; power entry enclosure 1117; voltage selection switch 1119; fuse holder panel mount 1121. The back panel of the water conditioner includes an upper control panel 1123 with various control nobs and switches, and a lower interface panel 1125 for input-output electrical and plumbing connectors.

### XII. Patient Platform

In one variation, the patient platform is designed to mount on top of a standard catheterization table, and it is used to facilitate patient treatments in the supine position. It includes an aperture through which the treatment module can be positioned and couple to the patient's posterior flank. The patient platform comprises a light-weight carbon fiber material. In one example, the patient platform is configured to support subjects weighing up to 180 kilograms. The patient platform may also include a remote targeting monitor, a remote treatment module camera monitor, four illuminators, a glide plate, two bridges, a set of pads and two arm rests.

In one variation, the patient platform has a height of 10 inches or less, such that when the patient lies on the patient platform he is elevated 10 inches or less off the catheterization table. In another variation, the patient platform elevates the patient off the catheterization table between approximately 8 to approximately 10 inches. In yet another variation, the patient platform elevates the patient off the cauterization table between 8 to 12 inches.

In one variation, the patient platform weighs 75 pounds or less with the treatment module on the patient platform. In one variation, the patient platform weighs 80 pounds or less with the treatment module. In another variation, the patient platform weighs 85 pounds or less with the treatment module. In yet another variation, the patient platform is configured to weigh between 45 and 75 pounds. In yet another variation, the patient platform is configured to weigh between 35 and 65 pounds. In yet another variation, the patient platform is configured to weigh less than 65 pounds.

In some variations, the patient platform extends the entire length of catheterization table. In some variations, the patient platform is configured to be accessible to the operator from both sides of the catheterization table, which may allow bilateral operation. The patient platform may be produced from a radiolucent material, and not cause excessive imaging artifacts. For certain procedures, such as one requiring catheterization, a radiolucent patient platform may be desirable. In one example, the patient platform is configured such that it allows the operator to visualize a vascular anatomy with a fluoroscope. In another example, the patient platform is configured such that it allows the operator to visualize the renal artery anatomy with fluoroscope. In some variations, the patient platform is configured with radiolucent materials such that targeting catheter positioned in a vessel can be visualized through fluoroscopy.

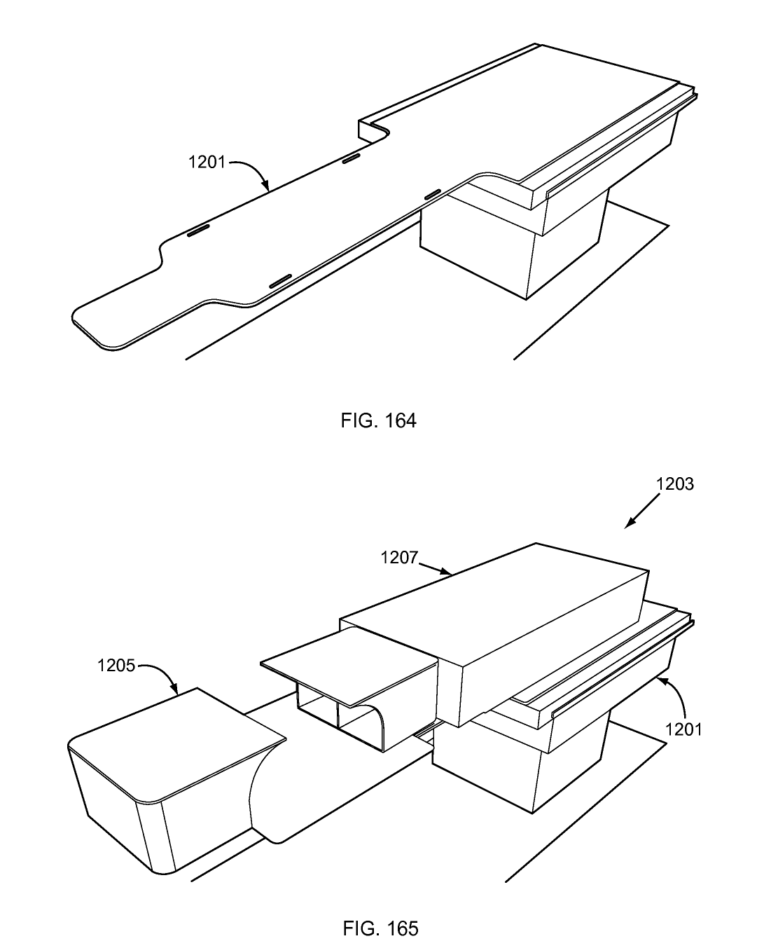

FIG. 164 depicts a standard catheterization table 1201 that is typical for a catheterization lab in a hospital. The catheterization table 1201 is shown with its padding and bedding removed. Referring to FIG. 165, a patient platform 1203 is shown positioned on the catheterization table 1201. In one variation, the patient platform comprises a main unit 1205 for supporting the torso of the patient, and an extension 1207 for supporting the legs.

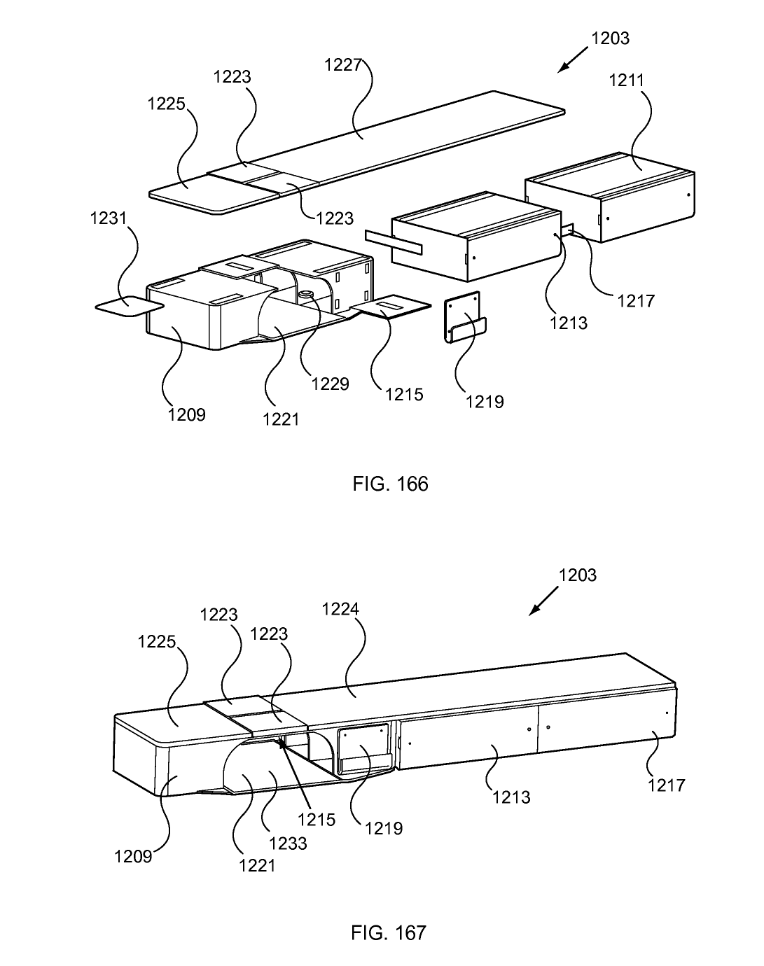

FIG. 166 is an exploded view illustrating the various components that comprises one variation of a patient platform 1203. In this variation, the patient platform includes: a torso support module 1209, two extender modules 1211, 1213, two bridges 1215, two extender module connectors 1217, two cable guide 1219, a glide plate 1221, two flank cushions 1223, a head cushion 1225, a body cushion 1227, four illuminators 1229 (such as touch lamps or lights), a headboard 1231, two arm boards, and two arm supports.

Referring to FIG. 167, one example process for assembling a patient platform 1203 on an existing catheterization table is described below. This may be accomplished by removing the existing cushions and bedding from the catheterization table, and placing the main unit (i.e., the torso support module) of the patient platform on the catheterization table surface, such that it is centered laterally, and its cranial end is flush with the cranial end of the catheterization table. The two extensions (i.e., extender modules) of the patient platform are then centered laterally on the catheterization table, and firmly pressed against the caudal end of the patient platform. The main unit of the patient platform and its extensions are attached to the catheterization table using three supplied straps—one around each end of the main unit and one around the caudal end of the extension. FIG. 165 shows the main unit 1209 of the patient platform installed on the cranial end of the catheterization table, and the extension 1213 is abutted to the caudal end of the main unit 1209.

The glide plate is then installed on the base of the patient platform, in the treatment module aperture. The glide plate 1221 may be oriented so that its side rails face downward and straddle the lateral edges of the patient platform base. In one variation, four battery-powered illuminators are provided and can be installed by pressing them on the velcro strips located on the ceiling of each of the four internal patient platform bays. The remote targeting monitor is then installed on one of the velcro strips located on the vertical surface that divides the two cranial-most bays, and the remote treatment module camera monitor installed in a similar fashion on the vertical surface that divides the two caudal-most bays. These monitors may be configured to face the subject's right side when treating the right renal artery, and the subject's left side when treating the left renal artery.

Finally, the left- and right-side patient platform bridges 1215 are installed so that they span the patient platform aperture 1233. The four patient platform pads 1223, 1225, 1227 are placed in their respective locations, atop the torso support module 1209, bridges 1215 and extender module 1213, 1217. Secure the patient arm rests under the patient platform pads in positions that are comfortable for the patient, and prevent the patient's arms from interfering with access to the treatment module.

### XIII. Treatment Module Positioning Template

A treatment module positioning template may be provided for marking the skin area on a patient's body, to identify a treatment window for facilitating placement of the treatment module. Proper placement of the treatment module ensures that the treatment transducer array housed within the applicator of the treatment module is positioned to provide treatment on the target tissue. The treatment window identifies where the treatment module can be positioned so as to avoid sonicating adjacent bony structures.

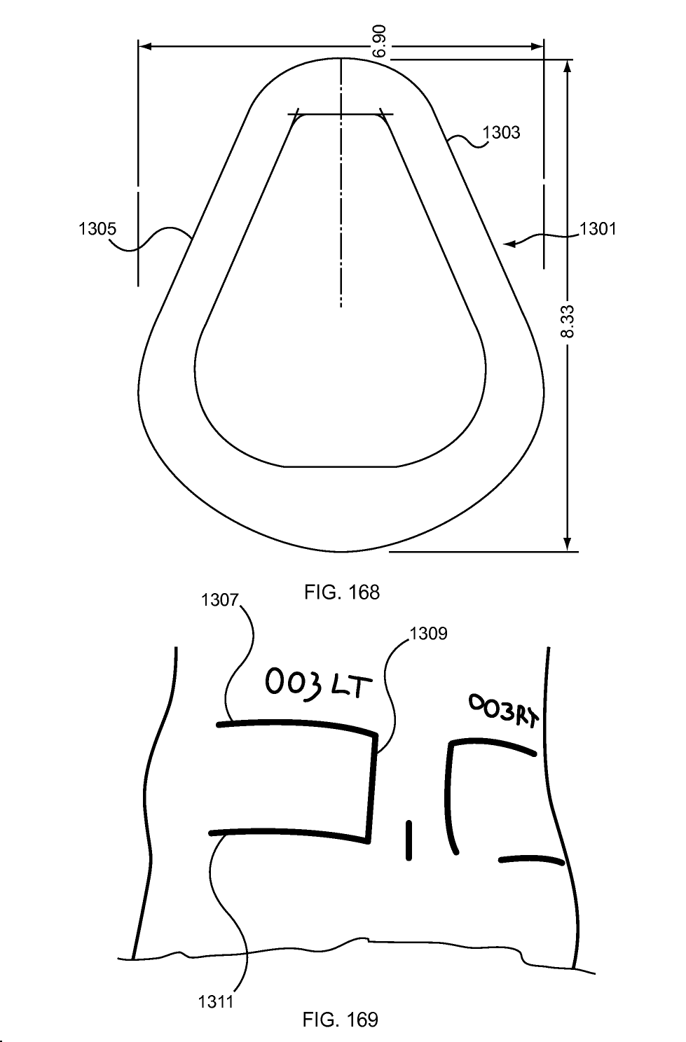

In one variation, the outer perimeter 1303 of a high-density polyethylene treatment module positioning template 1301 (as shown in FIG. 168) is physically identical in size and shape to the footprint of the treatment module nosecone. The inner perimeter 1305 of the treatment module positioning template indicates the outer limits of the focused therapeutic ultrasound, when delivering the focused ultrasound field into the patient's body.

In one example, the inner line or boundary of the treatment template is defined as the intersection of the transducer beam with the surface of the membrane, when the transducer is moved to the extremes at +/−10 degrees, about the x and y axis (referring to FIG. 148). The outer line or boundary of the treatment template is defined as the line formed when the plane parallel to the membrane intersects with the widest portion of the patient contact surface.

One example of a process to determine the treatment window for delivery focused ultrasound into the torso of a patient is described below. In preparation for administering focus ultrasound with the therapeutic system, right- and left-posterior flanks are to be depilated, as needed, and any residue cleaned away. While in the prone or sitting position, manual palpation is used to bilaterally identify the bony-structure boundaries (i.e., inferior edge of the 12^{th} rib 1307, lateral edge of the vertebral column transverse processes 1309, and superior edge of the iliac crest 1311) that bound the subject's posterior flank treatment windows (see FIG. 169). If the patient has already been treated on one side, only the contralateral posterior flank needs to be prepared.

In the event that the bony-structure boundaries cannot be adequately located using manual palpation, ultrasound imaging system may be used to locate the bone structures. For example, a commercial, duplex-ultrasound imaging system and probe, indicated for abdominal vascular imaging, and capable of operating in the 1.0-6.0 MHz frequency range may be employed. With the patient in the prone position, the imaging probe can be held vertically and then maneuvered around the entire perimeter of each acoustic window, so as to elucidate the edges of the bony-structure boundaries. Using a black or blue indelible-ink skin marker, the locations of these boundaries are drawn on the patient's skin.

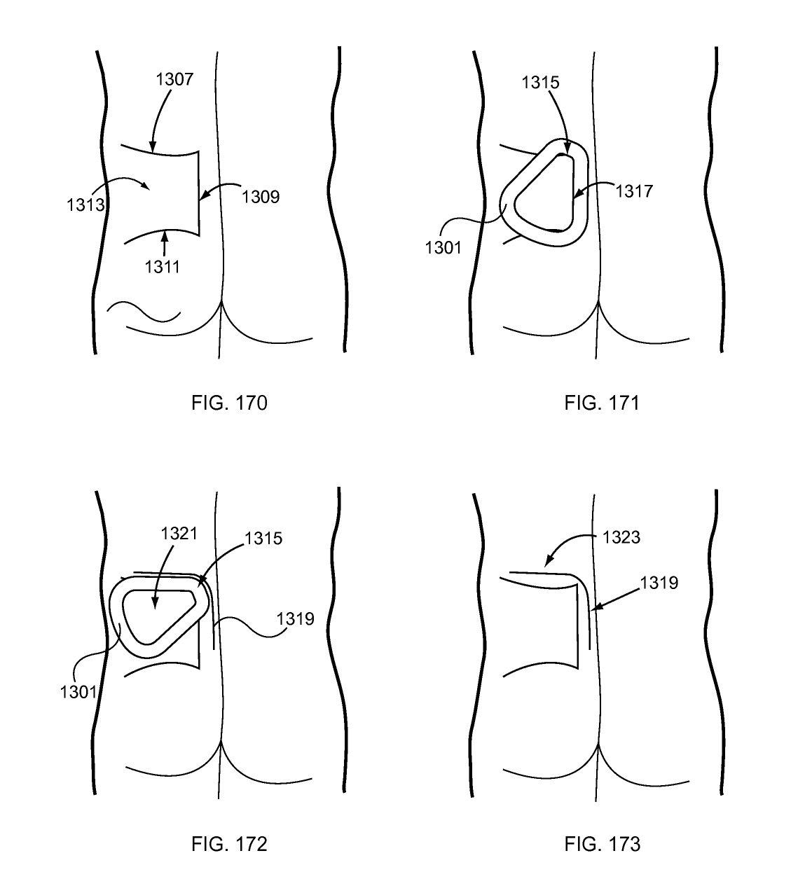

The template 1301 is to be placed on the patient's skin in the treatment window 1313 (FIG. 170) so that its apex 1315 points toward the costovertebral junction. The template 1301 is then maneuvered so that the inside straight edge 1317 nearest the spine is aligned with the marking that identifies the ipsilateral boundary of the spine 1309. While maintaining the spinal alignment, the template is to be translated parallel to the spine, until the inside perimeter at the apex 1315 of the template just overlaps the inferior edge 1307 of the rib (FIG. 171). While in this position, trace with red indelible ink 1319 around the outer perimeter of the template 1301 from its apex, to its base on the spine side.

Next, rotate the template 1301 about its apex 1315 so that its contralateral inside straight edge 1321 parallels the marking that identifies the inferior edge 1307 of the rib proximal to the spine. While maintaining this costal alignment, translate the template until the inside perimeter at the apex 1315 of the template just overlaps the edge of the spine (FIG. 172). While in this position, trace with red indelible ink 1323 around the outer perimeter of the template 1301 from its apex to its base on the rib side. The red tracing (FIG. 173), the outer boundary, is to be used during treatment module placement, as the boundary beyond which the treatment module nosecone (nosecone edge) shall not extend.

Following preparation for treatment, the patient is to be placed onto the patient platform, which elevates the patient above the surface of the catheterization table. In one example, the patient is elevated approximately 25 cm above the catheterization table. The patient is placed in the supine position with his marked posterior flank (treatment window) centered in the treatment aperture of the patient platform. Customary procedures may then be followed to improve patient comfort, and arrange the sterile draping to facilitate subsequent operator access to the space below the patient platform aperture.

### XIV. Dosing

In some variations, the therapeutic system may include a treatment planning and control subsystem, which generates information and actions for the therapeutic system to deliver a predetermined ultrasound dose to a series of therapeutic focal spots (or targets) on a series of specific tissue volumes at the target area, such as an area surrounding a renal artery. This subsystem works in concert with a target tracking subsystem. The target tracking subsystem may be (a) an ATOF subsystem (which provides location information and target tracking, that maintains focal spot position in the presence of patient movements, which may include breathing, muscle twitches, or voluntary movements) when a beacon is used for tracking, or (b) an imaging based non-invasive tracking system, or (c) a combination thereof.

In one variation, the treatment planning and control subsystem comprises the following functions: lesion geometry generation—the process of generating the coordinates of a pattern of therapeutic lesions at locations referenced to the target position (e.g., beacon position); dosimetry—the process of specifying and controlling the in situ acoustic power, system timing, exposure time, and inter-lesion “off” times; user interface—operator input and monitored parameters that are to be displayed on the system UI.

The therapeutic system may support an approach of delivering to specific target locations in the vicinity of the target tissue a series of independent thermal lesions. Each lesion's dosimetry is independent from the others in the series. Inter-lesion OFF times may be designed into the sequence to assure this approach, as well as to allow the interpath to cool between applications of acoustic power.

The acoustic dose delivered may raise the temperature of the target tissue volume by 10 to 40 degrees C. Actual ultrasonic dose can be operator selectable, but may be guided by pre-clinical data.

Software executed in the computing unit computes the requisite commands, and timing that control the tracking, and treatment transducer array energy delivery. The computing unit may be configured with sufficient computation power and data storage memory to store the output of the treatment planning and control subsystem, for incident review and error analysis.

In one variation, the treatment planning and control coordinate system utilized the same coordinate system as the ATOF coordinate system 363 as shown in FIG. 43. The center positions of the ATOF transmitter, or the targeting catheter's beacon, are defined as the center position of the treatment. The origin of the ATOF coordinate system is at the mechanical pivot point of the X-axis and Y-axis. In this example, the shallowest target is set at 80 mm below skin-line. The deepest target is set at 160 mm below skin-line. In one configuration, the maximum velocity of a target relative to Mover B is 35 mm/s. In another configuration, the maximum velocity of a target relative to Mover B is 25 mm/s. In yet another configuration, the maximum velocity of a target relative to Mover B is 45 mm/s.

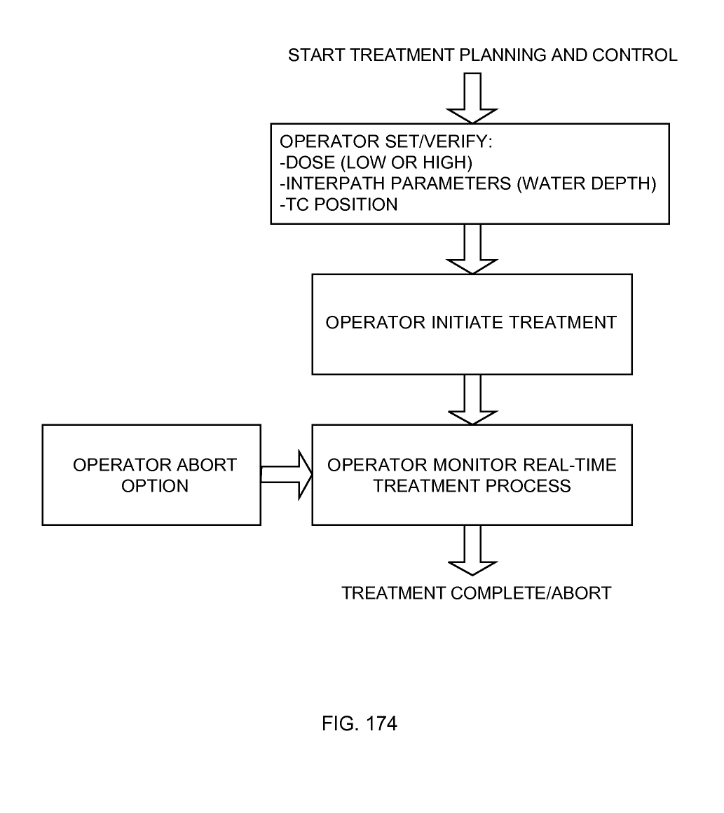

In one variation, the operator controls over the treatment planning and control subsystem is structured to implement the steps 1401 depicted in FIG. 174. In one variation, the Operator Abort is identical to Emergency Power Off on the UI.

The system may be configured such that the therapeutic focal spot is aligned in position with the computed ATOF position (for example within +/−1.0 mm over the full treatment range), referring to ATOF Volume 367, FIG. 43).

The system may also monitor DC power (Voltage and/or Current) consumed during therapy “on” times. In one variation, the system is set to notify the user if the electrical current consumed during the therapy is 10% beyond the expected current consumption define in the treatment plan.

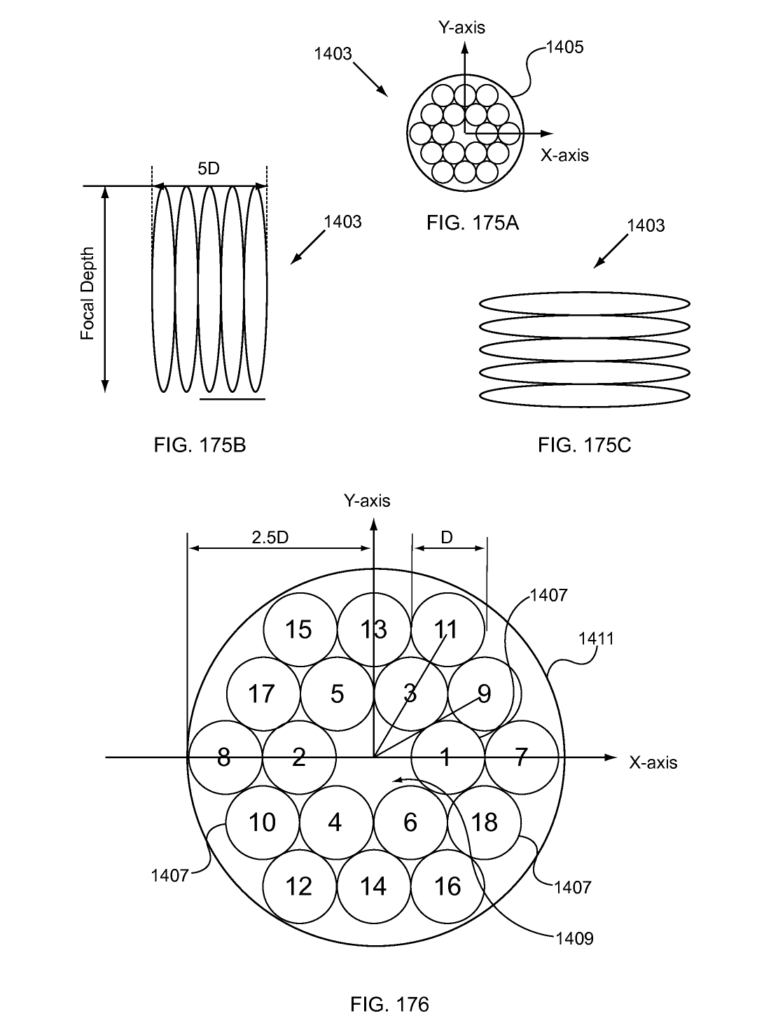

A single lesion pattern or a plurality of patterns may be used during a treatment session. In some variations, a single lesion pattern is applied to all the treatment locations during a treatment cycle for a patient. For example, lesion geometry 1403 can be generated such that N lesion centers are uniformly positioned over the cross section of a treatment cylinder 1405, as shown in FIGS. 175A-175C. This view is from the perspective of the virtual “acoustic” center of the therapeutic array (origin of axis Z′) and may be approximated by the position of the mechanical pivot point of Mover B (the origin of the ATOF reference axis).

The pattern of focal positions is constructed as in FIG. 176. In this example, to have flexible control of focal area and focal positions, eighteen positions 1407 are selected to produce a pattern or mesh of acoustic power distribution. The circles with a diameter of D are packed in hexagon shape. The center 1409 of the pattern is empty. All the focal positions are inside a 5D diameter circle 1411. The depth of the focal position in Z-axis may be determined from the ATOF position (also referencing FIG. 43).

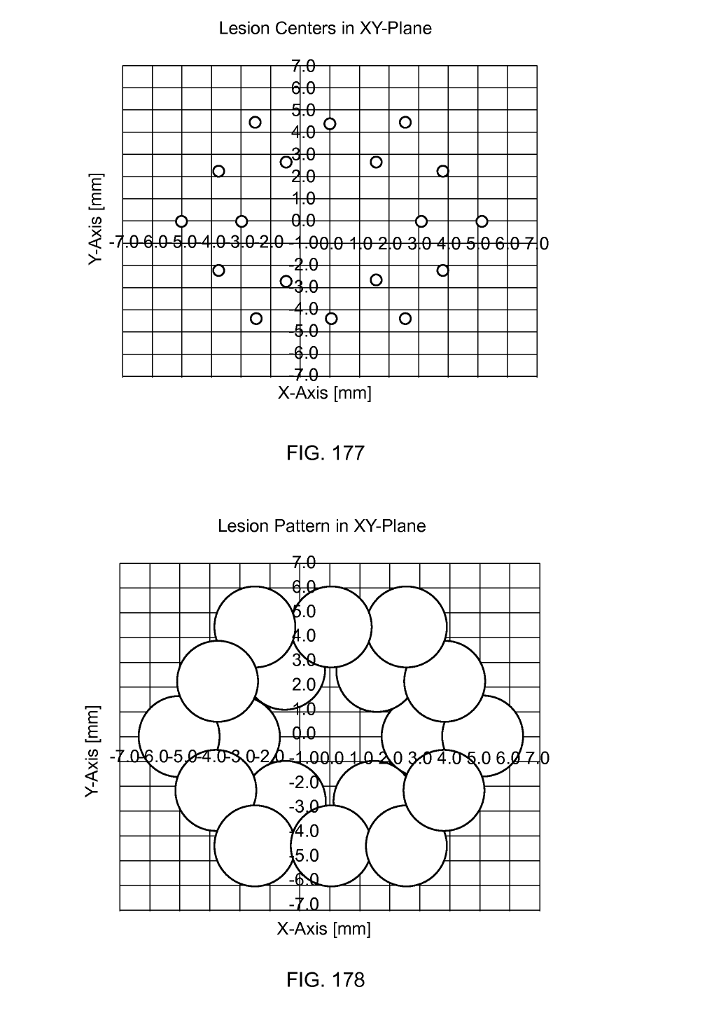

In another variation, a pattern of overlapping lesions is implemented. The focal position 1413 for each of the lesions is shown in FIG. 177. The expected patterns 1415 of the final lesions and their sizes are shown in FIG. 178.

One of ordinary skill in the art having the benefit of this disclosure would appreciate that additional lesion patterns can be generated by varying the number of the circles, the number of focal positions or overlapping of the circle. The data structure of the pattern and the focal positions can then be calculated for implementation by the system. The geometric coordinates of the lesions may be generated taking into account therapy focus and ATOF position alignment correction, and beacon (or targeting catheter) position information.

In one variation, the system calculates the acoustic output power level to be delivered from the therapeutic array for each lesion in the pattern based on the thermal dose input of the operator. Power calculation may be based on parameters from three sources: (i) operator input, (b) system derived data, and (c) treatment module specific information or parameters. In some variations, the power level is programed to be the same for all lesions in the pattern. In some variations, the power level is programed to vary depending on the location of the lesion within the pattern.

The output acoustic power calculation may be derived from the three input parameters: (a) thermal dose, (b) water depth, and (c) target depth. The system can be configured to allow the operator to verify the final output acoustic power and lesion positions before the treatment. The system may be calibrated in average power, within the focal spot in water in Watts. The calibration may be carried out when the therapy beam focus at the middle position of the full z range (for example, 80 to 160 mm from transducer surface). In some variations, the system is configured to be able to generate a maximum power of 320 W. In some variations, the acoustic power can be generated between 0.5 MHz to 1.5 MHz.

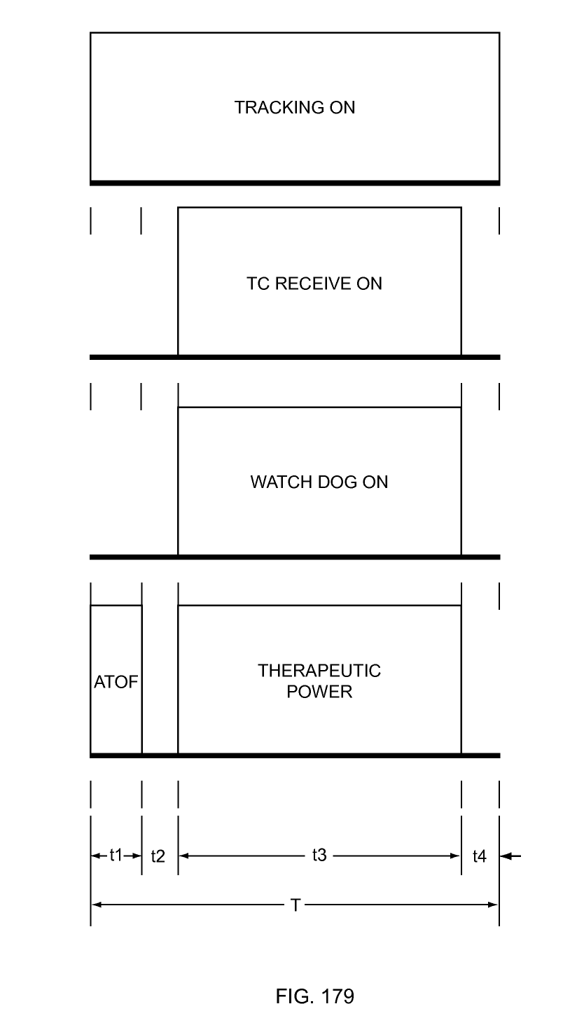

In one variation, for each lesion in the treatment plan, a sequence of consecutive intra-lesion cycles is executed. FIG. 179 depicts an example of an intra-lesion cycle 1417. In this example, tracking is running continuously during the entire treatment. During power on times, the targeting catheter (TC) can be set in a receive mode, monitoring an occurrence of the therapeutic beam impinging upon the targeting catheter. The use of the beacon as an ultrasound detector allows the system to verify that therapeutic ultrasound is indeed being delivered to the treatment area. If the system fails to detect ultrasound during the therapeutic power-on period, the system may alert the operator that the patient is not receiving the intended therapeutic energy at the treatment site.

During time t1, ATOF pulses are transmitted from the beacon. For example, four ATOF pulses may be transmitted during this time to provide target locating information. During time t2, the system receives the ATOF pulses and processes the pulses to determine position information. During time t3, therapy is being delivered to a specific target site to create a specific lesion. Time t4 is a time to allow the acoustic environment to quiet after therapy power is stopped. Between each inter-lesion cycle, the treatment plan commands the tracking subsystem to move to the coordinated of the next lesion in the plan. In one variation, the system is programed, such that lesions in the treatment plan are executed in an order that treats each sequential lesion at a location that is as far from that of the previous lesion. In another variation, the system is programed to execute the planed treatment regions according to the numerical order as listed in FIG. 176.

In one variation, data and items to be displayed to the operator during use of the treatment planning and control subsystem includes: lesion number, beacon position, pause/resume/cancel therapy, sensor temperature, loop rate, voltage, current, beacon delay and detected beacon signal amplitude.

Operator inputs may be checked for valid ranges. If an operator input is out of range, an input error condition can be indicated, and the value is not accepted. Graphical display of the treatment plan (lesion geometry) may be color coded. For example, White=no treatment yet; Light Red=in progress; Red=completed; and Black=aborted. A targeting and tracking quality table may be displayed on the user screen. For example the table may display for each lesion the mean deviation from intended position (vector magnitude) and the standard deviation of the tracking during exposure. The system may also display any interlocks, such as Watch Dog function (system operation status which verifies system is operating property or it is experiencing unexpected events), conditions status, and handling messages.

In some variations, the systems are configured to prevent administration of a dose higher than expected in the treatment plan. In one variation, the sequence of operations for setting and delivering a specific dose comprises: (A) The user selects a dose from a drop-down UI control. (B) The user may select an estimated water depth by entering a value. For example, between 10 and 50 in a text control on the UI. (C) The system determines an average Z depth (i.e., the Z direction depth from the treatment array to the target tissue) by averaging at of readings. For example, averaging reading over a 15 seconds period. (D) The power in watts is then calculated based on: (a) the selected dose; (b) the estimated water depth; (c) the average Z depth; (e) the lesion on time, which is set via a configuration file. (E) If the calculated watts is greater than the limit, the operator is shown a dialog box and given the choice of continuing at the limited watts or not. The system may be pre-programed with the watt limit: (a) the watt limit is set via a configuration file; (b) the actual watts calculated is displayed to the user in the dialog box when they are asked if they wish to proceed at the limit; (c) the watts to be used, limited or not, are then displayed to the user in the UI. For example, the watt limit may be set at 320 watts. (F) The electrical current expected for the watts calculated, is calculated from parameters specific to the transducer in use. Over-current limit may then be defined as follows: (a) the current expected is multiplied by the over-current percentage which is set via a configuration file; (b) the over-current limit is sent to the current monitoring processor on the electronic interface board. (G) The treatment plan (may also be referred to as the lesion plan) is created using the watts calculated or limited in step (E) above. (H) At the beginning of therapy the transceiver module is sent the watts calculated or limited in step (E). (I) The transceiver module uses parameters specific to the transducer being used to convert the watts into the therapy voltage. This voltage is used by the transmit circuitry during the power on time to deliver the expected dose. (J) During therapy, the current monitoring processor monitors the current being delivered to the array. If the current exceeds the limit set in step (F)(b) above, the current monitoring processor shall disable the power supply output. An over-current condition will occur, and the user is notified via a dialog box: (a) before the dialog is displayed the transceiver may be told to stop delivering power, even though the current monitoring processor has already disabled the power supply output, preventing power delivery; (b) the operator is requested to acknowledge the dialog. The application then shuts down on its own.

Additional safeguards or monitoring may be implemented to further prevent occurrence of out of bound parameter in the treatment process. For example, under step (C) above, the 15 second averaging can reduce the effect of small, quick, movements. The Z depths used in the calculation are shown in the treatment plan computed by the system, and should be close to that seen during positioning. The system may be configured to compare the calculated average Z depth to the to the Z depth used during positioning. If the difference is larger than a pre-determined number, the system will notify that operator of a potential error, and request the operator to verify the calculated Z-depth before proceeding further.

In another example, under step (E) above, the limit set via the configuration file is displayed in the dialog box and shown to the user. The actual watts to be used are shown to the user in the UI. The UI may also display the limit set in the configuration file, such that when the actual watts to be used is outside the limit set in the configuration file, the displayed limit may be shown in a different color, in a flashing mode, or other visual enhancers, to remind the operator that power being used is outside of the limit.

In another example, under step (F) above, the transducer specific data files available for use are limited to the one transducer associated with the system being used. The system may be configured to makes a run-time check against hardcoded transducer parameter values and prevents usage and/or terminate the treatment process if the check fails. Therefore, if an incorrect transducer array is used under step (F), the system is able to check against the hardcoded transducer parameter and prevent the therapeutic process from proceeding, if it detects inconsistencies.

In addition, the default current limit can be set to a value, much smaller than any therapy value, which would be used for the indication that the system is configure for treating. The current monitoring processor would immediately disable the power supply at the start of therapy, if the current monitoring processor fails to receive the over-current limit determined under step (F) above.

In another example, the transceiver can be configured to retain previous power level. Under such configuration, if previous power is lower than current power, output voltage and current will be less than expected. If previous power is higher than current power, output voltage and current will be higher than expected, and the current limit shall be triggered. For example, the trigger may be set such that the current limit will trigger if ≧10% than previous current. In this configuration, if the transceiver was not sent the calculated power, or failed to receive the calculated power, the system is prevented from delivering an unexpected high power.

In another example, under step (I), the system may be configured to calculated voltage to be used to drive the treatment transducer, based on both the transducer configuration file and based on the hard coded transducer parameter values. If the voltage values determined through the different approaches are inconsistent with each other, the system will prompt the user to verify the treatment parameter.

In another example, the system is configured to prevent unexpected power being delivered to the treatment array, if incorrect voltage is sent to the power supply. The current limit can be set to trigger if the actual current is higher than the expected current by a predefined margin. For example the current limit may be set to trigger if the actual current is ≧10% than intended current. Therefore, if incorrect voltage is lower than intended voltage, output current will be less than expected. If incorrect voltage is higher than intended voltage, output current will be higher than expected, and current limit will trigger if ≧10% than intended current.

In yet another example, the system may be configured to detect failure of the electronic interface board, and its current monitoring processor, in limiting the current driving the treatment transducer (e.g., the current limit is not used by the current monitoring processor). In one variation, the actual current being delivered during therapy is displayed to the operator in the UI. The operator can pause therapy, and thereby stop power delivery on a suspected over current. In another variation, boundary parameter is set for monitoring the actual current. If the actual current exceeds the boundary parameter, visual and or audio indicators may be provided to alert the user to check the current level.

### XV. Phase Aberration Correction

As discussed earlier in this disclosure, Phase Aberration Correction (PAC) may be applied to further refine the phase control of each ultrasound transceiver system channel output for the focal position at the target. This may be done, for example, before the dosage planning and system phase control parameter setting, as shown in FIG. 26. Phase aberration correction can provide adjustments in the generation of the ultrasound wave, in the phase and/or time domain, to account for the non-homogeneity of the materials in the pathway from the transducer elements in the treatment transducer array to the target tissue. This adjustment may be based on modeling of the physiology data, input of pre-treatment clinical analysis (such as CAT scan or other imaging data), and/or known calibration/excitation pulse sent and detected prior to the initiation of therapeutic ultrasound, for determination of tissue characteristics in the pathway. In another variation, the adjustment of the phase data is modified as a result of time-of-flight calculations done by targeting pulses generated by the beacon and received by the individual transducers on the treatment array. In this variation, during the treatment process, the treatment array is put into a listening mode, and the individual transducer elements in the treatment array detect the signal generated by the beacon. As will be discussed in more detail below, the beacon may generate PAC bursts, which may be different from the ATOF bursts for phase correction calculation. For example, the PAC bursts may have a frequency and/or amplitude that closely model the frequency and/or amplitude of the treatment ultrasound field. Based on PAC bursts' travel time from the beacon to each of the transducer elements on the treatment array, and the known position of the beacon, the system then makes phase adjustment in generating the treatment ultrasound from the treatment array.

A control button may be implemented in the system software user interface to start the phase aberration correction procedures, and input the control parameters for the PAC.

In one example, the control parameters include the following: (A) ATOF Control Parameters: (a) transmitter operation frequency and amplitude; (b) receiver delay and gain; (c) time delay between two adjacent pulses; and (d) number of cycles for each ATOF pulse. (B) Phase Aberration Correction Control Parameters: (a) transmitter operation frequency and amplitude; (b) receiver delay and gain; (c) time delay between two ODD and EVEN pulses; and (d) number of cycles for each PAC pulse.

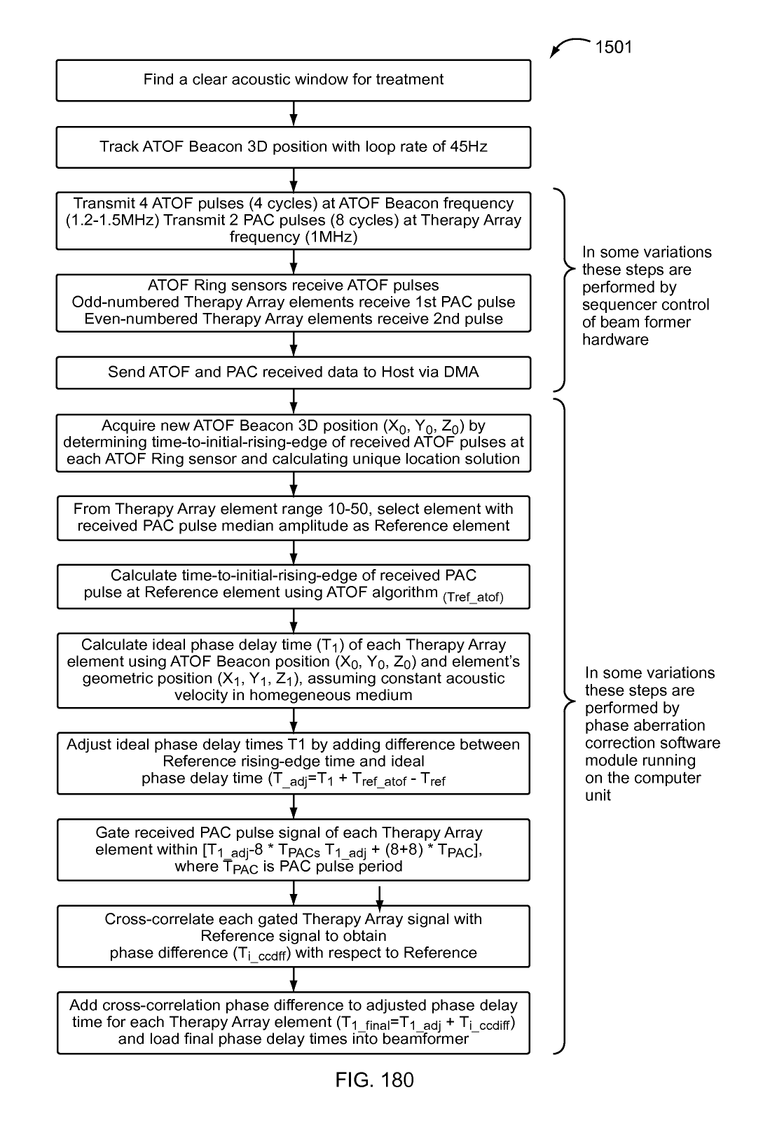

One example of the functional steps 1501 for phase aberration correction is illustrated in FIG. 180. These major steps can be divided into two main categories: the phase aberration correction RF signal acquisition, and phase aberration correction signal processing.

In one variation, the phase aberration correction process follows the steps set forth below. The treatment module (TM) is slid underneath a water tank of a Test Apparatus (TA) and makes an acoustic interface through its membrane on the bottom of the tank. Inside the tank is a fixture holding an ultrasound beacon, which the ATOF ring inside the TM locates and tracks to. The test apparatus provides a simulated environment for testing the acoustic transmission of the treatment module.

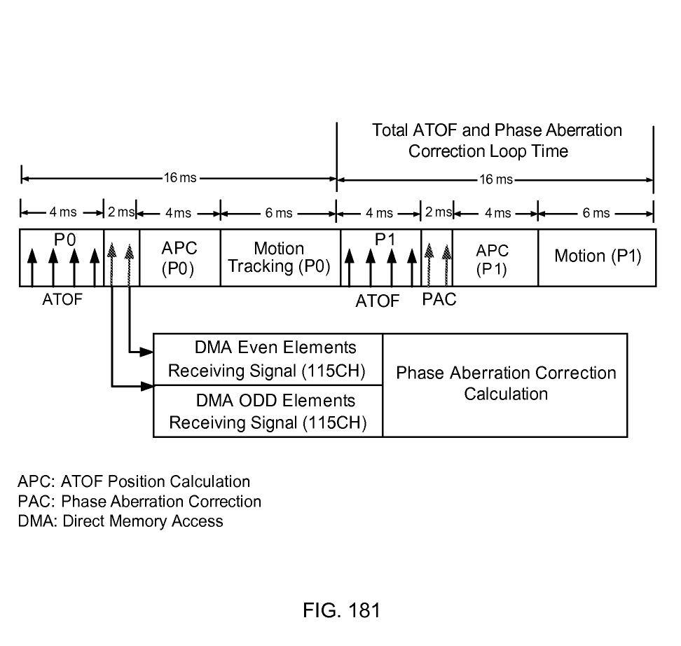

The time diagram 1503 for phase aberration correction RF signal acquisition is shown in FIG. 181. The system transmits four ATOF bursts 1505 followed immediately thereafter by two longer PAC bursts (>8 cycles) 1507. The 4 ATOF bursts (each burst for example, may have four cycles) operate at the ATOF operation frequency, which can be, for example, in the range of 1.3 MHz to 1.7 MHz. The transmitted ATOF bursts will be detected by the eight ATOF receivers located on the ATOF ring. The eight ATOF received RF signals are transferred from the ultrasound transceiver to CPU (i.e., computing unit) for beacon position calculation.

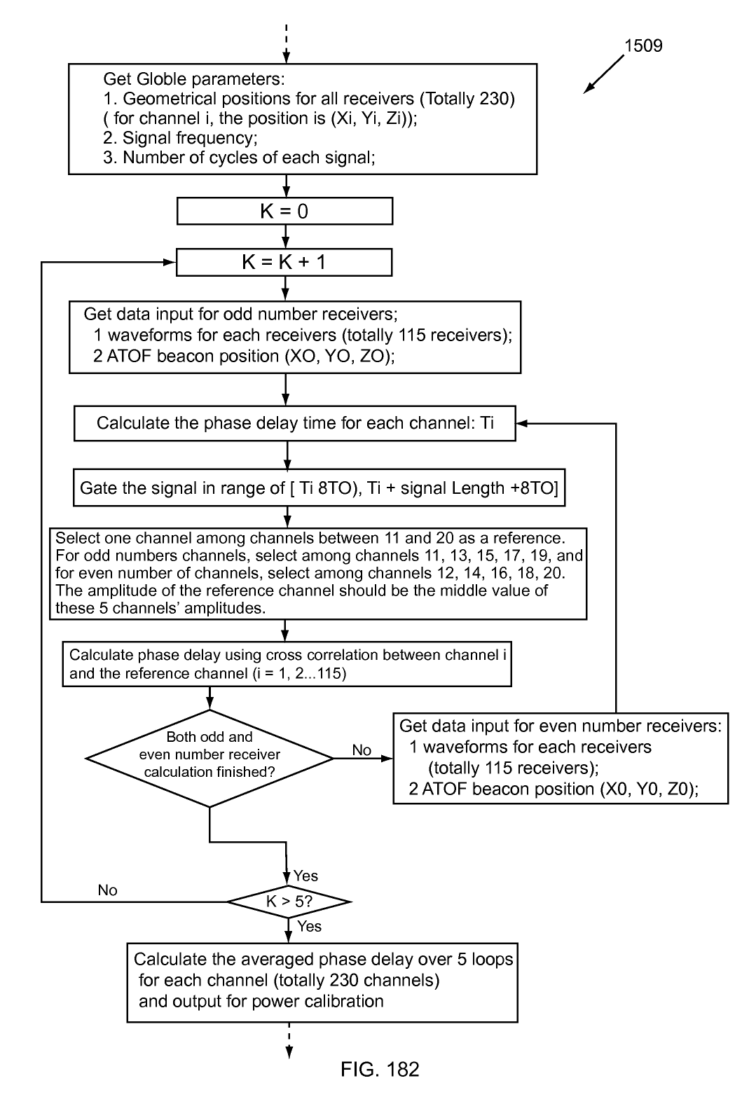

The two phase aberration correction burst may have at least eight cycles for each burst. The PAC bursts operate at the operation frequency of the therapy array, which can be, for example, in the range of 0.8 MHz to 1.2 MHz. The PAC bursts will be received by the array elements. The first PAC burst will be received by the elements with ODD numbers, and the second burst will be received by the elements with EVEN numbers. All received RF signals are transferred from the ultrasound transceiver to CPU for phase aberration correction processing. A software block diagram of an example of a computation process 1509 for phase aberration correction is shown in FIG. 182.

For one configuration, in the water tank, the following performance improvements may be observed: The signal amplitude measured from the ultrasound beacon at any focal position along the Z-axis of the treatment array, within the range from 80 mm to 160 mm, can increase at least 10% after the phase aberration correction. The signal amplitude measured from the ultrasound beacon at any focal position along the Z-axis of the treatment array, within the range from 80 mm to 160 mm, can reach the same level (within 5%) after the phase aberration correction for any initial phase settings with different sound velocities. Variations in sound velocity can be induced in a variety of ways, including covering all or part of the array with a thin layer of rubber (<5 mm). The focal spot size measured by a 20 mm by 20 mm scan around the focal region from the ultrasound beacon at any focal position along the Z-axis of the treatment array, within the range from 80 mm to 160 mm, can be smaller than the focal size before the phase aberration correction for any initial phase settings with different sound velocities.

It should be noted that one or more of the functions described herein may be performed by a processor. As used in this specification, the term “processor” may refer to one or more processing units, wherein a processing unit may be a hardware processor (e.g., an ASIC processor, a FPGA processor, a general purpose processor, a microprocessor, a signal processor, etc., or any of other types of processor) or any integrated circuit, may be a software module, or may be a combination of both hardware and software. In some embodiments, the processor may be coupled to one or more components (e.g., transducer, detector, etc.) of the therapeutic system.

Several examples of the embodiments have been described and portrayed. While the embodiments have been described in terms of particular variations and illustrative figures, those of ordinary skill in the art will recognize that the claimed invention is not limited to the variations or figures described. In addition, where methods and steps described above indicate certain events occurring in certain order, those of ordinary skill in the art will recognize that the ordering of certain steps may be modified and that such modifications are in accordance with the variations of covered within the scope of the claimed invention. Additionally, certain of the steps may be performed concurrently in a parallel process when possible, as well as performed sequentially as described above. Therefore, to the extent there are variations of one or more of the embodiments, which are within the spirit of the disclosure or equivalent to the claimed invention found in the claims, it is the intent that this patent and the claimed invention will cover those variations as well. Finally, all publications and patent applications cited in this specification are herein incorporated by reference in their entirety as if each individual publication or patent application were specifically and individually put forth herein.

What is claimed is:

1. A system to apply ultrasound energy to a region surrounding blood flow in a blood vessel from a position outside a patient comprising:
a therapeutic ultrasound transducer comprising a plurality of transducer elements; and
a processor configured to control the plurality of transducer elements;
wherein the processor is further configured to receive a first input regarding a first position of the blood vessel;
wherein the processor is configured to change phase inputs to the transducer elements to move a focus of the transducer at least 1 cm in a first plane which is substantially along a plane of the transducer elements of the therapeutic ultrasound transducer and at least 1 cm in a second plane orthogonal to the first plane; and
wherein the processor is further configured to position the focus of the transducer in sequential positions offset from the blood flow in the blood vessel according to a pattern pre-determined by an operator of the system.

2. The system of claim 1, wherein the plurality of transducer elements are arranged in a substantially random or non-uniform pattern.

3. The system of claim 1, wherein the therapeutic ultrasound transducer comprises a substrate with cutouts respectively for the transducer elements; and
wherein the transducer elements can be placed into the respective cutouts of the substrate and be coupled to the therapeutic ultrasound transducer via snap or press fit.

4. The system of claim 1, wherein the transducer elements of the therapeutic ultrasound transducer are arranged on a 3D substrate pointing to a defined point, line, area, or 3D region, in front of the therapeutic ultrasound transducer.

5. The system of claim 1, wherein at least one of the transducer elements of the therapeutic ultrasound transducer has a circle, square, hexagon, or rectangular shape.

6. The system of claim 1, wherein the transducer elements of the therapeutic ultrasound transducer are packaged with one size, two sizes, or multiple sizes of single element transducers.

7. The system of claim 1, wherein the transducer elements of the therapeutic ultrasound transducer are packaged with multiple sizes of single element transducers with multiple operational frequencies.

8. The system of claim 3, wherein the substrate of the transducer is formed from three-dimensional printing process with linear, planar, ellipsoid, spherical or other 3D geometry.

9. The system of claim 8, wherein the three dimensional printing process comprises one of: selective laser melting, direct metal laser sintering, selective laser sintering, fused deposition modeling, a polymer curing type process, and a stereolithography process.

10. The system of claim 1, wherein the processor is configured to receive a second input regarding a second position of the blood vessel within about 1-200 ms of the first position; and
wherein the processor is configured to adjust the focus of the therapeutic ultrasound transducer according to the second position.

11. The system of claim 1, further comprising an imaging probe to image a region including the blood vessel.

12. The system of claim 11, wherein the imaging probe comprises attached fiducials or 3D position sensors.

13. The system of claim 12, wherein the processor is configured to receive input associated with reflections or position signals from the fiducials or the position sensors, interpret the input, and produce an output for indicating imaging probe position and imaging probe orientation in a three-dimensional coordinate space.

14. The system of claim 13, wherein the processor is configured to determine a three dimensional position of the blood vessel based on the imaging probe position, the imaging probe orientation, and a position of the blood vessel.

15. The system of claim 12, wherein the processor is configured to receive input associated with reflections or position signals from the fiducials or the position sensors, and produce an output for positioning of the therapeutic ultrasound transducer.

16. The system of claim 1, wherein the processor is configured to associate a three dimensional position of a target on an image from an imaging probe with a three dimensional position of the therapeutic ultrasound transducer.

17. The system of claim 1, further comprising a graphical user interface coupled to the processor, wherein the graphical user interface is configured to display a target, and wherein the target as displayed is moveable by an operator of the system.

18. The system of claim 3, wherein the therapeutic ultrasound transducer comprises a series of ridges in which the transducer elements are press or snap fit with tolerance greater than 100 microns.

19. The system of claim 3, wherein the therapeutic ultrasound transducer comprises a series of ridges in which the transducer elements are press fit or snap fit with a tolerance greater than 50 microns.

20. The system of claim 1, wherein the system is configured to be calibrated automatically with a receiver placed at a distance from the therapeutic ultrasound transducer via a calibration procedure that determines an efficiency of the transducer elements.

21. The system of claim 1, wherein the system is configured to be calibrated automatically with a transmitter placed at a distance from the therapeutic ultrasound transducer via a calibration procedure that determines an efficiency of the transducer elements.

22. The system of claim 1, wherein the processor is also configured to track a region of interest in an ultrasound image.

23. The system of claim 22, wherein the region of interest is linked to a user defined target on the ultrasound image.

24. The system of claim 22, wherein the processor comprises an algorithm to compare a region of interest in an ultrasound frame to a region of interest in a previous ultrasound frame, and update a position of a target with respect to the therapeutic ultrasound transducer.

25. The system of claim 24, wherein the algorithm utilizes digitization of speckles for the comparison.

26. The system of claim 24, wherein the algorithm utilizes digitization of anatomic structures for the comparison.

27. The system of claim 24, wherein the algorithm utilizes both digitization of speckles and digitization of anatomic structures for the comparison.

28. The system of claim 1, wherein the processor is configured to utilize modulation of an output pulse to the therapeutic ultrasound transducer to account for a regional difference in power density along the transducer elements of the therapeutic ultrasound transducer.

29. The system of claim 1, wherein the processor is configured to utilize phase modulation of an output pulse to the with the therapeutic ultrasound transducer to account for phase aberration from inhomogeneous tissue structure.

30. The system of claim 1, wherein the processor is configured to utilize pulse width modulation of an output pulse to the therapeutic ultrasound transducer to account for performance due to transducer element size or a variation from manufacture processing of the transducer elements of the transducer.

31. The system of claim 1, further comprising an electromechanical mover coupled to the processor, wherein the electromechanical mover is configured to mechanically position the therapeutic ultrasound transducer to move the focus in response to control signals from the processor.

32. The system of claim 1, wherein the therapeutic ultrasound transducer is configured to provide high-intensity energy, moderate-intensity energy, low-intensity energy, or a combination thereof.

33. The system of claim 1, wherein the processor is configured to track a position of a specific target region or regions during a delivery of the ultrasound energy by the therapeutic ultrasound transducer.

34. The system of claim 1, wherein the processor is further configured to use ultrasound imaging and/or ultrasound signal beacon to track a location of a target region and to maintain the focus of the transducer at the target region during a treatment cycle.

35. The system of claim 1, wherein the ultrasound transducer is within an applicator and is moveable independent of an orientation of the applicator; and
wherein the system further comprises an actuator for controlling a movement of the ultrasound transducer, and a detector coupled to either the applicator or the ultrasound transducer, wherein the detector is configured to detect a treatment region in the patient and track a position of the treatment region while the treatment region moves within the patient.

36. The system of claim 35, wherein the detector comprises an ultrasound imaging transducer array.

37. The system of claim 35, wherein the detector comprises three or more ultrasound receivers for detecting a beacon positioned in proximity of the treatment region.

38. The system of claim 35, further comprising an ultrasound transceiver module connected to the transducer;
wherein the detector is connected to the ultrasound transceiver module and the actuator; and
wherein the processor is configured to determine the position of the treatment region relative to the transducer.

39. The system of claim 1, wherein at least one of the transducer elements has a semi-annular shape.

40. The system of claim 1, further comprising a first mover to which the therapeutic ultrasound transducer is connected, the first mover being configured to provide at least three degrees of freedom of movements for the therapeutic ultrasound transducer.

41. The system of claim 40, wherein the therapeutic ultrasound transducer is located within a housing of an applicator, and is coupled to a second mover, the ultrasound transducer array being at least partially immersed in a liquid contained in the housing of the applicator, and wherein the second mover is configured to provide at least two degrees of freedom in movements for the ultrasound transducer array.

42. The system of claim 1, wherein the therapeutic ultrasound transducer is a part of an applicator, and the applicator comprises a membrane for coupling the applicator to a patient's body.

43. The system of claim 1, further comprising a first orientation sensor coupled to the therapeutic ultrasound transducer.

44. The system of claim 43, wherein the therapeutic ultrasound transducer is a part of an applicator, and wherein the system further comprises a second orientation sensor coupled to a housing of the applicator.

45. The system of claim 44, further comprising a water conditioner to circulate a cooled fluid through a chamber in the housing of the applicator.

46. The system of claim 1, further comprising a detector with a plurality of ultrasound receivers configured for detecting a position of a beacon based on acoustic time of flight calculation, the detector being coupled to the processor.

47. The system of claim 1, further comprising a generator and an ultrasound transceiver configured to energize at least some of transducer elements in the transducer with varying phases of energy to focus the energy on a predetermined location in the patient.

48. The system of claim 1, wherein the processor is configured to access a treatment plan that prescribes delivery of energy to a plurality of treatment regions within a patient's body in accordance with the pattern.

49. The system of claim 1, wherein the processor is also configured to calculate a required movement of the ultrasound transducer for moving the focus of the ultrasound transducer from a first treatment region to a second treatment region.

50. The system of claim 1, wherein the processor is further configured to determine a required angular rotation of the ultrasound transducer to move the focus from a first treatment region to a second treatment region.

51. The system of claim 1, wherein the processor is configured to generate a phase table for at least some of the transducer elements, the phase table having values for operating the ultrasound transducer so that energy provided by the ultrasound transducer focuses at a target position in a patient's body.

52. The system of claim 1, wherein the processor is further configured to determine a power table for various power requirements for at least some of the transducer elements, the power table having values for calculating electrical energy to drive the at least some of the transducer elements so that a desired dosing at a target position can be achieved.

53. The system of claim 1, further comprising circuitry for pulse-width modulating electrical energy to drive at least some of the transducer elements in the transducer, to achieve a consistent power intensity across the transducer.

54. The system of claim 1 wherein at least two of the transducer elements have different respective surface areas.

55. The system of claim 1, wherein the transducer elements are arranged in a random configuration atop a substrate, the substrate being manufactured using a three dimensional printing process.

56. The system of claim 1, wherein the transducer elements are arranged in a concentric pattern.

57. The system of claim 1, wherein the processor is configured to use an algorithm to make adjustments to a pulse width modulation of electrical energy such that two or more of the transducer elements deliver same power density relative to a focal point of the transducer with the pulse width modulation, wherein the algorithm takes into account a distance from two or more of the transducer elements to the focal point of the transducer.

58. The system of claim 1, wherein the processor is configured to use an algorithm to make adjustments using pulse width modulation of electrical energy such that two or more of the transducer elements achieve a same power density relative to a focal point of the transducer, wherein the algorithm takes into account absorption and interference within a transmission path from each of the two or more transducers to the focal point of the transducer.

59. The system of claim 1 wherein at least two of the transducer elements have different respective sizes, the at least two of the transducer elements having a larger transducer element and a smaller transducer element, and wherein the processor is configured to use pulse width modulation to drive more electrical power to the larger transducer element, and less electrical power to the smaller transducer element.

60. The system of claim 1, wherein the processor is configured to track a position of a beacon in a coordinate system based on signals emitted by the beacon and received by ultrasound receivers coupled to the transducer, and based on an acoustic time of flight calculation.

61. The system of claim 1, wherein the transducer comprises five or more ultrasound receivers, and a sub-set of the five or more ultrasound receivers are activated for tracking a beacon.

62. The system of claim 61, wherein the processor is configured to use an algorithm to detect whether one or more activated ultrasound receivers have failed, and activate additional ultrasound receiver(s) if failure of the one or more activated receivers is detected.

63. The system of claim 1, wherein the transducer comprises lobes with the transducer elements being arranged in a diced configuration, the diced configuration forming a pie shape, wherein one of the transducer elements closer to a narrow segment of the pie shape has a larger surface area, while another one of the transducers further away from the narrow segment of the pie shape has a smaller surface area.

64. The system of claim 1, wherein the processor is further configured for monitoring an actual electrical current being consumed by the transducer, and determining whether the actual electrical current being consumed is higher than an expected electrical current consumption.

65. The system of claim 1, wherein the processor is also configured for locating a treatment region within the patient, tracking a position of the treatment region as the treatment region moves in the patient, calculating a distance between at least one of transducer elements and the target region, and generating a phase aberration correction factor based at least on the distance between the at least one of transducer elements and the target region.

66. The system of claim 1, wherein the transducer further comprising a housing which contains a channel in a wall of the housing to direct coupling fluid to flow over a surface of the transducer.

67. The system of claim 66, further comprising an image detector attached to the housing and positioned to capture images of a membrane through the coupling fluid.

68. The system of claim 67, wherein the image detector is configured to detect a spectrum of light that comprises infrared light.

69. The system of claim 67, wherein the image detector is configured to detect reflections from an interface between the membrane and a skin of the patient to determine a distance to the skin from the therapeutic ultrasound transducer.

70. The system of claim 1, further comprising an image detector for detecting bubbles located between an acoustic coupling interface of the transducer and a body of the patient.

71. The system of claim 1, further comprising a radiolucent frame having a torso segment for supporting a torso of the patient, and an extension segment for supporting legs of the patient.

72. The system of claim 24, wherein the algorithm utilizes digitalization of a flow parameter in the ultrasound image for the comparison.

73. The system of claim 24, wherein the algorithm uses a combination of digitalization of speckles, a flow parameter, and anatomic structural information for the comparison.

74. The system of claim 22, wherein the region of interest is a user-defined region of interest.

75. The system of claim 74, wherein the processor is configured to determine whether the region of interest is adequate for use in tracking based on an indicator signal.

76. The system of claim 1, wherein the sequential positions offset from the blood flow are within 5 mm of one another.

77. The system of claim 1, wherein the sequential positions offset from the blood flow are within 1 mm of one another.

78. The system of claim 1, wherein the sequential positions offset from the blood flow is substantially a same position.

79. The system of claim 1, further comprising a table for the patient, wherein the table comprises an opening which can be modified in size by an operator.

80. A system to apply ultrasound energy to a nerve region surrounding blood flow in a blood vessel from a position outside a patient comprising:
a therapeutic ultrasound transducer comprising a plurality of transducer elements;
an ultrasound imaging transducer with attached fiducials configured to indicate an orientation of the imaging transducer; and
a processor configured to control the plurality of transducer elements;
wherein the processor is further configured to receive data at a first time point from the fiducials to determine a three-dimensional coordinate of a target in an ultrasound image from the ultrasound imaging transducer.

81. The system of claim 80, wherein the processor is further configured to position a focus of the transducer elements at sequential positions offset from the blood flow in the blood vessel according to a pattern pre-determined by an operator of the system.

82. The system of claim 80, wherein the processor is configured to receive additional data from the fiducials at successive time points.

83. The system of claim 82, wherein the processor is configured to utilize the additional data to determine an updated coordinate of the target.

84. The system of claim 82, wherein the target comprises a user-defined region of interest.

85. The system of claim 84, wherein the target comprises speckles, anatomic features, or flow signals.
